SEQUENCE LISTING (158 sequences)

SEQ ID NO: 1 - protein, 10 aa, synthetic construct
  source 1..10: note = CXP1
  SITE 6: note = iodophenylalanine
  SITE 10: note = 4-aminopiperidine-4-carboxylic acid
KQKTSXGRGX

SEQ ID NO: 2 - protein, 10 aa, synthetic construct
  source 1..10: note = CXP2
  SITE 5: note = 2-amino-3-(naphthalen-2-yl)propanoic acid
  SITE 9: note = 2,4-diaminobutyric acid
NFKQXHAGXR

SEQ ID NO: 3 - protein, 10 aa, synthetic construct
  source 1..10: note = CXP3
  SITE 1: note = 2,4-diaminobutyric acid
  SITE 7: note = 2,4-diaminobutyric acid
XVARNNXTKG

SEQ ID NO: 4 - protein, 10 aa, synthetic construct
  source 1..10: note = CXP4
  SITE 1: note = 2-amino-5-ureidopentanoic acid
  SITE 5: note = 4-aminopiperidine-4-carboxylic acid
  SITE 6: note = 2,4-diaminobutyric acid
  SITE 7: note = 2,4-diaminobutyric acid
XMFGXXXKAP

SEQ ID NO: 5 - protein, 10 aa, synthetic construct
  source 1..10: note = CXP5
  SITE 1: note = 2,4-diaminobutyric acid
  SITE 4: note = 2,4-diaminobutyric acid
  SITE 8: note = 2,4-diaminobutyric acid
XPRXLHSXAT

SEQ ID NO: 6 - protein, 10 aa, synthetic construct
  source 1..10: note = CXP6
  SITE 2: note = 4-aminopiperidine-4-carboxylic acid
  SITE 4: note = 2,4-diaminobutyric acid
  SITE 6: note = 2,4-diaminobutyric acid
  SITE 9: note = iodophenylalanine
  SITE 10: note = 2-amino-3-(naphthalen-2-yl)propanoic acid
PXGXKXSVXX

SEQ ID NO: 7 - protein, 10 aa, synthetic construct
  source 1..10: note = CXP7
  SITE 4: note = iodophenylalanine
  SITE 6: note = 2,4-diaminobutyric acid
VQHXRXKEKN

SEQ ID NO: 8 - protein, 10 aa, synthetic construct
  source 1..10: note = CXP8
  SITE 7: note = 2,4-diaminobutyric acid
NFNNRSXGHK

SEQ ID NO: 9 - protein, 10 aa, synthetic construct
  source 1..10: note = CXP9
  SITE 8: note = 2-amino-3-(naphthalen-2-yl)propanoic acid
  SITE 10: note = 2,4-diaminobutyric acid
ARSEQHHXSX

SEQ ID NO: 10 - skipped

SEQ ID NO: 11 - protein, 6 aa, synthetic construct
  source 1..6: note = SB
  SITE 4: note = iodophenylalanine
KTSXGR

SEQ ID NO: 12 - protein, 5 aa, synthetic construct
  source 1..5: note = SC
KQKTS

SEQ ID NO: 13-14 - skipped (2 entries)

SEQ ID NO: 15 - protein, 8 aa, synthetic construct
  source 1..8: note = SF
  SITE 6: note = iodophenylalanine
KQKTSXGR

SEQ ID NO: 16-17 - skipped (2 entries)

SEQ ID NO: 18 - protein, 7 aa, synthetic construct
  source 1..7: note = SI
  SITE 5: note = iodophenylalanine
QKTSXGR

SEQ ID NO: 19 - protein, 8 aa, synthetic construct
  source 1..8: note = SJ
  SITE 6: note = iodophenylalanine
KQKTSXGR

SEQ ID NO: 20 - protein, 10 aa, synthetic construct
  source 1..10: note = peptide sequence
  SITE 6: note = iodophenylalanine
  SITE 10: note = 4-aminopiperidine-4-carboxylic acid
KQKTSXGRGX

SEQ ID NO: 21 - protein, 10 aa, synthetic construct
  source 1..10: note = peptide sequence
  SITE 2: note = 4-aminopiperidine-4-carboxylic acid
  SITE 4: note = 2,4-diaminobutyric acid
  SITE 6: note = 2,4-diaminobutyric acid
  SITE 9: note = iodophenylalanine
  SITE 10: note = 2-amino-3-(naphthalen-2-yl)propanoic acid
PXGXKXSVXX

SEQ ID NO: 22 - protein, 10 aa, synthetic construct
  source 1..10: note = peptide sequence
  SITE 4: note = iodophenylalanine
  SITE 6: note = 2,4-diaminobutyric acid
VQHXRXKEKN

SEQ ID NO: 23 - skipped

SEQ ID NO: 24 - protein, 6 aa, synthetic construct
  source 1..6: note = peptide sequence
  SITE 4: note = iodophenylalanine
KTSXGR

SEQ ID NO: 25 - skipped

SEQ ID NO: 26 - protein, 8 aa, synthetic construct
  source 1..8: note = peptide sequence
  SITE 6: note = iodophenylalanine
KQKTSXGR

SEQ ID NO: 27 - skipped

SEQ ID NO: 28 - protein, 7 aa, synthetic construct
  source 1..7: note = peptide sequence
  SITE 5: note = iodophenylalanine
QKTSXGR

SEQ ID NO: 29 - protein, 8 aa, synthetic construct
  source 1..8: note = peptide sequence
  SITE 6: note = iodophenylalanine
KQKTSXGR

SEQ ID NO: 30 - protein, 25 aa, synthetic construct
  source 1..25: note = peptide sequence
  SITE 6: note = halophenylalanine
  SITE 10: note = 4-aminopiperidine-4-carboxylic acid
  SITE 21: note = halophenylalanine
  SITE 25: note = 4-aminopiperidine-4-carboxylic acid
KQKTSXGRGXKWKKGKQKTSXGRGX

SEQ ID NO: 31 - protein, 25 aa, synthetic construct
  source 1..25: note = peptide sequence
  SITE 1: note = 2,4-diaminobutyric acid
  SITE 7: note = 2,4-diaminobutyric acid
  SITE 21: note = halophenylalanine
  SITE 25: note = 4-aminopiperidine-4-carboxylic acid
XVARNNXTKGKWKKGKQKTSXGRGX

SEQ ID NO: 32 - protein, 25 aa, synthetic construct
  source 1..25: note = peptide sequence
  SITE 1: note = 2-amino-5-ureidopentanoic acid
  SITE 5: note = 4-aminopiperidine-4-carboxylic acid
  SITE 25: note = 4-aminopiperidine-4-carboxylic acid
  SITE 6: note = 2,4-diaminobutyric acid
  SITE 7: note = 2,4-diaminobutyric acid
  SITE 21: note = halophenylalanine
XMFGXXXKAPKWKKGKQKTSXGRGX

SEQ ID NO: 33 - protein, 15 aa, synthetic construct
  source 1..15: note = peptide sequence
  SITE 1: note = halophenylalanine
  SITE 5: note = 4-aminopiperidine-4-carboxylic acid
  SITE 11: note = halophenylalanine
  SITE 15: note = 4-aminopiperidine-4-carboxylic acid
XGRGXKQKTSXGRGX

SEQ ID NO: 34 - protein, 16 aa, synthetic construct
  source 1..16: note = peptide sequence
  SITE 16: note = 4-aminopiperidine-4-carboxylic acid
  SITE 4: note = halophenylalanine
  SITE 12: note = halophenylalanine
KTSXGRKQKTSXGRGX

SEQ ID NO: 35 - protein, 15 aa, synthetic construct
  source 1..15: note = peptide sequence
  SITE 11: note = halophenylalanine
  SITE 15: note = 4-aminopiperidine-4-carboxylic acid
KQKTSKQKTSXGRGX

SEQ ID NO: 36 - protein, 13 aa, synthetic construct
  source 1..13: note = peptide sequence
  SITE 13: note = 4-aminopiperidine-4-carboxylic acid
  SITE 9: note = halophenylalanine
KWKKQKTSXGRGX

SEQ ID NO: 37 - protein, 14 aa, synthetic construct
  source 1..14: note = peptide sequence
  SITE 4: note = halophenylalanine
  SITE 12: note = halophenylalanine
KTSXGRKQKTSXGR

SEQ ID NO: 38 - protein, 18 aa, synthetic construct
  source 1..18: note = peptide sequence
  SITE 6: note = halophenylalanine
  SITE 16: note = halophenylalanine
KQKTSXGRKWKQKTSXGR

SEQ ID NO: 39 - protein, 25 aa, synthetic construct
  source 1..25: note = CXP1-CXP1
  SITE 6: note = iodophenylalanine
  SITE 21: note = iodophenylalanine
  SITE 25: note = 4-aminopiperidine-4-carboxylic acid
  SITE 10: note = 4-aminopiperidine-4-carboxylic acid
KQKTSXGRGXKWKKGKQKTSXGRGX

SEQ ID NO: 40 - protein, 25 aa, synthetic construct
  source 1..25: note = CXP3-CXP1
  SITE 1: note = 2,4-diaminobutyric acid
  SITE 7: note = 2,4-diaminobutyric acid
  SITE 21: note = iodophenylalanine
  SITE 25: note = 4-aminopiperidine-4-carboxylic acid
XVARNNXTKGKWKKGKQKTSXGRGX

SEQ ID NO: 41 - protein, 25 aa, synthetic construct
  source 1..25: note = CXP4-CXP1
  SITE 1: note = 2-amino-5-ureidopentanoic acid
  SITE 5: note = 4-aminopiperidine-4-carboxylic acid
  SITE 25: note = 4-aminopiperidine-4-carboxylic acid
  SITE 6: note = 2,4-diaminobutyric acid
  SITE 7: note = 2,4-diaminobutyric acid
  SITE 21: note = iodophenylalanine
XMFGXXXKAPKWKKGKQKTSXGRGX

SEQ ID NO: 42 - protein, 15 aa, synthetic construct
  source 1..15: note = SA-CXP1
  SITE 1: note = iodophenylalanine
  SITE 11: note = iodophenylalanine
  SITE 5: note = 4-aminopiperidine-4-carboxylic acid
  SITE 15: note = 4-aminopiperidine-4-carboxylic acid
XGRGXKQKTSXGRGX

SEQ ID NO: 43 - protein, 16 aa, synthetic construct
  source 1..16: note = SB-CXP1
  SITE 4: note = iodophenylalanine
  SITE 12: note = iodophenylalanine
  SITE 16: note = 4-aminopiperidine-4-carboxylic acid
KTSXGRKQKTSXGRGX

SEQ ID NO: 44 - protein, 15 aa, synthetic construct
  source 1..15: note = SC-CXP1
  SITE 11: note = iodophenylalanine
  SITE 15: note = 4-aminopiperidine-4-carboxylic acid
KQKTSKQKTSXGRGX

SEQ ID NO: 45 - protein, 13 aa, synthetic construct
  source 1..13: note = SD-CPX1
  SITE 13: note = 4-aminopiperidine-4-carboxylic acid
  SITE 9: note = iodophenylalanine
KWKKQKTSXGRGX

SEQ ID NO: 46 - protein, 14 aa, synthetic construct
  source 1..14: note = CXD1A
  SITE 4: note = iodophenylalanine
  SITE 12: note = iodophenylalanine
KTSXGRKQKTSXGR

SEQ ID NO: 47 - protein, 18 aa, synthetic construct
  source 1..18: note = CXD1B
  SITE 6: note = iodophenylalanine
  SITE 16: note = iodophenylalanine
KQKTSXGRKWKQKTSXGR

SEQ ID NO: 48 - protein, 14 aa, synthetic construct
  source 1..14: note = CXP1
  SITE 6: note = iodophenylalanine
  SITE 10: note = 4-aminopiperidine-4-carboxylic acid
KQKTSXGRGXKWKK

SEQ ID NO: 49 - protein, 14 aa, synthetic construct
  source 1..14: note = CXP2
  SITE 5: note = 2-amino-3-(naphthalen-2-yl)propanoic acid
  SITE 9: note = 2,4-diaminobutyric acid
NFKQXHAGXRKWKK

SEQ ID NO: 50 - protein, 14 aa, synthetic construct
  source 1..14: note = CXP3
  SITE 1: note = 2,4-diaminobutyric acid
  SITE 7: note = 2,4-diaminobutyric acid
XVARNNXTKGKWKK

SEQ ID NO: 51 - protein, 14 aa, synthetic construct
  source 1..14: note = CXP4
  SITE 1: note = 2-amino-5-ureidopentanoic acid
  SITE 5: note = 4-aminopiperidine-4-carboxylic acid
  SITE 6: note = 2,4-diaminobutyric acid
  SITE 7: note = 2,4-diaminobutyric acid
XMFGXXXKAPKWKK

SEQ ID NO: 52 - protein, 14 aa, synthetic construct
  source 1..14: note = CXP5
  SITE 1: note = 2,4-diaminobutyric acid
  SITE 4: note = 2,4-diaminobutyric acid
  SITE 8: note = 2,4-diaminobutyric acid
XPRXLHSXATKWKK

SEQ ID NO: 53 - protein, 14 aa, synthetic construct
  source 1..14: note = CXP6
  SITE 2: note = 4-aminopiperidine-4-carboxylic acid
  SITE 4: note = 2,4-diaminobutyric acid
  SITE 6: note = 2,4-diaminobutyric acid
  SITE 9: note = iodophenylalanine
  SITE 10: note = 2-amino-3-(naphthalen-2-yl)propanoic acid
PXGXKXSVXXKWKK

SEQ ID NO: 54 - protein, 14 aa, synthetic construct
  source 1..14: note = CXP7
  SITE 4: note = iodophenylalanine
  SITE 6: note = 2,4-diaminobutyric acid
VQHXRXKEKNKWKK

SEQ ID NO: 55 - protein, 14 aa, synthetic construct
  source 1..14: note = CXP8
  SITE 7: note = 2,4-diaminobutyric acid
NFNNRSXGHKKWKK

SEQ ID NO: 56 - protein, 14 aa, synthetic construct
  source 1..14: note = CXP9
  SITE 8: note = 2-amino-3-(naphthalen-2-yl)propanoic acid
  SITE 10: note = 2,4-diaminobutyric acid
ARSEQHHXSXKWKK

SEQ ID NO: 57 - protein, 9 aa, synthetic construct
  source 1..9: note = SA
  SITE 1: note = iodophenylalanine
  SITE 5: note = 4-aminopiperidine-4-carboxylic acid
XGRGXKWKK

SEQ ID NO: 58 - protein, 10 aa, synthetic construct
  source 1..10: note = SB
  SITE 4: note = iodophenylalanine
KTSXGRKWKK

SEQ ID NO: 59 - protein, 9 aa, synthetic construct
  source 1..9: note = SC
KQKTSKWKK

SEQ ID NO: 60 - protein, 7 aa, synthetic construct
  source 1..7: note = SD
KWKKWKK

SEQ ID NO: 61 - protein, 7 aa, synthetic construct
  source 1..7: note = SE
  SITE 1: note = iodophenylalanine
XGRKWKK

SEQ ID NO: 62 - protein, 12 aa, synthetic construct
  source 1..12: note = SF
  SITE 6: note = iodophenylalanine
KQKTSXGRKWKK

SEQ ID NO: 63 - protein, 8 aa, synthetic construct
  source 1..8: note = SG
  SITE 2: note = iodophenylalanine
SXGRKWKK

SEQ ID NO: 64 - protein, 7 aa, synthetic construct
  source 1..7: note = SH
KTSKWKK

SEQ ID NO: 65 - protein, 11 aa, synthetic construct
  source 1..11: note = SI
  SITE 5: note = iodophenylalanine
QKTSXGRKWKK

SEQ ID NO: 66 - protein, 12 aa, synthetic construct
  source 1..12: note = SJ
  SITE 6: note = iodophenylalanine
KQKTSXGRKWKK

SEQ ID NO: 67 - protein, 29 aa, synthetic construct
  source 1..29: note = CXP1-CXP1
  SITE 6: note = iodophenylalanine
  SITE 10: note = 4-aminopiperidine-4-carboxylic acid
  SITE 21: note = iodophenylalanine
  SITE 25: note = 4-aminopiperidine-4-carboxylic acid
KQKTSXGRGXKWKKGKQKTSXGRGXKWKK

SEQ ID NO: 68 - protein, 29 aa, synthetic construct
  source 1..29: note = CXP3-CXP1
  SITE 1: note = 2,4-diaminobutyric acid
  SITE 7: note = 2,4-diaminobutyric acid
  SITE 21: note = iodophenylalanine
  SITE 25: note = 4-aminopiperidine-4-carboxylic acid
XVARNNXTKGKWKKGKQKTSXGRGXKWKK

SEQ ID NO: 69 - protein, 29 aa, synthetic construct
  source 1..29: note = CXP4-CXP1
  SITE 1: note = 2-amino-5-ureidopentanoic acid
  SITE 5: note = 4-aminopiperidine-4-carboxylic acid
  SITE 6: note = 2,4-diaminobutyric acid
  SITE 7: note = 2,4-diaminobutyric acid
  SITE 21: note = iodophenylalanine
  SITE 25: note = 4-aminopiperidine-4-carboxylic acid
XMFGXXXKAPKWKKGKQKTSXGRGXKWKK

SEQ ID NO: 70 - protein, 19 aa, synthetic construct
  source 1..19: note = SA-CXP1
  SITE 1: note = iodophenylalanine
  SITE 5: note = 4-aminopiperidine-4-carboxylic acid
  SITE 11: note = iodophenylalanine
  SITE 15: note = 4-aminopiperidine-4-carboxylic acid
XGRGXKQKTSXGRGXKWKK

SEQ ID NO: 71 - protein, 20 aa, synthetic construct
  source 1..20: note = SB-CXP1
  SITE 4: note = iodophenylalanine
  SITE 12: note = iodophenylalanine
  SITE 16: note = 4-aminopiperidine-4-carboxylic acid
KTSXGRKQKTSXGRGXKWKK

SEQ ID NO: 72 - protein, 19 aa, synthetic construct
  source 1..19: note = SC-CXP1
  SITE 11: note = iodophenylalanine
  SITE 15: note = 4-aminopiperidine-4-carboxylic acid
KQKTSKQKTSXGRGXKWKK

SEQ ID NO: 73 - protein, 17 aa, synthetic construct
  source 1..17: note = SD-CXP1
  SITE 9: note = iodophenylalanine
  SITE 13: note = 4-aminopiperidine-4-carboxylic acid
KWKKQKTSXGRGXKWKK

SEQ ID NO: 74 - protein, 18 aa, synthetic construct
  source 1..18: note = CXD1A
  SITE 4: note = iodophenylalanine
  SITE 12: note = iodophenylalanine
KTSXGRKQKTSXGRKWKK

SEQ ID NO: 75 - protein, 22 aa, synthetic construct
  source 1..22: note = CXD1B
  SITE 6: note = iodophenylalanine
  SITE 16: note = iodophenylalanine
KQKTSXGRKWKQKTSXGRKWKK

SEQ ID NO: 76 - protein, 16 aa, synthetic construct
  source 1..16: note = CPP penetratin
RQIKIWFQNRRMKWKK

SEQ ID NO: 77 - DNA, 18 bp, synthetic construct
  source 1..18: mol_type = other DNA; note = sequence of nucleobases
gctattaccttaacccag

SEQ ID NO: 78 - protein, 14 aa, synthetic construct
  source 1..14: note = CXP1X
  SITE 10: note = 4-aminopiperidine-4-carboxylic acid
KQKTSFGRGXKWKK

SEQ ID NO: 79 - protein, 14 aa, synthetic construct
  source 1..14: note = CXP1Y
  SITE 6: note = iodophenylalanine
KQKTSXGRGKKWKK

SEQ ID NO: 80 - protein, 14 aa, synthetic construct
  source 1..14: note = CXP1Z
KQKTSFGRGKKWKK

SEQ ID NO: 81 - protein, 14 aa, synthetic construct
  source 1..14: note = CXP1AA
  SITE 6: note = iodophenylalanine
  SITE 10: note = 4-aminopiperidine-4-carboxylic acid
KQKTSXGKGXKWKK

SEQ ID NO: 82 - protein, 14 aa, synthetic construct
  source 1..14: note = CXP1P
  SITE 6: note = iodophenylalanine
  SITE 10: note = 4-aminopiperidine-4-carboxylic acid
KQKTSXARAXKWKK

SEQ ID NO: 83 - protein, 14 aa, synthetic construct
  source 1..14: note = CXP1B
  SITE 10: note = 4-aminopiperidine-4-carboxylic acid
  SITE 6: note = iodophenylalanine
AQKTSXGRGXKWKK

SEQ ID NO: 84 - protein, 14 aa, synthetic construct
  source 1..14: note = CXP1C
  SITE 6: note = iodophenylalanine
  SITE 10: note = 4-aminopiperidine-4-carboxylic acid
KAKTSXGRGXKWKK

SEQ ID NO: 85 - protein, 14 aa, synthetic construct
  source 1..14: note = CXP1D
  SITE 10: note = 4-aminopiperidine-4-carboxylic acid
  SITE 6: note = iodophenylalanine
KQATSXGRGXKWKK

SEQ ID NO: 86 - protein, 14 aa, synthetic construct
  source 1..14: note = CXP1E
  SITE 6: note = iodophenylalanine
  SITE 10: note = 4-aminopiperidine-4-carboxylic acid
KQKASXGRGXKWKK

SEQ ID NO: 87 - protein, 14 aa, synthetic construct
  source 1..14: note = CXP1F
  SITE 10: note = 4-aminopiperidine-4-carboxylic acid
  SITE 6: note = iodophenylalanine
KQKTAXGRGXKWKK

SEQ ID NO: 88 - protein, 14 aa, synthetic construct
  source 1..14: note = CXP1G
  SITE 10: note = 4-aminopiperidine-4-carboxylic acid
KQKTSAGRGXKWKK

SEQ ID NO: 89 - protein, 14 aa, synthetic construct
  source 1..14: note = CXP1H
  SITE 6: note = iodophenylalanine
  SITE 10: note = 4-aminopiperidine-4-carboxylic acid
KQKTSXARGXKWKK

SEQ ID NO: 90 - protein, 14 aa, synthetic construct
  source 1..14: note = CXP1I
  SITE 10: note = 4-aminopiperidine-4-carboxylic acid
  SITE 6: note = iodophenylalanine
KQKTSXGAGXKWKK

SEQ ID NO: 91 - protein, 14 aa, synthetic construct
  source 1..14: note = CXP1J
  SITE 6: note = iodophenylalanine
  SITE 10: note = 4-aminopiperidine-4-carboxylic acid
KQKTSXGRAXKWKK

SEQ ID NO: 92 - protein, 14 aa, synthetic construct
  source 1..14: note = CXP1K
  SITE 6: note = iodophenylalanine
KQKTSXGRGAKWKK

SEQ ID NO: 93 - protein, 14 aa, synthetic construct
  source 1..14: note = CXP1L
  SITE 6: note = iodophenylalanine
  SITE 10: note = 4-aminopiperidine-4-carboxylic acid
KQKTSXGRGXAWKK

SEQ ID NO: 94 - protein, 14 aa, synthetic construct
  source 1..14: note = CXP1M
  SITE 10: note = 4-aminopiperidine-4-carboxylic acid
  SITE 6: note = iodophenylalanine
KQKTSXGRGXKAKK

SEQ ID NO: 95 - protein, 14 aa, synthetic construct
  source 1..14: note = CXP1N
  SITE 6: note = iodophenylalanine
  SITE 10: note = 4-aminopiperidine-4-carboxylic acid
KQKTSXGRGXKWAK

SEQ ID NO: 96 - protein, 18 aa, synthetic construct
  source 1..18: note = CXP1O
  SITE 10: note = 4-aminopiperidine-4-carboxylic acid
  SITE 6: note = iodophenylalanine
KQKTSXGRGXKWKAKWKK

SEQ ID NO: 97 - protein, 14 aa, synthetic construct
  source 1..14: note = CXP1Q
  SITE 1: note = iodophenylalanine
  SITE 6: note = iodophenylalanine
  SITE 10: note = 4-aminopiperidine-4-carboxylic acid
XQKTSXGRGXKWKK

SEQ ID NO: 98 - protein, 14 aa, synthetic construct
  source 1..14: note = CXP1R
  SITE 10: note = 4-aminopiperidine-4-carboxylic acid
  SITE 6: note = iodophenylalanine
  SITE 3: note = iodophenylalanine
KQXTSXGRGXKWKK

SEQ ID NO: 99 - protein, 14 aa, synthetic construct
  source 1..14: note = CXP1S
  SITE 6: note = iodophenylalanine
  SITE 8: note = iodophenylalanine
  SITE 10: note = 4-aminopiperidine-4-carboxylic acid
KQKTSXGXGXKWKK

SEQ ID NO: 100 - protein, 14 aa, synthetic construct
  source 1..14: note = CXP1T
  SITE 6: note = iodophenylalanine
  SITE 10: note = iodophenylalanine
KQKTSXGRGXKWKK

SEQ ID NO: 101 - protein, 14 aa, synthetic construct
  source 1..14: note = CXP1U
  SITE 6: note = iodophenylalanine
  SITE 11: note = iodophenylalanine
  SITE 10: note = 4-aminopiperidine-4-carboxylic acid
KQKTSXGRGXXWKK

SEQ ID NO: 102 - protein, 18 aa, synthetic construct
  source 1..18: note = CXP1V
  SITE 6: note = iodophenylalanine
  SITE 10: note = 4-aminopiperidine-4-carboxylic acid
  SITE 13: note = iodophenylalanine
KQKTSXGRGXKWXKKWKK

SEQ ID NO: 103 - protein, 18 aa, synthetic construct
  source 1..18: note = CXP1W
  SITE 10: note = 4-aminopiperidine-4-carboxylic acid
  SITE 14: note = iodophenylalanine
  SITE 6: note = iodophenylalanine
KQKTSXGRGXKWKXKWKK

SEQ ID NO: 104 - protein, 18 aa, synthetic construct
  source 1..18: note = CXP1AC
  SITE 1: note = 2,4-diaminobutyric acid
  SITE 3: note = 2,4-diaminobutyric acid
  SITE 11: note = 2,4-diaminobutyric acid
  SITE 13..14: note = 2,4-diaminobutyric acid
  SITE 10: note = 4-aminopiperidine-4-carboxylic acid
  SITE 6: note = iodophenylalanine
XQXTSXGRGXXWXXKWKK

SEQ ID NO: 105 - protein, 18 aa, synthetic construct
  source 1..18: note = CXP1AD
  SITE 6: note = iodophenylalanine
  SITE 10: note = 4-aminopiperidine-4-carboxylic acid
  SITE 1: note = 2,4-diaminobutyric acid
  SITE 3: note = 2,4-diaminobutyric acid
  SITE 8: note = 2,4-diaminobutyric acid
  SITE 11: note = 2,4-diaminobutyric acid
  SITE 13..14: note = 2,4-diaminobutyric acid
XQXTSXGXGXXWXXKWKK

SEQ ID NO: 106 - protein, 18 aa, synthetic construct
  source 1..18: note = CXP1AE
  SITE 1: note = 2,4-diaminobutyric acid
  SITE 3: note = 2,4-diaminobutyric acid
  SITE 10..11: note = 2,4-diaminobutyric acid
  SITE 13..14: note = 2,4-diaminobutyric acid
  SITE 6: note = iodophenylalanine
XQXTSXGRGXXWXXKWKK

SEQ ID NO: 107 - protein, 18 aa, synthetic construct
  source 1..18: note = CXP1AF
  SITE 6: note = iodophenylalanine
  SITE 1: note = 2,4-diaminobutyric acid
  SITE 3: note = 2,4-diaminobutyric acid
  SITE 8: note = 2,4-diaminobutyric acid
  SITE 10..11: note = 2,4-diaminobutyric acid
  SITE 13..14: note = 2,4-diaminobutyric acid
XQXTSXGXGXXWXXKWKK

SEQ ID NO: 108 - protein, 10 aa, synthetic construct
  source 1..10: note = CXP1X
  SITE 10: note = 4-aminopiperidine-4-carboxylic acid
KQKTSFGRGX

SEQ ID NO: 109 - protein, 10 aa, synthetic construct
  source 1..10: note = CXP1Y
  SITE 6: note = iodophenylalanine
KQKTSXGRGK

SEQ ID NO: 110 - protein, 10 aa, synthetic construct
KQKTSFGRGK

SEQ ID NO: 111 - protein, 10 aa, synthetic construct
  source 1..10: note = CXP1AA
  SITE 10: note = 4-aminopiperidine-4-carboxylic acid
  SITE 6: note = iodophenylalanine
KQKTSXGKGX

SEQ ID NO: 112 - protein, 10 aa, synthetic construct
  source 1..10: note = CXP1P
  SITE 6: note = iodophenylalanine
  SITE 10: note = 4-aminopiperidine-4-carboxylic acid
KQKTSXARAX

SEQ ID NO: 113 - protein, 10 aa, synthetic construct
  source 1..10: note = CXP1B
  SITE 10: note = 4-aminopiperidine-4-carboxylic acid
  SITE 6: note = iodophenylalanine
AQKTSXGRGX

SEQ ID NO: 114 - protein, 10 aa, synthetic construct
  source 1..10: note = CXP1C
  SITE 6: note = iodophenylalanine
  SITE 10: note = 4-aminopiperidine-4-carboxylic acid
KAKTSXGRGX

SEQ ID NO: 115 - protein, 10 aa, synthetic construct
  source 1..10: note = CXP1D
  SITE 10: note = 4-aminopiperidine-4-carboxylic acid
  SITE 6: note = iodophenylalanine
KQATSXGRGX

SEQ ID NO: 116 - protein, 10 aa, synthetic construct
  source 1..10: note = CXP1E
  SITE 6: note = iodophenylalanine
  SITE 10: note = 4-aminopiperidine-4-carboxylic acid
KQKASXGRGX

SEQ ID NO: 117 - protein, 10 aa, synthetic construct
  source 1..10: note = CXP1F
  SITE 10: note = 4-aminopiperidine-4-carboxylic acid
  SITE 6: note = iodophenylalanine
KQKTAXGRGX

SEQ ID NO: 118 - protein, 10 aa, synthetic construct
  source 1..10: note = CXP1G
  SITE 10: note = 4-aminopiperidine-4-carboxylic acid
KQKTSAGRGX

SEQ ID NO: 119 - protein, 10 aa, synthetic construct
  source 1..10: note = CXP1H
  SITE 6: note = iodophenylalanine
  SITE 10: note = 4-aminopiperidine-4-carboxylic acid
KQKTSXARGX

SEQ ID NO: 120 - protein, 10 aa, synthetic construct
  source 1..10: note = CXP1I
  SITE 10: note = 4-aminopiperidine-4-carboxylic acid
  SITE 6: note = iodophenylalanine
KQKTSXGAGX

SEQ ID NO: 121 - protein, 10 aa, synthetic construct
  source 1..10: note = CXP1J
  SITE 6: note = iodophenylalanine
  SITE 10: note = 4-aminopiperidine-4-carboxylic acid
KQKTSXGRAX

SEQ ID NO: 122 - protein, 10 aa, synthetic construct
  source 1..10: note = CXP1K
  SITE 6: note = iodophenylalanine
KQKTSXGRGA

SEQ ID NO: 123 - protein, 11 aa, synthetic construct
  source 1..11: note = CXP1L
  SITE 6: note = iodophenylalanine
  SITE 10: note = 4-aminopiperidine-4-carboxylic acid
KQKTSXGRGXA

SEQ ID NO: 124 - protein, 12 aa, synthetic construct
  source 1..12: note = CXP1M
  SITE 10: note = 4-aminopiperidine-4-carboxylic acid
  SITE 6: note = iodophenylalanine
KQKTSXGRGXKA

SEQ ID NO: 125 - protein, 13 aa, synthetic construct
  source 1..13: note = CXP1N
  SITE 6: note = iodophenylalanine
  SITE 10: note = 4-aminopiperidine-4-carboxylic acid
KQKTSXGRGXKWA

SEQ ID NO: 126 - protein, 14 aa, synthetic construct
  source 1..14: note = CXP1O
  SITE 10: note = 4-aminopiperidine-4-carboxylic acid
  SITE 6: note = iodophenylalanine
KQKTSXGRGXKWKA

SEQ ID NO: 127 - protein, 10 aa, synthetic construct
  source 1..10: note = CXP1Q
  SITE 1: note = iodophenylalanine
  SITE 6: note = iodophenylalanine
  SITE 10: note = 4-aminopiperidine-4-carboxylic acid
XQKTSXGRGX

SEQ ID NO: 128 - protein, 10 aa, synthetic construct
  source 1..10: note = CXP1R
  SITE 10: note = 4-aminopiperidine-4-carboxylic acid
  SITE 3: note = iodophenylalanine
  SITE 6: note = iodophenylalanine
KQXTSXGRGX

SEQ ID NO: 129 - protein, 10 aa, synthetic construct
  source 1..10: note = CXP1S
  SITE 6: note = iodophenylalanine
  SITE 8: note = iodophenylalanine
  SITE 10: note = 4-aminopiperidine-4-carboxylic acid
KQKTSXGXGX

SEQ ID NO: 130 - protein, 10 aa, synthetic construct
  source 1..10: note = CXP1T
  SITE 6: note = iodophenylalanine
  SITE 10: note = iodophenylalanine
KQKTSXGRGX

SEQ ID NO: 131 - protein, 14 aa, synthetic construct
  source 1..14: note = CXP1U
  SITE 6: note = iodophenylalanine
  SITE 11: note = iodophenylalanine
  SITE 10: note = 4-aminopiperidine-4-carboxylic acid
KQKTSXGRGXXWKK

SEQ ID NO: 132 - protein, 14 aa, synthetic construct
  source 1..14: note = CXP1V
  SITE 10: note = 4-aminopiperidine-4-carboxylic acid
  SITE 6: note = iodophenylalanine
  SITE 13: note = iodophenylalanine
KQKTSXGRGXKWXK

SEQ ID NO: 133 - protein, 14 aa, synthetic construct
  source 1..14: note = CXP1W
  SITE 6: note = iodophenylalanine
  SITE 14: note = iodophenylalanine
  SITE 10: note = 4-aminopiperidine-4-carboxylic acid
KQKTSXGRGXKWKX

SEQ ID NO: 134 - protein, 14 aa, synthetic construct
  source 1..14: note = CXP1AC
  SITE 1: note = 2,4-diaminobutyric acid
  SITE 3: note = 2,4-diaminobutyric acid
  SITE 6: note = iodophenylalanine
  SITE 10: note = 4-aminopiperidine-4-carboxylic acid
  SITE 11: note = 2,4-diaminobutyric acid
  SITE 13..14: note = 2,4-diaminobutyric acid
XQXTSXGRGXXWXX

SEQ ID NO: 135 - protein, 14 aa, synthetic construct
  source 1..14: note = CXP1AD
  SITE 13..14: note = 2,4-diaminobutyric acid
  SITE 11: note = 2,4-diaminobutyric acid
  SITE 10: note = 4-aminopiperidine-4-carboxylic acid
  SITE 6: note = iodophenylalanine
  SITE 1: note = 2,4-diaminobutyric acid
  SITE 3: note = 2,4-diaminobutyric acid
  SITE 8: note = 2,4-diaminobutyric acid
XQXTSXGXGXXWXX

SEQ ID NO: 136 - protein, 14 aa, synthetic construct
  source 1..14: note = CXP1AE
  SITE 1: note = 2,4-diaminobutyric acid
  SITE 3: note = 2,4-diaminobutyric acid
  SITE 6: note = iodophenylalanine
  SITE 10..11: note = 2,4-diaminobutyric acid
  SITE 13..14: note = 2,4-diaminobutyric acid
XQXTSXGRGXXWXX

SEQ ID NO: 137 - protein, 14 aa, synthetic construct
  source 1..14: note = CXP1AF
  SITE 1: note = 2,4-diaminobutyric acid
  SITE 3: note = 2,4-diaminobutyric acid
  SITE 6: note = iodophenylalanine
  SITE 8: note = 2,4-diaminobutyric acid
  SITE 10..11: note = 2,4-diaminobutyric acid
  SITE 13..14: note = 2,4-diaminobutyric acid
XQXTSXGXGXXWXX

SEQ ID NO: 138-142 - skipped (5 entries)

SEQ ID NO: 143 - protein, 4 aa, synthetic construct
  source 1..4: note = motif amino acid sequence
KWKK

SEQ ID NO: 144 - protein, 4 aa, synthetic construct
  source 1..4: note = motif amino acid sequence
KKWK

SEQ ID NO: 145 - protein, 5 aa, synthetic construct
  source 1..5: note = motif amino acid sequence
KWWKK

SEQ ID NO: 146 - protein, 4 aa, synthetic construct
  source 1..4: note = motif amino acid sequence
WWKK

SEQ ID NO: 147 - protein, 4 aa, synthetic construct
  source 1..4: note = motif amino acid sequence
KKKK

SEQ ID NO: 148 - DNA, 25 bp, synthetic construct
  source 1..25: mol_type = other DNA; note = M23D PMO
ggccaaacctcggcttacctgaaat

SEQ ID NO: 149 - protein, 10 aa, synthetic construct
  source 1..10: note = peptide sequence
  SITE 6: note = halophenylalanine
  SITE 10: note = 4-aminopiperidine-4-carboxylic acid
KQKTSXGRGX

SEQ ID NO: 150 - protein, 10 aa, synthetic construct
  source 1..10: note = peptide sequence
  SITE 2: note = 4-aminopiperidine-4-carboxylic acid
  SITE 4: note = 2,4-diaminobutyric acid
  SITE 6: note = 2,4-diaminobutyric acid
  SITE 9: note = halophenylalanine
  SITE 10: note = 2-amino-3-(naphthalen-2-yl)propanoic acid
PXGXKXSVXX

SEQ ID NO: 151 - protein, 10 aa, synthetic construct
  source 1..10: note = peptide sequence
  SITE 4: note = halophenylalanine
  SITE 6: note = 2,4-diaminobutyric acid
VQHXRXKEKN

SEQ ID NO: 152 - skipped

SEQ ID NO: 153 - protein, 6 aa, synthetic construct
  source 1..6: note = peptide sequence
  SITE 4: note = halophenylalanine
KTSXGR

SEQ ID NO: 154 - skipped

SEQ ID NO: 155 - protein, 8 aa, synthetic construct
  source 1..8: note = peptide sequence
  SITE 6: note = halophenylalanine
KQKTSXGR

SEQ ID NO: 156 - skipped

SEQ ID NO: 157 - protein, 7 aa, synthetic construct
  source 1..7: note = peptide sequence
  SITE 5: note = halophenylalanine
QKTSXGR

SEQ ID NO: 158 - protein, 8 aa, synthetic construct
  source 1..8: note = peptide sequence
  SITE 6: note = halophenylalanine
KQKTSXGR

(19) United States
(12) Patent Application Publication
(10) Pub. No.: US 2025/0171776 A1
(43) Pub. Date: May 29, 2025

(54) NON-CANONICAL CELL-PENETRATING PEPTIDES FOR ANTISENSE OLIGOMER DELIVERY

(71) Applicants: Sarepta Therapeutics, Inc., Cambridge, MA (US); Massachusetts Institute of Technology, Cambridge, MA (US)

(72) Inventors: Charlotte E. FARQUHAR, Cambridge, MA (US); Bradley L. PENTELUTE, Cambridge, MA (US); Annika B. MALMBERG, Cambridge, MA (US); Carly SCHISSEL, Cambridge, MA (US); Kathy Ye MORGAN, Cambridge, MA (US); Anirban BARDHAN, Cambridge, MA (US); Ryan OLIVER, Cambridge, MA (US); Vincent GUERLAVAIS, Cambridge, MA (US); Tushare JINADASA, Cambridge, MA (US); Alex CALLAHAN, Cambridge, MA (US)

(73) Assignees: Sarepta Therapeutics, Inc., Cambridge, MA (US); Massachusetts Institute of Technology, Cambridge, MA (US)

(21) Appl. No.: 18/792,964

(22) Filed: Aug. 2, 2024

Related U.S. Application Data
(60) Provisional application No. 63/530,363, filed on Aug. 2, 2023.

(51) Int. Cl. C12N 15/113; A61K 47/64

(52) U.S. Cl. CPC C12N 15/113; A61K 47/6455; C12N 2310/11; C12N 2310/3513

(57) ABSTRACT

Provided herein are antisense oligomer conjugates. Also provided herein are methods of treating a muscle disease, a viral infection, or a bacterial infection in a subject in need thereof, comprising administering to the subject one or more of the antisense oligomer conjugates described herein.

### RELATED APPLICATIONS

This application claims priority under 35 U.S.C. § 119(e) to U.S. Provisional Application No. 63/530,363, filed on Aug. 2, 2023, the disclosure of which is incorporated herein by reference in its entirety.

### REFERENCE TO A SEQUENCE LISTING SUBMITTED ELECTRONICALLY VIA EFS-WEB

The content of the electronically submitted sequence listing in ASCII text file (Name: 4140_1080002_SequenceListing_ST26.xml; Size: 274,623 bytes; and Date of Creation: Aug. 2, 2024) is herein incorporated by reference in its entirety.

### BACKGROUND

Antisense technology provides a means for modulating the expression of one or more specific gene products, including alternative splice products, and is uniquely useful in a number of therapeutic, diagnostic, and research applications. The principle behind antisense technology is that an antisense compound, e.g., an oligonucleotide, which hybridizes to a target nucleic acid, modulates gene expression activities such as transcription, splicing, or translation through any one of a number of antisense mechanisms. The sequence specificity of antisense compounds makes them attractive as tools for target validation and gene functionalization, as well as therapeutics, to selectively modulate the expression of genes involved in disease.

Although significant progress has been made in the field of antisense technology, there remains a need in the art for oligonucleotides and antisense oligomer conjugates.

### SUMMARY OF THE INVENTION

Provided herein are antisense oligomer conjugates comprising an antisense oligomer covalently bound to a cell-penetrating peptide, wherein the cell-penetrating peptide comprises at least one non-canonical amino acid.

The antisense oligomer can be a phosphorodiamidate morpholino oligomer and the peptide can be any of the peptides provided herein. The antisense oligomer conjugates are useful for the treatment of various diseases in a subject in need thereof, including, but not limited to, neuromuscular diseases such as Duchenne muscular dystrophy. Certain antisense oligomer conjugates disclosed herein exhibit improved antisense or antigen performance.

In an aspect provided herein is an antisense oligomer conjugate of structural Formula (III):

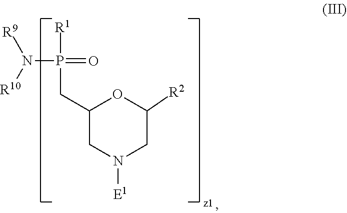

or a pharmaceutically acceptable salt thereof, wherein:
wherein the portion of the structure bracketed by z1 is a morpholine oligonucleotide as described herein; and —NR⁹R¹⁰ is

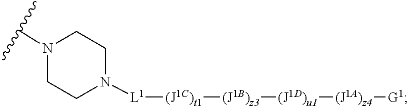

or E¹ is -L²-(J^{2C})₁₂-(J^{2B})_{z5}-(J^{2D})ᵤ₂-(J^{2A})_{z6}-G²; or both of these conditions apply. These two groups constitute novel peptide-linker portions comprising at least one non-canonical amino acid, wherein the peptide-linker is covalently attached to the morpholino oligonucleotide as described herein.

Representative of the antisense oligomer conjugates disclosed herein are conjugates of Formula (VI) and Formula (VII):

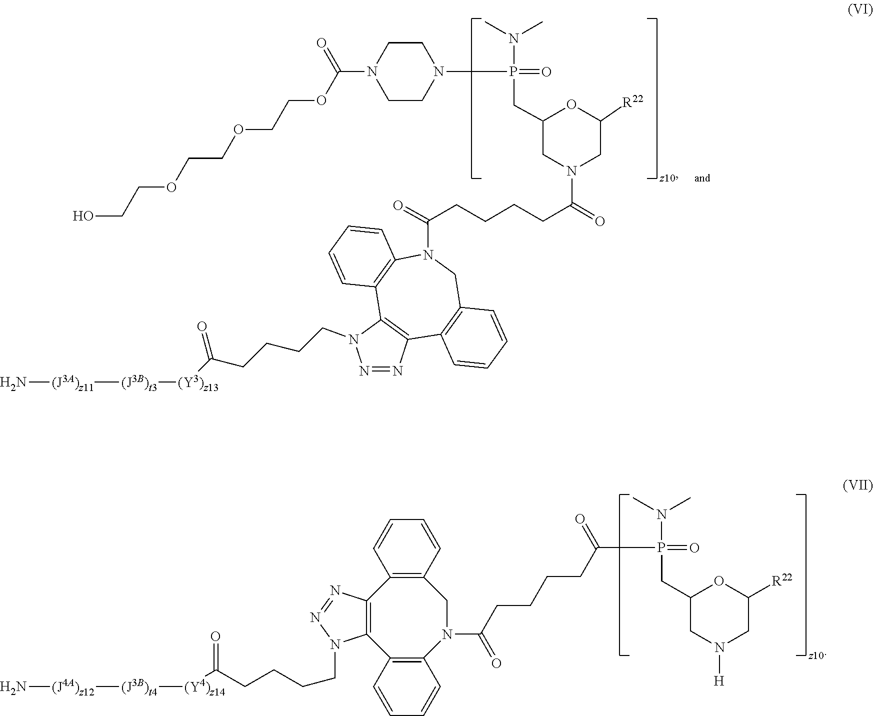

Representative non-canonical amino acids that can be within J^{1C}, J^{1D}, J^{2C}, J^{2D}, J^{3B} or J^{4B} include 2,4-diaminobutyric acid (Dab), 2-amino-5-ureidopentanoic acid (citrulline (Cit)), 2-amino-3-(naphthalen-2-yl)propanoic acid (Nap), 4-aminopiperidine-4-carboxylic acid (Pip), 2-amino-3-(pyridin-3-yl)propanoic acid (Pyr), or

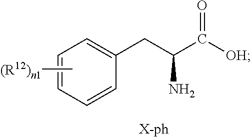

where R²⁴ is —F, —Cl, —Br, or —I.

Representative canonical peptide sequences formed by the J^{1B}, J^{1A} J^{2B}, J^{2A}, J^{3A}, or J^{4A} is the amino acid sequence Lys-Typ motifs, for example, Lys-Trp-Lys-Lys.

In another aspect, provided herein is a pharmaceutical composition comprising an antisense oligomer conjugate provided herein and a pharmaceutically acceptable carrier.

Also provided herein are methods of treating a neuromuscular disease comprising administering to a subject in need thereof one or more of the antisense oligomer conjugates provided herein.

In some embodiments, the antisense oligomer conjugates as described herein can be used for treating muscular dystrophy in a patient suffering from Duchenne muscular dystrophy (DMD).

### BRIEF DESCRIPTION OF THE FIGURES

FIG. 1 Shows the design and monomer set of a CPP library disclosed herein. The glycine at the N-terminus separates the N-terminal azide from the variable region (each variable amino acid is represented by “X”). The C-terminal “lysine tryptophan” motif, Lys-Trp-Lys-Lys, is amidated.

FIG. 2 Shows the chromatogram of the peptides separation of the CPP library that were resolved through cation exchange chromatography in ammonium acetate buffer (pH 5) with a 20-500 mM gradient over 75 minutes. In addition, FIG. 2 shows eGFP fluorescence of HeLa 654 cells treated with 5 or 20 μM total PMO-peptide conjugate from the library fractions. The eGFP fluorescence of each PMO-peptide fraction relative to the fluorescence of 5 or 20 μM of PMO 654 alone determined via flow cytometry is superimposed on the chromatogram from the CX separation of the library peptides.

FIG. 3 A) Shows nine peptide sequences chosen from pool 10. B) Shows the eGFP fluorescence of each PMO-peptide conjugate relative to the fluorescence of 20 μM of IVS2-654 PMO alone determined via flow cytometry. All PMO-peptides improved PMO delivery significantly over the IVS2-654 PMO alone at 20 μM (p<0.05). Penetratin, a known CPP, was included as a positive control.

FIG. 4 A) Shows the mean eGFP fluorescence of HeLa 654 cells treated with 1, 2.5, 5, 10, 25, or 50 μM of PMO-CPP conjugate relative to the fluorescence of cells treated with vehicle only for 22 h prior to flow cytometry. Bars represent mean±SD, N=3. B) Shows the percentage of LDH release above vehicle from the cell supernatant of A) relative to fully lysed cells.

FIG. 5 A) Shows the sequence and structure of the chimeric PMO-CXP1-CXP1 (top) conjugate and singular PMO-CXP1 conjugate (bottom). B) Shows the mean eGFP fluorescence of HeLa 654 cells treated with 1, 2.5, 5, 10, 25, or 50 μM PMO-CPP relative to the fluorescence of cells treated with vehicle only for 22 h prior to flow cytometry. Bars represent mean±SD, N=3.

FIG. 6 A) Shows the sequence and structure of the chimeric PMO-Segment (SA, SB, SC, or SD)-CXP1 conjugates. B) Shows the mean eGFP fluorescence of HeLa 654 cells treated with 2.5 μM PMO-CPP conjugates relative to the fluorescence of cells treated with vehicle only for 22 h prior to flow cytometry. Bars represent mean±SD, N=3. C) Shows the percentage of LDH release above vehicle from the cell supernatant of B) at 25 μM relative to fully lysed cells.

FIG. 7 A) Shows the sequence of the chimeric peptides, CXD1A and CXD1B. B) Shows the mean eGFP fluorescence of HeLa 654 cells treated with 1 μM of PMO-CPP conjugate relative to the fluorescence of cells treated with vehicle only for 22 h prior to flow cytometry. Results are given as Bars represent mean±SD, N=3. C) Shows the percentage of LDH release above vehicle from the cell supernatant of B) at 50 μM relative to fully lysed cells.

FIG. 8 A-M) Shows the therapeutic window studies for all of the PMO-CPP conjugates. PMO-delivery was measured through the HeLa 654 eGFP assay, and toxicity was measured through LDH release at 18 hours in TH-1 renal cells.

FIG. 9 A) Shows the efficacy of PMO-CXP1 relative to PMO-Bpeptide in the quadriceps, in the eGFP-654 mouse model. B) shows the dose response of various concentrations of PMO-CXP1 and PMO-Bpeptide intravenously delivered to eGFP-654 mice. Tissues were collected seven days after injection.

FIG. 10 Shows the PMO-CXP1 exposure in the quadriceps of an eGFP-654 mouse model, where the PPMO was delivered at various concentrations intravenously. Tissues were collected seven after injection.

FIG. 11 A) Shows the efficacy of PMO-CXP1 relative to PMO-Bpeptide in the heart of an eGFP-654 mouse model. B) shows the dose response of various concentrations of PMO-CXP1 and PMO-Bpeptide intravenously delivered to eGFP-654 mice. Tissues were collected seven days after injection.

FIG. 12 Shows the concentration of PMO-CXP1 and PMO-Bpeptide exposure in the heart of an eGFP-654 mouse model, where the PPMO was delivered at various concentrations intravenously. Tissues were collected seven days after injection.

FIG. 13 Shows the efficacy of PMO-CXP1 relative to PMO-Bpeptide in the diaphragm of an eGFP-654 mouse model. The PPMO was delivered intravenously at various concentrations to the eGFP-654 mice and tissues were collected seven days after.

FIG. 14 Shows the concentration of PMO-CXP1 and PMO-Bpeptide exposure in the diaphragm of an eGFP-654 mouse model, where the PPMO was delivered at various concentrations intravenously to the eGFP-654 mice and tissues were collected seven days after injection.

FIG. 15 Shows the efficacy of PMO-CXP1 relative to PMO-Bpeptide in the kidney of an eGFP-654 mouse model. The PPMO was delivered intravenously at various concentrations to the eGFP-654 miceand and tissues were collected seven days after injection.

FIG. 16 Shows the concentration of PMO-CXP1 and PMO-Bpeptide exposure in the kidney of an eGFP-654 mouse model, where the PPMO was delivered at various concentrations intravenously to the eGFP-654 mice and tissues were collected seven days after injection.

FIG. 17 Shows the exposure duration of PMO-CXP1 in the eGFP-654 mouse model. PMO-CXP1 was delivered intravenously to the eGFP-654 mice at 20 mg/kg and tissues were collected for analysis weekly for 8 weeks.

FIG. 18 Shows the exon skipping by various PPMOs in C2C12 cells. C2C12s cells were seeded and differentiated for 3 days, before being transfected with PPMO. Cells were collected after 24 hrs of incubation and the percentage exon skipping was calculated to be the percentage of skipped transcript to total transcript shown by endpoint PCR.

FIG. 19 A) Shows the percentage exon skipping in the quadriceps in a mdx mouse model by M23DPMO-CXP1 relative to M23DPMO-R6G at various concentrations. B) Shows the dose response in the quadriceps in an mdx mouse model by M23DPMO-CXP1 relative to M23DPMO-R6G at various concentrations. PPMO was delivered intravenously to mdx mice and tissues were collected seven days after injection. Droplet Digital PCR (ddPCR) was used to quantify skipped transcripts versus unskipped transcripts. The percentage of Exon 23 skipping was calculated to be the number of skipped transcripts over total transcripts.

FIG. 20 Shows the percentage of dystrophin in the quadriceps in a mdx mouse model by M23DPMO-CXP1 relative to M23DPMO-R6G at various concentrations. PPMO was delivered intravenously to mdx mice and tissues were collected seven days after injection. Dystrophin expression was determined using an automated western blot system (Proteinsimple's Jess) and the percentage of dystrophin observed in mdx tissue was calculated relative to a wild type protein standard curve.

FIG. 21 Shows the concentration of M23DPMO-CXP1 relative to M23DPMO-R6G in the quadriceps of a mdx mouse model at various concentrations. PPMO was delivered intravenously to mdx mice and tissues were collected seven days after injection. The compound concentration was determined with an enzyme-linked oligonucleotide hybridization assay.

FIG. 22 A) Shows the percentage of exon 23 skipping in the heart of a mdx mouse model by M23DPMO-CXP1 relative to M23DPMO-R6G at various concentrations. B) Shows the PPMO dose response in the heart of a mdx mouse model by M23DPMO-CXP1 relative to M23DPMO-R6G at various concentrations. PPMO was delivered intravenously to mdx mice and tissues were collected seven days after injection. RNA was extracted and Droplet Digital PCR (ddPCR) was used to quantify skipped transcripts versus unskipped transcripts. The percentage of Exon 23 skipping was calculated to be the number of skipped transcripts over total transcripts.

FIG. 23 Shows the percentage of dystrophin in the heart of a mdx mouse model by M23DPMO-CXP1 relative to M23DPMO-R6G PPMO at various concentrations. PPMO was delivered intravenously to mdx mice and tissues were collected seven days after injection. The dystrophin expression was determined using an automated western blot system (Proteinsimple's Jess) and the percentage of dystrophin observed in mdx tissue was calculated relative to a wild type protein standard curve.

FIG. 24 Shows the concentration of M23DPMO-CXP1 relative to M23DPMO-R6G in the heart of a mdx mouse model at various concentrations. PPMO was delivered intravenously to mdx mice and tissues were collected seven days after injection. The compound concentration was determined with an enzyme-linked oligonucleotide hybridization assay.

FIG. 25 A) Shows the effect of M23DPMO-CXP1 relative to M23DPMO-R6G on the serum biomarker creatine Kinase (CK) in a mdx mouse model at various concentrations. B) Shows the effect of M23DPMO-CXP1 relative to M23DPMO-R6G on the serum biomarker lactate dehydrogenase (LDH) in a mdx mouse model at various concentrations. PPMO was delivered intravenously to mdx mice and tissues were collected seven days after injection. Blood serum was collected from the mdx mice and serum biomarkers were analyzed.

FIG. 26 Shows the efficacy of various PPMOs relative to PMO-Bpeptide in the quadriceps of an eGFP-654 mouse model at various concentrations. PPMO was delivered intravenously to eGFP-654 mice and tissues were collected seven days after injection.

FIG. 27 Shows the efficacy of various PPMOs relative to PMO-Bpeptide in the heart of an eGFP-654 mouse model at various concentrations. PPMO was delivered intravenously to eGFP-654 mice and tissues were collected seven days after injection.

FIG. 28 Shows the efficacy of various PPMOs relative to PMO-Bpeptide in the diaphragm of an eGFP-654 mouse model at various concentrations. PPMO was delivered intravenously to eGFP-654 mice and tissues were collected seven days after injection.

FIG. 29 Shows the efficacy of various PPMOs relative to PMO-Bpeptide in the kidney of an eGFP-654 mouse model at various concentrations. PPMO was delivered intravenously to eGFP-654 mice and tissues were collected seven days after injection.

FIG. 30 Shows the percentage of GFP positive HELA-654 cells after being dosed with various PPMOs at 20 μM for 72 hours. HELA-654 cells were imaged at 72 hours with an incucyte and the number of GFP positive cells versus total cell counts were calculated with the incucyte image analysis software. Statistical analysis was conducted using a one-way analysis of variance (ANOVA) test with Dunnett's multiple comparison test. Bars represent mean±standard deviation (SD), with P<0.05 was considered to indicate statistical significance, (**** p<0.0001, *** p<0.005, ** p<0.01, * p<0.05).

FIG. 31 Shows the percentage of LDH release in HELA-654 cells that were dosed with various PPMOs at 250 μM for 16 hours. The media from the cells was analyzed for LDH release with an LDH assay and the percentage of LDH release was calculated relative to fully lysed cells.

FIG. 32 Shows the percentage of LDH release in TH-1 renal cells dosed with various PPMOs at 250 μM for 16 hours. The media from the cells was analyzed for LDH release with an LDH assay and the percentage of LDH release was calculated relative to fully lysed cells.

FIG. 33 Shows the directionality of the peptide attachment to the oligonucleotide to form PPMOs where the peptide is attached either to the 3′-end, PMO-CXP1, or the 5′-end, CXP1AB, of the oligonucleotide.

FIG. 34 Shows the percentage of GFP positive HELA-654 cells after varying doses of 3′-attached peptide, PMO-CXP1, 5′-attached peptide, CXP1AB, relative to PMO-Bpeptide after 72 hours. HELA-654 cells were imaged at 72 hours with an incucyte and the number of GFP positive cells versus total cell counts were calculated with the incucyte image analysis software.

FIG. 35 Shows the efficacy of 3′-attached peptide, PMO-CXP1, 5′-attached peptide, CXP1AB, relative to PMO-Bpeptide in the quadriceps of an eGFP-654 mouse model at various concentrations. PPMO was delivered intravenously to eGFP-654 mice and tissues were collected seven days after injection.

FIG. 36 Shows the percentage of GFP positive HELA-654 cells after being dosed with various PPMOs at 30 μM for 72 hours. HELA-654 cells were imaged at 72 hours with an incucyte and the number of GFP positive cells versus total cell counts were calculated with the incucyte image analysis software. Statistical analysis was conducted using a one-way analysis of variance (ANOVA) test with Dunnett's multiple comparison test. Bars represent mean±standard deviation (SD), with P<0.05 was considered to indicate statistical significance, (**** p<0.0001, *** p<0.005, ** p<0.01, * p<0.05).

FIG. 37 Shows the percentage of GFP positive HELA-654 cells after variable doses with various PPMOs for 72 hours. HELA-654 cells were imaged at 72 hours with an incucyte and the number of GFP positive cells versus total cell counts were calculated with the incucyte image analysis software.

FIG. 38 Shows the percentage of LDH release in TH-1 renal cells with various PPMOs at 250 μM for 16 hours. The media from the cells was analyzed for LDH release with an LDH assay and the percentage of LDH release was calculated relative to fully lysed cells.

FIG. 39 Shows the efficacy of PMO-CXP1 analogs relative to PMO-CXP1 in the quadriceps of an eGFP-654 mouse model at various concentrations. PPMO was delivered intravenously to eGFP-654 mice and tissues were collected seven days after injection.

FIG. 40 Shows the percentage of GFP positive HELA-654 cells at varying doses of PPMO Dab analogs relative to PMO-CXP1 after 72 hours. HELA-654 cells were imaged at 72 hours with an incucyte and the number of GFP positive cells versus total cell counts were calculated with the incucyte image analysis software.

### DETAILED DESCRIPTION

### I. Aspects of the Disclosure

Aspect 1, provided herein is an antisense oligomer conjugate of Formula (III):

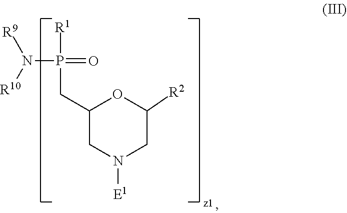

or a pharmaceutically acceptable salt thereof,
wherein:
z1 is an integer from 8 to 40;
each R¹ is independently —OH or —NR³R⁴;
each R³ and each R⁴ are independently hydrogen or C₁₋₆alkyl;
E¹ is hydrogen, —C₁₋₆alkyl, —C(O)C₁₋₆alkyl, stearoyl, trityl, monomethoxytrityl, dimethoxytrityl, trimethoxytrityl, -L²-(J^{2C})ₜ₂-(J^{2B})_{z5}-(J^{2D})ᵤ₂-(J^{2A})_{z6}-G², or

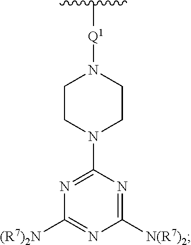

Q¹ is —C(O)(CH₂)₆C(O)— or —C(O)(CH₂)₂S₂(CH₂)₂C(O)—;
each R⁷ is independently —[(CH₂)₂OC(O)]N[(CH₂)₆NHC(═NH)NH₂]₂;
R⁹ is hydrogen or —C₁₋₆alkyl;
R¹⁰ is —CH₂C(O)NH₂, or R⁹ and R¹⁰ together with the nitrogen to which they are attached form a ring selected from:

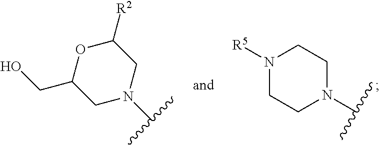

each R² is independently hydrogen, or an unsubstituted or R⁸-substituted nucleobase comprising a pyridine, pyrimidine, purine, or deaza-purine,
wherein R⁸ is a protecting group;
R⁵ is hydrogen, —C(O)R^{5A}, —C(O)OR^{5B}, R⁶-substituted or unsubstituted C₁₋₆alkyl, R⁶-substituted or unsubstituted —C₁₋₆heteroalkyl, R⁶-substituted or unsubstituted aryl, R⁶-substituted or unsubstituted heteroaryl, trityl, monomethoxytrityl, —C(O)[O(CH₂)₂₋₆]_{z2}—OH, or -L¹-(J^{1C})ₜ₁-(J^{1B})_{z3}-(J^{1A})_{z4}-G¹;
R^{5A} is hydrogen or substituted or unsubstituted C₁₋₆alkyl;
R^{5B} is hydrogen, substituted or unsubstituted C₁₋₆alkyl, substituted or unsubstituted aryl, or substituted or unsubstituted heteroaryl;
R⁶ is selected from —OR^{6A}, —SR^{6A}, —NHR^{6A};
R^{6A} is hydrogen or a solid support;
L¹ is —C(O)(CH₂)₁₋₈C(O)-heteroaryl-(CH₂)₁₋₈C(O)—[Y¹]_{z7};
L² is —C(O)(CH₂)₁₋₈C(O)-heteroaryl-(CH₂)₁₋₈C(O)—[Y²]_{z8};
Y¹ and Y² are each independently a linking amino acid;
z2 is an integer from 3 to 10;
z3, z4, z5, and z6 are each independently 0 or 1;
z7 and z8 are each independently 0, 1, or 2;
t1 and t2 are each independently an integer from 3 to 20;
u1 and u2 are each independently an integer selected from 0 and 3 to 20;
each J^{1A}, J^{1B}, J^{2A}, and J^{2B} is independently selected from Lys-Lys (SEQ ID NO: 138), Lys-Trp (SEQ ID NO: 139), Lys-Trp-Lys (SEQ ID NO: 140), Trp-Lys-Lys (SEQ ID NO: 141), Lys-Lys-Trp (SEQ ID NO: 142), Lys-Trp-Lys-Lys (SEQ ID NO: 143), Lys-Lys-Trp-Lys (SEQ ID NO: 144), Lys-Trp-Trp-Lys-Lys (SEQ ID NO: 145), Trp-Trp-Lys-Lys (SEQ ID NO: 146), and Lys-Lys-Lys-Lys (SEQ ID NO: 147);
each J^{1C}, J^{1D}, J^{2C}, and J^{2D} is independently Arg, His, Lys, Asp, Glu, Ser, Thr, Asn, Gln, Gly, Met, Pro, Val, Ala, Leu, Phe, Trp, Cys, Ile, Tyr, 2,4-diaminobutanoic acid dihydrochloride (Dab), 2-amino-5-ureidopentanoic acid (citrulline (Cit)), 2-amino-3-(naphthalen-2-yl)propanoic acid (Nap), 4-aminopiperidine-4-carboxylic acid (Pip), 2-amino-3-(pyridin-3-yl)propanoic acid (Pyr), or

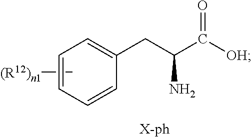

n1 is an integer from 1 to 5;
R¹² is —F, —Cl, —Br, or —I;
G¹ is hydrogen, —C(O)R^{13A}, —NR^{13A}R^{13B}, benzoyl, and stearoyl;
G² is hydrogen, —C(O)R^{14A}, —NR^{14A}R^{14B}, benzoyl, and stearoyl; and
R^{13A}, R^{13B}, R^{14A}, and R^{14B} are each independently hydrogen or alkyl;
provided that:
i) when u2 and z6 are each independently zero, or where u1 and z4 are each independently zero, then at least one of J^{1C} and J^{2C} is 2-amino-5-ureidopentanoic acid (citrulline (Cit)), 2-amino-3-(pyridin-3-yl)propanoic acid or

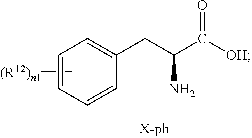

ii) E¹ is -L²-(J^{2C})ₜ₂-(J^{2B})_{z5}-(J^{2D})ᵤ₂-(J^{2A})_{z6}-G²; or R⁹ and R¹⁰ together with the nitrogen to which they are attached form:

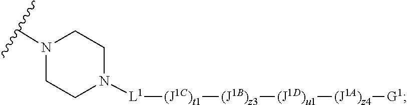

and
iii) at least one R² is not hydrogen.

Aspect 2, The antisense oligomer conjugate of Aspect 1 is an antisense oligomer conjugate of Formula (IIIa):

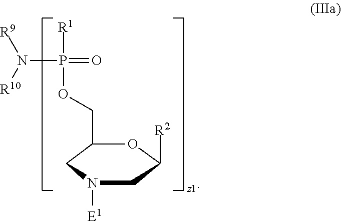

Aspect 3, The antisense oligomer conjugate of Aspect 1, where u2 and z6 are both zero, and u1 and z4 are both zero. This aspect provides for singular or non-chimeric peptide constructs. In an alternative aspect, at least one of u2 and z6 is not zero, and at least one of u1 and z4 are not zero. This aspect provides chimeric peptide constructs.

Aspect 4, provided herein is an antisense oligomer conjugate has Formula (IV):

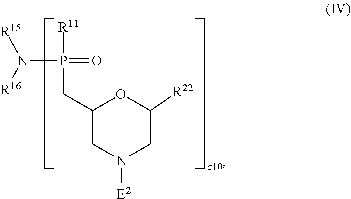

or a pharmaceutically acceptable salt thereof,
wherein:
z10 is an integer from 8 to 40;
each R¹¹ is independently —OH or —NR¹⁷R¹⁸,
each R¹⁷ and each R¹⁸ are independently hydrogen or C₁₋₆alkyl;
R¹⁵ is hydrogen or —C₁₋₆alkyl;
R¹⁶ is —CH₂C(O)NH₂, or R¹⁵ together and R¹⁶ together with the nitrogen to which they are attached join to form a ring selected from:

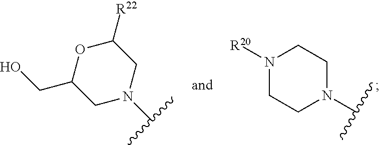

R²⁰ is hydrogen, —C(O)R^{20A}, —C(O)OR^{20B}, R²¹-substituted or unsubstituted C₁₋₆alkyl, R²¹-substituted or unsubstituted —C₁₋₆heteroalkyl, R²¹-substituted or unsubstituted aryl, R21-substituted or unsubstituted heteroaryl, trityl, monomethoxytrityl, —C(O)[O(CH₂)₂₋₆]_{z9}—OH, or -L⁴-(J^{4B})ₜ₄-(J^{4A})_{z12}-G⁴;
z9 is an integer from 3 to 10;
R^{20A} is hydrogen or substituted or unsubstituted C₁₋₆alky;
R^{20B} is hydrogen, substituted or unsubstituted C₁₋₆alkyl, substituted or unsubstituted aryl, or substituted or unsubstituted heteroaryl;
R²¹ is selected from —OR^{21A}, —SR^{21A}, —NHR^{21A};
R^{21A} is hydrogen or a solid support;
each R²² is independently hydrogen or R²³-substituted or unsubstituted nucleobase comprising a pyridine, pyrimidine, purine, or deaza-purine,
R²³ is a protecting group;
E² is hydrogen, —C₁₋₆alkyl, —C(O)C₁₋₆alkyl, stearoyl, trityl, monomethoxytrityl, dimethoxytrityl, trimethoxytrityl, or -L³-(J^{3B})ₜ₃-(J^{3A})_{z11}-G³, or

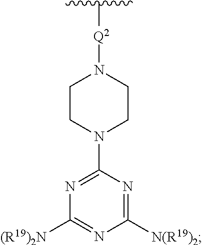

each R¹⁹ is independently —[(CH₂)₂OC(O)]N[(CH₂)₆NHC(═NH)NH₂]₂;
Q² is —C(O)(CH₂)₆C(O)— or —C(O)(CH₂)₂S₂(CH₂)₂C(O)—;
L³ is —C(O)(CH₂)₁₋₈C(O)-heteroaryl-(CH₂)₁₋₃C(O)—[Y³]_{z13};
L⁴ is —C(O)(CH₂)₁₋₈C(O)-heteroaryl-(CH₂)₁₋₈C(O)—[Y⁴]_{z14};
Y³ and Y⁴ are each independently a linking amino acid;
z11 and z12 are each independently 0 or 1;
z13 and z14 are each independently 0, 1, or 2;
t3 and t4 are each independently an integer from 3 to 20;
J^{3A} and J^{4A} are each independently selected from Lys-Lys (SEQ ID NO: 138), Lys-Trp (SEQ ID NO: 139), Lys-Trp-Lys (SEQ ID NO: 140), Trp-Lys-Lys (SEQ ID NO: 141), Lys-Lys-Trp (SEQ ID NO: 142), Lys-Trp-Lys-Lys (SEQ ID NO: 143), Lys-Lys-Trp-Lys (SEQ ID NO: 144), Lys-Trp-Trp-Lys-Lys (SEQ ID NO: 145), Trp-Trp-Lys-Lys (SEQ ID NO: 146), and Lys-Lys-Lys-Lys (SEQ ID NO: 147);
each J^{3B} and each J^{4B} is independently an L-amino acid selected from Arg, His, Lys, Asp, Glu, Ser, Thr, Asn, Gln, Gly, Met, Pro, Val, Ala, Leu, Phe, Trp, Cys, Ile, Tyr, 2,4-diaminobutyric acid (Dab), 2-amino-5-ureidopentanoic acid (citrulline (Cit)), 2-amino-3-(naphthalen-2-yl)propanoic acid (Nap), 4-aminopiperidine-4-carboxylic acid (Pip), 2-amino-3-(pyridin-3-yl)propanoic acid (Pyr), and

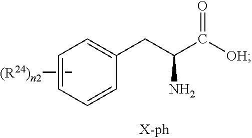

n2 is an integer from 1 to 5;
R²⁴ is —F, —Cl, —Br, or —I;
G³ is hydrogen, —C(O)R^{25A}, —NR^{25A}R^{25B}, benzoyl, and stearoyl;
G⁴ is hydrogen, —C(O)R^{26A}, —NR^{26A}R^{26B}, benzoyl, and stearoyl;
R^{25A}, R^{25B}, R^{26A}, and R^{26B} are each independently hydrogen or alkyl; and provided that:
i) E² is -L³-(J^{3B})ₜ₃-(J^{3A})_{z11}-G³; or R¹⁵ and R¹⁶ together with the nitrogen to which they are attached join to form

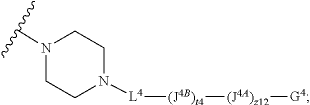

ii) at least one R²² is not hydrogen; and
iii) each J^{3B} and each J^{4B} are not: a D-amino acid, 9-aminohexanoic acid, γ-aminobutyric acid (Abu), homoleucine (Hle), norleucine (Nle), Dpa (diphenylalanine), aminomethylphenylalanine (Amf), or 2-amino-4-guanidinobutanoic acid (Gba).

Aspect 5, In certain embodiments of Aspect 4, the antisense oligomer conjugate of Formula (IV), or a pharmaceutically acceptable salt thereof, is an antisense oligomer conjugate of Formula (IVa):

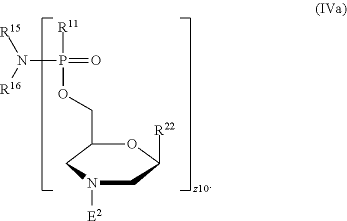

Aspect 6, The antisense oligomer conjugate of Aspect 4, or a pharmaceutically acceptable salt thereof, where -(J^{3B})ₜ₃-(J^{3A})_{z11}- or -(J^{4B})ₜ₄-(J^{4A})_{z12}- each independently form a peptide sequence selected from:

| SEQ ID | Peptide | |
|---|---|---|
| NO. | Name | Sequence |
| 48 | CXP1 | Lys-Gln-Lys-Thr-Ser-Iph-Gly-Arg-Gly-Pip-Lys-Trp-Lys-Lys; |
| 49 | CXP2 | Asn-Phe-Lys-Gln-Nap-His-Ala-Gly-Dab-Arg-Lys-Trp-Lys-Lys; |
| 50 | CXP3 | Dab-Val-Ala-Arg-Asn-Asn-Dab-Thr-Lys-Gly-Lys-Trp-Lys-Lys; |
| 51 | CXP4 | Cit-Met-Phe-Gly-Pip-Dab-Dab-Lys-Ala-Pro-Lys-Trp-Lys-Lys; |
| 52 | CXP5 | Dab-Pro-Arg-Dab-Leu-His-Ser-Dab-Ala-Thr-Lys-Trp-Lys-Lys; |
| 53 | CXP6 | Pro-Pip-Gly-Dab-Lys-Dab-Ser-Val-Iph-Nap-Lys-Trp-Lys-Lys; |
| 54 | CXP7 | Val-Gln-His-Iph-Arg-Dab-Lys-Glu-Lys-Asn-Lys-Trp-Lys-Lys; |
| 55 | CXP8 | Asn-Phe-Asn-Asn-Arg-Ser-Dab-Gly-His-Lys-Lys-Trp-Lys-Lys; |
| 56 | CXP9 | Ala-Arg-Ser-Glu-Gln-His-His-Nap-Ser-Dab-Lys-Trp-Lys-Lys; |
| 57 | SA | Iph-Gly-Arg-Gly-Pip-Lys-Trp-Lys-Lys; |
| 58 | SB | Lys-Thr-Ser-Iph-Gly-Arg-Lys-Trp-Lys-Lys; |
| 61 | SE | Iph-Gly-Arg-Lys-Trp-Lys-Lys; |
| 62 | SF | Lys-Gln-Lys-Thr-Ser-Iph-Gly-Arg-Lys-Trp-Lys-Lys; |
| 63 | SG | Ser-Iph-Gly-Arg-Lys-Trp-Lys-Lys; |
| 65 | SI | Gln-Lys-Thr-Ser-Iph-Gly-Arg-Lys-Trp-Lys-Lys; |
| 66 | SJ | Lys-Gln-Lys-Thr-Ser-Iph-Gly-Arg-Lys-Trp-Lys-Lys; |
| 67 | CXP1- | Lys-Gln-Lys-Thr-Ser-Iph-Gly-Arg-Gly-Pip-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 68 | CXP3- | Dab-Val-Ala-Arg-Asn-Asn-Dab-Thr-Lys-Gly-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 69 | CXP4- | Cit-Met-Phe-Gly-Pip-Dab-Dab-Lys-Ala-Pro-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 70 | SA-CXP1 | Iph-Gly-Arg-Gly-Pip-Lys-Gln-Lys-Thr-Ser-Iph-Gly-Arg-Gly-Pip-Lys- |
| | | Trp-Lys-Lys; |
| 71 | SB-CXP1 | Lys-Thr-Ser-Iph-Gly-Arg-Lys-Gln-Lys-Thr-Ser-Iph-Gly-Arg-Gly-Pip- |
| | | Lys-Trp-Lys-Lys; |
| 72 | SC-CXP1 | Lys-Gln-Lys-Thr-Ser-Lys-Gln-Lys-Thr-Ser-Iph-Gly-Arg-Gly-Pip- |
| | | Lys-Trp-Lys-Lys; |
| 73 | SD-CXP1 | Lys-Trp-Lys-Lys-Gln-Lys-Thr-Ser-Iph-Gly-Arg-Gly-Pip-Lys-Trp- |
| | | Lys-Lys; |
| 74 | CXD1A | Lys-Thr-Ser-Iph-Gly-Arg-Lys-Gln-Lys-Thr-Ser-Iph-Gly-Arg-Lys- |
| | | Trp-Lys-Lys; |
| 75 | CXD1B | Lys-Gln-Lys-Thr-Ser-Iph-Gly-Arg-Lys-Trp-Lys-Gln-Lys-Thr-Ser- |
| | | Iph-Gly-Arg-Lys-Trp-Lys-Lys; |
| 78 | CXP1X | Lys-Gln-Lys-Thr-Ser-Phe-Gly-Arg-Gly-Pip-Lys-Trp-Lys-Lys; |
| 79 | CXP1Y | Lys-Gln-Lys-Thr-Ser-Iph-Gly-Arg-Gly-Lys-Lys-Trp-Lys-Lys; |
| 80 | CXP1Z | Lys-Gln-Lys-Thr-Ser-Phe-Gly-Arg-Gly-Lys-Lys-Trp-Lys-Lys; |
| 81 | CXP1AA | Lys-Gln-Lys-Thr-Ser-Iph-Gly-Lys-Gly-Pip-Lys-Trp-Lys-Lys; |
| 82 | CXP1P | Lys-Gln-Lys-Thr-Ser-Iph-Ala-Arg-Ala-Pip-Lys-Trp-Lys-Lys; |
| 83 | CXP1B | Ala-Gln-Lys-Thr-Ser-Iph-Gly-Arg-Gly-Pip-Lys-Trp-Lys-Lys; |
| 84 | CXP1C | Lys-Ala-Lys-Thr-Ser-Iph-Gly-Arg-Gly-Pip-Lys-Trp-Lys-Lys; |
| 85 | CXP1D | Lys-Gln-Ala-Thr-Ser-Iph-Gly-Arg-Gly-Pip-Lys-Trp-Lys-Lys; |
| 86 | CXP1E | Lys-Gln-Lys-Ala-Ser-Iph-Gly-Arg-Gly-Pip-Lys-Trp-Lys-Lys; |
| 87 | CXP1F | Lys-Gln-Lys-Thr-Ala-Iph-Gly-Arg-Gly-Pip-Lys-Trp-Lys-Lys; |
| 88 | CXP1G | Lys-Gln-Lys-Thr-Ser-Ala-Gly-Arg-Gly-Pip-Lys-Trp-Lys-Lys; |
| 89 | CXP1H | Lys-Gln-Lys-Thr-Ser-Iph-Ala-Arg-Gly-Pip-Lys-Trp-Lys-Lys; |
| 90 | CXP1I | Lys-Gln-Lys-Thr-Ser-Iph-Gly-Ala-Gly-Pip-Lys-Trp-Lys-Lys; |
| 91 | CXP1J | Lys-Gln-Lys-Thr-Ser-Iph-Gly-Arg-Ala-Pip-Lys-Trp-Lys-Lys; |
| 92 | CXP1K | Lys-Gln-Lys-Thr-Ser-Iph-Gly-Arg-Gly-Ala-Lys-Trp-Lys-Lys; |
| 93 | CXP1L | Lys-Gln-Lys-Thr-Ser-Iph-Gly-Arg-Gly-Pip-Ala-Trp-Lys-Lys; |
| 94 | CXP1M | Lys-Gln-Lys-Thr-Ser-Iph-Gly-Arg-Gly-Pip-Lys-Ala-Lys-Lys; |
| 95 | CXP1N | Lys-Gln-Lys-Thr-Ser-Iph-Gly-Arg-Gly-Pip-Lys-Trp-Ala-Lys; |
| 96 | CXP1O | Lys-Gln-Lys-Thr-Ser-Iph-Gly-Arg-Gly-Pip-Lys-Trp-Lys-Ala- |
| | | Lys-Trp-Lys-Lys; |
| 97 | CXP1Q | Iph-Gln-Lys-Thr-Ser-Iph-Gly-Arg-Gly-Pip-Lys-Trp-Lys-Lys; |
| 98 | CXP1R | Lys-Gln-Iph-Thr-Ser-Iph-Gly-Arg-Gly-Pip-Lys-Trp-Lys-Lys; |
| 99 | CXP1S | Lys-Gln-Lys-Thr-Ser-Iph-Gly-Iph-Gly-Pip-Lys-Trp-Lys-Lys; |
| 100 | CXP1T | Lys-Gln-Lys-Thr-Ser-Iph-Gly-Arg-Gly-Iph-Lys-Trp-Lys-Lys; |
| 101 | CXP1U | Lys-Gln-Lys-Thr-Ser-Iph-Gly-Arg-Gly-Pip-Iph-Trp-Lys-Lys; |
| 102 | CXP1V | Lys-Gln-Lys-Thr-Ser-Iph-Gly-Arg-Gly-Pip-Lys-Trp-Iph-Lys- |
| | | Lys-Trp-Lys-Lys; |
| 103 | CXP1W | Lys-Gln-Lys-Thr-Ser-Iph-Gly-Arg-Gly-Pip-Lys-Trp-Lys-Iph- |
| | | Lys-Trp-Lys-Lys; |
| 104 | CXP1AC | Dab-Gln-Dab-Thr-Ser-Iph-Gly-Arg-Gly-Pip-Dab-Trp-Dab-Dab-Lys- |
| | | Trp-Lys-Lys; |
| 105 | CXP1AD | Dab-Gln-Dab-Thr-Ser-Iph-Gly-Dab-Gly-Pip-Dab-Trp-Dab-Dab-Lys- |
| | | Trp-Lys-Lys; |
| 106 | CXP1AE | Dab-Gln-Dab-Thr-Ser-Iph-Gly-Arg-Gly-Dab-Dab-Trp-Dab-Dab- |
| | | Lys-Trp-Lys-Lys; and |
| 107 | CXP1AF | Dab-Gln-Dab-Thr-Ser-Iph-Gly-Dab-Gly-Dab-Dab-Trp-Dab-Dab- |
| | | Lys-Trp-Lys-Lys. |

Aspect 7, The antisense oligomer conjugate of Aspect 4 or 5, or a pharmaceutically acceptable salt thereof, wherein -(J^{3B})ₜ₃- and -(J^{4B})ₜ₄- each independently form a peptide sequence selected from:

| SEQ ID | Peptide | |
|---|---|---|
| NO. | Name | Sequence |
| 1 | CXP1 | Lys-Gln-Lys-Thr-Ser-Iph-Gly-Arg-Gly-Pip; |
| 2 | CXP2 | Asn-Phe-Lys-Gln-Nap-His-Ala-Gly-Dab-Arg; |
| 3 | CXP3 | Dab-Val-Ala-Arg-Asn-Asn-Dab-Thr-Lys-Gly; |
| 4 | CXP4 | Cit-Met-Phe-Gly-Pip-Dab-Dab-Lys-Ala-Pro; |
| 5 | CXP5 | Dab-Pro-Arg-Dab-Leu-His-Ser-Dab-Ala-Thr; |
| 6 | CXP6 | Pro-Pip-Gly-Dab-Lys-Dab-Ser-Val-Iph-Nap; |
| 7 | CXP7 | Val-Gln-His-Iph-Arg-Dab-Lys-Glu-Lys-Asn; |
| 8 | CXP8 | Asn-Phe-Asn-Asn-Arg-Ser-Dab-Gly-His-Lys; |
| 9 | CXP9 | Ala-Arg-Ser-Glu-Gln-His-His-Nap-Ser-Dab; |
| 10 | SA | Iph-Gly-Arg-Gly-Pip; |
| 11 | SB | Lys-Thr-Ser-Iph-Gly-Arg; |
| 12 | SC | Lys-Gln-Lys-Thr-Ser; |
| 13 | SD | Lys-Trp-Lys; |
| 14 | SE | Iph-Gly-Arg; |
| 15 | SF | Lys-Gln-Lys-Thr-Ser-Iph-Gly-Arg; |
| 16 | SG | Ser-Iph-Gly-Arg; |
| 18 | SI | Gln-Lys-Thr-Ser-Iph-Gly-Arg; |
| 19 | SJ | Lys-Gln-Lys-Thr-Ser-Iph-Gly-Arg; |
| 39 | CXP1- | Lys-Gln-Lys-Thr-Ser-Iph-Gly-Arg-Gly-Pip-Lys-Trp-Lys-Lys-Gly- |
| | CXP1 | Lys-Gln-Lys-Thr-Ser-Iph-Gly-Arg-Gly-Pip; |
| 40 | CXP3- | Dab-Val-Ala-Arg-Asn-Asn-Dab-Thr-Lys-Gly-Lys-Trp-Lys-Lys-Gly- |
| | CXP1 | Lys-Gln-Lys-Thr-Ser-Iph-Gly-Arg-Gly-Pip; |
| 41 | CXP4- | Cit-Met-Phe-Gly-Pip-Dab-Dab-Lys-Ala-Pro-Lys-Trp-Lys-Lys-Gly- |
| | CXP1 | Lys-Gln-Lys-Thr-Ser-Iph-Gly-Arg-Gly-Pip; |
| 42 | SA-CXP1 | Iph-Gly-Arg-Gly-Pip-Lys-Gln-Lys-Thr-Ser-Iph-Gly-Arg-Gly-Pip; |
| 43 | SB-CXP1 | Lys-Thr-Ser-Iph-Gly-Arg-Lys-Gln-Lys-Thr-Ser-Iph-Gly-Arg-Gly- |
| | | Pip; |
| 44 | SC-CXP1 | Lys-Gln-Lys-Thr-Ser-Lys-Gln-Lys-Thr-Ser-Iph-Gly-Arg-Gly-Pip; |
| 45 | SD-CPX1 | Lys-Trp-Lys-Lys-Gln-Lys-Thr-Ser-Iph-Gly-Arg-Gly-Pip; |
| 46 | CXD1A | Lys-Thr-Ser-Iph-Gly-Arg-Lys-Gln-Lys-Thr-Ser-Iph-Gly-Arg; |
| 47 | CXD1B | Lys-Gln-Lys-Thr-Ser-Iph-Gly-Arg-Lys-Trp-Lys-Gln-Lys-Thr-Ser- |
| | | Iph-Gly-Arg; |
| 108 | CXP1X | Lys-Gln-Lys-Thr-Ser-Phe-Gly-Arg-Gly-Pip; |
| 109 | CXP1Y | Lys-Gln-Lys-Thr-Ser-Iph-Gly-Arg-Gly-Lys; |
| 110 | CXP1Z | Lys-Gln-Lys-Thr-Ser-Phe-Gly-Arg-Gly-Lys; |
| 111 | CXP1AA | Lys-Gln-Lys-Thr-Ser-Iph-Gly-Lys-Gly-Pip; |
| 112 | CXP1P | Lys-Gln-Lys-Thr-Ser-Iph-Ala-Arg-Ala-Pip; |
| 113 | CXP1B | Ala-Gln-Lys-Thr-Ser-Iph-Gly-Arg-Gly-Pip; |
| 114 | CXP1C | Lys-Ala-Lys-Thr-Ser-Iph-Gly-Arg-Gly-Pip; |
| 115 | CXP1D | Lys-Gln-Ala-Thr-Ser-Iph-Gly-Arg-Gly-Pip; |
| 116 | CXP1E | Lys-Gln-Lys-Ala-Ser-Iph-Gly-Arg-Gly-Pip; |
| 117 | CXP1F | Lys-Gln-Lys-Thr-Ala-Iph-Gly-Arg-Gly-Pip; |
| 118 | CXP1G | Lys-Gln-Lys-Thr-Ser-Ala-Gly-Arg-Gly-Pip; |
| 119 | CXP1H | Lys-Gln-Lys-Thr-Ser-Iph-Ala-Arg-Gly-Pip; |
| 120 | CXP1I | Lys-Gln-Lys-Thr-Ser-Iph-Gly-Ala-Gly-Pip; |
| 121 | CXP1J | Lys-Gln-Lys-Thr-Ser-Iph-Gly-Arg-Ala-Pip; |
| 122 | CXP1K | Lys-Gln-Lys-Thr-Ser-Iph-Gly-Arg-Gly-Ala; |
| 123 | CXP1L | Lys-Gln-Lys-Thr-Ser-Iph-Gly-Arg-Gly-Pip-Ala; |
| 124 | CXP1M | Lys-Gln-Lys-Thr-Ser-Iph-Gly-Arg-Gly-Pip-Lys-Ala; |
| 125 | CXP1N | Lys-Gln-Lys-Thr-Ser-Iph-Gly-Arg-Gly-Pip-Lys-Trp-Ala; |
| 126 | CXP1O | Lys-Gln-Lys-Thr-Ser-Iph-Gly-Arg-Gly-Pip-Lys-Trp-Lys-Ala; |
| 127 | CXP1Q | Iph-Gln-Lys-Thr-Ser-Iph-Gly-Arg-Gly-Pip; |
| 128 | CXP1R | Lys-Gln-Iph-Thr-Ser-Iph-Gly-Arg-Gly-Pip; |
| 129 | CXP1S | Lys-Gln-Lys-Thr-Ser-Iph-Gly-Iph-Gly-Pip; |
| 130 | CXP1T | Lys-Gln-Lys-Thr-Ser-Iph-Gly-Arg-Gly-Iph; |
| 131 | CXP1U | Lys-Gln-Lys-Thr-Ser-Iph-Gly-Arg-Gly-Pip-Iph-Trp-Lys-Lys; |
| 132 | CXP1V | Lys-Gln-Lys-Thr-Ser-Iph-Gly-Arg-Gly-Pip-Lys-Trp-Iph-Lys; |
| 133 | CXP1W | Lys-Gln-Lys-Thr-Ser-Iph-Gly-Arg-Gly-Pip-Lys-Trp-Lys-Iph; |
| 134 | CXP1AC | Dab-Gln-Dab-Thr-Ser-Iph-Gly-Arg-Gly-Pip-Dab-Trp-Dab-Dab; |
| 135 | CXP1AD | Dab-Gln-Dab-Thr-Ser-Iph-Gly-Dab-Gly-Pip-Dab-Trp-Dab-Dab; |
| 136 | CXP1AE | Dab-Gln-Dab-Thr-Ser-Iph-Gly-Arg-Gly-Dab-Dab-Trp-Dab-Dab; |
| | | and |
| 137 | CXP1AF | Dab-Gln-Dab-Thr-Ser-Iph-Gly-Dab-Gly-Dab-Dab-Trp-Dab-Dab. |

Aspect 8, The antisense oligomer conjugate of Aspect 4 or 5, or a pharmaceutically acceptable salt thereof, wherein X-ph has the formula:

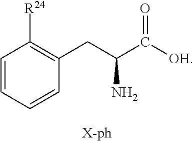

Aspect 9, The antisense oligomer conjugate of Aspect 8, or a pharmaceutically acceptable salt thereof, where R²⁴ is —I.

Aspect 10, The antisense oligomer conjugate of any one of Aspects 4, 5, or 8 to 9, or a pharmaceutically acceptable salt thereof, wherein J^{1B} or J^{2B} is the amino acid sequence Lys-Trp-Lys-Lys (SEQ ID NO: 143).

Aspect 11, The antisense oligomer conjugate of any one of Aspects 4 to 10, or a pharmaceutically acceptable salt thereof, wherein
R¹⁵ is —C₁₋₆alkyl; and
R¹⁶ is —CH₂C(O)NH₂, or R¹⁵ and R¹⁶ together with the nitrogen to which they are attached join to form a ring selected from

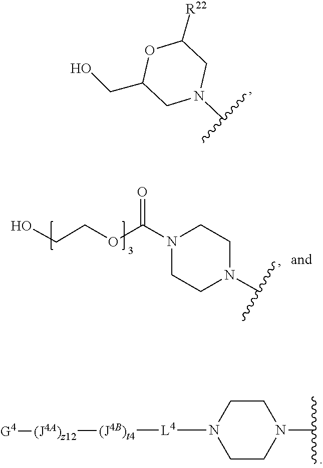

Aspect 12, The antisense oligomer conjugate of any one of Aspects 4 to 11, or a pharmaceutically acceptable salt thereof, wherein E² is selected from hydrogen, —C(O)CH₃, benzoyl, stearoyl, trityl, 4-methoxytrityl, and -L³-(J^{3B})ₜ₃-(J^{3A})_{z11}-G³.

Aspect 13, The antisense oligomer conjugate of any one of Aspects 4 to 12, or a pharmaceutically acceptable salt thereof, wherein the antisense oligomer conjugate is selected from Formula (Va) and (Vb):

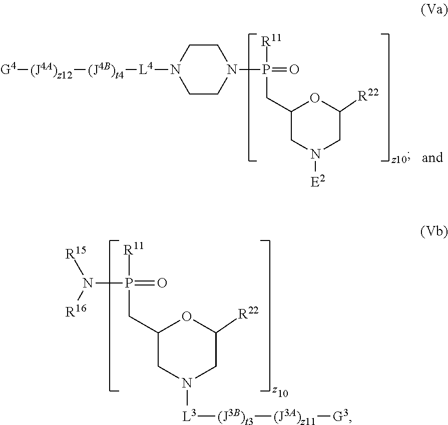

wherein:
E² is selected from hydrogen, —C₁₋₆alkyl, —C(O)CH₃, benzoyl, and stearoyl; and
R¹⁵ together and R¹⁶ together with the nitrogen to which they are attached join to form a ring selected from:

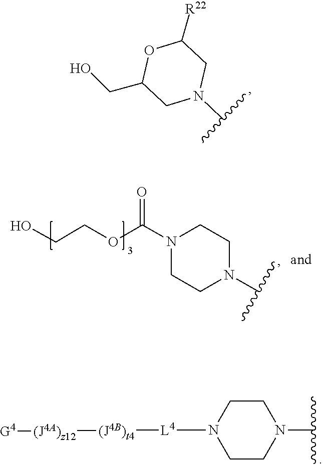

Aspect 14, The antisense oligomer conjugate of any one of Aspects 4 to 13, or a pharmaceutically acceptable salt thereof, wherein the antisense oligomer conjugate is of Formula (Vc) or (Vd):

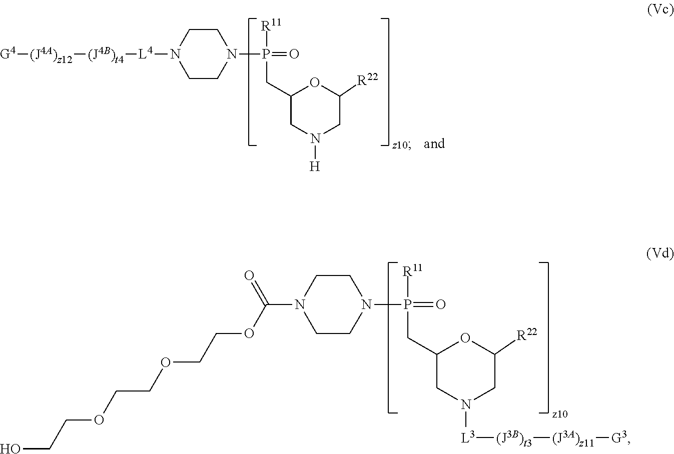

Aspect 15, The antisense oligomer conjugate of Aspect 13, or a pharmaceutically acceptable salt thereof, wherein the antisense oligomer conjugate is of Formula (Va).

Aspect 16, The antisense oligomer conjugate of Aspect 13, or a pharmaceutically acceptable salt thereof, wherein the antisense oligomer conjugate is of Formula (Vb).

Aspect 17, The antisense oligomer conjugate of Aspect 14, or a pharmaceutically acceptable salt thereof, wherein the antisense oligomer conjugate is of Formula (Vc).

Aspect 18, The antisense oligomer conjugate of Aspect 14, or a pharmaceutically acceptable salt thereof, wherein the antisense oligomer conjugate is of Formula (Vd).

Aspect 19, The antisense oligomer conjugate of any one of Aspects 4 to 18, or a pharmaceutically acceptable salt thereof, wherein Y³ and Y⁴ are each independently selected from Gly and Ala. Aspect 20, in certain embodiments Y³ is Gly. Aspect 21, in certain embodiments Y⁴ is Gly.

Aspect 22, The antisense oligomer conjugate of any one of Aspects 4 to 21, or a pharmaceutically acceptable salt thereof, wherein each R¹¹ is —N(CH₃)₂.

Aspect 23, The antisense oligomer conjugate of any one of Aspects 4 to 22, or a pharmaceutically acceptable salt thereof, wherein each R²² is a nucleobase, independently at each occurrence, selected from adenine, guanine, cytosine, 5-methyl-cytosine, thymine, uracil, and hypoxanthine.

Aspect 24, The antisense oligomer conjugate of any one of Aspects 4 to 23, or a pharmaceutically acceptable salt thereof, wherein L³ is

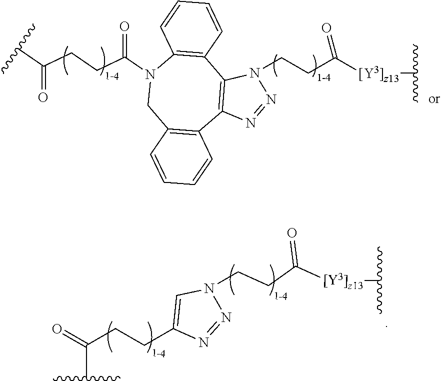

Aspect 25, The antisense oligomer conjugate of any one of Aspects 4 to 23, or a pharmaceutically acceptable salt thereof, wherein L⁴ is

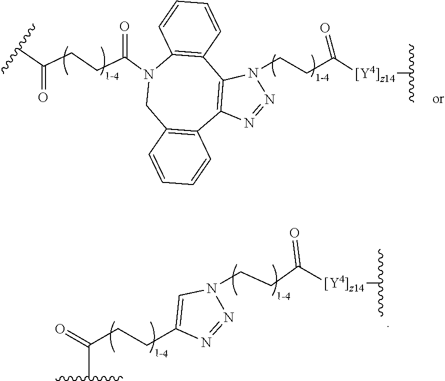

Aspect 26, The antisense oligomer conjugate of any one of Aspects 4 to 23, or a pharmaceutically acceptable salt thereof, wherein L³ is

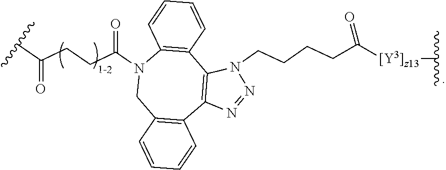

Aspect 27, The antisense oligomer conjugate of any one of Aspects 4 to 23, or a pharmaceutically acceptable salt thereof, wherein L³ is

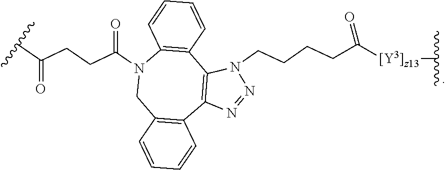

Aspect 28, The antisense oligomer conjugate of any one of Aspects 4 to 23, or a pharmaceutically acceptable salt thereof, wherein L³ is

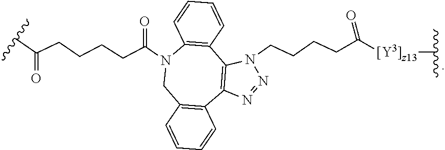

Aspect 29, The antisense oligomer conjugate of any one of Aspects 4 to 28, or a pharmaceutically acceptable salt thereof, wherein G³ is selected from hydrogen, —C(O)CH₃, —NH₂, benzoyl, and stearoyl. Aspect 30, in certain embodiments G³ is —NH₂.

Aspect 31, The antisense oligomer conjugate of any one of Aspects 4, 5, or 8 to 30, or a pharmaceutically acceptable salt thereof, wherein each J^{3B} and each J^{4B} is independently an L-amino acid selected from Arg, His, Lys, Asp, Glu, Ser, Thr, Asn, Gln, Gly, Met, Pro, Val, Ala, Leu, Phe, Trp, 2,4-diaminobutyric acid (Dab), 2-amino-5-ureidopentanoic acid (citrulline (Cit)), 2-amino-3-(naphthalen-2-yl)propanoic acid (Nap), 4-aminopiperidine-4-carboxylic acid (Pip), 2-amino-3-(pyridin-3-yl)propanoic acid (Pyr), and

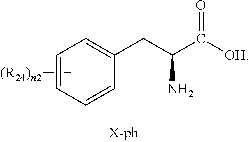

Aspect 32, The antisense oligomer conjugate of any one of Aspects 4, 5, or 8 to 31, or a pharmaceutically acceptable salt thereof, wherein the peptide of J^{3B} or J^{4B} each independently comprise the sequence -(X-ph)-Gly-Arg.

Aspect 33, The antisense oligomer conjugate of any one of Aspects 4, 5, or 8 to 31, or a pharmaceutically acceptable salt thereof, wherein the peptide of J^{3B} or J^{4B} each independently comprise at least one unnatural amino acid selected from 2,4-diaminobutyric acid (Dab), 2-amino-5-ureidopentanoic acid (citrulline (Cit)), 2-amino-3-(naphthalen-2-yl)propanoic acid (Nap), 4-aminopiperidine-4-carboxylic acid (Pip), 2-amino-3-(pyridin-3-yl)propanoic acid (Pyr), and

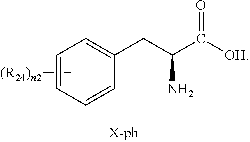

Aspect 34, The antisense oligomer conjugate of any one of Aspects 4, 5, or 8 to 31, or a pharmaceutically acceptable salt thereof, wherein the peptide of J^{3B} or J^{4B} each independently comprise at least one unnatural amino acid selected from 2-amino-5-ureidopentanoic acid (citrulline (Cit)), 2-amino-3-(pyridin-3-yl)propanoic acid (Pyr), and

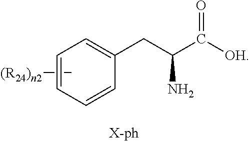

Aspect 35, The antisense oligomer conjugate of any one of Aspects 4, 5, or 8-31, or a pharmaceutically acceptable salt thereof, wherein the peptide of J^{3B} or J^{4B} each independently comprise at least one

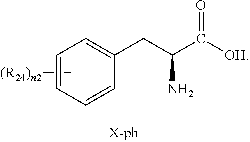

Aspect 36, The antisense oligomer conjugate of Aspect 35, or a pharmaceutically acceptable salt thereof, wherein the peptide of J^{3B} or J^{4B} each independently further comprises at least one further comprises at least one 2,4-diaminobutyric acid (Dab) or 4-aminopiperidine-4-carboxylic acid (Pip).

Aspect 37, The antisense oligomer conjugate of any one of Aspects 4 to 36, or a pharmaceutically acceptable salt thereof, wherein the antisense oligomer conjugate is an antisense oligomer conjugate of Formula (VI):

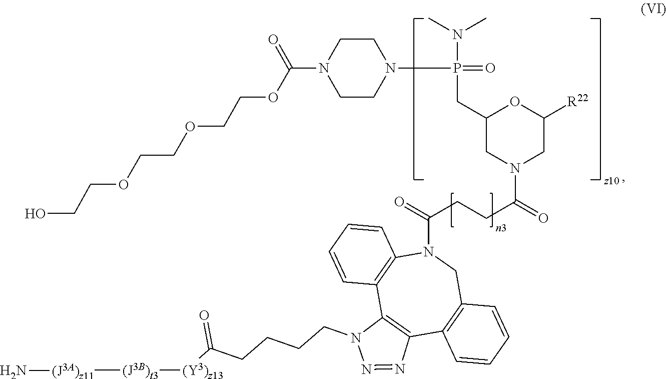

wherein n3 is 1 or 2.

Aspect 38, The antisense oligomer conjugate of Aspect 37, w or a pharmaceutically acceptable salt thereof, herein the antisense oligomer conjugate is an antisense oligomer conjugate of Formula (VIA):

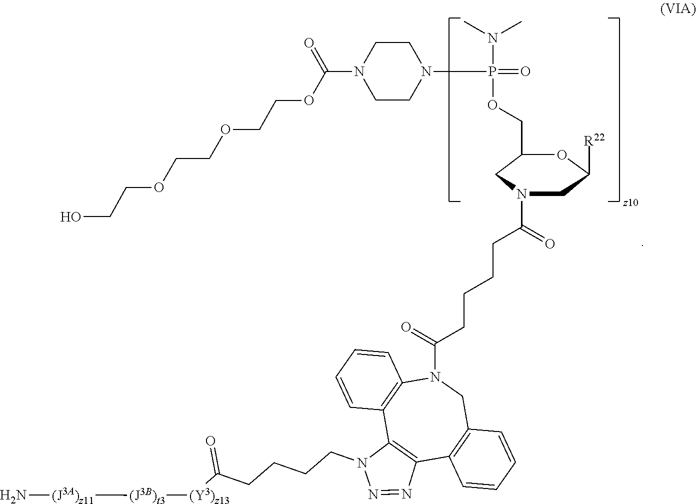

Aspect 39, The antisense oligomer conjugate of Aspect 37, or a pharmaceutically acceptable salt thereof, wherein n3 is 1.

Aspect 40, The antisense oligomer conjugate of Aspect 37, or a pharmaceutically acceptable salt thereof, wherein n3 is 2.

Aspect 41, The antisense oligomer conjugate of any one of Aspects 4 to 36, or a pharmaceutically acceptable salt thereof, wherein the antisense oligomer conjugate is an antisense oligomer conjugate of Formula (VII):

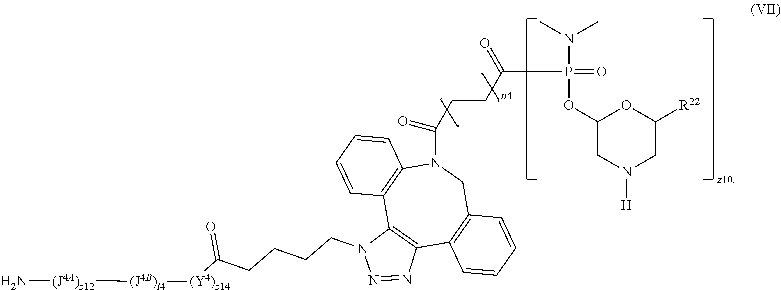

wherein n4 is 1 or 2.

Aspect 42, The antisense oligomer conjugate of Aspect 41, or a pharmaceutically acceptable salt thereof, wherein the antisense oligomer conjugate is an antisense oligomer conjugate of Formula (VIIA):

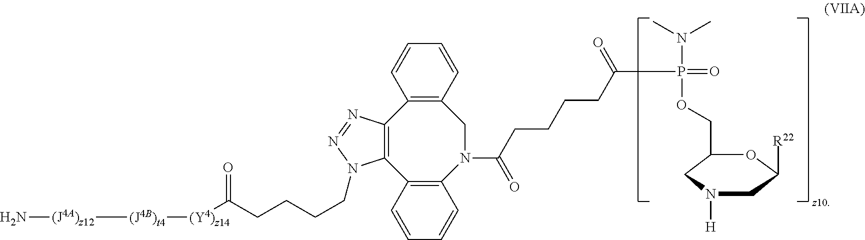

Aspect 43, The antisense oligomer conjugate of Aspect 41, or a pharmaceutically acceptable salt thereof, wherein n4 is 1.

Aspect 44, The antisense oligomer conjugate of Aspect 41, or a pharmaceutically acceptable salt thereof, wherein n4 is 2.

Aspect 45, The antisense oligomer conjugate of any one of Aspects 4 to 24 or 26 to 40, or a pharmaceutically acceptable salt thereof, wherein z13 is 1.

Aspect 46, The antisense oligomer conjugate of any one of Aspects 4 to 23, 25, 29 to 36, or 41 to 44, or a pharmaceutically acceptable salt thereof, wherein z14 is 1.

Aspect 47, The antisense oligomer conjugate of any one of Aspects 4 to 24, 26 to 40, or 45, or a pharmaceutically acceptable salt thereof, wherein Y³ is selected from Gly and Ala.

Aspect 49, in certain embodiments Y³ is Gly.

Aspect 48, The antisense oligomer conjugate of any one of Aspects 4 to 23, 25, 29 to 36, 41 to 44, or 46, or a pharmaceutically acceptable salt thereof, wherein Y⁴ is selected from Gly and Ala. Aspect 50, in certain embodiments Y⁴ is Gly.

Aspect 51, The antisense oligomer conjugate of any one of Aspects 4, 5, or 8 to 50, or a pharmaceutically acceptable salt thereof, wherein -(J^{3B})ₜ₃-(J^{3A})_{z11}-and-(J^{4B})ₜ₄-(J^{4A})_{z12}- each independently form a peptide sequence selected from:

| SEQ ID | Peptide | |
|---|---|---|
| NO. | Name | Sequence |
| 48 | CXP1 | Lys-Gln-Lys-Thr-Ser-Iph-Gly-Arg-Gly-Pip-Lys-Trp-Lys-Lys; |
| 49 | CXP2 | Asn-Phe-Lys-Gln-Nap-His-Ala-Gly-Dab-Arg-Lys-Trp-Lys-Lys; |
| 50 | CXP3 | Dab-Val-Ala-Arg-Asn-Asn-Dab-Thr-Lys-Gly-Lys-Trp-Lys-Lys; |
| 51 | CXP4 | Cit-Met-Phe-Gly-Pip-Dab-Dab-Lys-Ala-Pro-Lys-Trp-Lys-Lys; |
| 52 | CXP5 | Dab-Pro-Arg-Dab-Leu-His-Ser-Dab-Ala-Thr-Lys-Trp-Lys-Lys; |
| 53 | CXP6 | Pro-Pip-Gly-Dab-Lys-Dab-Ser-Val-Iph-Nap-Lys-Trp-Lys-Lys; |
| 54 | CXP7 | Val-Gln-His-Iph-Arg-Dab-Lys-Glu-Lys-Asn-Lys-Trp-Lys-Lys; |
| 55 | CXP8 | Asn-Phe-Asn-Asn-Arg-Ser-Dab-Gly-His-Lys-Lys-Trp-Lys-Lys; |
| 56 | CXP9 | Ala-Arg-Ser-Glu-Gln-His-His-Nap-Ser-Dab-Lys-Trp-Lys-Lys; |
| 57 | SA | Iph-Gly-Arg-Gly-Pip-Lys-Trp-Lys-Lys; |
| 58 | SB | Lys-Thr-Ser-Iph-Gly-Arg-Lys-Trp-Lys-Lys; |
| 61 | SE | Iph-Gly-Arg-Lys-Trp-Lys-Lys; |
| 62 | SF | Lys-Gln-Lys-Thr-Ser-Iph-Gly-Arg-Lys-Trp-Lys-Lys; |
| 63 | SG | Ser-Iph-Gly-Arg-Lys-Trp-Lys-Lys; |
| 65 | SI | Gln-Lys-Thr-Ser-Iph-Gly-Arg-Lys-Trp-Lys-Lys; |
| 66 | SJ | Lys-Gln-Lys-Thr-Ser-Iph-Gly-Arg-Lys-Trp-Lys-Lys; |
| 67 | CXP1- | Lys-Gln-Lys-Thr-Ser-Iph-Gly-Arg-Gly-Pip-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 68 | CXP3- | Dab-Val-Ala-Arg-Asn-Asn-Dab-Thr-Lys-Gly-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 69 | CXP4- | Cit-Met-Phe-Gly-Pip-Dab-Dab-Lys-Ala-Pro-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 70 | SA-CXP1 | Iph-Gly-Arg-Gly-Pip-Lys-Gln-Lys-Thr-Ser-Iph-Gly-Arg-Gly-Pip-Lys- |
| | | Trp-Lys-Lys; |
| 71 | SB-CXP1 | Lys-Thr-Ser-Iph-Gly-Arg-Lys-Gln-Lys-Thr-Ser-Iph-Gly-Arg-Gly-Pip- |
| | | Lys-Trp-Lys-Lys; |
| 72 | SC-CXP1 | Lys-Gln-Lys-Thr-Ser-Lys-Gln-Lys-Thr-Ser-Iph-Gly-Arg-Gly-Pip- |
| | | Lys-Trp-Lys-Lys; |
| 73 | SD-CXP1 | Lys-Trp-Lys-Lys-Gln-Lys-Thr-Ser-Iph-Gly-Arg-Gly-Pip-Lys-Trp- |
| | | Lys-Lys; |
| 74 | CXD1A | Lys-Thr-Ser-Iph-Gly-Arg-Lys-Gln-Lys-Thr-Ser-Iph-Gly-Arg-Lys- |
| | | Trp-Lys-Lys; |
| 75 | CXD1B | Lys-Gln-Lys-Thr-Ser-Iph-Gly-Arg-Lys-Trp-Lys-Gln-Lys-Thr-Ser- |
| | | Iph-Gly-Arg-Lys-Trp-Lys-Lys; |
| 78 | CXP1X | Lys-Gln-Lys-Thr-Ser-Phe-Gly-Arg-Gly-Pip-Lys-Trp-Lys-Lys; |
| 79 | CXP1Y | Lys-Gln-Lys-Thr-Ser-Iph-Gly-Arg-Gly-Lys-Lys-Trp-Lys-Lys; |
| 80 | CXP1Z | Lys-Gln-Lys-Thr-Ser-Phe-Gly-Arg-Gly-Lys-Lys-Trp-Lys-Lys; |
| 81 | CXP1AA | Lys-Gln-Lys-Thr-Ser-Iph-Gly-Lys-Gly-Pip-Lys-Trp-Lys-Lys; |
| 82 | CXP1P | Lys-Gln-Lys-Thr-Ser-Iph-Ala-Arg-Ala-Pip-Lys-Trp-Lys-Lys; |
| 83 | CXP1B | Ala-Gln-Lys-Thr-Ser-Iph-Gly-Arg-Gly-Pip-Lys-Trp-Lys-Lys; |
| 84 | CXP1C | Lys-Ala-Lys-Thr-Ser-Iph-Gly-Arg-Gly-Pip-Lys-Trp-Lys-Lys; |
| 85 | CXP1D | Lys-Gln-Ala-Thr-Ser-Iph-Gly-Arg-Gly-Pip-Lys-Trp-Lys-Lys; |
| 86 | CXP1E | Lys-Gln-Lys-Ala-Ser-Iph-Gly-Arg-Gly-Pip-Lys-Trp-Lys-Lys; |
| 87 | CXP1F | Lys-Gln-Lys-Thr-Ala-Iph-Gly-Arg-Gly-Pip-Lys-Trp-Lys-Lys; |
| 88 | CXP1G | Lys-Gln-Lys-Thr-Ser-Ala-Gly-Arg-Gly-Pip-Lys-Trp-Lys-Lys; |
| 89 | CXP1H | Lys-Gln-Lys-Thr-Ser-Iph-Ala-Arg-Gly-Pip-Lys-Trp-Lys-Lys; |
| 90 | CXP1I | Lys-Gln-Lys-Thr-Ser-Iph-Gly-Ala-Gly-Pip-Lys-Trp-Lys-Lys; |
| 91 | CXP1J | Lys-Gln-Lys-Thr-Ser-Iph-Gly-Arg-Ala-Pip-Lys-Trp-Lys-Lys; |
| 92 | CXP1K | Lys-Gln-Lys-Thr-Ser-Iph-Gly-Arg-Gly-Ala-Lys-Trp-Lys-Lys; |
| 93 | CXP1L | Lys-Gln-Lys-Thr-Ser-Iph-Gly-Arg-Gly-Pip-Ala-Trp-Lys-Lys; |
| 94 | CXP1M | Lys-Gln-Lys-Thr-Ser-Iph-Gly-Arg-Gly-Pip-Lys-Ala-Lys-Lys; |
| 95 | CXP1N | Lys-Gln-Lys-Thr-Ser-Iph-Gly-Arg-Gly-Pip-Lys-Trp-Ala-Lys; |
| 96 | CXP1O | Lys-Gln-Lys-Thr-Ser-Iph-Gly-Arg-Gly-Pip-Lys-Trp-Lys-Ala-Lys-Trp- |
| | | Lys-Lys; |
| 97 | CXP1Q | Iph-Gln-Lys-Thr-Ser-Iph-Gly-Arg-Gly-Pip-Lys-Trp-Lys-Lys; |
| 98 | CXP1R | Lys-Gln-Iph-Thr-Ser-Iph-Gly-Arg-Gly-Pip-Lys-Trp-Lys-Lys; |
| 99 | CXP1S | Lys-Gln-Lys-Thr-Ser-Iph-Gly-Iph-Gly-Pip-Lys-Trp-Lys-Lys; |
| 100 | CXP1T | Lys-Gln-Lys-Thr-Ser-Iph-Gly-Arg-Gly-Iph-Lys-Trp-Lys-Lys; |
| 101 | CXP1U | Lys-Gln-Lys-Thr-Ser-Iph-Gly-Arg-Gly-Pip-Iph-Trp-Lys-Lys; |
| 102 | CXP1V | Lys-Gln-Lys-Thr-Ser-Iph-Gly-Arg-Gly-Pip-Lys-Trp-Iph-Lys-Lys- |
| | | Trp-Lys-Lys; |
| 103 | CXP1W | Lys-Gln-Lys-Thr-Ser-Iph-Gly-Arg-Gly-Pip-Lys-Trp-Lys-Iph-Lys; |
| 104 | CXP1AC | Dab-Gln-Dab-Thr-Ser-Iph-Gly-Arg-Gly-Pip-Dab-Trp-Dab-Dab-Lys- |
| | | Trp-Lys-Lys; |
| 105 | CXP1AD | Dab-Gln-Dab-Thr-Ser-Iph-Gly-Dab-Gly-Pip-Dab-Trp-Dab-Dab-Lys- |
| | | Trp-Lys-Lys; |
| 106 | CXP1AE | Dab-Gln-Dab-Thr-Ser-Iph-Gly-Arg-Gly-Dab-Dab-Trp-Dab-Dab- |
| | | Lys-Trp-Lys-Lys; and |
| 107 | CXP1AF | Dab-Gln-Dab-Thr-Ser-Iph-Gly-Dab-Gly-Dab-Dab-Trp-Dab-Dab- |
| | | Lys-Trp-Lys-Lys. |

Aspect 52, The antisense oligomer conjugate of any one of Aspects 4, 5, or 8 to 50, or a pharmaceutically acceptable salt thereof, where -(J^{3B})ₜ₃- and -(J^{4B})ₜ₄- each independently form a peptide sequence selected from:

| SEQ ID | Peptide | |
|---|---|---|
| NO. | Name | Sequence |
| 1 | CXP1 | Lys-Gln-Lys-Thr-Ser-Iph-Gly-Arg-Gly-Pip; |
| 2 | CXP2 | Asn-Phe-Lys-Gln-Nap-His-Ala-Gly-Dab-Arg; |
| 3 | CXP3 | Dab-Val-Ala-Arg-Asn-Asn-Dab-Thr-Lys-Gly; |
| 4 | CXP4 | Cit-Met-Phe-Gly-Pip-Dab-Dab-Lys-Ala-Pro; |
| 5 | CXP5 | Dab-Pro-Arg-Dab-Leu-His-Ser-Dab-Ala-Thr; |
| 6 | CXP6 | Pro-Pip-Gly-Dab-Lys-Dab-Ser-Val-Iph-Nap; |
| 7 | CXP7 | Val-Gln-His-Iph-Arg-Dab-Lys-Glu-Lys-Asn; |
| 8 | CXP8 | Asn-Phe-Asn-Asn-Arg-Ser-Dab-Gly-His-Lys; |
| 9 | CXP9 | Ala-Arg-Ser-Glu-Gln-His-His-Nap-Ser-Dab; |
| 10 | SA | Iph-Gly-Arg-Gly-Pip; |
| 11 | SB | Lys-Thr-Ser-Iph-Gly-Arg; |
| 12 | SC | Lys-Gln-Lys-Thr-Ser; |
| 13 | SD | Lys-Trp-Lys; |
| 14 | SE | Iph-Gly-Arg; |
| 15 | SF | Lys-Gln-Lys-Thr-Ser-Iph-Gly-Arg; |
| 16 | SG | Ser-Iph-Gly-Arg; |
| 18 | SI | Gln-Lys-Thr-Ser-Iph-Gly-Arg; |
| 19 | SJ | Lys-Gln-Lys-Thr-Ser-Iph-Gly-Arg; |
| 39 | CXP1- | Lys-Gln-Lys-Thr-Ser-Iph-Gly-Arg-Gly-Pip-Lys-Trp-Lys-Lys-Gly- |
| | CXP1 | Lys-Gln-Lys-Thr-Ser-Iph-Gly-Arg-Gly-Pip; |
| 40 | CXP3- | Dab-Val-Ala-Arg-Asn-Asn-Dab-Thr-Lys-Gly-Lys-Trp-Lys-Lys-Gly- |
| | CXP1 | Lys-Gln-Lys-Thr-Ser-Iph-Gly-Arg-Gly-Pip; |
| 41 | CXP4- | Cit-Met-Phe-Gly-Pip-Dab-Dab-Lys-Ala-Pro-Lys-Trp-Lys-Lys-Gly- |
| | CXP1 | Lys-Gln-Lys-Thr-Ser-Iph-Gly-Arg-Gly-Pip; |
| 42 | SA-CXP1 | Iph-Gly-Arg-Gly-Pip-Lys-Gln-Lys-Thr-Ser-Iph-Gly-Arg-Gly-Pip; |
| 43 | SB-CXP1 | Lys-Thr-Ser-Iph-Gly-Arg-Lys-Gln-Lys-Thr-Ser-Iph-Gly-Arg-Gly- |
| | | Pip; |
| 44 | SC-CXP1 | Lys-Gln-Lys-Thr-Ser-Lys-Gln-Lys-Thr-Ser-Iph-Gly-Arg-Gly-Pip; |
| 45 | SD-CPX1 | Lys-Trp-Lys-Lys-Gln-Lys-Thr-Ser-Iph-Gly-Arg-Gly-Pip; |
| 46 | CXD1A | Lys-Thr-Ser-Iph-Gly-Arg-Lys-Gln-Lys-Thr-Ser-Iph-Gly-Arg; |
| 47 | CXD1B | Lys-Gln-Lys-Thr-Ser-Iph-Gly-Arg-Lys-Trp-Lys-Gln-Lys-Thr-Ser- |
| | | Iph-Gly-Arg; |
| 108 | CXP1X | Lys-Gln-Lys-Thr-Ser-Phe-Gly-Arg-Gly-Pip; |
| 109 | CXP1Y | Lys-Gln-Lys-Thr-Ser-Iph-Gly-Arg-Gly-Lys; |
| 110 | CXP1Z | Lys-Gln-Lys-Thr-Ser-Phe-Gly-Arg-Gly-Lys; |
| 111 | CXP1AA | Lys-Gln-Lys-Thr-Ser-Iph-Gly-Lys-Gly-Pip; |
| 112 | CXP1P | Lys-Gln-Lys-Thr-Ser-Iph-Ala-Arg-Ala-Pip; |
| 113 | CXP1B | Ala-Gln-Lys-Thr-Ser-Iph-Gly-Arg-Gly-Pip; |
| 114 | CXP1C | Lys-Ala-Lys-Thr-Ser-Iph-Gly-Arg-Gly-Pip; |
| 115 | CXP1D | Lys-Gln-Ala-Thr-Ser-Iph-Gly-Arg-Gly-Pip; |
| 116 | CXP1E | Lys-Gln-Lys-Ala-Ser-Iph-Gly-Arg-Gly-Pip; |
| 117 | CXP1F | Lys-Gln-Lys-Thr-Ala-Iph-Gly-Arg-Gly-Pip; |
| 118 | CXP1G | Lys-Gln-Lys-Thr-Ser-Ala-Gly-Arg-Gly-Pip; |
| 119 | CXP1H | Lys-Gln-Lys-Thr-Ser-Iph-Ala-Arg-Gly-Pip; |
| 120 | CXP1I | Lys-Gln-Lys-Thr-Ser-Iph-Gly-Ala-Gly-Pip; |
| 121 | CXP1J | Lys-Gln-Lys-Thr-Ser-Iph-Gly-Arg-Ala-Pip; |
| 122 | CXP1K | Lys-Gln-Lys-Thr-Ser-Iph-Gly-Arg-Gly-Ala; |
| 123 | CXP1L | Lys-Gln-Lys-Thr-Ser-Iph-Gly-Arg-Gly-Pip-Ala; |
| 124 | CXP1M | Lys-Gln-Lys-Thr-Ser-Iph-Gly-Arg-Gly-Pip-Lys-Ala; |
| 125 | CXP1N | Lys-Gln-Lys-Thr-Ser-Iph-Gly-Arg-Gly-Pip-Lys-Trp-Ala; |
| 126 | CXP1O | Lys-Gln-Lys-Thr-Ser-Iph-Gly-Arg-Gly-Pip-Lys-Trp-Lys-Ala; |
| 127 | CXP1Q | Iph-Gln-Lys-Thr-Ser-Iph-Gly-Arg-Gly-Pip; |
| 128 | CXP1R | Lys-Gln-Iph-Thr-Ser-Iph-Gly-Arg-Gly-Pip; |
| 129 | CXP1S | Lys-Gln-Lys-Thr-Ser-Iph-Gly-Iph-Gly-Pip; |
| 130 | CXP1T | Lys-Gln-Lys-Thr-Ser-Iph-Gly-Arg-Gly-Iph; |
| 131 | CXP1U | Lys-Gln-Lys-Thr-Ser-Iph-Gly-Arg-Gly-Pip-Iph-Trp-Lys-Lys; |
| 132 | CXP1V | Lys-Gln-Lys-Thr-Ser-Iph-Gly-Arg-Gly-Pip-Lys-Trp-Iph-Lys; |
| 133 | CXP1W | Lys-Gln-Lys-Thr-Ser-Iph-Gly-Arg-Gly-Pip-Lys-Trp-Lys-Iph; |
| 134 | CXP1AC | Dab-Gln-Dab-Thr-Ser-Iph-Gly-Arg-Gly-Pip-Dab-Trp-Dab-Dab; |
| 135 | CXP1AD | Dab-Gln-Dab-Thr-Ser-Iph-Gly-Dab-Gly-Pip-Dab-Trp-Dab-Dab; |
| 136 | CXP1AE | Dab-Gln-Dab-Thr-Ser-Iph-Gly-Arg-Gly-Dab-Dab-Trp-Dab-Dab; |
| | | and |
| 137 | CXP1AF | Dab-Gln-Dab-Thr-Ser-Iph-Gly-Dab-Gly-Dab-Dab-Trp-Dab-Dab. |

Aspect 53, The antisense oligomer conjugate of any one of Aspects 1 to 52, or a pharmaceutically acceptable salt thereof, wherein the salt is an acetate salt or a chloride salt.

Aspect 54, The antisense oligomer conjugate of any one of Aspects 1 to 52, or a pharmaceutically acceptable salt thereof, wherein the antisense oligomer conjugate is an antisense oligomer conjugate of Formula (VIIIa) or (VIIIb):

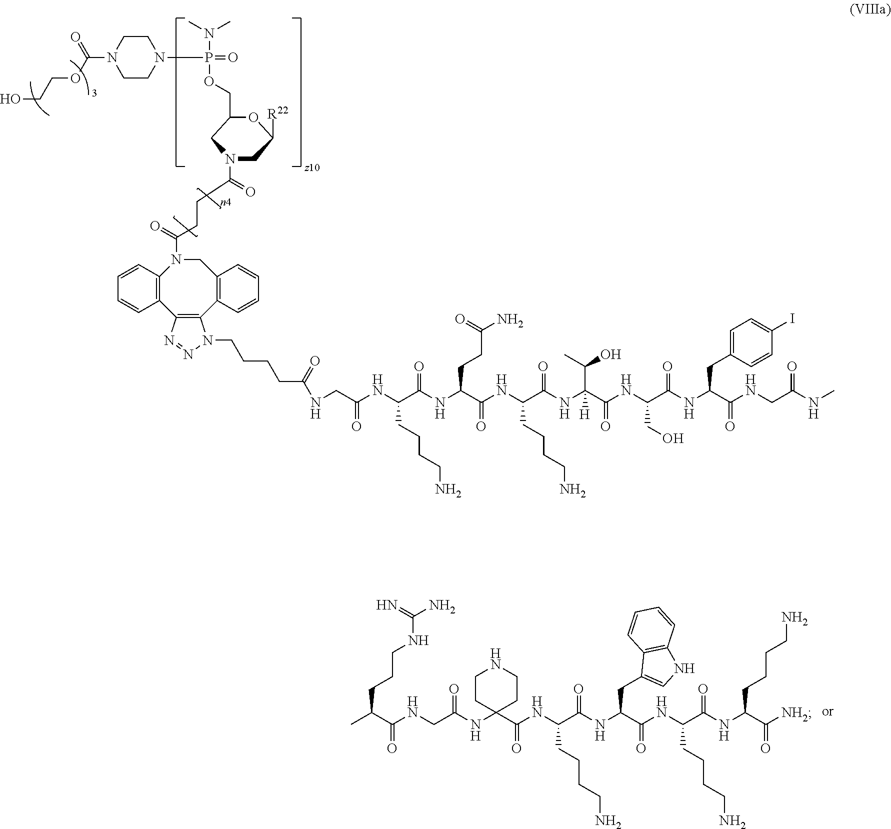

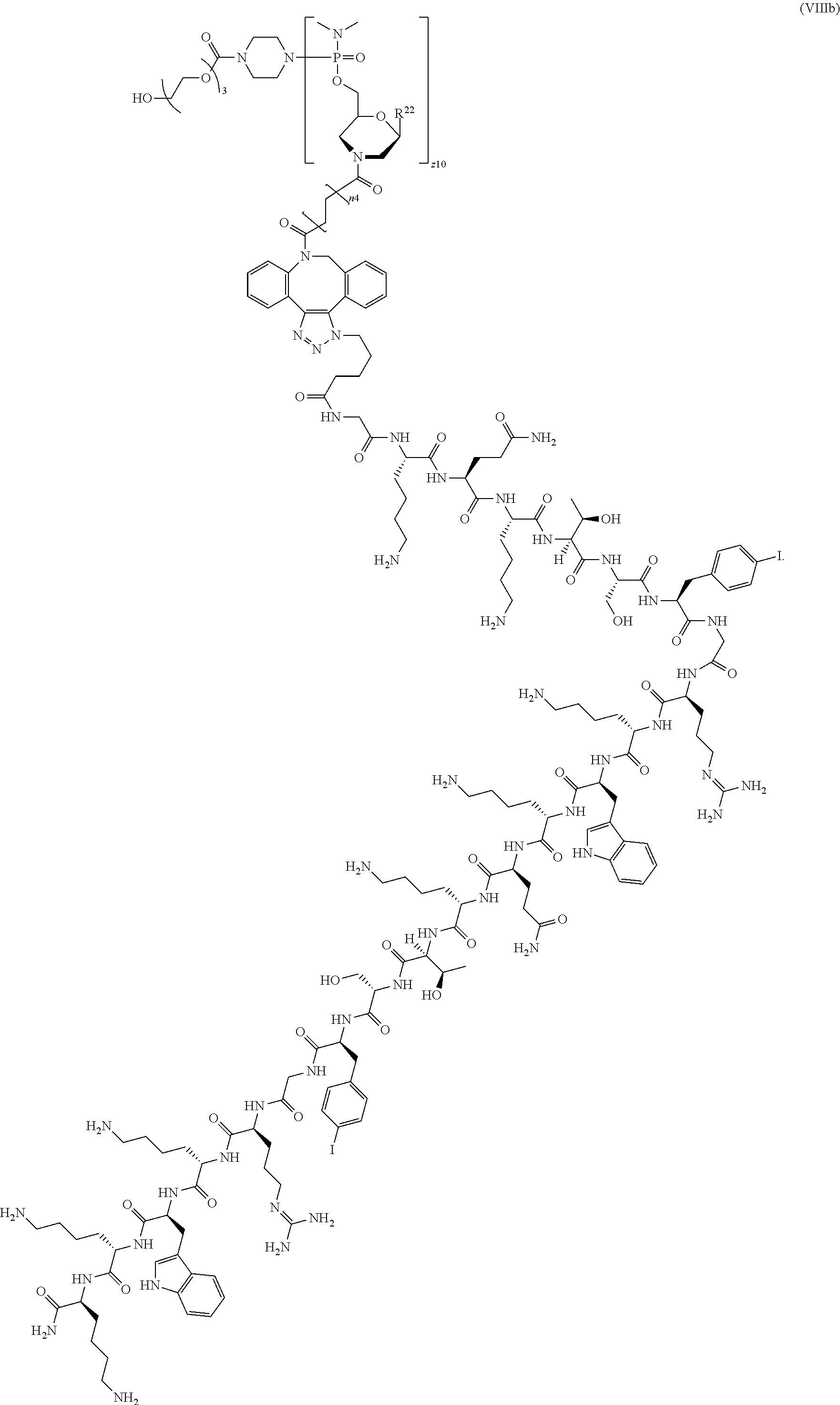

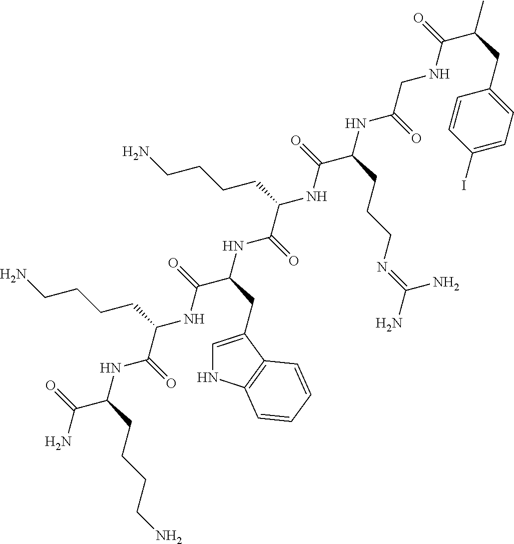 [SMILES: C[C@@H](Cc1ccc(I)cc1)C(=O)NCC(=O)N[C@@H](CCCN=C(N)N)C(=O)N[C@@H](CCCCN)C(=O)N[C@@H](Cc1c[nH]c2ccccc12)C(=O)N[C@@H](CCCCN)C(=O)N[C@@H](CCCCN)C(N)=O]

Aspect 55, provided herein is a pharmaceutical composition comprising an antisense oligomer conjugate of any one of Aspects 1 to 54, or a pharmaceutically acceptable salt thereof, and at least one pharmaceutically acceptable carrier.

Aspect 56, provided herein is a method of treating a disease in a subject in need thereof, comprising administering to the subject an antisense oligomer conjugate of any one of Aspects 1 to 54, or a pharmaceutically acceptable salt thereof, or a composition of Aspect 55 to the subject. Aspect 57, in certain embodiments the disease is a neuromuscular disease. Aspect 58, in certain embodiments the neuromuscular disease is Duchenne muscular dystrophy (DMD).

Aspect, 59, provided herein is the use of an antisense oligomer conjugate of any one of Aspects 1 to 54 or a pharmaceutically acceptable salt thereof, in the manufacture of a medicament for treating a disease.

Aspect 60, provided herein is an antisense oligomer conjugate, or a pharmaceutically acceptable salt thereof, of any one of Aspects 1 to 54, or a composition of Aspect 55, for use in treating a disease.

Additional aspects appear prior to the claims.

Cell-penetrating peptides (CPPs) can help treat disease by enhancing the delivery of cell-impermeable cargo. CPPs are a class of peptides 5-30 amino acid residues in length that are capable of directly entering the cell cytosol (Wolfe Justin M.; Fadzen Colin M.; Holden Rebecca L.; Yao Monica; Hanson Gunnar J.; Pentelute Bradley L. Angew. Chem. Int. Ed. 2018, 57, 4756-4759.; Oehlke, J.; Scheller, A.; Wiesner, B.; Krause, E.; Beyermann, M.; Klauschenz, E.; Melzig, M.; Bienert, M. Biochim. Biophys. Acta BBA—Biomembr. 1998, 1414 (1), 127-139; Margus, H.; Padari, K.; Pooga, M. Mol. Ther. J. Am. Soc. Gene Ther. 2012, 20 (3), 525-533). These sequences can deliver covalently bound cargo, offering therapeutic potential to macromolecules otherwise restricted to extracellular targets. Although CPPs have been widely studied since their discovery, the field lacks robust methodology to quantify cell entry and penetration efficacy. This dearth of knowledge is due to the complicated mechanisms of CPP cell entry and the many variables that affect CPP efficacy in any given assay—such as peptide concentration, cell type, temperature, treatment time, and cargo (Reissmann Siegmund. J. Pept. Sci. 2014, 20 (10), 760-784) For the well-studied CPP penetratin (RQIKIWFQNRRMKWKK (SEQ ID NO: 76)), the reported ratio between intracellular and extracellular concentration ranges from 0.6:1.0 to 95.0:1.0 (Fischer, R.; Waizenegger, T.; Köhler, K.; Brock, R. A Biochim. Biophys. Acta BBA—Biomembr. 2002, 1564 (2), 365-374; Lindgren, M. E.; Hallbrink, M. M.; Elmquist, A. M.; Langel, U. Biochem. J. 2004, 377 (Pt 1), 69-76.) In addition, it is challenging to determine subcellular localization once a peptide is internalized, despite advances in fluorescence, immunoblot, and mass spectrometry detection (Illien, F.; Rodriguez, N.; Amoura, M.; Joliot, A.; Pallerla, M.; Cribier, S.; Burlina, F.; Sagan, S. Sci. Rep. 2016, 6, 36938.). The choice of CPP cargo adds an additional confounding factor. The cell-penetrating ability of more than ten common CPPs differs when bound to a cyanine dye versus a macromolecular drug, with no discernable trend having been previously described (Wolfe, J. M.; Fadzen, C. M.; Choo, Z.-N.; Holden, R. L.; Yao, M.; Hanson, G. J.; Pentelute, B. L. ACS Cent. Sci. 2018, 4 (4), 512-520). As a result, effective development of CPPs require a new methodology for understanding CPP cell entry and subcellular localization that can be carried out on the CPP-cargo conjugate.

There are several limitations that have slowed the clinical advancement of CPPs. Historically, CPPs, also known as protein transduction domains (PTDs), were derived from transmembrane portions of viral and transcriptional proteins. For example, the polyarginine peptide TAT was derived from the HIV-transactivator of transcription protein and was found to penetrate the nucleus and target gene expression (Frankel, A. D.; Pabo, C. O. Cell 1988, 55 (6), 1189-1193; Green, M.; Loewenstein, P. M. Cell 1988, 55 (6), 1179-1188). From this and similar sequences, synthetic peptides could be designed, including some tailored for delivery of PMO cargo such as Bpeptide, which relies on arginine to trigger uptake and the unnatural residues β-alanine and 6-amino-hexanoic acid to trigger endosomal escape (Jearawiriyapaisarn, N. et al. Molecular Therapy 2008, 16 (9), 1624-1629). Beyond empirical design using derivatives of polyarginine sequences, the rational design of new sequences remains challenging. Methods involving some rational design include synthetic molecular evolution (Wimley, W. C. In Cell Penetrating Peptides: Methods and Protocols; Langel, Ü., Ed.; Methods in Molecular Biology; Springer US: New York, NY, 2022; pp 73-89; Kauffman, W. B. et al. Nature Communications 2018, 9 (1), 2568).

Other methods involving rational designs include in silico methods (Porosk, L. et al. Expert Opin Drug Discov 2021, 16 (5), 553-565; Lee, E. Y. et al. Bioorganic & medicinal chemistry 2018, 26 (10), 2708-2718; Manavalan, B. et al. J. Proteome Res. 2018, 17 (8), 2715-2726); Pandey, P. et al. J. Proteome Res. 2018, 17 (9), 3214-3222).

Additional previously discovered methods leverage machine learning to design new sequences using a model trained with a combinatorial library tested for the desired activity: nuclear localization (Schissel, C. K. et al. Nat. Chem. 2021, 13 (10), 992-1000; López-Vidal, E. M. et al. JACS Au 2021; Wolfe, J. M. et al. ACS Cent Sci 2018, 4 (4), 512-520). Finally, another common strategy involves screening platforms employing libraries from phage or mRNA display. However, these methods have limited advancements for the discovery of peptides that deliver cargo to subcellular compartments.

These methods advanced to biologically relevant conditions in on-cell selection platforms for the discovery of new ligands with an affinity for the external surface of cells and tissues (Beck, S. et al. Biomaterials 2011, 32 (33), 8518-8528; Wu, C.-H. et al. Science Translational Medicine 2015, 7 (290); Wu, C.-H. et al. Journal of Biomedical Science 2016, 23 (1), 8). However, biological display techniques are restricted to the use of mostly natural amino acids, limiting the resulting library diversity and proteolytic stability (Ren, Y. et al. Mol Pharm 2018, 15 (2), 592-601; Wei, X. et al. Angew Chem Int Ed Engl 2015, 54 (10), 3023-3027), and even those mirror image techniques that allow D-peptide discovery still have difficulty incorporating non-canonical residues (Huang, L. et al. Mol Pharm 2017, 14 (5), 1742-1753; Eckert, D. M. et al. Cell 1999, 99 (1), 103-115).

Peptides are a promising strategy to improve the delivery of PMO to the nucleus. Cell-penetrating peptides (CPPs) in particular are relatively short sequences of 5-40 amino acids that ideally access the cytosol and can promote the intracellular delivery of cargo. For example, when conjugated to PMO, oligoarginine peptides have been some of the most effective peptides in promoting PMO delivery.

Accordingly, provided herein are antisense oligomer conjugates comprising an antisense oligomer covalently bound to a cell-penetrating peptide, wherein the cell-penetrating peptide comprises at least one non-canonical amino acid. The peptide also comprises canonical amino acids.

Also provided herein are methods of treating a disease in a subject in need thereof, comprising administering to the subject one or more of the antisense oligomer conjugates described herein. Certain of the cell-penetrating peptides, and thereby the antisense oligomer conjugates, described herein display one or more of increased efficacy and lower toxicity.

In particular, provided herein are methods for treating neuromuscular diseases using one or more of the antisense oligomer conjugates described herein.

### II. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the subject matter of the present disclosure, preferred methods and materials are described. For the purposes of the present disclosure, the following terms are defined below.

The term “about” will be understood by persons of ordinary skill in the art and will vary to some extent on the context in which it is used. As used herein when referring to a measurable value such as an amount, a temporal duration, and the like, the term “about” is meant to encompass variations of ±10%.

The term “alkyl” refers to saturated, straight- or branched-chain hydrocarbon moieties containing, in certain embodiments, between one and six, or one and eight carbon atoms, respectively. Examples of C₁₋₆alkyl moieties include, but are not limited to, methyl, ethyl, propyl, isopropyl, n-butyl, tert-butyl, neopentyl, n-hexyl moieties; and examples of C₁₋₈alkyl moieties include, but are not limited to, methyl, ethyl, propyl, isopropyl, n-butyl, tert-butyl, neopentyl, n-hexyl, heptyl, and octyl moieties.

The number of carbon atoms in an alkyl substituent can be indicated by the prefix “C_{x-y},” where x is the minimum and y is the maximum number of carbon atoms in the substituent. Likewise, a Cₓ chain means an alkyl chain containing x carbon atoms.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂—CH₂—S(═O)—CH₃. Up to two heteroatoms may be consecutive, such as, for example, —CH₂—NH—OCH₃, or —CH₂—CH₂—S—S—CH₃.

The term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. In various embodiments, examples of an aryl group may include phenyl (e.g., C₆aryl) and biphenyl (e.g., C₁₂aryl). In some embodiments, aryl groups have from six to sixteen carbon atoms. In some embodiments, aryl groups have from six to twelve carbon atoms (e.g., C₆₋₁₂aryl). In some embodiments, aryl groups have six carbon atoms (e.g., C₆aryl).

As used herein, the term “heteroaryl” or “heteroaromatic” refers to a heterocycle having aromatic character. Heteroaryl substituents may be defined by the number of carbon atoms, e.g., C₁₋₉heteroaryl indicates the number of carbon atoms contained in the heteroaryl group without including the number of heteroatoms. For example, a C₁₋₉heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl. For purposes of the present disclosure, heteroaryl includes DBCO, 8,9-dihydro-3H-dibenzo[b,f][1,2,3]triazolo[4,5-d]azocine.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 8,9-dihydro-3H-dibenzo[b,f][1,2,3]triazolo[4,5-d]azocine. 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

The acronym DBCO refers to a dibenzocyclooctyne moiety. In particular, as used herein, the acronym DBCO derivative refers to 8,9-dihydro-3H-dibenzo[b,f][1,2,3]triazolo[4,5-d]azocine.

The term “protecting group” or “chemical protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, monomethoxytrityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and fluorenylmethyloxycarbonyl (Fmoc) groups, which are base labile. Carboxylic acid moieties may be blocked with base labile groups such as, without limitation, methyl, or ethyl, and hydroxy reactive moieties may be blocked with base labile groups such as acetyl in the presence of amines blocked with acid labile groups such as tert-butyl carbamate or with carbamates that are both acid and base stable but hydrolytically removable.

Carboxylic acid and hydroxyl reactive moieties may also be blocked with hydrolytically removable protective groups such as the benzyl group, while amine groups may be blocked with base labile groups such as fluorenylmethyloxycarbonyl (Fmoc). A particularly useful amine protecting group for the synthesis of compounds of Formula (I) is trifluoroacetamide. Carboxylic acid reactive moieties may be blocked with oxidatively-removable protective groups such as 2,4-dimethoxybenzyl, while coexisting amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(0)-catalyzed reaction in the presence of acid labile t-butyl carbamate or base-labile acetate amine protecting groups. Yet another form of protecting group is a resin to which a compound or intermediate may be attached. Provided the residue is attached to the resin, that functional group is blocked and cannot react. Once released from the resin, the functional group is available to react.

The terms “nucleobase,” “base pairing moiety,” “nucleobase-pairing moiety,” or “base” refer to the heterocyclic ring portion of a nucleoside, nucleotide, and/or morpholino subunit. Nucleobases may be naturally occurring (e.g., uracil, thymine, adenine, cytosine, and guanine), or may be modified or analogs of these naturally occurring nucleobases, e.g., one or more nitrogen atoms of the nucleobase may be independently at each occurrence replaced by carbon. Exemplary analogs include hypoxanthine (the base component of the nucleoside inosine); 2, 6-diaminopurine; 5-methyl cytosine; C5-propynyl-modified pyrimidines; 10-(9-(aminoethoxy)phenoxazinyl) (G-clamp) and the like.

Further examples of base pairing moieties include, but are not limited to, uracil, thymine, adenine, cytosine, guanine and hypoxanthine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in Chiu and Rana (2003) RNA 9:1034-1048, Limbach et al. (1994) Nucleic Acids Res. 22:2183-2196 and Revankar and Rao, Comprehensive Natural Products Chemistry, vol. 7, 313, are also contemplated, the contents of which are incorporated herein by reference.

Further examples of base pairing moieties include, but are not limited to, expanded-size nucleobases in which one or more benzene rings have been added. Nucleic base replacements described in the Glen Research catalog (www.glenresearch.com); Krueger A T et al. (2007) Acc. Chem. Res. 40:141-150; Kool E T (2002) Acc. Chem. Res. 35:936-943; Benner S A et al. (2005) Nat. Rev. Genet. 6:553-543; Romesberg F E et al. (2003) Curr. Opin. Chem. Biol. 7:723-733; Hirao, 1 (2006) Curr. Opin. Chem. Biol. 10:622-627, the contents of which are incorporated herein by reference, are contemplated as useful for the synthesis of the oligonucleotides described herein. Examples of expanded-size nucleobases are shown below:

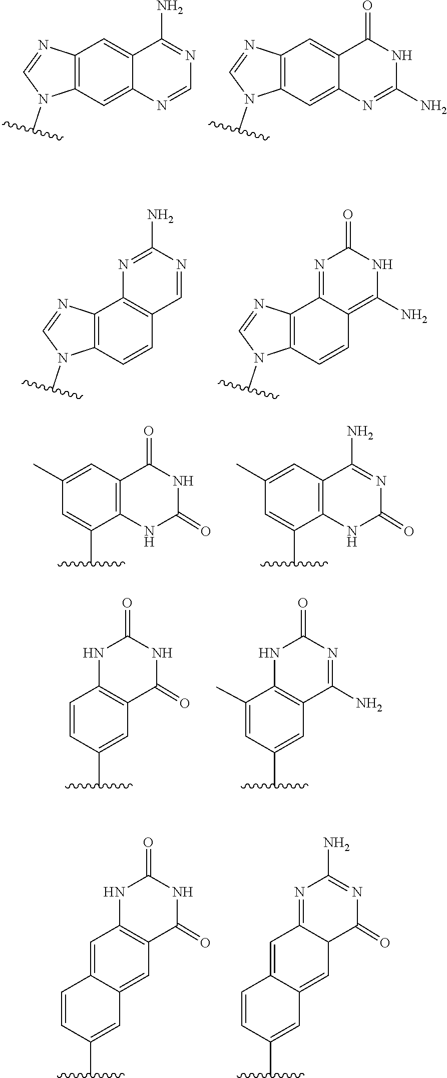

The terms “oligonucleotide” or “oligomer” refer to a compound comprising a plurality of linked nucleosides, nucleotides, or a combination of both nucleosides and nucleotides. In specific embodiments provided herein, an oligonucleotide is a morpholino oligonucleotide.

An antisense oligomer “specifically hybridizes” to a target polynucleotide if the oligonucleotide hybridizes to the target under physiological conditions, with a Tm greater than 37° C., greater than 45° C., preferably at least 50° C., and typically 60° C.-80° C. or higher. The “Tm” of an oligonucleotide is the temperature at which 50% hybridizes to a complementary polynucleotide. Tm is determined under standard conditions in physiological saline, as described, for example, in Miyada et al. (1987) Methods Enzymol. 154:94-107. Such hybridization may occur with “near” or “substantial” complementarity of the antisense oligomer to the target sequence, as well as with exact complementarity.

The terms “complementary” and “complementarity” refer to oligonucleotides (i.e., a sequence of nucleotides) related by base-pairing rules. For example, the sequence “T-G-A (5′-3′)” is complementary to the sequence “T-C-A (5-3′).” Complementarity may be “partial,” in which only some of the nucleic acids' bases are matched according to base pairing rules. Or there may be “complete,” “total,” or “perfect” (100%) complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Such hybridization may occur with “near” or “substantial” complementarity of the antisense oligomer to the target sequence, as well as with exact complementarity. In some embodiments, an oligonucleotide may hybridize to a target sequence at about 50%, 55%, 60%, 65%, 70%, 75%, 80%, 85%, 90%, 95%, 99% or 100% complementarity. Variations at any location within the oligonucleotide are included. In certain embodiments, variations in sequence near the termini of an oligonucleotide are generally preferable to variations in the interior, and if present are typically within about 6, 5, 4, 3, 2, or 1 nucleotides of the 5-terminus, 3′-terminus, or both termini.

Naturally occurring nucleotide bases include adenine, guanine, cytosine, thymine, and uracil, which have the symbols A, G, C, T, and U, respectively. Nucleotide bases can also encompass analogs of naturally occurring nucleotide bases. Base pairing typically occurs between purine A and pyrimidine T or U, and between purine G and pyrimidine C.

Oligonucleotides may also include nucleobase (often referred to in the art simply as “base”) modifications or substitutions. Oligonucleotides containing a modified or substituted base include oligonucleotides in which one or more purine or pyrimidine bases most commonly found in nucleic acids are replaced with less common or non-natural bases. In some embodiments, the nucleobase is covalently linked at the N9 atom of the purine base, or at the N1 atom of the pyrimidine base, to the morpholine ring of a nucleotide or nucleoside.

Purine bases comprise a pyrimidine ring fused to an imidazole ring, as described by the general formula:

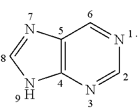

Adenine and guanine are the two purine nucleobases most commonly found in nucleic acids. These may be substituted with other naturally occurring purines, including but not limited to N6-methyladenine, N2-methylguanine, hypoxanthine, and 7-methylguanine.

Pyrimidine bases comprise a six-membered pyrimidine ring as described by the general formula:

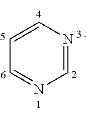

Cytosine, uracil, and thymine are the pyrimidine bases most commonly found in nucleic acids. These may be substituted with other naturally occurring pyrimidines, including but not limited to 5-methylcytosine, 5-hydroxymethylcytosine, pseudouracil, and 4-thiouracil. In one embodiment, the oligonucleotides described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, I-deoxy-2-O-methylribose; or pyrrolidine derivatives in which the ring oxygen has been replaced with nitrogen (azaribose)). Pseudouracil is a naturally occurring isomerized version of uracil, with a C-glycoside rather than the regular N-glycoside as in uridine.

Certain modified or substituted nucleobases are particularly useful for increasing the binding affinity of the antisense oligonucleotides of the disclosure. These include 5-substituted pyrimidines, 6-azapyrimidines and N-2, N-6 and 0-6 substituted purines, including 2-aminopropyladenine, 5-propynyluracil and 5-propynylcytosine. In various embodiments, nucleobases may include 5-methylcytosine substitutions, which have been shown to increase nucleic acid duplex stability by 0.6° C.-1.2° C.

In some embodiments, modified or substituted nucleobases are useful for facilitating purification of antisense oligonucleotides. For example, in certain embodiments, antisense oligonucleotides may contain three or more (e.g., 3, 4, 5, 6 or more) consecutive guanine bases. In certain antisense oligonucleotides, a string of three or more consecutive guanine bases can result in aggregation of the oligonucleotides, complicating purification. In such antisense oligonucleotides, one or more of the consecutive guanines can be substituted with hypoxanthine. The substitution of hypoxanthine for one or more guanines in a string of three or more consecutive guanine bases can reduce aggregation of the antisense oligonucleotide, thereby facilitating purification.

The oligonucleotides provided herein are synthesized and do not include antisense compositions of biological origin. The antisense oligomer conjugates of the disclosure may be mixed, encapsulated, conjugated, or otherwise associated with other molecules, molecule structures or mixtures of compounds, as for example, but not limited to, liposomes and receptor targeted molecules.

As used herein, a “nucleic acid analog” refers to a non-naturally occurring nucleic acid molecule. A nucleic acid is a polymer of nucleotide subunits linked together into a linear structure. Each nucleotide consists of a nitrogen-containing aromatic base attached to a pentose (five-carbon) sugar, which is in turn attached to a phosphate group. Successive phosphate groups are linked together through phosphodiester bonds to form the polymer. The two common forms of naturally occurring nucleic acids are deoxyribonucleic acid (DNA) and ribonucleic acid (RNA). One end of the chain carries a free phosphate group attached to the 5-carbon atom of a sugar moiety; this is called the 5′ end of the molecule. The other end has a free hydroxyl (—OH) group at the 3′-carbon of a sugar moiety and is called the 3′ end of the molecule. A nucleic acid analog includes one or more non-naturally occurring nucleobases, sugars, and/or internucleotide linkages, for example, a phosphorodiamidate morpholino oligomer (PMO). As disclosed herein, in certain embodiments, a “nucleic acid analog” is a PMO, and in certain embodiments, a “nucleic acid analog” is a positively charged cationic PMO.

A “morpholino oligomer” refers to a polymeric molecule having a backbone that supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. An exemplary “morpholino” oligomer comprises morpholino subunit structures linked together by phosphoramidate or phosphorodiamidate linkages, joining the morpholino nitrogen of one subunit to the 5′ exocyclic carbon of an adjacent subunit, each subunit comprising a purine or pyrimidine base-pairing moiety effective to bind, by base-specific hydrogen bonding, to a base in a polynucleotide. Morpholino oligomers (including antisense oligomers) are detailed, for example, in U.S. Pat. Nos. 5,034,506; 5,142,047; 5,166,315; 5,185,444; 5,217,866; 5,506,337; 5,521,063; 5,698,685; 8,076,476; and 8,299,206; and PCT publication number WO 2009/064471, all of which are incorporated herein by reference in their entirety.

A preferred morpholino oligomer is a phosphorodiamidate-linked morpholino oligomer, referred to herein as a PMO. Such oligomers are composed of morpholino subunit structures such as those shown below:

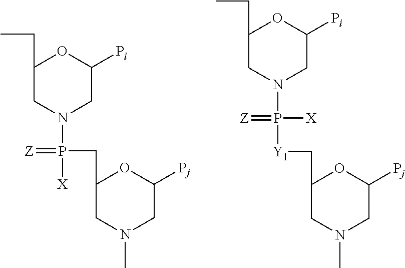

where X is NH₂, NHR, or NR₂ (where R is lower alkyl, preferably methyl), Y₁ is O, and Z is O, and Pᵢ and Pⱼ are purine or pyrimidine base-pairing moieties effective to bind, by base-specific hydrogen bonding, to a base in a polynucleotide. Also preferred are structures having an alternate phosphorodiamidate linkage, where X is lower alkoxy, such as methoxy or ethoxy, Y₁ is NH or NR, where R is lower alkyl, and Z is O.

Representative PMOs include PMOs wherein the intersubunit linkages are linkage (A1). See Table 1.

TABLE 1
Representative Intersubunit Linkages

| No. | Name | Structure |
|---|---|---|
| A1 | PMO | 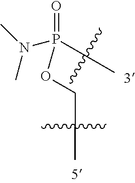 [SMILES: *COP(*)(=O)N(C)C] |
| A2 | PMO⁺ (unprotonated form depicted) | 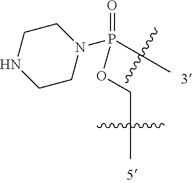 [SMILES: *COP(*)(=O)N1CCNCC1] |
| A3 | PMO⁺ (+) | 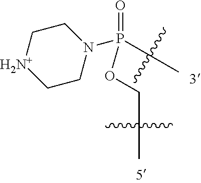 [SMILES: *COP(*)(=O)N1CC[NH2+]CC1] |

A “phosphoramidate” group comprises phosphorus having three attached oxygen atoms and one attached nitrogen atom, while a “phosphorodiamidate” group comprises phosphorus having two attached oxygen atoms and two attached nitrogen atoms. A representative phosphorodiamidate example is below:

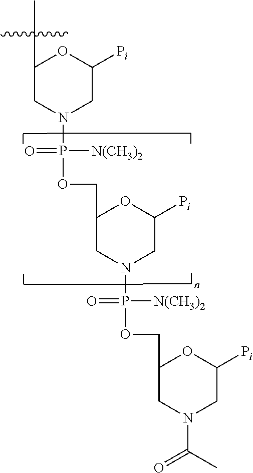

each Pᵢ is independently selected from H, a nucleobase, and a nucleobase functionalized with a chemical protecting-group, wherein the nucleobase independently at each occurrence comprises a C₃₋₆ heterocyclic ring selected from pyridine, pyrimidine, triazinane, purine, and deaza-purine; and n is an integer of 6-38.

In the uncharged or the modified intersubunit linkages of the oligonucleotides described herein, one nitrogen is always pendant to the backbone chain. The second nitrogen, in a phosphorodiamidate linkage, is typically the ring nitrogen in a morpholino ring structure.

“Charged,” “uncharged,” “cationic,” and “anionic” as used herein refer to the predominant state of a chemical moiety at near-neutral pH, e.g., about 6 to 8. For example, the term may refer to the predominant state of the chemical moiety at physiological pH, that is, about 7.4.

A “cationic PMO” or “PMO+” refers to a phosphorodiamidate morpholino oligomer comprising any number of (1-piperazino)phosphinylideneoxy, (1-(4-(ω-guanidino-alkanoyl))-piperazino)phosphinylideneoxy linkages (A2 and A3; see Table 1) that have been described previously (see e.g., PCT publication WO 2008/036127 which is incorporated herein by reference in its entirety).

The “backbone” of an oligonucleotide analog (e.g., an uncharged oligonucleotide analogue) refers to the structure supporting the base-pairing moieties; e.g., for a morpholino oligomer, as described herein, the “backbone” includes morpholino ring structures connected by intersubunit linkages (e.g., phosphorus-containing linkages). A “substantially uncharged backbone” refers to the backbone of an oligonucleotide analogue wherein less than 50% of the intersubunit linkages are charged at near-neutral pH. For example, a substantially uncharged backbone may comprise less than 50%, less than 40%, less than 30%, less than 20%, less than 10%, less than 5% or even 0% intersubunit linkages which are charged at near neutral pH. In some embodiments, the substantially uncharged backbone comprises at most one charged (at physiological pH) intersubunit linkage for every four uncharged (at physiological pH) linkages, at most one for every eight or at most one for every sixteen uncharged linkages. In some embodiments, the nucleic acid analogs described herein are fully uncharged.

The term “targeting base sequence” or simply “targeting sequence” is the sequence in the nucleic acid analog that is complementary (meaning, in addition, substantially complementary) to a target sequence, e.g., a target sequence in the RNA genome of human. The entire sequence, or only a portion, of the analog compound may be complementary to the target sequence. For example, in an analog having 20 bases, only 12-14 may be targeting sequences. Typically, the targeting sequence is formed of contiguous bases in the analog but may alternatively be formed of non-contiguous sequences that when placed together, e.g., from opposite ends of the analog, constitute sequence that spans the target sequence.

As used herein, a “cell-penetrating peptide” (CPP) or “carrier peptide” is a relatively short peptide capable of promoting uptake of PMOs by cells, thereby delivering the PMOs to the interior (cytoplasm) of the cells. The CPP or carrier peptide typically is about 3 to about 80 amino acids long. The length of the carrier peptide is not particularly limited and varies in different embodiments. In some embodiments, the carrier peptide comprises up to 70 amino acids. In some embodiments, the carrier peptide comprises at least 3 amino acids. In some embodiments, the carrier peptide comprises 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, or 70 amino acids. In some embodiments, the carrier peptide comprises from 3 to 80 amino acids. In other embodiments, the carrier peptide comprises from 3 to 70, from 3 to 60, from 3 to 50, from 3 to 40, from 3 to 30, from 3 to 20, from 8 to 25 or from 10 to 20 amino acids. In other embodiments, the carrier peptide comprises from 5 to 70, from 6 to 70, from 8 to 70, from 10 to 70, from 15 to 70, from 20 to 70, from 25 to 70, from 30 to 70, from 35 to 70, or from 40 to 70 amino acids. In other embodiments, the carrier peptide comprises from 5 to 40, from 10 to 30, from 15 to 30, or from 20 to 30 amino acids.

As used herein, a “peptide-conjugated phosphorodiamidate-linked morpholino oligomer,” “PMO-peptide conjugate,” “PPMO,” or “PMO conjugate” refers to a PMO covalently linked to a peptide, such as a cell-penetrating peptide (CPP) or carrier peptide. The cell-penetrating peptide promotes uptake of the PMO by cells, thereby delivering the PMO to the interior (cytoplasm) of the cells. Depending on its amino acid sequence, a CPP can be generally effective, or it can be specifically or selectively effective for PMO delivery to a particular type or particular types of cells. PMOs and CPPs are typically linked at their ends, e.g., the N-terminal end of the CPP can be linked to the 5 end of the PMO, or the 3′ end of the PMO can be linked to the N-terminal end of the CPP. PPMOs can include uncharged PMOs, charged (e.g., cationic) PMOs, and mixtures thereof.

An “amino acid” when used to describe an amino acid in the peptide portion of the antisense oligomers disclosed herein is generally an α-amino acid residue (—CO—CHR—NH—); but may also be a β- or other amino acid residue (e.g., —CO—CH₂CHR—NH—), where R is an amino acid side chain.

The term “naturally occurring amino acid,” “canonical amino acid,” or “essential amino acid” refers to an amino acid present in proteins found in nature; examples include, but are not limited to, arginine (Arg or R), histidine (His or H), lysine (Lys or K), aspartic acid (Asp or D), glutamic acid (Glu or E), serine (Ser or S), threonine (Thr or T), asparagine (Asn or N), glutamine (Gln or Q), glycine (Gly or G), methionine (Met or M), proline (Pro or P), valine (Val or V), alanine (Ala or A), leucine (Leu or L), phenylalanine (Phe or F), and tryptophan (Trp or W).

The terms “non-canonical amino acid,” “non-natural amino acid,” “unnatural amino acid,” and the like refer to non-essential amino acids that are not present in proteins found in nature. Representative examples include, but are not limited to, 2,4-diaminobutyric acid (Dab), 2-amino-5-ureidopentanoic acid (citrulline (Cit)), 2-amino-3-(naphthalen-2-yl)propanoic acid (Nap), halo-phenylalanine (X-ph) (e.g., 4-iodo-L-phenylalanine (Iph)), 4-aminopiperidine-4-carboxylic acid (Pip), 2-amino-3-(pyridin-3-yl)propanoic acid (Pyr).

In some embodiments, the non-canonical amino acid is a racemic mixture and is selected from 2,4-diaminobutyric acid (Dab), 2-amino-5-ureidopentanoic acid (citrulline (Cit)), 2-amino-3-(naphthalen-2-yl)propanoic acid (Nap), 4-aminopiperidine-4-carboxylic acid (Pip), 2-amino-3-(pyridin-3-yl)propanoic acid (Pyr), and halophenylalanine (X-ph).

In some embodiments, the non-canonical amino acid has defined stereochemistry and is selected from 2,4-diaminobutyric acid (Dab) (e.g., L-2,4-diaminobutyric acid, (S)-2,4-diaminobutanoic acid, etc.), 2-amino-5-ureidopentanoic acid (citrulline (Cit)) (e.g., L-citrulline, (S)-2-amino-5-ureidopentanoic acid, etc.), 2-amino-3-(naphthalen-2-yl)propanoic acid (Nap) (e.g., (S)-2-amino-3-(naphthalen-2-yl)propanoic acid, 3-(2-naphthyl)-L-alanine, etc.), 4-aminopiperidine-4-carboxylic acid (Pip), 2-amino-3-(pyridin-3-yl)propanoic acid (Pyr) (e.g., (S)-2-amino-3-(pyridin-3-yl)propanoic acid, 3-(3-pyridyl)-L-alanine), and halophenylalanine (X-ph) (e.g., iodophenylalanine (Iph), 4-iodo-L-phenylalanine, (2S)-2-amino-3-(4-iodophenyl)propanoic acid).

In some embodiments, the non-canonical amino acid is a racemic mixture and has the structure selected from

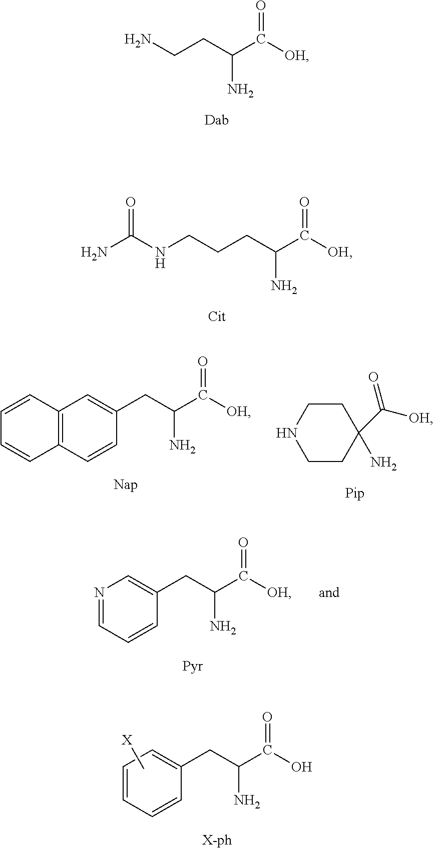

where X is halogen. In certain embodiments, the non-canonical amino acid is a racemic mixture and has the structure

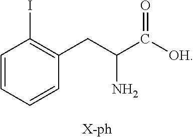 [SMILES: NC(Cc1ccccc1I)C(=O)O]

In some embodiments, the non-canonical amino acid has defined stereochemistry and has the structure from

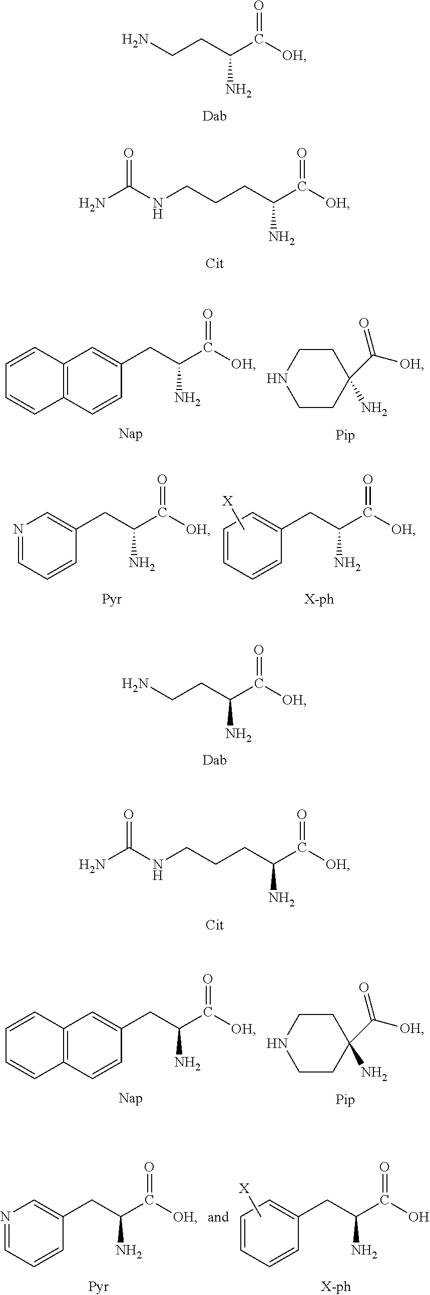

where X is halogen. In certain embodiments, the non-canonical amino acid has defined stereochemistry and has the structure

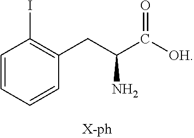 [SMILES: N[C@@H](Cc1ccccc1I)C(=O)O]

In certain embodiments, the non-canonical amino acid has defined stereochemistry and has the structure

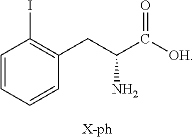 [SMILES: N[C@H](Cc1ccccc1I)C(=O)O]

As used herein, an “effective amount” refers to any amount of a substance that is sufficient to achieve a desired biological result. A “therapeutically effective amount” refers to any amount of a substance that is sufficient to achieve a desired therapeutic result.

The term “treatment” or “treating” of an individual (e.g., a mammal, such as a human) or a cell is any type of intervention used to alter the natural course of the individual or cell. Treatment includes, but is not limited to, administration of a pharmaceutical composition, and may be performed either prophylactically or subsequent to the initiation of a pathologic event or contact with an etiologic agent.

The terms “prevent,” “preventing,” and “prevention” as used herein, refer to a decrease or abatement in the occurrence of disease symptoms in a patient. The prevention may be complete (no detectable symptoms) or partial, such that fewer symptoms are observed than would likely occur absent treatment. In embodiments, prevent refers to slowing the progression of the disease, disorder or condition or inhibiting progression thereof to a harmful or otherwise undesired state.

As used herein, “subject” refers to an animal, preferably a mammal, and in particular a human or a non-human animal including livestock animals and domestic animals including, but not limited to, cattle, horses, sheep, swine, goats, rabbits, cats, dogs, rodents, non-human primates, humans, and other mammals in need of treatment. In some embodiments, the subject is a human.

As used herein, the term “administration” and variants thereof (e.g., “administering”) in reference to the compounds of Formulae I, Ia Ib, Ic, Id, II, IIa, IIb, III, IIIa, IV, IVa, Va, Vb, Vc, Vd, VI, VIa, VII, VIIa, VIIIa, and/or VIIIb means providing the compound to a subject in need of treatment, including, but not limited to, various systemic routes, including oral and parenteral routes, e.g., intravenous, subcutaneous, intraperitoneal, and intramuscular, as well as inhalation, transdermal and topical delivery.

Administering of the antisense oligomer conjugate of Formulae I, Ia Ib, Ic, Id, II, IIa, IIb, III, IIIa, IV, IVa, Va, Vb, Vc, Vd, VI, VIa, VII, VIIa, VIIIa, and/or VIIIb to the subject includes both self-administration and administration to the subject by another. The subject may be in need of, or desire, treatment for an existing disease or medical condition, or may be in need of or desire prophylactic treatment to prevent or reduce the risk of occurrence of the disease or medical condition. As used herein, a subject “in need” of treatment of an existing condition or of prophylactic treatment encompasses both a determination of need by a medical professional as well as the desire of a patient for such treatment.

### III. Antisense Oligomer Conjugates

In an embodiment, provided herein are antisense oligomer conjugates comprising an antisense oligomer covalently linked to a cell-penetrating peptide, wherein the peptide comprises at least one non-canonical amino acid. In an embodiment, at least one non-canonical amino acid is selected from Cit, X-ph, and Pyr. In an embodiment, at least one non-canonical amino acid is X-ph. In an embodiment, the X is I. In an embodiment, the X-ph is Iph. In some embodiments, the antisense oligomer conjugate comprises one or more of the chemistries described herein.

As discussed, the carrier peptides provided herein comprise at least one non-canonical amino acid. In an embodiment, at least one non-canonical amino acid is selected from Cit, X-ph, and Pyr.

In certain embodiments, the CPP is referred to as a singular CPP. These CPPs include a region of amino acids having one or more non-canonical amino acids and optionally a lysine-tryptophan motif. In other embodiments, the CPP is referred to as a chimeric CPP. These CPPs include two repeating regions having one or more non-canonical amino acids, where each region is optionally followed by a lysine-tryptophan motif. In certain embodiments, the non-canonical amino acid is selected from a hydrophobic amino acid, such as Nap, Xph or Iph (defined herein); a charged, hydrophobic amino acid, such as Pip or Pyr; and a hydrophilic amino acid, such as Dab or Cit.

In certain embodiments, a linking moiety attaches the carrier peptide to an antisense oligomer to form the antisense oligomer conjugate. The carrier peptide may be linked to the nucleic acid analog either directly or via an optional linker, e.g., one or more additional naturally occurring amino acids, e.g., cysteine (C), glycine (G), alanine (A), or proline (P), or additional amino acid analogs, e.g., 6-aminohexanoic acid (X), beta-alanine (B), or XB. Useful linking moieties herein attach the carrier peptide to an antisense oligomer either directly or via a linker. One such linker is a linker that includes a covalent bridge formed by copper-free click-chemistry reactive groups that covalently bond the peptide part and oligonucleotide part together. Other linking strategies are known in the art. In certain embodiments, a linking moiety includes an additional linking amino acid that attaches the carrier peptide to a click chemistry handle to allow bonding to an antisense oligomer to form the antisense oligomer conjugate. In certain embodiments, the linking amino acid is selected from Gly and Ala. In certain embodiments, the linking amino acid is Ala. In certain embodiments, a linking amino acid is Gly.

In certain embodiments, the carrier peptide, when conjugated to an antisense oligomer, is effective to enhance the binding of the antisense oligomer to its target sequence, relative to the antisense oligomer in unconjugated form, as evidenced by:
(i) a decrease in expression of an encoded protein, relative to that provided by the unconjugated antisense oligomer, when binding of the antisense oligomer to its target sequence is effective to block a translation start codon for the encoded protein, or
(ii) an increase in expression of an encoded protein, relative to that provided by the unconjugated antisense oligomer, when binding of the antisense oligomer to its target sequence is effective to block an aberrant splice site in a pre-mRNA which encodes said protein when correctly spliced. Assays suitable for measurement of these effects are described further below. In one embodiment, conjugation of the peptide provides this activity in a cell-free translation assay, as described herein. In some embodiments, activity is enhanced by a factor of at least two, a factor of at least five or a factor of at least ten.

Alternatively, or in addition, the carrier peptide is effective to enhance the transport of the nucleic acid analog into a cell, relative to the analog in unconjugated nucleic acid analog form. In certain embodiments, transport is enhanced by a factor of at least two, a factor of at least two, a factor of at least five or a factor of at least ten.

In certain embodiments, R¹ is —OH or —NR³R⁴.

In an embodiment, R¹ is N(CH₃)₂.

In certain embodiments, each R² is a nucleobase, independently at each occurrence, selected from adenine, guanine, cytosine, 5-methyl-cytosine, thymine, uracil, and hypoxanthine.

In certain embodiments, L¹ or L² is:

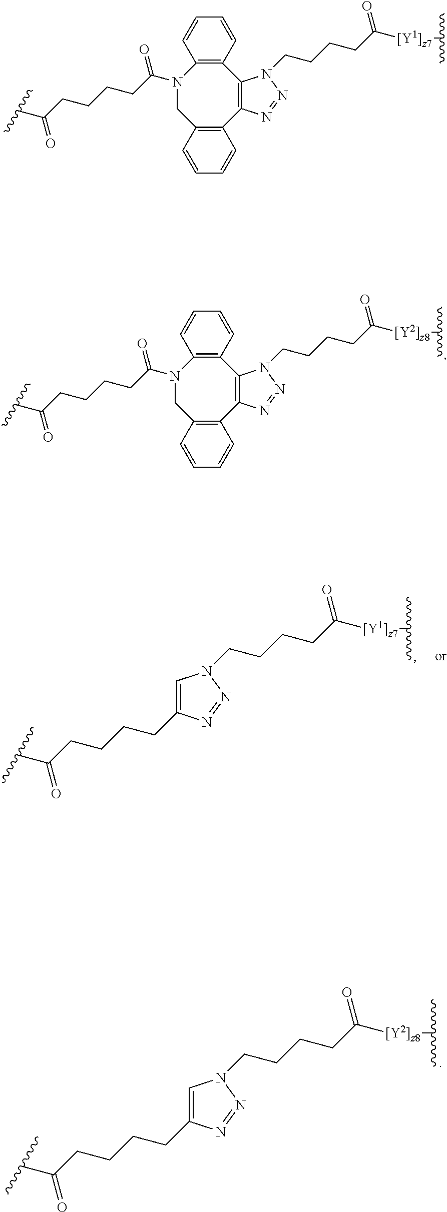

In a further embodiment, L¹ or L² is:

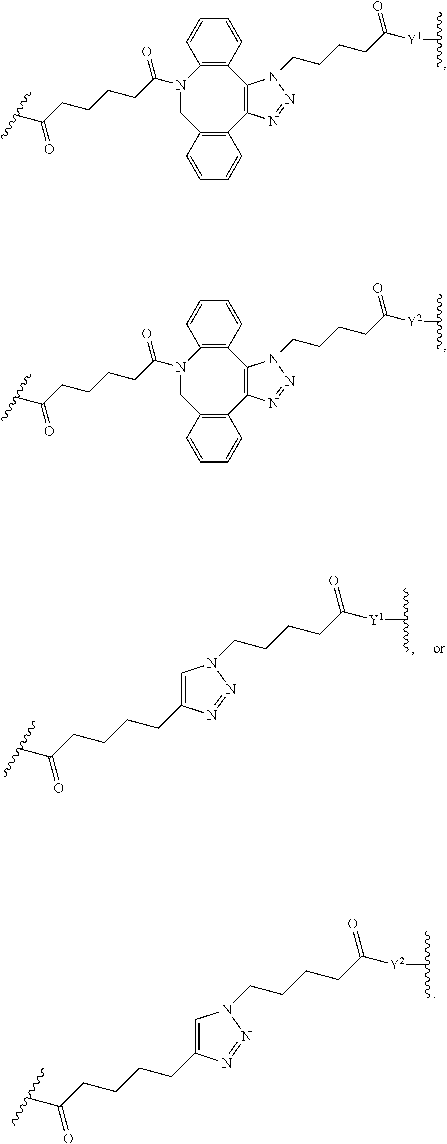

In a further embodiment, L¹ is:

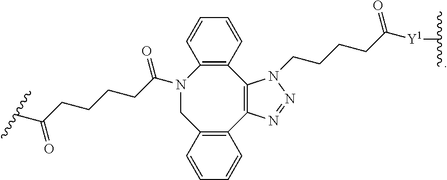

In a further embodiment, L² is:

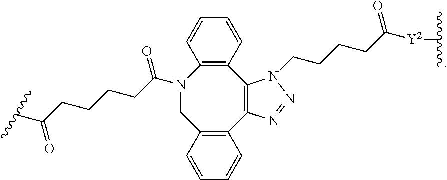

In certain embodiments, Y¹ or Y² is selected from Gly and Ala. In a further embodiment, Y¹ or Y² is Gly. In a further embodiment, Y¹ or Y² is Ala.

In still another embodiment, G¹ or G² is selected from hydrogen, —C(O)CH₃, —NH₂, benzoyl, and stearoyl. In a further embodiment, G¹ or G² is —NH.

In certain embodiments, -(J^{1C})ₜ₁-(J^{1B})_{z3}, -(J^{1D})ᵤ₁-(J^{1A})_{z4}, -(J^{2C})ₜ₂-(J^{2B})_{z5}-, or -(J^{2D})ᵤ₂-(J^{2A})_{z6} form a peptide sequence that has a region with one or more non-canonical amino acids and a region with a Lys-Trp motif, the sequence selected from:

| SEQ ID | Peptide | |
|---|---|---|
| NO. | Name | Sequence |
| 48 | CXP1 | Lys-Gln-Lys-Thr-Ser-Iph-Gly-Arg-Gly-Pip-Lys-Trp-Lys-Lys; |
| 49 | CXP2 | Asn-Phe-Lys-Gln-Nap-His-Ala-Gly-Dab-Arg-Lys-Trp-Lys-Lys; |
| 50 | CXP3 | Dab-Val-Ala-Arg-Asn-Asn-Dab-Thr-Lys-Gly-Lys-Trp-Lys-Lys; |
| 51 | CXP4 | Cit-Met-Phe-Gly-Pip-Dab-Dab-Lys-Ala-Pro-Lys-Trp-Lys-Lys; |
| 52 | CXP5 | Dab-Pro-Arg-Dab-Leu-His-Ser-Dab-Ala-Thr-Lys-Trp-Lys-Lys; |
| 53 | CXP6 | Pro-Pip-Gly-Dab-Lys-Dab-Ser-Val-Iph-Nap-Lys-Trp-Lys-Lys; |
| 54 | CXP7 | Val-Gln-His-Iph-Arg-Dab-Lys-Glu-Lys-Asn-Lys-Trp-Lys-Lys; |
| 55 | CXP8 | Asn-Phe-Asn-Asn-Arg-Ser-Dab-Gly-His-Lys-Lys-Trp-Lys-Lys; |
| 56 | CXP9 | Ala-Arg-Ser-Glu-Gln-His-His-Nap-Ser-Dab-Lys-Trp-Lys-Lys; |
| 57 | SA | Iph-Gly-Arg-Gly-Pip-Lys-Trp-Lys-Lys; |
| 58 | SB | Lys-Thr-Ser-Iph-Gly-Arg-Lys-Trp-Lys-Lys; |
| 61 | SE | Iph-Gly-Arg-Lys-Trp-Lys-Lys; |
| 62 | SF | Lys-Gln-Lys-Thr-Ser-Iph-Gly-Arg-Lys-Trp-Lys-Lys; |
| 63 | SG | Ser-Iph-Gly-Arg-Lys-Trp-Lys-Lys; |
| 65 | SI | Gln-Lys-Thr-Ser-Iph-Gly-Arg-Lys-Trp-Lys-Lys; |
| 66 | SJ | Lys-Gln-Lys-Thr-Ser-Iph-Gly-Arg-Lys-Trp-Lys-Lys; |
| 67 | CXP1- | Lys-Gln-Lys-Thr-Ser-Iph-Gly-Arg-Gly-Pip-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 68 | CXP3- | Dab-Val-Ala-Arg-Asn-Asn-Dab-Thr-Lys-Gly-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 69 | CXP4- | Cit-Met-Phe-Gly-Pip-Dab-Dab-Lys-Ala-Pro-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 70 | SA-CXP1 | Iph-Gly-Arg-Gly-Pip-Lys-Gln-Lys-Thr-Ser-Iph-Gly-Arg-Gly-Pip-Lys- |
| | | Trp-Lys-Lys; |
| 71 | SB-CXP1 | Lys-Thr-Ser-Iph-Gly-Arg-Lys-Gln-Lys-Thr-Ser-Iph-Gly-Arg-Gly-Pip- |
| | | Lys-Trp-Lys-Lys; |
| 72 | SC-CXP1 | Lys-Gln-Lys-Thr-Ser-Lys-Gln-Lys-Thr-Ser-Iph-Gly-Arg-Gly-Pip- |
| | | Lys-Trp-Lys-Lys; |
| 73 | SD-CXP1 | Lys-Trp-Lys-Lys-Gln-Lys-Thr-Ser-Iph-Gly-Arg-Gly-Pip-Lys-Trp- |
| | | Lys-Lys; |
| 74 | CXD1A | Lys-Thr-Ser-Iph-Gly-Arg-Lys-Gln-Lys-Thr-Ser-Iph-Gly-Arg-Lys- |
| | | Trp-Lys-Lys; |
| 75 | CXD1B | Lys-Gln-Lys-Thr-Ser-Iph-Gly-Arg-Lys-Trp-Lys-Gln-Lys-Thr-Ser- |
| | | Iph-Gly-Arg-Lys-Trp-Lys-Lys; |
| 78 | CXP1X | Lys-Gln-Lys-Thr-Ser-Phe-Gly-Arg-Gly-Pip-Lys-Trp-Lys-Lys; |
| 79 | CXP1Y | Lys-Gln-Lys-Thr-Ser-Iph-Gly-Arg-Gly-Lys-Lys-Trp-Lys-Lys; |
| 80 | CXP1Z | Lys-Gln-Lys-Thr-Ser-Phe-Gly-Arg-Gly-Lys-Lys-Trp-Lys-Lys; |
| 81 | CXP1AA | Lys-Gln-Lys-Thr-Ser-Iph-Gly-Lys-Gly-Pip-Lys-Trp-Lys-Lys; |
| 82 | CXP1P | Lys-Gln-Lys-Thr-Ser-Iph-Ala-Arg-Ala-Pip-Lys-Trp-Lys-Lys; |
| 83 | CXP1B | Ala-Gln-Lys-Thr-Ser-Iph-Gly-Arg-Gly-Pip-Lys-Trp-Lys-Lys; |
| 84 | CXP1C | Lys-Ala-Lys-Thr-Ser-Iph-Gly-Arg-Gly-Pip-Lys-Trp-Lys-Lys; |
| 85 | CXP1D | Lys-Gln-Ala-Thr-Ser-Iph-Gly-Arg-Gly-Pip-Lys-Trp-Lys-Lys; |
| 86 | CXP1E | Lys-Gln-Lys-Ala-Ser-Iph-Gly-Arg-Gly-Pip-Lys-Trp-Lys-Lys; |
| 87 | CXP1F | Lys-Gln-Lys-Thr-Ala-Iph-Gly-Arg-Gly-Pip-Lys-Trp-Lys-Lys; |
| 88 | CXP1G | Lys-Gln-Lys-Thr-Ser-Ala-Gly-Arg-Gly-Pip-Lys-Trp-Lys-Lys; |
| 89 | CXP1H | Lys-Gln-Lys-Thr-Ser-Iph-Ala-Arg-Gly-Pip-Lys-Trp-Lys-Lys; |
| 90 | CXP1I | Lys-Gln-Lys-Thr-Ser-Iph-Gly-Ala-Gly-Pip-Lys-Trp-Lys-Lys; |
| 91 | CXP1J | Lys-Gln-Lys-Thr-Ser-Iph-Gly-Arg-Ala-Pip-Lys-Trp-Lys-Lys; |
| 92 | CXP1K | Lys-Gln-Lys-Thr-Ser-Iph-Gly-Arg-Gly-Ala-Lys-Trp-Lys-Lys; |
| 93 | CXP1L | Lys-Gln-Lys-Thr-Ser-Iph-Gly-Arg-Gly-Pip-Ala-Trp-Lys-Lys; |
| 94 | CXP1M | Lys-Gln-Lys-Thr-Ser-Iph-Gly-Arg-Gly-Pip-Lys-Ala-Lys-Lys; |
| 95 | CXP1N | Lys-Gln-Lys-Thr-Ser-Iph-Gly-Arg-Gly-Pip-Lys-Trp-Ala-Lys; |
| 96 | CXP1O | Lys-Gln-Lys-Thr-Ser-Iph-Gly-Arg-Gly-Pip-Lys-Trp-Lys-Ala-Lys-Trp- |
| | | Lys-Lys; |
| 97 | CXP1Q | Iph-Gln-Lys-Thr-Ser-Iph-Gly-Arg-Gly-Pip-Lys-Trp-Lys-Lys; |
| 98 | CXP1R | Lys-Gln-Iph-Thr-Ser-Iph-Gly-Arg-Gly-Pip-Lys-Trp-Lys-Lys; |
| 99 | CXP1S | Lys-Gln-Lys-Thr-Ser-Iph-Gly-Iph-Gly-Pip-Lys-Trp-Lys-Lys; |
| 100 | CXP1T | Lys-Gln-Lys-Thr-Ser-Iph-Gly-Arg-Gly-Iph-Lys-Trp-Lys-Lys; |
| 101 | CXP1U | Lys-Gln-Lys-Thr-Ser-Iph-Gly-Arg-Gly-Pip-Iph-Trp-Lys-Lys |
| 102 | CXP1V | Lys-Gln-Lys-Thr-Ser-Iph-Gly-Arg-Gly-Pip-Lys-Trp-Iph-Lys-Lys-Trp- |
| | | Lys-Lys; |
| 103 | CXP1W | Lys-Gln-Lys-Thr-Ser-Iph-Gly-Arg-Gly-Pip-Lys-Trp-Lys-Iph-Lys-Trp- |
| | | Lys-Lys; |
| 104 | CXP1AC | Dab-Gln-Dab-Thr-Ser-Iph-Gly-Arg-Gly-Pip-Dab-Trp-Dab-Dab-Lys- |
| | | Trp-Lys-Lys; |
| 105 | CXP1AD | Dab-Gln-Dab-Thr-Ser-Iph-Gly-Dab-Gly-Pip-Dab-Trp-Dab-Dab-Lys- |
| | | Trp-Lys-Lys; |
| 106 | CXP1AE | Dab-Gln-Dab-Thr-Ser-Iph-Gly-Arg-Gly-Dab-Dab-Trp-Dab-Dab- |
| | | Lys-Trp-Lys-Lys; and |
| 107 | CXP1AF | Dab-Gln-Dab-Thr-Ser-Iph-Gly-Dab-Gly-Dab-Dab-Trp-Dab-Dab- |
| | | Lys-Trp-Lys-Lys. |

In certain embodiments, -(J^{1C})ₜ₁-(J¹)₃, -(J^{1D})ᵤ₁-(J^{1A})_{z4}, -(J^{2C})ₜ₂-(J^{2B})_{z5}-, or -(J^{2D})ᵤ₂-(J^{2A})_{z6} each independently form a peptide sequence. In other words the J^{1C}, J^{1B}, J^{1D}, J^{1A}, J^{2C}, J^{2B}, J^{2D}, or J^{2A} represents either a canonical or non-canonical amino acid and t1, z3, u1, z4, t2, z5, u2, or z6 indicate the number of canonical or non-canonical amino acids that form the peptide sequence, e.g., when t1 is 10 and J^{1C} is the sequence of Amino Acids, Lys, Gln, Lys, Thr, Ser, Iph, Gly, Arg, Gly, and Pip, and when z3 is 4 and J^{1B} is the sequence of Amino Acids, Lys, Trp, Lys, and Lys, then the full -(J^{1C})ₜ₁-(J^{1B})_{z3}- peptide is CXP1, SEQ ID NO.: 48, with the full sequence of Lys-Gln-Lys-Thr-Ser-Iph-Gly-Arg-Gly-Pip-Lys-Trp-Lys-Lys.

In another embodiment, -(J^{1C})ₜ-(J^{1B})_{z3}-, or -(J^{2C})ₜ₂-(J^{1B})_{z5}- form a peptide sequence that has a region with one or more non-canonical amino acids and a region with a Lys-Trp motif, the sequence selected from:

| SEQ ID | Peptide | |
|---|---|---|
| NO. | Name | Sequence |
| 48 | CXP1 | Lys-Gln-Lys-Thr-Ser-Iph-Gly-Arg-Gly-Pip-Lys-Trp-Lys-Lys; |
| 49 | CXP2 | Asn-Phe-Lys-Gln-Nap-His-Ala-Gly-Dab-Arg-Lys-Trp-Lys-Lys; |
| 50 | CXP3 | Dab-Val-Ala-Arg-Asn-Asn-Dab-Thr-Lys-Gly-Lys-Trp-Lys-Lys; |
| 51 | CXP4 | Cit-Met-Phe-Gly-Pip-Dab-Dab-Lys-Ala-Pro-Lys-Trp-Lys-Lys; |
| 52 | CXP5 | Dab-Pro-Arg-Dab-Leu-His-Ser-Dab-Ala-Thr-Lys-Trp-Lys-Lys; |
| 53 | CXP6 | Pro-Pip-Gly-Dab-Lys-Dab-Ser-Val-Iph-Nap-Lys-Trp-Lys-Lys; |
| 54 | CXP7 | Val-Gln-His-Iph-Arg-Dab-Lys-Glu-Lys-Asn-Lys-Trp-Lys-Lys; |
| 55 | CXP8 | Asn-Phe-Asn-Asn-Arg-Ser-Dab-Gly-His-Lys-Lys-Trp-Lys-Lys; |
| 56 | CXP9 | Ala-Arg-Ser-Glu-Gln-His-His-Nap-Ser-Dab-Lys-Trp-Lys-Lys; |
| 57 | SA | Iph-Gly-Arg-Gly-Pip-Lys-Trp-Lys-Lys; |
| 58 | SB | Lys-Thr-Ser-Iph-Gly-Arg-Lys-Trp-Lys-Lys; |
| 61 | SE | Iph-Gly-Arg-Lys-Trp-Lys-Lys; |
| 62 | SF | Lys-Gln-Lys-Thr-Ser-Iph-Gly-Arg-Lys-Trp-Lys-Lys; |
| 63 | SG | Ser-Iph-Gly-Arg-Lys-Trp-Lys-Lys; |
| 65 | SI | Gln-Lys-Thr-Ser-Iph-Gly-Arg-Lys-Trp-Lys-Lys; |
| 66 | SJ | Lys-Gln-Lys-Thr-Ser-Iph-Gly-Arg-Lys-Trp-Lys-Lys; |
| 67 | CXP1- | Lys-Gln-Lys-Thr-Ser-Iph-Gly-Arg-Gly-Pip-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 68 | CXP3- | Dab-Val-Ala-Arg-Asn-Asn-Dab-Thr-Lys-Gly-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 69 | CXP4- | Cit-Met-Phe-Gly-Pip-Dab-Dab-Lys-Ala-Pro-Lys-Trp-Lys-Lys-Gly- |
| | CXP1 | Lys-Gln-Lys-Thr-Ser-Iph-Gly-Arg-Gly-Pip-Lys-Trp-Lys-Lys; |
| 70 | SA-CXP1 | Iph-Gly-Arg-Gly-Pip-Lys-Gln-Lys-Thr-Ser-Iph-Gly-Arg-Gly-Pip-Lys- |
| | | Trp-Lys-Lys; |
| 71 | SB-CXP1 | Lys-Thr-Ser-Iph-Gly-Arg-Lys-Gln-Lys-Thr-Ser-Iph-Gly-Arg-Gly-Pip- |
| | | Lys-Trp-Lys-Lys; |
| 72 | SC-CXP1 | Lys-Gln-Lys-Thr-Ser-Lys-Gln-Lys-Thr-Ser-Iph-Gly-Arg-Gly-Pip- |
| | | Lys-Trp-Lys-Lys; |
| 73 | SD-CXP1 | Lys-Trp-Lys-Lys-Gln-Lys-Thr-Ser-Iph-Gly-Arg-Gly-Pip-Lys-Trp- |
| | | Lys-Lys; |
| 74 | CXD1A | Lys-Thr-Ser-Iph-Gly-Arg-Lys-Gln-Lys-Thr-Ser-Iph-Gly-Arg-Lys- |
| | | Trp-Lys-Lys; |
| 75 | CXD1B | Lys-Gln-Lys-Thr-Ser-Iph-Gly-Arg-Lys-Trp-Lys-Gln-Lys-Thr-Ser- |
| | | Iph-Gly-Arg-Lys-Trp-Lys-Lys; |
| 78 | CXP1X | Lys-Gln-Lys-Thr-Ser-Phe-Gly-Arg-Gly-Pip-Lys-Trp-Lys-Lys; |
| 79 | CXP1Y | Lys-Gln-Lys-Thr-Ser-Iph-Gly-Arg-Gly-Lys-Lys-Trp-Lys-Lys; |
| 80 | CXP1Z | Lys-Gln-Lys-Thr-Ser-Phe-Gly-Arg-Gly-Lys-Lys-Trp-Lys-Lys; |
| 81 | CXP1AA | Lys-Gln-Lys-Thr-Ser-Iph-Gly-Lys-Gly-Pip-Lys-Trp-Lys-Lys; |
| 82 | CXP1P | Lys-Gln-Lys-Thr-Ser-Iph-Ala-Arg-Ala-Pip-Lys-Trp-Lys-Lys; |
| 83 | CXP1B | Ala-Gln-Lys-Thr-Ser-Iph-Gly-Arg-Gly-Pip-Lys-Trp-Lys-Lys; |
| 84 | CXP1C | Lys-Ala-Lys-Thr-Ser-Iph-Gly-Arg-Gly-Pip-Lys-Trp-Lys-Lys; |
| 85 | CXP1D | Lys-Gln-Ala-Thr-Ser-Iph-Gly-Arg-Gly-Pip-Lys-Trp-Lys-Lys; |
| 86 | CXP1E | Lys-Gln-Lys-Ala-Ser-Iph-Gly-Arg-Gly-Pip-Lys-Trp-Lys-Lys; |
| 87 | CXP1F | Lys-Gln-Lys-Thr-Ala-Iph-Gly-Arg-Gly-Pip-Lys-Trp-Lys-Lys; |
| 88 | CXP1G | Lys-Gln-Lys-Thr-Ser-Ala-Gly-Arg-Gly-Pip-Lys-Trp-Lys-Lys; |
| 89 | CXP1H | Lys-Gln-Lys-Thr-Ser-Iph-Ala-Arg-Gly-Pip-Lys-Trp-Lys-Lys; |
| 90 | CXP1I | Lys-Gln-Lys-Thr-Ser-Iph-Gly-Ala-Gly-Pip-Lys-Trp-Lys-Lys; |
| 91 | CXP1J | Lys-Gln-Lys-Thr-Ser-Iph-Gly-Arg-Ala-Pip-Lys-Trp-Lys-Lys; |
| 92 | CXP1K | Lys-Gln-Lys-Thr-Ser-Iph-Gly-Arg-Gly-Ala-Lys-Trp-Lys-Lys; |
| 93 | CXP1L | Lys-Gln-Lys-Thr-Ser-Iph-Gly-Arg-Gly-Pip-Ala-Trp-Lys-Lys; |
| 94 | CXP1M | Lys-Gln-Lys-Thr-Ser-Iph-Gly-Arg-Gly-Pip-Lys-Ala-Lys-Lys; |
| 95 | CXP1N | Lys-Gln-Lys-Thr-Ser-Iph-Gly-Arg-Gly-Pip-Lys-Trp-Ala-Lys; |
| 96 | CXP1O | Lys-Gln-Lys-Thr-Ser-Iph-Gly-Arg-Gly-Pip-Lys-Trp-Lys-Ala-Lys-Trp- |
| | | Lys-Lys; |
| 97 | CXP1Q | Iph-Gln-Lys-Thr-Ser-Iph-Gly-Arg-Gly-Pip-Lys-Trp-Lys-Lys; |
| 98 | CXP1R | Lys-Gln-Iph-Thr-Ser-Iph-Gly-Arg-Gly-Pip-Lys-Trp-Lys-Lys; |
| 99 | CXP1S | Lys-Gln-Lys-Thr-Ser-Iph-Gly-Iph-Gly-Pip-Lys-Trp-Lys-Lys; |
| 100 | CXP1T | Lys-Gln-Lys-Thr-Ser-Iph-Gly-Arg-Gly-Iph-Lys-Trp-Lys-Lys; |
| 101 | CXP1U | Lys-Gln-Lys-Thr-Ser-Iph-Gly-Arg-Gly-Pip-Iph-Trp-Lys-Lys |
| 102 | CXP1V | Lys-Gln-Lys-Thr-Ser-Iph-Gly-Arg-Gly-Pip-Lys-Trp-Iph-Lys-Lys-Trp- |
| | | Lys-Lys; |
| 103 | CXP1W | Lys-Gln-Lys-Thr-Ser-Iph-Gly-Arg-Gly-Pip-Lys-Trp-Lys-Iph-Lys-Trp- |
| | | Lys-Lys; |
| 104 | CXP1AC | Dab-Gln-Dab-Thr-Ser-Iph-Gly-Arg-Gly-Pip-Dab-Trp-Dab-Dab-Lys- |
| | | Trp-Lys-Lys; |
| 105 | CXP1AD | Dab-Gln-Dab-Thr-Ser-Iph-Gly-Dab-Gly-Pip-Dab-Trp-Dab-Dab-Lys- |
| | | Trp-Lys-Lys; |
| 106 | CXP1AE | Dab-Gln-Dab-Thr-Ser-Iph-Gly-Arg-Gly-Dab-Dab-Trp-Dab-Dab- |
| | | Lys-Trp-Lys-Lys; and |
| 107 | CXP1AF | Dab-Gln-Dab-Thr-Ser-Iph-Gly-Dab-Gly-Dab-Dab-Trp-Dab-Dab- |
| | | Lys-Trp-Lys-Lys. |

In certain embodiments, the portion of the CPP defined by -(J^{1C})ₜ₁-, -(J^{1D})ᵤ₁-, -(J^{2C})ₜ₂-, or -(J^{2D})ᵤ₂- has a peptide sequence selected from:

| SEQ ID | Peptide | |
|---|---|---|
| NO. | portion | Sequence |
| 1 | CXP1 | Lys-Gln-Lys-Thr-Ser-Iph-Gly-Arg-Gly-Pip; |
| 2 | CXP2 | Asn-Phe-Lys-Gln-Nap-His-Ala-Gly-Dab-Arg; |
| 3 | CXP3 | Dab-Val-Ala-Arg-Asn-Asn-Dab-Thr-Lys-Gly; |
| 4 | CXP4 | Cit-Met-Phe-Gly-Pip-Dab-Dab-Lys-Ala-Pro; |
| 5 | CXP5 | Dab-Pro-Arg-Dab-Leu-His-Ser-Dab-Ala-Thr; |
| 6 | CXP6 | Pro-Pip-Gly-Dab-Lys-Dab-Ser-Val-Iph-Nap; |
| 7 | CXP7 | Val-Gln-His-Iph-Arg-Dab-Lys-Glu-Lys-Asn; |
| 8 | CXP8 | Asn-Phe-Asn-Asn-Arg-Ser-Dab-Gly-His-Lys; |
| 9 | CXP9 | Ala-Arg-Ser-Glu-Gln-His-His-Nap-Ser-Dab; |
| 10 | SA | Iph-Gly-Arg-Gly-Pip; |
| 11 | SB | Lys-Thr-Ser-Iph-Gly-Arg; |
| 12 | SC | Lys-Gln-Lys-Thr-Ser; |
| 13 | SD | Lys-Trp-Lys; |
| 14 | SE | Iph-Gly-Arg; |
| 15 | SF | Lys-Gln-Lys-Thr-Ser-Iph-Gly-Arg; |
| 16 | SG | Ser-Iph-Gly-Arg; |
| 18 | SI | Gln-Lys-Thr-Ser-Iph-Gly-Arg; |
| 19 | SJ | Lys-Gln-Lys-Thr-Ser-Iph-Gly-Arg; |
| 39 | CXP1-CXP1 | Lys-Gln-Lys-Thr-Ser-Iph-Gly-Arg-Gly-Pip-Lys-Trp-Lys-Lys-Gly- |
| | | Lys-Gln-Lys-Thr-Ser-Iph-Gly-Arg-Gly-Pip; |
| 40 | CXP3-CXP1 | Dab-Val-Ala-Arg-Asn-Asn-Dab-Thr-Lys-Gly-Lys-Trp-Lys-Lys- |
| | | Gly-Lys-Gln-Lys-Thr-Ser-Iph-Gly-Arg-Gly-Pip; |
| 41 | CXP4-CXP1 | Cit-Met-Phe-Gly-Pip-Dab-Dab-Lys-Ala-Pro-Lys-Trp-Lys-Lys-Gly- |
| | | Lys-Gln-Lys-Thr-Ser-Iph-Gly-Arg-Gly-Pip; |
| 42 | SA-CXP1 | Iph-Gly-Arg-Gly-Pip-Lys-Gln-Lys-Thr-Ser-Iph-Gly-Arg-Gly-Pip; |
| 43 | SB-CXP1 | Lys-Thr-Ser-Iph-Gly-Arg-Lys-Gln-Lys-Thr-Ser-Iph-Gly-Arg-Gly- |
| | | Pip; |
| 44 | SC-CXP1 | Lys-Gln-Lys-Thr-Ser-Lys-Gln-Lys-Thr-Ser-Iph-Gly-Arg-Gly-Pip; |
| 45 | SD-CPX1 | Lys-Trp-Lys-Lys-Gln-Lys-Thr-Ser-Iph-Gly-Arg-Gly-Pip; |
| 46 | CXD1A | Lys-Thr-Ser-Iph-Gly-Arg-Lys-Gln-Lys-Thr-Ser-Iph-Gly-Arg; |
| 47 | CXD1B | Lys-Gln-Lys-Thr-Ser-Iph-Gly-Arg-Lys-Trp-Lys-Gln-Lys-Thr-Ser- |
| | | Iph-Gly-Arg; |
| 108 | CXP1X | Lys-Gln-Lys-Thr-Ser-Phe-Gly-Arg-Gly-Pip; |
| 109 | CXP1Y | Lys-Gln-Lys-Thr-Ser-Iph-Gly-Arg-Gly-Lys; |
| 110 | CXP1Z | Lys-Gln-Lys-Thr-Ser-Phe-Gly-Arg-Gly-Lys; |
| 111 | CXP1AA | Lys-Gln-Lys-Thr-Ser-Iph-Gly-Lys-Gly-Pip; |
| 112 | CXP1P | Lys-Gln-Lys-Thr-Ser-Iph-Ala-Arg-Ala-Pip; |
| 113 | CXP1B | Ala-Gln-Lys-Thr-Ser-Iph-Gly-Arg-Gly-Pip; |
| 114 | CXP1C | Lys-Ala-Lys-Thr-Ser-Iph-Gly-Arg-Gly-Pip; |
| 115 | CXP1D | Lys-Gln-Ala-Thr-Ser-Iph-Gly-Arg-Gly-Pip; |
| 116 | CXP1E | Lys-Gln-Lys-Ala-Ser-Iph-Gly-Arg-Gly-Pip; |
| 117 | CXP1F | Lys-Gln-Lys-Thr-Ala-Iph-Gly-Arg-Gly-Pip; |
| 118 | CXP1G | Lys-Gln-Lys-Thr-Ser-Ala-Gly-Arg-Gly-Pip; |
| 119 | CXP1H | Lys-Gln-Lys-Thr-Ser-Iph-Ala-Arg-Gly-Pip; |
| 120 | CXP1I | Lys-Gln-Lys-Thr-Ser-Iph-Gly-Ala-Gly-Pip; |
| 121 | CXP1J | Lys-Gln-Lys-Thr-Ser-Iph-Gly-Arg-Ala-Pip; |
| 122 | CXP1K | Lys-Gln-Lys-Thr-Ser-Iph-Gly-Arg-Gly-Ala; |
| 123 | CXP1L | Lys-Gln-Lys-Thr-Ser-Iph-Gly-Arg-Gly-Pip-Ala; |
| 124 | CXP1M | Lys-Gln-Lys-Thr-Ser-Iph-Gly-Arg-Gly-Pip-Lys-Ala; |
| 125 | CXP1N | Lys-Gln-Lys-Thr-Ser-Iph-Gly-Arg-Gly-Pip-Lys-Trp-Ala; |
| 126 | CXP1O | Lys-Gln-Lys-Thr-Ser-Iph-Gly-Arg-Gly-Pip-Lys-Trp-Lys-Ala; |
| 127 | CXP1Q | Iph-Gln-Lys-Thr-Ser-Iph-Gly-Arg-Gly-Pip; |
| 128 | CXP1R | Lys-Gln-Iph-Thr-Ser-Iph-Gly-Arg-Gly-Pip; |
| 129 | CXP1S | Lys-Gln-Lys-Thr-Ser-Iph-Gly-Iph-Gly-Pip; |
| 130 | CXP1T | Lys-Gln-Lys-Thr-Ser-Iph-Gly-Arg-Gly-Iph; |
| 131 | CXP1U | Lys-Gln-Lys-Thr-Ser-Iph-Gly-Arg-Gly-Pip-Iph-Trp-Lys-Lys; |
| 132 | CXP1V | Lys-Gln-Lys-Thr-Ser-Iph-Gly-Arg-Gly-Pip-Lys-Trp-Iph-Lys; |
| 133 | CXP1W | Lys-Gln-Lys-Thr-Ser-Iph-Gly-Arg-Gly-Pip-Lys-Trp-Lys-Iph; |
| 134 | CXP1AC | Dab-Gln-Dab-Thr-Ser-Iph-Gly-Arg-Gly-Pip-Dab-Trp-Dab-Dab; |
| 135 | CXP1AD | Dab-Gln-Dab-Thr-Ser-Iph-Gly-Dab-Gly-Pip-Dab-Trp-Dab-Dab; |
| 136 | CXP1AE | Dab-Gln-Dab-Thr-Ser-Iph-Gly-Arg-Gly-Dab-Dab-Trp-Dab-Dab; |
| | | and |
| 137 | CXP1AF | Dab-Gln-Dab-Thr-Ser-Iph-Gly-Dab-Gly-Dab-Dab-Trp-Dab-Dab. |

In another embodiment, the portion of the CPP defined by -(J^{1C})ₜ₁- or -(J^{2C})ₜ₂- has a peptide sequence selected from:

| SEQ ID | Peptide | |
|---|---|---|
| NO. | portion | Sequence |
| 1 | CXP1 | Lys-Gln-Lys-Thr-Ser-Iph-Gly-Arg-Gly-Pip; |
| 2 | CXP2 | Asn-Phe-Lys-Gln-Nap-His-Ala-Gly-Dab-Arg; |
| 3 | CXP3 | Dab-Val-Ala-Arg-Asn-Asn-Dab-Thr-Lys-Gly; |
| 4 | CXP4 | Cit-Met-Phe-Gly-Pip-Dab-Dab-Lys-Ala-Pro; |
| 5 | CXP5 | Dab-Pro-Arg-Dab-Leu-His-Ser-Dab-Ala-Thr; |
| 6 | CXP6 | Pro-Pip-Gly-Dab-Lys-Dab-Ser-Val-Iph-Nap; |
| 7 | CXP7 | Val-Gln-His-Iph-Arg-Dab-Lys-Glu-Lys-Asn; |
| 8 | CXP8 | Asn-Phe-Asn-Asn-Arg-Ser-Dab-Gly-His-Lys; |
| 9 | CXP9 | Ala-Arg-Ser-Glu-Gln-His-His-Nap-Ser-Dab; |
| 10 | SA | Iph-Gly-Arg-Gly-Pip; |
| 11 | SB | Lys-Thr-Ser-Iph-Gly-Arg; |
| 12 | SC | Lys-Gln-Lys-Thr-Ser; |
| 13 | SD | Lys-Trp-Lys; |
| 14 | SE | Iph-Gly-Arg; |
| 15 | SF | Lys-Gln-Lys-Thr-Ser-Iph-Gly-Arg; |
| 16 | SG | Ser-Iph-Gly-Arg; |
| 18 | SI | Gln-Lys-Thr-Ser-Iph-Gly-Arg; |
| 19 | SJ | Lys-Gln-Lys-Thr-Ser-Iph-Gly-Arg; |
| 39 | CXP1-CXP1 | Lys-Gln-Lys-Thr-Ser-Iph-Gly-Arg-Gly-Pip-Lys-Trp-Lys-Lys-Gly- |
| | | Lys-Gln-Lys-Thr-Ser-Iph-Gly-Arg-Gly-Pip; |
| 40 | CXP3-CXP1 | Dab-Val-Ala-Arg-Asn-Asn-Dab-Thr-Lys-Gly-Lys-Trp-Lys-Lys- |
| | | Gly-Lys-Gln-Lys-Thr-Ser-Iph-Gly-Arg-Gly-Pip; |
| 41 | CXP4-CXP1 | Cit-Met-Phe-Gly-Pip-Dab-Dab-Lys-Ala-Pro-Lys-Trp-Lys-Lys-Gly- |
| | | Lys-Gln-Lys-Thr-Ser-Iph-Gly-Arg-Gly-Pip; |
| 42 | SA-CXP1 | Iph-Gly-Arg-Gly-Pip-Lys-Gln-Lys-Thr-Ser-Iph-Gly-Arg-Gly-Pip; |
| 43 | SB-CXP1 | Lys-Thr-Ser-Iph-Gly-Arg-Lys-Gln-Lys-Thr-Ser-Iph-Gly-Arg-Gly- |
| | | Pip; |
| 44 | SC-CXP1 | Lys-Gln-Lys-Thr-Ser-Lys-Gln-Lys-Thr-Ser-Iph-Gly-Arg-Gly-Pip; |
| 45 | SD-CPX1 | Lys-Trp-Lys-Lys-Gln-Lys-Thr-Ser-Iph-Gly-Arg-Gly-Pip; |
| 46 | CXD1A | Lys-Thr-Ser-Iph-Gly-Arg-Lys-Gln-Lys-Thr-Ser-Iph-Gly-Arg; |
| 47 | CXD1B | Lys-Gln-Lys-Thr-Ser-Iph-Gly-Arg-Lys-Trp-Lys-Gln-Lys-Thr-Ser- |
| | | Iph-Gly-Arg; |
| 108 | CXP1X | Lys-Gln-Lys-Thr-Ser-Phe-Gly-Arg-Gly-Pip; |
| 109 | CXP1Y | Lys-Gln-Lys-Thr-Ser-Iph-Gly-Arg-Gly-Lys; |
| 110 | CXP1Z | Lys-Gln-Lys-Thr-Ser-Phe-Gly-Arg-Gly-Lys; |
| 111 | CXP1AA | Lys-Gln-Lys-Thr-Ser-Iph-Gly-Lys-Gly-Pip; |
| 112 | CXP1P | Lys-Gln-Lys-Thr-Ser-Iph-Ala-Arg-Ala-Pip; |
| 113 | CXP1B | Ala-Gln-Lys-Thr-Ser-Iph-Gly-Arg-Gly-Pip; |
| 114 | CXP1C | Lys-Ala-Lys-Thr-Ser-Iph-Gly-Arg-Gly-Pip; |
| 115 | CXP1D | Lys-Gln-Ala-Thr-Ser-Iph-Gly-Arg-Gly-Pip; |
| 116 | CXP1E | Lys-Gln-Lys-Ala-Ser-Iph-Gly-Arg-Gly-Pip; |
| 117 | CXP1F | Lys-Gln-Lys-Thr-Ala-Iph-Gly-Arg-Gly-Pip; |
| 118 | CXP1G | Lys-Gln-Lys-Thr-Ser-Ala-Gly-Arg-Gly-Pip; |
| 119 | CXP1H | Lys-Gln-Lys-Thr-Ser-Iph-Ala-Arg-Gly-Pip; |
| 120 | CXP1I | Lys-Gln-Lys-Thr-Ser-Iph-Gly-Ala-Gly-Pip; |
| 121 | CXP1J | Lys-Gln-Lys-Thr-Ser-Iph-Gly-Arg-Ala-Pip; |
| 122 | CXP1K | Lys-Gln-Lys-Thr-Ser-Iph-Gly-Arg-Gly-Ala; |
| 123 | CXP1L | Lys-Gln-Lys-Thr-Ser-Iph-Gly-Arg-Gly-Pip-Ala; |
| 124 | CXP1M | Lys-Gln-Lys-Thr-Ser-Iph-Gly-Arg-Gly-Pip-Lys-Ala; |
| 125 | CXP1N | Lys-Gln-Lys-Thr-Ser-Iph-Gly-Arg-Gly-Pip-Lys-Trp-Ala; |
| 126 | CXP1O | Lys-Gln-Lys-Thr-Ser-Iph-Gly-Arg-Gly-Pip-Lys-Trp-Lys-Ala; |
| 127 | CXP1Q | Iph-Gln-Lys-Thr-Ser-Iph-Gly-Arg-Gly-Pip; |
| 128 | CXP1R | Lys-Gln-Iph-Thr-Ser-Iph-Gly-Arg-Gly-Pip; |
| 129 | CXP1S | Lys-Gln-Lys-Thr-Ser-Iph-Gly-Iph-Gly-Pip; |
| 130 | CXP1T | Lys-Gln-Lys-Thr-Ser-Iph-Gly-Arg-Gly-Iph; |
| 131 | CXP1U | Lys-Gln-Lys-Thr-Ser-Iph-Gly-Arg-Gly-Pip-Iph-Trp-Lys-Lys; |
| 132 | CXP1V | Lys-Gln-Lys-Thr-Ser-Iph-Gly-Arg-Gly-Pip-Lys-Trp-Iph-Lys; |
| 133 | CXP1W | Lys-Gln-Lys-Thr-Ser-Iph-Gly-Arg-Gly-Pip-Lys-Trp-Lys-Iph; |
| 134 | CXP1AC | Dab-Gln-Dab-Thr-Ser-Iph-Gly-Arg-Gly-Pip-Dab-Trp-Dab-Dab; |
| 135 | CXP1AD | Dab-Gln-Dab-Thr-Ser-Iph-Gly-Dab-Gly-Pip-Dab-Trp-Dab-Dab; |
| 136 | CXP1AE | Dab-Gln-Dab-Thr-Ser-Iph-Gly-Arg-Gly-Dab-Dab-Trp-Dab-Dab; |
| | | and |
| 137 | CXP1AF | Dab-Gln-Dab-Thr-Ser-Iph-Gly-Dab-Gly-Dab-Dab-Trp-Dab-Dab. |

In certain embodiments, -(J^{1C})ₜ₁-(J^{1B})_{z3}-(J^{1D})ᵤ₁ or -(J^{2C})ₜ₂-(J^{2B})_{z5}-(J^{2D})ᵤ₂ form a peptide sequence selected from:

| SEQ ID | |
|---|---|
| NO. | Sequence |
| 30 | Lys-Gln-Lys-Thr-Ser-(X-ph)-Gly-Arg-Gly-Pip-Lys-Trp-Lys-Lys-Gly-Lys-Gln-Lys- |
| | Thr-Ser-(X-ph)-Gly-Arg-Gly-Pip; |
| 31 | Dab-Val-Ala-Arg-Asn-Asn-Dab-Thr-Lys-Gly-Lys-Trp-Lys-Lys-Gly-Lys-Gln-Lys- |
| | Thr-Ser-(X-ph)-Gly-Arg-Gly-Pip; |
| 32 | Cit-Met-Phe-Gly-Pip-Dab-Dab-Lys-Ala-Pro-Lys-Trp-Lys-Lys-Gly-Lys-Gln-Lys-Thr- |
| | Ser-(X-ph)-Gly-Arg-Gly-Pip; |
| 33 | (X-ph)-Gly-Arg-Gly-Pip-Lys-Gln-Lys-Thr-Ser-(X-ph)-Gly-Arg-Gly-Pip; |
| 34 | Lys-Thr-Ser-(X-ph)-Gly-Arg-Lys-Gln-Lys-Thr-Ser-(X-ph)-Gly-Arg-Gly-Pip; |
| 35 | Lys-Gln-Lys-Thr-Ser-Lys-Gln-Lys-Thr-Ser-(X-ph)-Gly-Arg-Gly-Pip; |
| 36 | Lys-Trp-Lys-Lys-Gln-Lys-Thr-Ser-(X-ph)-Gly-Arg-Gly-Pip; |
| 37 | Lys-Thr-Ser-(X-ph)-Gly-Arg-Lys-Gln-Lys-Thr-Ser-(X-ph)-Gly-Arg; and |
| 38 | Lys-Gln-Lys-Thr-Ser-(X-ph)-Gly-Arg-Lys-Trp-Lys-Gln-Lys-Thr-Ser-(X-ph)-Gly-Arg. |

In a further embodiment, -(J^{1C})ₜ₁-(J^{1B})_{z3}-(J^{1D})ᵤ₁ or -(J^{2C})ₜ₂-(J^{2B})_{z5}-(J^{2D})ᵤ₂ form a peptide sequence selected from:

| SEQ ID | |
|---|---|
| NO. | Sequence |
| 39 | Lys-Gln-Lys-Thr-Ser-Iph-Gly-Arg-Gly-Pip-Lys-Trp-Lys-Lys-Gly-Lys-Gln-Lys-Thr- |
| | Ser-Iph-Gly-Arg-Gly-Pip; |
| 40 | Dab-Val-Ala-Arg-Asn-Asn-Dab-Thr-Lys-Gly-Lys-Trp-Lys-Lys-Gly-Lys-Gln-Lys- |
| | Thr-Ser-Iph-Gly-Arg-Gly-Pip; |
| 41 | Cit-Met-Phe-Gly-Pip-Dab-Dab-Lys-Ala-Pro-Lys-Trp-Lys-Lys-Gly-Lys-Gln-Lys-Thr- |
| | Ser-Iph-Gly-Arg-Gly-Pip; |
| 42 | Iph-Gly-Arg-Gly-Pip-Lys-Gln-Lys-Thr-Ser-Iph-Gly-Arg-Gly-Pip; |
| 43 | Lys-Thr-Ser-Iph-Gly-Arg-Lys-Gln-Lys-Thr-Ser-Iph-Gly-Arg-Gly-Pip; |
| 44 | Lys-Gln-Lys-Thr-Ser-Lys-Gln-Lys-Thr-Ser-Iph-Gly-Arg-Gly-Pip; |
| 45 | Lys-Trp-Lys-Lys-Gln-Lys-Thr-Ser-Iph-Gly-Arg-Gly-Pip; |
| 46 | Lys-Thr-Ser-Iph-Gly-Arg-Lys-Gln-Lys-Thr-Ser-Iph-Gly-Arg; and |
| 47 | Lys-Gln-Lys-Thr-Ser-Iph-Gly-Arg-Lys-Trp-Lys-Gln-Lys-Thr-Ser-Iph-Gly-Arg. |

### IV. Oligomer Chemistry Features

Also provided herein are antisense oligomer conjugates, wherein the antisense oligomer is a modified antisense oligomer. Examples of modified antisense oligomers include, without limitation, morpholino oligomers. In some embodiments, the nucleobases of the modified antisense oligomer are linked to morpholino ring structures, wherein the morpholino ring structures are joined by phosphorous containing intersubunit linkages joining a morpholino nitrogen of one ring structure to a 5′ exocyclic carbon of an adjacent ring structure.

### V. Target Sequences

In some embodiments for antisense applications, the oligonucleotide can be 100% complementary to the nucleic acid target sequence, or it may include mismatches, e.g., to accommodate variants, as long as a heteroduplex formed between the oligonucleotide and nucleic acid target sequence is sufficiently stable to withstand the action of cellular nucleases and other modes of degradation which may occur in vivo. Mismatches, if present, are less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an antisense oligomer is not necessarily 100% complementary to the nucleic acid target sequence, it is effective to stably and specifically bind to the target sequence, such that a biological activity of the nucleic acid target, e.g., expression of encoded protein(s), is modulated.

The stability of the duplex formed between an oligonucleotide and the target sequence is a function of the binding Tₘ and the susceptibility of the duplex to cellular enzymatic cleavage. The Tₘ of an oligonucleotide with respect to complementary-sequence RNA may be measured by conventional methods, such as those described by Hames et al., Nucleic Acid Hybridization, IRL Press, 1985, pp. 107-108 or as described in Miyada C G. and Wallace R B (1987) Oligonucleotide hybridization techniques, Methods Enzymol. Vol. 154 pp. 94-107.

In some embodiments, each antisense oligomer has a binding Tₘ, with respect to a complementary-sequence RNA, of greater than body temperature or in other embodiments greater than 50° C. In other embodiments Tₘ's are in the range 60-80° C. or greater. According to well known principles, the Tₘ of an oligonucleotide, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligonucleotide. For this reason, oligonucleotides that show high Tₘ (50° C. or greater) at a length of 20 bases or less are generally preferred over those requiring greater than 20 bases for high Tₘ values. For some applications, longer oligomers, for example longer than 20 bases, may have certain advantages.

The targeting sequence bases may be normal DNA bases or analogues thereof, e.g., uracil and inosine that are capable of Watson-Crick base pairing to target-sequence RNA bases.

An antisense oligomer can be designed to block or inhibit or modulate translation of mRNA or to inhibit or modulate pre-mRNA splice processing, or induce degradation of targeted mRNAs, and may be said to be “directed to” or “targeted against” a target sequence with which it hybridizes. In certain embodiments, the target sequence includes a region including a 3′ or 5′ splice site of a pre-processed mRNA, a branch point, or other sequence involved in the regulation of splicing. The target sequence may be within an exon or within an intron or spanning an intron/exon junction.

An antisense oligomer having a sufficient sequence complementarity to a target RNA sequence to modulate splicing of the target RNA means that the oligonucleotide has a sequence sufficient to trigger the masking of a binding site for a native protein that would otherwise modulate splicing and/or alters the three-dimensional structure of the targeted RNA. Likewise, an oligonucleotide having a sufficient sequence complementary to a target RNA sequence to modulate splicing of the target RNA means that the oligonucleotide reagent has a sequence sufficient to trigger the masking of a binding site for a native protein that would otherwise modulate splicing and/or alters the three-dimensional structure of the targeted RNA.

In certain embodiments, the degree of complementarity between the target sequence and antisense targeting sequence is sufficient to form a stable duplex. The region of complementarity of the antisense oligomers with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligomer of about 14-15 bases is generally long enough to have a unique complementary sequence. In certain embodiments, a minimum length of complementary bases may be required to achieve the requisite binding Tm, as discussed herein.

In certain embodiments, oligomers as long as 40 bases may be suitable, where at least a minimum number of bases, e.g., 10-12 bases, are complementary to the target sequence. In some embodiments, facilitated or active uptake in cells is optimized at oligomer lengths of less than about 30 bases. For PMOs, described further herein, an optimum balance of binding stability and uptake generally occurs at lengths of 18-25 bases. Included in the disclosure are antisense oligomers (e.g., PMOs) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases, in which at least about 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous or non-contiguous bases are complementary to the desired target sequences.

In certain embodiments, antisense oligomers may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligomers may have substantial complementarity, meaning, about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligomer backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an antisense oligomer is not necessarily 100% complementary to the target sequence, it is effective to stably and specifically bind to the target sequence, such that splicing of the target pre-RNA is modulated.

The stability of the duplex formed between an oligonucleotide and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligomer Hybridization Techniques, Methods Enzymol. Vol. 154 pp. 94-107. In certain embodiments, antisense oligomers may have a binding Tm, with respect to a complementary-sequence RNA, of greater than body temperature and preferably greater than about 45° C. or 50° C. Tm's in the range 60-80° C. or greater are also included. According to well-known principles, the Tm of an oligonucleotide, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligonucleotide. For this reason, oligonucleotides that show high Tm (45-50° C. or greater) at a length of 25 bases or less are generally preferred over those requiring greater than 25 bases for high Tm values.

In one aspect, the disclosure provides an antisense oligomer conjugate, or a pharmaceutically acceptable salt thereof, capable of binding a selected target to induce exon skipping in the human dystrophin gene, wherein the antisense oligomer conjugate, or a pharmaceutically acceptable salt thereof, comprises a sequence of bases that is complementary to an exon target region of the dystrophin pre-mRNA designated as an annealing site, wherein each nucleobase R², as recited in Formulae I, Ia, Ib, Ic, Id, II, IIa, IIb, III, IIIa and R²² as recited in Formulae IV, IVa, Va, Vb, Vc, Vd, VI, VIa, VII, VIIa, VIIIa, VIIIb, and IX, and 5′ to 3′ can be selected from:

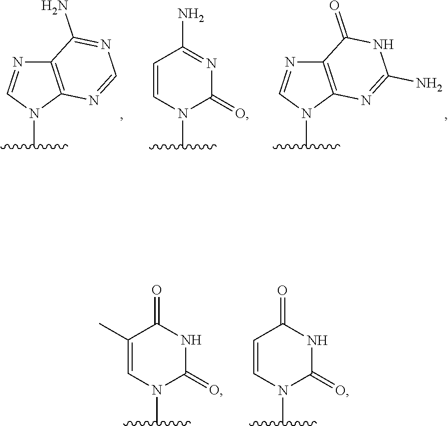

methylated guanine, methylated adenine, and

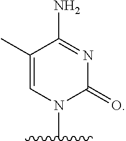 [SMILES: *n1cc(C)c(N)nc1=O]

In an embodiment, the sequence for the oligonucleotide of the antisense oligomer conjugate is GCTATTACCTTAACCCAG (SEQ ID NO.: 77).

In an embodiment, provided herein is an antisense oligonucleotide, or a pharmaceutically acceptable salt thereof, wherein the antisense oligomer conjugate is of Formula (VII):

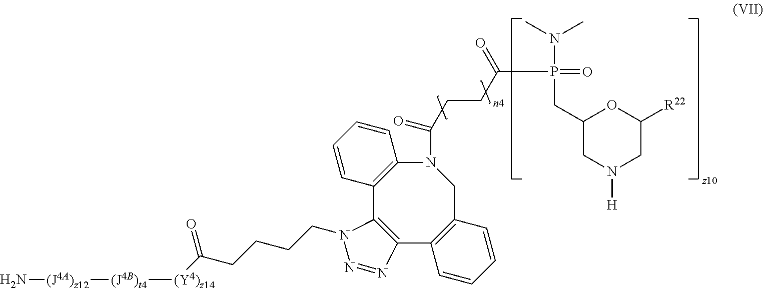

wherein z10 is 18 and R²² is a sequence of nucleobases having the sequence of GCTATTACCTTAACCCAG (SEQ ID NO.: 77). This antisense oligomer conjugate is also referred to herein as “PMO IVS2-654.”

### VI. Cell Penetrating Peptides (CPPs)

As described, cell-penetrating peptides (CPP) within the scope of substituents -(J^{1C})ₜ₁-(J^{1B})_{z3}-, -(J^{2C})ₜ₂-(J^{2B})_{z5}-, -(J^{3B})ₜ₃-(J^{3A})_{z11}-, or -(J⁴B)ₜ₄-(J^{4A})_{z12}- and chimeric cell-penetrating peptides within the scope of substituents -(J^{1C})ₜ₁-(J^{1B})_{z3}-(J^{1D})ᵤ₁-(J^{1A})_{z4}-, or -(J^{2C})ₜ₂-(J^{2B})_{z5}-(J^{2D})ᵤ₂-(J^{2A})_{z6}- have been shown to be effective in enhancing penetration of antisense oligomers into a cell and to cause exon skipping in different muscle groups in animal models.

Exemplary peptides are given below in Table 2.

TABLE 2
Peptide Sequences

| SEQ ID | Peptide | |
|---|---|---|
| NO. | Portion | Sequence |
| 1 | CXP1 | Lys-Gln-Lys-Thr-Ser-Iph-Gly-Arg-Gly-Pip; |
| 2 | CXP2 | Asn-Phe-Lys-Gln-Nap-His-Ala-Gly-Dab-Arg; |
| 3 | CXP3 | Dab-Val-Ala-Arg-Asn-Asn-Dab-Thr-Lys-Gly; |
| 4 | CXP4 | Cit-Met-Phe-Gly-Pip-Dab-Dab-Lys-Ala-Pro; |
| 5 | CXP5 | Dab-Pro-Arg-Dab-Leu-His-Ser-Dab-Ala-Thr; |
| 6 | CXP6 | Pro-Pip-Gly-Dab-Lys-Dab-Ser-Val-Iph-Nap; |
| 7 | CXP7 | Val-Gln-His-Iph-Arg-Dab-Lys-Glu-Lys-Asn; |
| 8 | CXP8 | Asn-Phe-Asn-Asn-Arg-Ser-Dab-Gly-His-Lys; |
| 9 | CXP9 | Ala-Arg-Ser-Glu-Gln-His-His-Nap-Ser-Dab; |
| 10 | SA | Iph-Gly-Arg-Gly-Pip; |
| 11 | SB | Lys-Thr-Ser-Iph-Gly-Arg; |
| 12 | SC | Lys-Gln-Lys-Thr-Ser; |
| 13 | SD | Lys-Trp-Lys; |
| 14 | SE | Iph-Gly-Arg; |
| 15 | SF | Lys-Gln-Lys-Thr-Ser-Iph-Gly-Arg; |
| 16 | SG | Ser-Iph-Gly-Arg; |
| 18 | SI | Gln-Lys-Thr-Ser-Iph-Gly-Arg; |
| 19 | SJ | Lys-Gln-Lys-Thr-Ser-Iph-Gly-Arg; |
| 39 | CXP1-CXP1 | Lys-Gln-Lys-Thr-Ser-Iph-Gly-Arg-Gly-Pip-Lys-Trp-Lys-Lys-Gly- |
| | | Lys-Gln-Lys-Thr-Ser-Iph-Gly-Arg-Gly-Pip; |
| 40 | CXP3-CXP1 | Dab-Val-Ala-Arg-Asn-Asn-Dab-Thr-Lys-Gly-Lys-Trp-Lys-Lys- |
| | | Gly-Lys-Gln-Lys-Thr-Ser-Iph-Gly-Arg-Gly-Pip; |
| 41 | CXP4-CXP1 | Cit-Met-Phe-Gly-Pip-Dab-Dab-Lys-Ala-Pro-Lys-Trp-Lys-Lys-Gly- |
| | | Lys-Gln-Lys-Thr-Ser-Iph-Gly-Arg-Gly-Pip; |
| 42 | SA-CXP1 | Iph-Gly-Arg-Gly-Pip-Lys-Gln-Lys-Thr-Ser-Iph-Gly-Arg-Gly-Pip; |
| 43 | SB-CXP1 | Lys-Thr-Ser-Iph-Gly-Arg-Lys-Gln-Lys-Thr-Ser-Iph-Gly-Arg-Gly- |
| | | Pip; |
| 44 | SC-CXP1 | Lys-Gln-Lys-Thr-Ser-Lys-Gln-Lys-Thr-Ser-Iph-Gly-Arg-Gly-Pip; |
| 45 | SD-CPX1 | Lys-Trp-Lys-Lys-Gln-Lys-Thr-Ser-Iph-Gly-Arg-Gly-Pip; |
| 46 | CXD1A | Lys-Thr-Ser-Iph-Gly-Arg-Lys-Gln-Lys-Thr-Ser-Iph-Gly-Arg; |
| 47 | CXD1B | Lys-Gln-Lys-Thr-Ser-Iph-Gly-Arg-Lys-Trp-Lys-Gln-Lys-Thr-Ser- |
| | | Iph-Gly-Arg; |
| 108 | CXP1X | Lys-Gln-Lys-Thr-Ser-Phe-Gly-Arg-Gly-Pip; |
| 109 | CXP1Y | Lys-Gln-Lys-Thr-Ser-Iph-Gly-Arg-Gly-Lys; |
| 110 | CXP1Z | Lys-Gln-Lys-Thr-Ser-Phe-Gly-Arg-Gly-Lys; |
| 111 | CXP1AA | Lys-Gln-Lys-Thr-Ser-Iph-Gly-Lys-Gly-Pip; |
| 112 | CXP1P | Lys-Gln-Lys-Thr-Ser-Iph-Ala-Arg-Ala-Pip; |
| 113 | CXP1B | Ala-Gln-Lys-Thr-Ser-Iph-Gly-Arg-Gly-Pip; |
| 114 | CXP1C | Lys-Ala-Lys-Thr-Ser-Iph-Gly-Arg-Gly-Pip; |
| 115 | CXP1D | Lys-Gln-Ala-Thr-Ser-Iph-Gly-Arg-Gly-Pip; |
| 116 | CXP1E | Lys-Gln-Lys-Ala-Ser-Iph-Gly-Arg-Gly-Pip; |
| 117 | CXP1F | Lys-Gln-Lys-Thr-Ala-Iph-Gly-Arg-Gly-Pip; |
| 118 | CXP1G | Lys-Gln-Lys-Thr-Ser-Ala-Gly-Arg-Gly-Pip; |
| 119 | CXP1H | Lys-Gln-Lys-Thr-Ser-Iph-Ala-Arg-Gly-Pip; |
| 120 | CXP1I | Lys-Gln-Lys-Thr-Ser-Iph-Gly-Ala-Gly-Pip; |
| 121 | CXP1J | Lys-Gln-Lys-Thr-Ser-Iph-Gly-Arg-Ala-Pip; |
| 122 | CXP1K | Lys-Gln-Lys-Thr-Ser-Iph-Gly-Arg-Gly-Ala; |
| 123 | CXP1L | Lys-Gln-Lys-Thr-Ser-Iph-Gly-Arg-Gly-Pip-Ala; |
| 124 | CXP1M | Lys-Gln-Lys-Thr-Ser-Iph-Gly-Arg-Gly-Pip-Lys-Ala; |
| 125 | CXP1N | Lys-Gln-Lys-Thr-Ser-Iph-Gly-Arg-Gly-Pip-Lys-Trp-Ala; |
| 126 | CXP1O | Lys-Gln-Lys-Thr-Ser-Iph-Gly-Arg-Gly-Pip-Lys-Trp-Lys-Ala; |
| 127 | CXP1Q | Iph-Gln-Lys-Thr-Ser-Iph-Gly-Arg-Gly-Pip; |
| 128 | CXP1R | Lys-Gln-Iph-Thr-Ser-Iph-Gly-Arg-Gly-Pip; |
| 129 | CXP1S | Lys-Gln-Lys-Thr-Ser-Iph-Gly-Iph-Gly-Pip; |
| 130 | CXP1T | Lys-Gln-Lys-Thr-Ser-Iph-Gly-Arg-Gly-Iph; |
| 131 | CXP1U | Lys-Gln-Lys-Thr-Ser-Iph-Gly-Arg-Gly-Pip-Iph-Trp-Lys-Lys; |
| 132 | CXP1V | Lys-Gln-Lys-Thr-Ser-Iph-Gly-Arg-Gly-Pip-Lys-Trp-Iph-Lys; |
| 133 | CXP1W | Lys-Gln-Lys-Thr-Ser-Iph-Gly-Arg-Gly-Pip-Lys-Trp-Lys-Iph; |
| 134 | CXP1AC | Dab-Gln-Dab-Thr-Ser-Iph-Gly-Arg-Gly-Pip-Dab-Trp-Dab-Dab; |
| 135 | CXP1AD | Dab-Gln-Dab-Thr-Ser-Iph-Gly-Dab-Gly-Pip-Dab-Trp-Dab-Dab; |
| 136 | CXP1AE | Dab-Gln-Dab-Thr-Ser-Iph-Gly-Arg-Gly-Dab-Dab-Trp-Dab-Dab; |
| | | and |
| 137 | CXP1AF | Dab-Gln-Dab-Thr-Ser-Iph-Gly-Dab-Gly-Dab-Dab-Trp-Dab-Dab. |

**The peptide sequences are optionally covalently linked by an amide bond to a linking amino acid/s (defined herein as Y¹, Y², Y³, or Y⁴ in the definition of L¹, L², L³, or L⁴).

In some embodiments, the peptide sequence of the cell-penetrating peptides further comprises an amino acid sequence selected from Lys-Trp-Lys-Lys, Lys-Lys-Trp-Lys, Lys-Trp-Trp-Lys-Lys, Trp-Trp-Lys-Lys, Lys-Trp-Lys, Trp-Lys-Lys, Lys-Lys-Trp, Lys-Lys-Lys-Lys, Lys-Lys, and Lys-Trp at the C-terminus.

Exemplary peptides are given below in Table 3.

TABLE 3
Peptide Sequences comprising a Lys-Trp-Lys-Lys amino acid sequence at the C-
terminus.

| Compound | Peptide | |
|---|---|---|
| Name | Name | Peptide Sequence |
| CXP1 | 48 | Lys-Gln-Lys-Thr-Ser-Iph-Gly-Arg-Gly-Pip-Lys-Trp-Lys-Lys; |
| CXP2 | 49 | Asn-Phe-Lys-Gln-Nap-His-Ala-Gly-Dab-Arg-Lys-Trp-Lys-Lys; |
| CXP3 | 50 | Dab-Val-Ala-Arg-Asn-Asn-Dab-Thr-Lys-Gly-Lys-Trp-Lys-Lys; |
| CXP4 | 51 | Cit-Met-Phe-Gly-Pip-Dab-Dab-Lys-Ala-Pro-Lys-Trp-Lys-Lys; |
| CXP5 | 52 | Dab-Pro-Arg-Dab-Leu-His-Ser-Dab-Ala-Thr-Lys-Trp-Lys-Lys; |
| CXP6 | 53 | Pro-Pip-Gly-Dab-Lys-Dab-Ser-Val-Iph-Nap-Lys-Trp-Lys-Lys; |
| CXP7 | 54 | Val-Gln-His-Iph-Arg-Dab-Lys-Glu-Lys-Asn-Lys-Trp-Lys-Lys; |
| CXP8 | 55 | Asn-Phe-Asn-Asn-Arg-Ser-Dab-Gly-His-Lys-Lys-Trp-Lys-Lys; |
| CXP9 | 56 | Ala-Arg-Ser-Glu-Gln-His-His-Nap-Ser-Dab-Lys-Trp-Lys-Lys; |
| SA | 57 | Iph-Gly-Arg-Gly-Pip-Lys-Trp-Lys-Lys; |
| SB | 58 | Lys-Thr-Ser-Iph-Gly-Arg-Lys-Trp-Lys-Lys; |
| SC | 59 | Lys-Gln-Lys-Thr-Ser-Lys-Trp-Lys-Lys; |
| SD | 60 | Lys-Trp-Lys-Lys-Trp-Lys-Lys; |
| SE | 61 | Iph-Gly-Arg-Lys-Trp-Lys-Lys; |
| SF | 62 | Lys-Gln-Lys-Thr-Ser-Iph-Gly-Arg-Lys-Trp-Lys-Lys; |
| SG | 63 | Ser-Iph-Gly-Arg-Lys-Trp-Lys-Lys; |
| SH | 64 | Lys-Thr-Ser-Lys-Trp-Lys-Lys; |
| SI | 65 | Gln-Lys-Thr-Ser-Iph-Gly-Arg-Lys-Trp-Lys-Lys; |
| SJ | 66 | Lys-Gln-Lys-Thr-Ser-Iph-Gly-Arg-Lys-Trp-Lys-Lys; |
| CXP1-CXP1 | 67 | Lys-Gln-Lys-Thr-Ser-Iph-Gly-Arg-Gly-Pip-Lys-Trp-Lys-Lys-Gly- |
| | | Lys-Gln-Lys-Thr-Ser-Iph-Gly-Arg-Gly-Pip-Lys-Trp-Lys-Lys; |
| CXP3-CXP1 | 68 | Dab-Val-Ala-Arg-Asn-Asn-Dab-Thr-Lys-Gly-Lys-Trp-Lys-Lys- |
| | | Gly-Lys-Gln-Lys-Thr-Ser-Iph-Gly-Arg-Gly-Pip-Lys-Trp-Lys-Lys; |
| CXP4-CXP1 | 69 | Cit-Met-Phe-Gly-Pip-Dab-Dab-Lys-Ala-Pro-Lys-Trp-Lys-Lys- |
| | | Gly-Lys-Gln-Lys-Thr-Ser-Iph-Gly-Arg-Gly-Pip-Lys-Trp-Lys-Lys; |
| SA-CXP1 | 70 | Iph-Gly-Arg-Gly-Pip-Lys-Gln-Lys-Thr-Ser-Iph-Gly-Arg-Gly-Pip- |
| | | Lys-Trp-Lys-Lys; |
| SB-CXP1 | 71 | Lys-Thr-Ser-Iph-Gly-Arg-Lys-Gln-Lys-Thr-Ser-Iph-Gly-Arg-Gly- |
| | | Pip-Lys-Trp-Lys-Lys; |
| SC-CXP1 | 72 | Lys-Gln-Lys-Thr-Ser-Lys-Gln-Lys-Thr-Ser-Iph-Gly-Arg-Gly-Pip- |
| | | Lys-Trp-Lys-Lys; |
| SD-CXP1 | 73 | Lys-Trp-Lys-Lys-Gln-Lys-Thr-Ser-Iph-Gly-Arg-Gly-Pip -- Lys- |
| | | Trp-Lys-Lys; |
| CXD1A | 74 | Lys-Thr-Ser-Iph-Gly-Arg-Lys-Gln-Lys-Thr-Ser-Iph-Gly-Arg-Lys- |
| | | Trp-Lys-Lys; |
| CXD1B | 75 | Lys-Gln-Lys-Thr-Ser-Iph-Gly-Arg-Lys-Trp-Lys-Gln-Lys-Thr-Ser- |
| | | Iph-Gly-Arg-Lys-Trp-Lys-Lys. |
| CXP1X | 78 | Lys-Gln-Lys-Thr-Ser-Phe-Gly-Arg-Gly-Pip-Lys-Trp-Lys-Lys; |
| CXP1Y | 79 | Lys-Gln-Lys-Thr-Ser-Iph-Gly-Arg-Gly-Lys-Lys-Trp-Lys-Lys; |
| CXP1Z | 80 | Lys-Gln-Lys-Thr-Ser-Phe-Gly-Arg-Gly-Lys-Lys-Trp-Lys-Lys; |
| CXP1AA | 81 | Lys-Gln-Lys-Thr-Ser-Iph-Gly-Lys-Gly-Pip-Lys-Trp-Lys-Lys; |
| CXP1P | 82 | Lys-Gln-Lys-Thr-Ser-Iph-Ala-Arg-Ala-Pip-Lys-Trp-Lys-Lys; |
| CXP1B | 83 | Ala-Gln-Lys-Thr-Ser-Iph-Gly-Arg-Gly-Pip-Lys-Trp-Lys-Lys; |
| CXP1C | 84 | Lys-Ala-Lys-Thr-Ser-Iph-Gly-Arg-Gly-Pip-Lys-Trp-Lys-Lys; |
| CXP1D | 85 | Lys-Gln-Ala-Thr-Ser-Iph-Gly-Arg-Gly-Pip-Lys-Trp-Lys-Lys; |
| CXP1E | 86 | Lys-Gln-Lys-Ala-Ser-Iph-Gly-Arg-Gly-Pip-Lys-Trp-Lys-Lys; |
| CXP1F | 87 | Lys-Gln-Lys-Thr-Ala-Iph-Gly-Arg-Gly-Pip-Lys-Trp-Lys-Lys; |
| CXP1G | 88 | Lys-Gln-Lys-Thr-Ser-Ala-Gly-Arg-Gly-Pip-Lys-Trp-Lys-Lys; |
| CXP1H | 89 | Lys-Gln-Lys-Thr-Ser-Iph-Ala-Arg-Gly-Pip-Lys-Trp-Lys-Lys; |
| CXP1I | 90 | Lys-Gln-Lys-Thr-Ser-Iph-Gly-Ala-Gly-Pip-Lys-Trp-Lys-Lys; |
| CXP1J | 91 | Lys-Gln-Lys-Thr-Ser-Iph-Gly-Arg-Ala-Pip-Lys-Trp-Lys-Lys; |
| CXP1K | 92 | Lys-Gln-Lys-Thr-Ser-Iph-Gly-Arg-Gly-Ala-Lys-Trp-Lys-Lys; |
| CXP1L | 93 | Lys-Gln-Lys-Thr-Ser-Iph-Gly-Arg-Gly-Pip-Ala-Trp-Lys-Lys; |
| CXP1M | 94 | Lys-Gln-Lys-Thr-Ser-Iph-Gly-Arg-Gly-Pip-Lys-Ala-Lys-Lys; |
| CXP1N | 95 | Lys-Gln-Lys-Thr-Ser-Iph-Gly-Arg-Gly-Pip-Lys-Trp-Ala-Lys; |
| CXP1O | 96 | Lys-Gln-Lys-Thr-Ser-Iph-Gly-Arg-Gly-Pip-Lys-Trp-Lys-Ala-Lys- |
| | | Trp-Lys-Lys; |
| CXP1Q | 97 | Iph-Gln-Lys-Thr-Ser-Iph-Gly-Arg-Gly-Pip-Lys-Trp-Lys-Lys; |
| CXP1R | 98 | Lys-Gln-Iph-Thr-Ser-Iph-Gly-Arg-Gly-Pip-Lys-Trp-Lys-Lys; |
| CXP1S | 99 | Lys-Gln-Lys-Thr-Ser-Iph-Gly-Iph-Gly-Pip-Lys-Trp-Lys-Lys; |
| CXP1T | 100 | Lys-Gln-Lys-Thr-Ser-Iph-Gly-Arg-Gly-Iph-Lys-Trp-Lys-Lys; |
| CXP1U | 101 | Lys-Gln-Lys-Thr-Ser-Iph-Gly-Arg-Gly-Pip-Iph-Trp-Lys-Lys |
| CXP1V | 102 | Lys-Gln-Lys-Thr-Ser-Iph-Gly-Arg-Gly-Pip-Lys-Trp-Iph-Lys; |
| CXP1W | 103 | Lys-Gln-Lys-Thr-Ser-Iph-Gly-Arg-Gly-Pip-Lys-Trp-Lys-Iph-Lys- |
| | | Trp-Lys-Lys; |
| CXP1AC | 104 | Dab-Gln-Dab-Thr-Ser-Iph-Gly-Arg-Gly-Pip-Dab-Trp-Dab-Dab- |
| | | Lys-Trp-Lys-Lys; |
| CXP1AD | 105 | Dab-Gln-Dab-Thr-Ser-Iph-Gly-Dab-Gly-Pip-Dab-Trp-Dab-Dab- |
| | | Lys-Trp-Lys-Lys; |
| CXP1AE | 106 | Dab-Gln-Dab-Thr-Ser-Iph-Gly-Arg-Gly-Dab-Dab-Trp-Dab-Dab- |
| | | Lys-Trp-Lys-Lys; and |
| CXP1AF | 107 | Dab-Gln-Dab-Thr-Ser-Iph-Gly-Dab-Gly-Dab-Dab-Trp-Dab- |
| | | Dab-Lys-Trp-Lys-Lys. |

**The peptide sequences are optionally covalently linked by an amide bond to a linking amino acid/s (defined herein as Y¹, Y², Y³, or Y⁴ in the definition of L¹, L², L³, or L⁴).

Exemplary C-terminal “Lys-Trp” motif peptides are given below in Table 4.

TABLE 4
“Lys-Trp” Motif Amino Acid Sequences.

| SEQ ID | |
|---|---|
| NO. | Sequence |
| 138 | Lys-Lys; |
| 139 | Lys-Trp; |
| 140 | Lys-Trp-Lys; |
| 141 | Trp-Lys-Lys; |
| 142 | Lys-Lys-Trp; |
| 143 | Lys-Trp-Lys-Lys; |
| 144 | Lys-Lys-Trp-Lys; |
| 145 | Lys-Trp-Trp-Lys-Lys; |
| 146 | Trp-Trp-Lys-Lys; and |
| 147 | Lys-Lys-Lys-Lys. |

In some embodiments, the cell-penetrating peptides can be 3 to 40 amino acids (excluding the linking amino acid/s at the N-terminus (Y¹, Y², Y³, or Y⁴ in the definition of L¹, L², L³, or L⁴)). Included in the disclosure are cell penetrating peptides comprises from 3 to 80 amino acids selected from canonical and non-canonical amino acids. In other embodiments, the cell penetrating peptides comprises from 3 to 70, from 3 to 60, from 3 to 50, from 3 to 40, from 3 to 30, from 3 to 20, from 8 to 25 or from 10 to 20 amino acids. In other embodiments, the carrier peptide comprises from 5 to 70, from 6 to 70, from 8 to 70, from 10 to 70, from 15 to 70, from 20 to 70, from 25 to 70, from 30 to 70, from 35 to 70, or from 40 to 70 amino acids selected from canonical and non-canonical amino acids. In other embodiments, the cell penetrating peptides comprises from 5 to 40, from 10 to 30, from 15 to 30, or from 20 to 30 amino acids selected from canonical and non-canonical amino acids.

In an embodiment, -(J^{1C})ₜ₁-, -(J^{1D})ᵤ₁-, -(J^{2C})ₜ₂, -(J^{2D})ᵤ₂-, -(J^{3B})ₜ₃-, or -(J^{4B})ₜ₄- is independently at each occurrence selected from the canonical amino acids: arginine, histidine, lysine, aspartic acid, glutamic acid, serine, threonine, asparagine, glutamine, glycine, methionine, proline, valine, alanine, leucine, phenylalanine, and tryptophan; and the non-canonical amino acids:

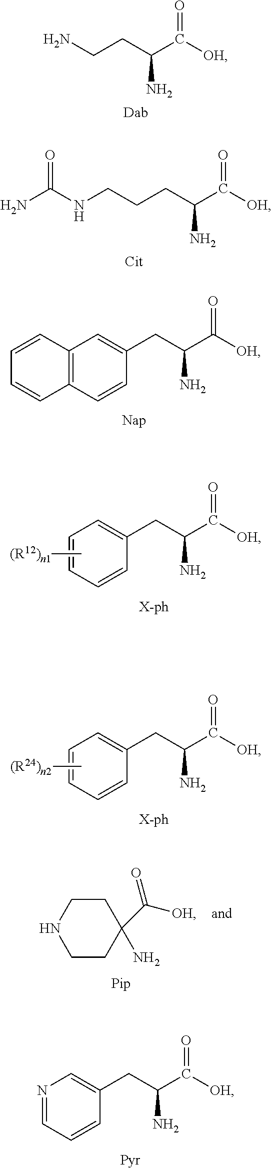

wherein n1 and n2 are each independently an integer from 1 to 5 and X is halogen. In a further embodiment, X is I.

CPPs within the scope of substituents -(J^{1B})_{z3}-, -(J^{1A})_{z4}-, -(J^{2B})_{z5}-, -(J^{2A})_{z6}-, -(J^{3A})_{z11}-, or -(J^{4A})_{z12}.

In certain embodiments, -(J^{1B})_{z3}-, -(J^{1A})_{z4}-, -(J^{2B})_{z5}-, -(J^{2A})_{z6}-, -(J^{3A})_{z11}-, or -(J^{4A})_{z12} is absent or the amino acid sequence selected from Lys-Trp-Lys-Lys, Lys-Lys-Trp-Lys, Lys-Trp-Trp-Lys-Lys, Trp-Trp-Lys-Lys, Lys-Trp-Lys, Trp-Lys-Lys, Lys-Lys-Trp, Lys-Lys-Lys-Lys, Lys-Lys, and Lys-Trp. In another embodiment, -(J^{1B})_{z3}-, -(J^{1A})_{z4}-, -(J^{2B})_{z5}-, -(J^{2A})_{z6}-, -(J^{3A})_{z11}-, or -(J^{4A})_{z12} is absent. In another embodiment, -(J^{1B})_{z3}-, -(J^{1A})_{z4}-, -(J^{2B})_{z5}-, -(J^{2A})_{z6}-, -(J^{3A})_{z11}-, or -(J^{4A})_{z12} is the amino acid sequence selected from Lys-Trp-Lys-Lys, Lys-Lys-Trp-Lys, Lys-Trp-Trp-Lys-Lys, Trp-Trp-Lys-Lys, Lys-Trp-Lys, Trp-Lys-Lys, Lys-Lys-Trp, Lys-Lys-Lys-Lys, Lys-Lys, and Lys-Trp. In yet another embodiment, -(J^{1B})_{z3}-, -(J^{1A})_{z4}-, -(J^{2B})_{z5}-, -(J^{2A})_{z6}-, -(J^{3A})_{z11}-, or -(J^{4A})_{z12} is Lys-Trp-Lys-Lys.

In another embodiment, -(J^{1C})ₜ₁-, -(J^{2C})ₜ₂-, -(J^{3B})ₜ₃, or -(J^{4B})ₜ₄- is a peptide sequence selected from:

| SEQ ID NO. | Peptide Sequence |
|---|---|
| 149 | Lys-Gln-Lys-Thr-Ser-(X-ph)-Gly-Arg-Gly-Pip; |
| 2 | Asn-Phe-Lys-Gln-Nap-His-Ala-Gly-Dab-Arg; |
| 3 | Dab-Val-Ala-Arg-Asn-Asn-Dab-Thr-Lys-Gly; |
| 4 | Cit-Met-Phe-Gly-Pip-Dab-Dab-Lys-Ala-Pro; |
| 5 | Dab-Pro-Arg-Dab-Leu-His-Ser-Dab-Ala-Thr; |
| 150 | Pro-Pip-Gly-Dab-Lys-Dab-Ser-Val-(X-ph)-Nap; |
| 151 | Val-Gln-His-(X-ph)-Arg-Dab-Lys-Glu-Lys-Asn; |
| 8 | Asn-Phe-Asn-Asn-Arg-Ser-Dab-Gly-His-Lys; |
| 9 | Ala-Arg-Ser-Glu-Gln-His-His-Nap-Ser-Dab; |
| 152 | (X-ph)-Gly-Arg-Gly-Pip |
| 153 | Lys-Thr-Ser-(X-ph)-Gly-Arg |
| 154 | (X-ph)-Gly-Arg |
| 155 | Lys-Gln-Lys-Thr-Ser-(X-ph)-Gly-Arg |
| 156 | Ser-(X-ph)-Gly-Arg |
| 157 | Gln-Lys-Thr-Ser-(X-ph)-Gly-Arg |
| 158 | Lys-Gln-Lys-Thr-Ser-(X-ph)-Gly-Arg. |

In another embodiment, -(J^{1C})ₜ₁-, -(J^{2C})ₜ₂-, -(J^{3B})ₜ₃-, or -(J^{4B})ₜ₄- is a peptide sequence selected from:

| SEQ | |
|---|---|
| ID | |
| NO. | Peptide Sequence |
| 1 | Lys-Gln-Lys-Thr-Ser-Iph-Gly-Arg-Gly-Pip; |
| 2 | Asn-Phe-Lys-Gln-Nap-His-Ala-Gly-Dab-Arg; |
| 3 | Dab-Val-Ala-Arg-Asn-Asn-Dab-Thr-Lys-Gly; |
| 4 | Cit-Met-Phe-Gly-Pip-Dab-Dab-Lys-Ala-Pro; |
| 5 | Dab-Pro-Arg-Dab-Leu-His-Ser-Dab-Ala-Thr; |
| 6 | Pro-Pip-Gly-Dab-Lys-Dab-Ser-Val-Iph-Nap; |
| 7 | Val-Gln-His-Iph-Arg-Dab-Lys-Glu-Lys-Asn; |
| 8 | Asn-Phe-Asn-Asn-Arg-Ser-Dab-Gly-His-Lys; |
| 9 | Ala-Arg-Ser-Glu-Gln-His-His-Nap-Ser-Dab; |
| 10 | Iph-Gly-Arg-Gly-Pip |
| 11 | Lys-Thr-Ser-Iph-Gly-Arg |
| 14 | Iph-Gly-Arg |
| 15 | Lys-Gln-Lys-Thr-Ser-Iph-Gly-Arg |
| 16 | Ser-Iph-Gly-Arg |
| 18 | Gln-Lys-Thr-Ser-Iph-Gly-Arg |
| 19 | Lys-Gln-Lys-Thr-Ser-Iph-Gly-Arg. |

In a further embodiment,

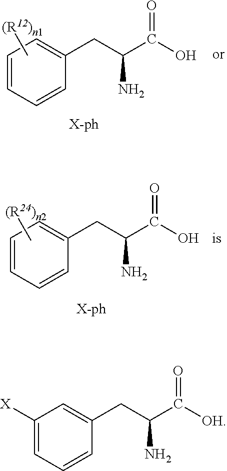

embodiment, X is I.

In other embodiments, -(J^{1C})ₜ₁-, -(J^{2C})ₜ₂-, -(J^{3B})ₜ₃-, or -(J^{4B})ₜ₄- comprises an (X-ph)-Gly-Arg motif. In another embodiment, -(J^{1C})ₜ₁-, -(J^{2C})ₜ₂-, -(J^{3B})ₜ₃-, or -(J^{4B})ₜ₄- comprises an Iph-Gly-Arg motif. In a further embodiment, -(J^{1C})ₜ₁-, -(J^{2C})ₜ₂-, (J^{3B})ₜ₃-, or -(J^{4B})ₜ₄- is Lys-Gln-Lys-Thr-Ser-Iph-Gly-Arg-Gly-Pip (SEQ ID NO.: 20).

Chimeric CPPs within the scope of substituents -(J^{1C})-(J^{1B})_{z3}-(J^{1D})ᵤ₁-(J^{1A})_{z4}-, or -(J^{2C})ₜ₁-(J^{2B})_{z5}-(J^{2D})ᵤ₂-(J^{2A})_{z6}

In some embodiments, the chimeric cell penetrating peptide is of the formula: -(J^{1C})ₜ₁-(J^{1B})_{z3}-(J^{1D})ᵤ₁-(J^{1A})_{z4}-, or -(J^{2C})ₜ₂-(J^{2B})_{z5}-(J^{2D})ᵤ₂-(J^{2A})_{z6}-, wherein t1 and t2 are each independently an integer from 3 to 20, z3, z4, z5, and z6 are each independently 0 or 1, and u1 and u2 are each independently an integer selected from 0 and 3 to 20.

In an embodiment, -(J^{1B})_{z3}-, or -(J^{2B})_{z5}- is absent, or -(J^{1B})_{z3}-, or -(J^{2B})_{z5}- is the amino acid sequence selected from: Lys-Trp-Lys-Lys, Lys-Lys-Trp-Lys, Lys-Trp-Trp-Lys-Lys, Trp-Trp-Lys-Lys, Lys-Trp-Lys, Trp-Lys-Lys, Lys-Lys-Trp, Lys-Lys-Lys-Lys, Lys-Lys, and Lys-Trp.

In an embodiment, the chimeric cell penetrating peptide further comprises a linking amino acid, e.g., Y¹, Y², Y³, or Y⁴. In a further embodiment, the linking amino acid is selected from Gly or Alaproline. In another embodiment, the linking amino acid is Gly.

In another embodiment, u is 3-20. In a further embodiment, -(J^{1C})ₜ₁-, -(J^{1D})ᵤ₁-, -(J^{2C})ₜ₂-, and -(J^{2D})ᵤ₂- are each independently a peptide sequence selected from:

| SEQ | |
|---|---|
| ID | |
| NO. | Peptide Sequence |
| 149 | Lys-Gln-Lys-Thr-Ser-(X-ph)-Gly-Arg-Gly-Pip; |
| 2 | Asn-Phe-Lys-Gln-Nap-His-Ala-Gly-Dab-Arg; |
| 3 | Dab-Val-Ala-Arg-Asn-Asn-Dab-Thr-Lys-Gly; |
| 4 | Cit-Met-Phe-Gly-Pip-Dab-Dab-Lys-Ala-Pro; |
| 5 | Dab-Pro-Arg-Dab-Leu-His-Ser-Dab-Ala-Thr; |
| 150 | Pro-Pip-Gly-Dab-Lys-Dab-Ser-Val-(X-ph)-Nap; |
| 151 | Val-Gln-His-(X-ph)-Arg-Dab-Lys-Glu-Lys-Asn; |
| 8 | Asn-Phe-Asn-Asn-Arg-Ser-Dab-Gly-His-Lys; |
| 9 | Ala-Arg-Ser-Glu-Gln-His-His-Nap-Ser-Dab; |
| 152 | (X-ph)-Gly-Arg-Gly-Pip |
| 153 | Lys-Thr-Ser-(X-ph)-Gly-Arg |
| 154 | (X-ph)-Gly-Arg |
| 155 | Lys-Gln-Lys-Thr-Ser-(X-ph)-Gly-Arg |
| 156 | Ser-(X-ph)-Gly-Arg |
| 157 | Gln-Lys-Thr-Ser-(X-ph)-Gly-Arg |
| 158 | Lys-Gln-Lys-Thr-Ser-(X-ph)-Gly-Arg. |

In a further embodiment,

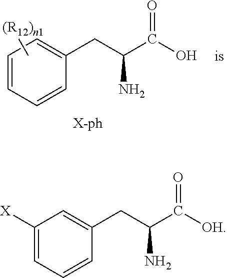

In another embodiment, X is I.

In other embodiments, at least one, -(J^{1C})ₜ₁-, -(J^{1D})ᵤ₁-, -(J^{2C})ₜ₂-, or -(J^{2D})ᵤ₂- is:

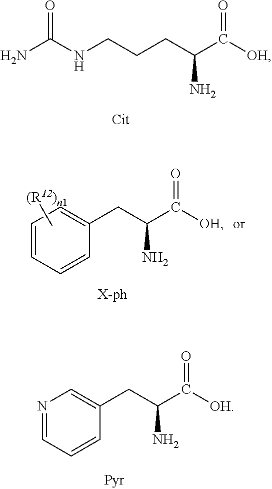

In another embodiment, -(J^{1C})ₜ₁-, -(J^{1D})ᵤ₁-, -(J^{2C})ₜ₂-, or -(J^{2D})ᵤ₂- comprises an (X-ph)-Gly-Arg motif. In another embodiment, the chimeric cell penetrating peptide of -(J^{1C})ₜ₁-, -(J^{1D})ᵤ₁-, -(J^{2C})ₜ₂- or -(J^{2D})ᵤ₂- comprises an (X-ph)-Gly-Arg motif.

In still another embodiment, -(J¹)ₜ₁-(J^{1B})_{z3}-(J^{1D})ᵤ₁-, or -(J^{1C})ₜ₂-(J^{2B})_{z5}-(J^{2D})ᵤ₂- is a peptide sequence selected from:

| SEQ ID | |
|---|---|
| NO. | Sequence |
| 39 | Lys-Gln-Lys-Thr-Ser-Iph-Gly-Arg-Gly-Pip-Lys-Trp-Lys-Lys-Gly-Lys-Gln-Lys- |
| | Thr-Ser-Iph-Gly-Arg-Gly-Pip; |
| 40 | Dab-Val-Ala-Arg-Asn-Asn-Dab-Thr-Lys-Gly-Lys-Trp-Lys-Lys-Gly-Lys-Gln-Lys- |
| | Thr-Ser-Iph-Gly-Arg-Gly-Pip; |
| 41 | Cit-Met-Phe-Gly-Pip-Dab-Dab-Lys-Ala-Pro-Lys-Trp-Lys-Lys-Gly-Lys-Gln-Lys- |
| | Thr-Ser-Iph-Gly-Arg-Gly-Pip; |
| 42 | Iph-Gly-Arg-Gly-Pip-Lys-Gln-Lys-Thr-Ser-Iph-Gly-Arg-Gly-Pip; |
| 43 | Lys-Thr-Ser-Iph-Gly-Arg-Lys-Gln-Lys-Thr-Ser-Iph-Gly-Arg-Gly-Pip; |
| 44 | Lys-Gln-Lys-Thr-Ser-Lys-Gln-Lys-Thr-Ser-Iph-Gly-Arg-Gly-Pip; |
| 45 | Lys-Trp-Lys-Lys-Gln-Lys-Thr-Ser-Iph-Gly-Arg-Gly-Pip; |
| 46 | Lys-Thr-Ser-Iph-Gly-Arg-Lys-Gln-Lys-Thr-Ser-Iph-Gly-Arg; |
| 47 | Lys-Gln-Lys-Thr-Ser-Iph-Gly-Arg-Lys-Trp-Lys-Gln-Lys-Thr-Ser-Iph-Gly-Arg. |

In yet another embodiment, the chimeric cell penetrating peptide of -(J^{1C})ₜ₁-(J^{1B})ᵤ₁-(J^{1D})ᵤ₁-, or -(J^{2C})ₜ₂-(J^{2B})_{z5}-(J^{2C})₂ᵤ- comprises at least two Iph-Gly-Arg motifs. In a further embodiment, -(J^{1C})ₜ₁-(J^{1B})_{z3}-(J^{1D})ᵤ₁-, or -(J^{2B})ₜ₂-(J^{2B})_{z5}-(J^{2D})ᵤ₂- is a peptide sequence selected from:

| SEQ ID | |
|---|---|
| NO. | Sequence |
| 39 | Lys-Gln-Lys-Thr-Ser-Iph-Gly-Arg-Gly-Pip-Lys-Trp-Lys-Lys-Gly-Lys-Gln-Lys- |
| | Thr-Ser-Iph-Gly-Arg-Gly-Pip; |
| 42 | Iph-Gly-Arg-Gly-Pip-Lys-Gln-Lys-Thr-Ser-Iph-Gly-Arg-Gly-Pip; |
| 43 | Lys-Thr-Ser-Iph-Gly-Arg-Lys-Gln-Lys-Thr-Ser-Iph-Gly-Arg-Gly-Pip; |
| 46 | Lys-Thr-Ser-Iph-Gly-Arg-Lys-Gln-Lys-Thr-Ser-Iph-Gly-Arg; |
| 47 | Lys-Gln-Lys-Thr-Ser-Iph-Gly-Arg-Lys-Trp-Lys-Gln-Lys-Thr-Ser-Iph-Gly-Arg. |

### VII. Pharmaceutical Compositions

The present disclosure also provides for formulation and delivery of the disclosed conjugates (e.g., conjugates of Formulae I, Ia Ib, Ic, Id, II, IIa, IIb, III, IIa, IV, IVa, Va, Vb, Vc, Vd, VI, VIa, VII, VIIa, VIIIa, and/or VIIIb. Accordingly, an aspect of the present disclosure is a pharmaceutical composition comprising one or more antisense oligomer conjugates as disclosed herein and a pharmaceutically acceptable carrier.

Effective delivery of the antisense oligomer conjugates to the target nucleic acid is an important aspect of treatment. Routes of delivery of the antisense oligomer conjugate include, but are not limited to, various systemic routes, including oral and parenteral routes, e.g., intravenous, subcutaneous, intraperitoneal, and intramuscular, as well as inhalation, transdermal and topical delivery. The appropriate route may be determined by one of skill in the art, as appropriate to the condition of the subject under treatment.

The antisense oligomer conjugate can be administered in any convenient vehicle which is physiologically and/or pharmaceutically acceptable. Such a composition can include any of a variety of standard pharmaceutically acceptable carriers employed by those of ordinary skill in the art. Examples include, but are not limited to, saline, phosphate buffered saline (PBS), water (e.g., sterile water for injection), aqueous ethanol, emulsions such as oil/water emulsions or triglyceride emulsions, tablets, and capsules. The choice of suitable physiologically acceptable carrier will vary dependent upon the chosen mode of administration.

The antisense oligomer conjugates can generally be utilized as the free acid or free base. Alternatively, the antisense oligomer conjugate may be used in the form of acid or base addition salts. Acid addition salts of the free amino compounds may be prepared by methods well known in the art and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of Formulae I, Ia Ib, Ic, Id, II, IIa, IIb, III, IIIa, IV, IVa, Va, Vb, Vc, Vd, VI, VIa, VII, VIIa, VIIIa, and/or VIIIb are intended to encompass any and all acceptable salt forms.

### VIII. Methods of Treatment

Provided herein are methods of treating a neuromuscular disease. The methods comprise administering to a patient in need thereof a therapeutically effective amount of one or more antisense oligomer conjugates disclosed herein, or a pharmaceutical composition thereof. In an embodiment, the neuromuscular disease is Duchenne muscular dystrophy.

In certain embodiments, the one or more antisense oligomer conjugates is administered to a mammalian subject, e.g., a human or a laboratory or domestic animal, optionally with one or more suitable pharmaceutical carriers.

In certain embodiments, the one or more antisense oligomer conjugates is administered to a mammalian subject, e.g., a human or laboratory or domestic animal, together with one or more additional agents. The one or more antisense oligomer conjugates and the one or more additional agents can be administered simultaneously or sequentially, via the same or different routes and/or sites of administration. In certain embodiments, the one or more antisense oligomer conjugates and the one or more additional agents can be co-formulated and administered together. In certain embodiments, the one or more antisense oligomer conjugates and the one or more additional agents can be provided together in a kit.

In one embodiment, the one or more antisense oligomer conjugates, formulated with a pharmaceutically acceptable carrier, is parenterally administered. In one embodiment, the one or more antisense oligomer conjugates, contained in a pharmaceutically acceptable carrier, is delivered intravenously (i.v.).

In an embodiment, the one or more antisense oligomer conjugates is administered in an amount and manner effective to result in a peak blood concentration of at least 200 nM of total antisense oligomer conjugates. In one embodiment, the one or more antisense oligomers conjugate is administered in an amount and manner effective to result in a peak plasma concentration of at least 200 nM of total antisense oligomer conjugates. In one embodiment, the one or more antisense oligomer conjugates is administered in an amount and manner effective to result in a peak serum concentration of at least 200 nM of the antisense oligomer conjugates.

In an embodiment, the one or more antisense oligomer conjugates is administered in an amount and manner effective to result in a peak blood concentration of at least 400 nM of total antisense oligomer conjugates. In one embodiment, the one or more antisense oligomer conjugates is administered in an amount and manner effective to result in a peak plasma concentration of at least 400 nM total antisense oligomer conjugates. In one embodiment, the one or more antisense oligomer conjugates is administered in an amount and manner effective to result in a peak serum concentration of at least 400 nM total antisense oligomer conjugates.

Typically, one or more doses of the one or more antisense oligomer conjugates are administered, generally at regular intervals, for a period of about one to 12 weeks. Preferred doses for oral administration are from about 0.01 to about 15 mg antisense oligomer conjugate per kg body weight. In some cases, doses of greater than 15 mg antisense oligomer conjugate/kg may be necessary.

For i.v. administration, preferred doses are from about 0.01 mg to about 15 mg antisense oligomer conjugate per kg body weight. The antisense oligomer conjugate may be administered at regular intervals for the lifetime of the patient, e.g., weekly, bi-weekly, monthly, or every 8-12 weeks or more. In some cases the antisense oligomer conjugate is administered intermittently over a longer period of time. The treatment regimen may be adjusted (dose, frequency, route, etc.) as indicated, based on the results of immunoassays, other biochemical tests, and physiological examination of the subject under treatment.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

Examples have been set forth below for the purpose of illustration and to describe certain specific embodiments of the disclosure. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations or methods of the disclosure may be made without departing from the spirit of the disclosure and the scope of the appended claims. Definitions of the variables in the structures in the schemes herein are commensurate with those of corresponding positions in the formulae presented herein.

### Example 1—General Method for Peptide Preparation and Purification

A diverse library of individual peptide sequences, containing both canonical and non-natural amino acids was generated through a split-pool technique via solid-phase peptide synthesis. The library focused on reduced arginine content was created to screen for potent, low arginine containing peptides (and subsequently PPMOs) pool separated by charge through cation exchange chromatography.

Fast-Flow Peptide Synthesis: Peptides were synthesized on a 0.1 mmol scale using a semi-automated fast-flow peptide synthesizer. 1 mmol of amino acid was combined with 2.5 mL of 0.4 M hexafluorophosphate azabenzotriazole tetramethyl uronium (HATU) and 500 μL of N,N-diisopropylethylamine (DIEA) and mixed before being delivered to the reactor containing resin via syringe pump at 6 mL/min. The reactor was submerged in a water bath heated to 70° C. An HPLC pump delivered either dimethylformamide (DMF) (20 mL) for washing or 20% piperidine/DMF (6.7 mL) for fluorenylmethyloxycarbonyl (Fmoc) deprotection, at 20 mL/min.

Peptide Cleavage and Deprotection: Each peptide was subjected to simultaneous global sidechain deprotection and cleavage from resin by treatment with 5 mL of 94% trifluoroacetic acid (TFA), 2.5% thioanisole, 2.5% water, and 1% triisopropylsilane (TIPS) (v/v) at room temperature for 2 to 4 h. The cleavage cocktail was first concentrated by bubbling N₂ through the mixture, and cleaved peptide was precipitated and triturated with 40 mL of cold ether (chilled in dry ice). The crude product was pelleted by centrifugation for three minutes at 4,000 rpm and the ether was decanted. This wash step was repeated two more times. After the third wash, the pellet was dissolved in 50% water and 50% acetonitrile containing 0.1% TFA, filtered through a fritted syringe to remove the resin and lyophilized.

Peptide Purification: The peptides were dissolved in water and acetonitrile containing 0.1% TFA, filtered through a 0.22 μm nylon filter and purified by mass-directed semi-preparative reversed-phase HPLC. Solvent A was water with 0.1% TFA additive and Solvent B was acetonitrile with 0.1% TFA additive. A linear gradient from 5 to 45% B that changed at a rate of 0.5% B/min was used. Most of the peptides were purified on an Agilent Zorbax SB C18 column: 9.4×250 mm, 5 μm. Based on target ion mass data recorded for each fraction, only pure fractions were pooled and lyophilized.

Click Moiety Attachment and Copper Free Click Reaction: The connection of the oligonucleotide to the peptide was carried out by the following general scheme. First, the oligonucleotides were dissolved in DMSO. Then 8,9-dihydro-3H-dibenzo[b,f][1,2,3]triazolo[4,5-d]azocine-acid (DBCO-acid) was mixed with 2.5 mL of 0.4 M hexafluorophosphate azabenzotriazole tetramethyl uronium (HATU) and 500 μL of N,N-diisopropylethylamine (DIEA) and mixed before being combined with the oligonucleotide. The peptide, with an N-terminal azide functional group was combined with the oligonucleotide to form the DBCO click ring that connects the oligonucleotide to the peptide.

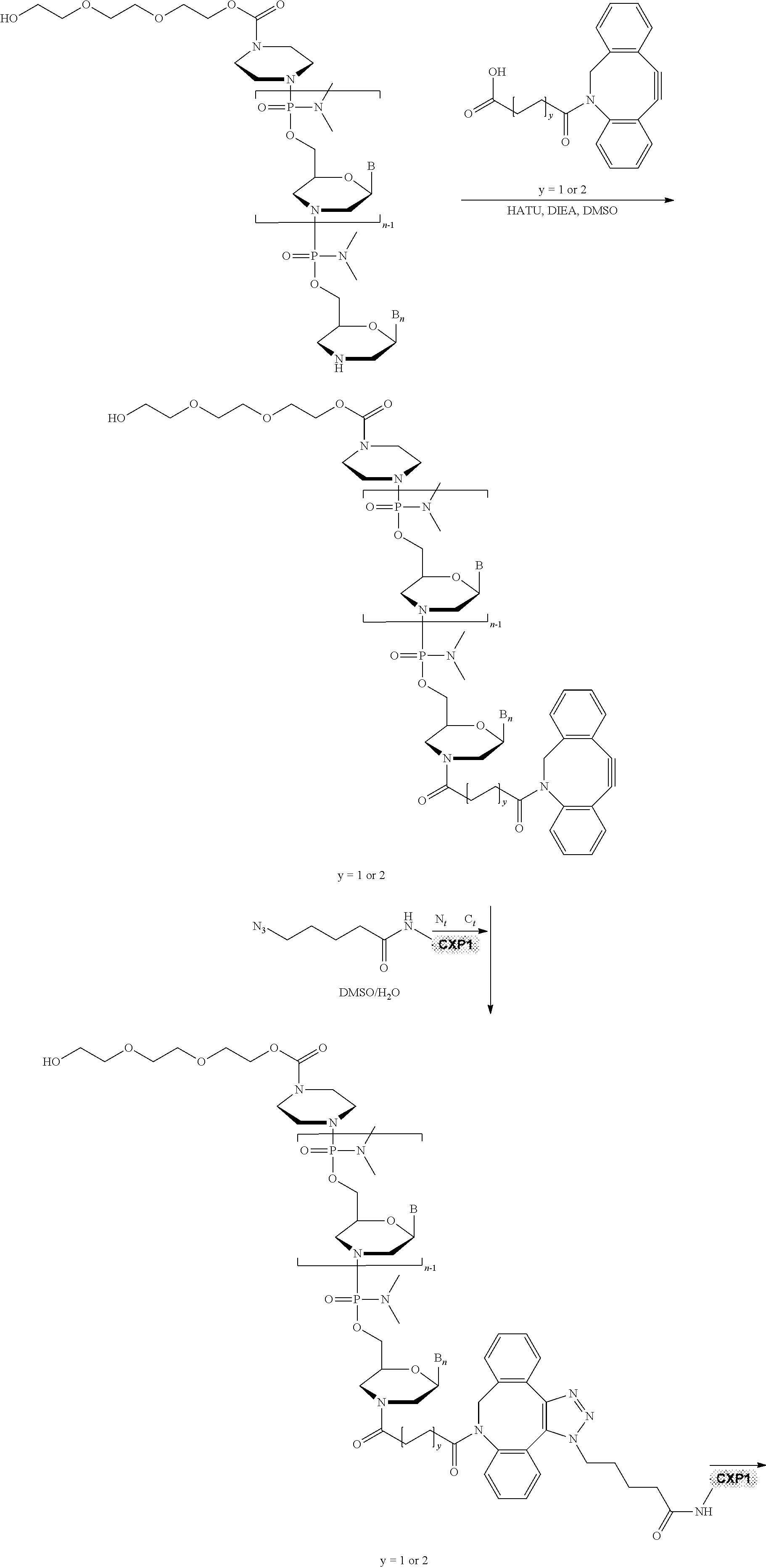

### Example 2—Liquid Chromatography-Mass Spectrometry (LC-MS)

The purity of each fraction pool was confirmed by LC-MS.

LC-MS analyses: Analysis was performed on an Agilent 6550 Funnel Q-TOF LC-MS system (abbreviated as 6550) coupled to an Agilent 1290 Infinity HPLC system. Mobile phases were: 0.1% formic acid in water (solvent A) and 0.1% formic acid in acetonitrile (solvent B). The following LC-MS method was used for characterization:
1-61% B over 6 min, Zorbax C3 column (6550)
LC: Agilent EclipsePlus C18 RRHD column: 2.1×50 mm, 1.8 μm, column temperature: 40° C., gradient: 0-1 min 1% B, 1-6 min, 1-61% B, 6-7 min, 91% B, 7-8 min, 1% B; flow rate: 0.5 mL/min.
MS: Positive electrospray ionization (ESI) extended dynamic range mode in mass range 300-3000 m/z. MS is on from 1 to 6 min.
   All data were processed using Agilent MassHunter software package. Y-axis in all chromatograms shown represents total ion current (TIC) unless noted.

Orbitrap LC-MS/MS: Analysis was performed on an EASY-nLC 1200 (Thermo Fisher Scientific) nano-liquid chromatography handling system connected to an Orbitrap Fusion Lumos Tribrid Mass Spectrometer (Thermo Fisher Scientific). Samples were run on a PepMap RSLC C18 column (2 μm particle size, 15 cm×50 μm ID; Thermo Fisher Scientific, P/N ES901). A nanoViper Trap Column (C18, 3 μm particle size, 100 Å pore size, 20 mm×75 μm ID; Thermo Fisher Scientific, P/N 164946) was used for desalting. The standard nano-LC method was run at 40° C. and a flow rate of 300 nL/min with the following gradient: 1% solvent B in solvent A ramping linearly to 41% B in A over 55 min, where solvent A=water (0.1% FA), and solvent B=80% acetonitrile, 20% water (0.1% FA). Positive ion spray voltage was set to 2200 V. Orbitrap detection was used for primary MS, with the following parameters: resolution=120,000; quadrupole isolation; scan range=150-1200 m/z; RF lens=30%; AGC target=250%; maximum injection time=100 ms; 1 microscan. Acquisition of secondary MS spectra was done in a data-dependent manner: dynamic exclusion was employed such that a precursor was excluded for 30 s if it was detected four or more times within 30 s (mass tolerance: 10.00 ppm): monoisotopic precursor selection used to select for peptides; intensity threshold was set to 2×10⁴; charge states 2-10 were selected; and precursor selection range was set to 200-1400 m/z. The top 15 most intense precursors that met the preceding criteria were subjected to subsequent fragmentation. Two fragmentation modes—higher-energy collisional dissociation (HCD), and electron-transfer/higher-energy collisional dissociation (EThcD)—were used for acquisition of secondary MS spectra. Detection was performed in the Orbitrap (resolution=30,000; quadrupole isolation; isolation window=1.3 m/z; AGC target=2×10⁴; maximum injection time=100 ms; 1 microscan). For HCD, a stepped collision energy of 3, 5, or 7% was used. For EThcD, a supplemental activation collision energy of 25% was used.

### Example 3—Preparation of Peptide Libraries

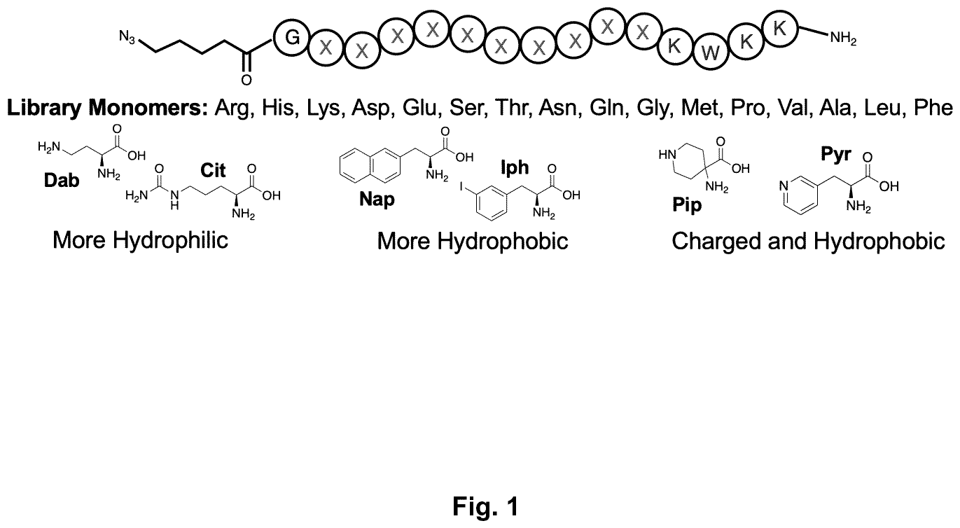

Library Design and Monomer Set of the CPP Library: The library was prepared with a C-terminal Lys-Trp-Lys-Lys motif (derived from the established cell-penetrating peptide penetratin), a glycine at the N-terminus, and ten variable positions containing any of the 22 monomers selected from natural amino acids and noncanonical amino acids (FIG. 1). The library includes natural residues, 16 natural amino acids (arginine, histidine, lysine, aspartic acid, glutamic acid, serine, threonine, asparagine, glutamine, glycine, methionine, proline, valine, alanine, leucine, phenylalanine) and 6 non-canonical amino acids (2,4-diaminobutyric acid (Dab), 2-amino-5-ureidopentanoic acid (citrulline (Cit)), 2-amino-3-(naphthalen-2-yl)propanoic acid (Nap), 4-iodo-L-phenylalaine (Iph), 4-aminopiperidine-4-carboxylic acid (Pip), and 2-amino-3-(pyridin-3-yl)propanoic acid (Pyr)). The glycine at the N-terminus separates the N-terminal azide from the variable region and the peptides of the library are amidated at the C-terminus.

De novo peptide sequencing and filtering: De novo peptide sequencing of the acquired data was performed in PEAKS 8 (BioInformatics Solutions Inc.). Using PEAKS, spectra were prefiltered to remove noise, and sequenced. All non-canonical amino acids were sequenced as post-translational modifications based on the canonical amino acid most closely matching their molecular mass. Twenty candidate sequence assignments were created for each secondary scan.

### Example 4—Split-and-Pool Synthesis

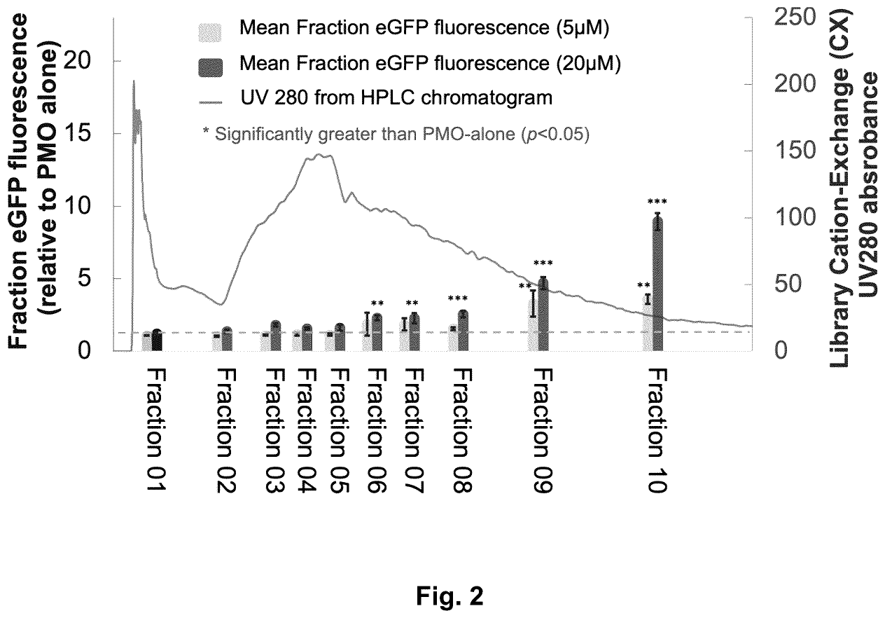

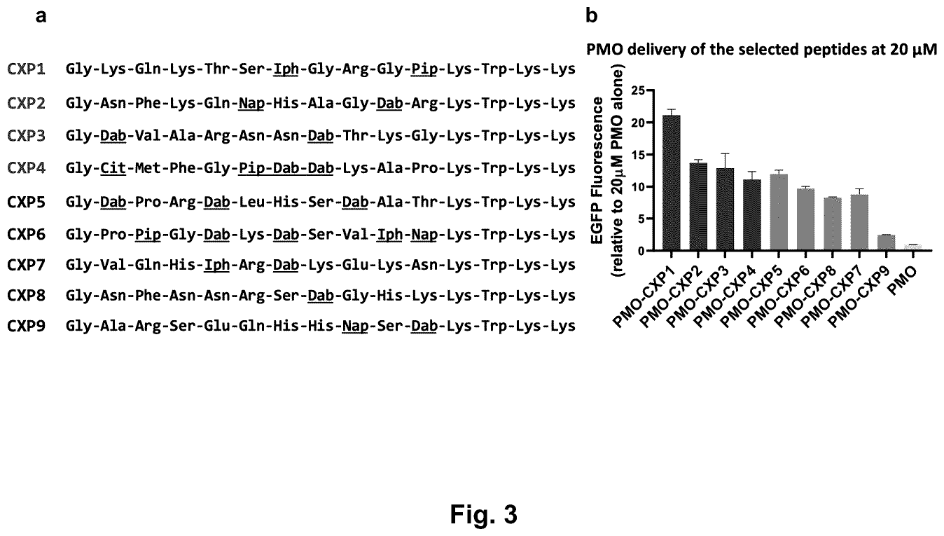

Split-and-pool synthesis via solid-phase peptide synthesis afforded a 3,000-member library. This 3,000-member library was then separated by cation-exchange chromatography across an ammonium acetate salt gradient, and peptides were collected in small fractions as they eluted off the column (FIG. 2). These smaller fractions were combined into 10 pools, each containing about 300 individual peptide sequences based on UV quantification of the tryptophan residue.

Split-and-pool synthesis was carried out on 180 μm TentaGel resin (0.28 mmol/g) for a 50,000-member library. Splits were performed by suspending the resin in dichloromethane (DCM) and dividing it evenly (via pipetting) among 22 plastic fritted syringes on a vacuum manifold. Couplings were carried out as follows: solutions of Fmoc-protected amino acids (10 equivalents relative to the resin loading), 7-azabenzotriazol-1-yloxy)tripyrrolidinophosphonium hexafluorophosphate (PyAOP) (0.38 M in DMF; 0.95 eq, relative to amino acid), and DIEA (1.1 eq. for histidine; 3 eq. for all other amino acids) were each added to individual portions of resin. Couplings were allowed to proceed for 60 min. Resin portions were recombined and washed with DCM and DMF. fluorenylmethyloxycarbonyl (Fmoc) removal was carried out by treatment of the resin with 20% piperidine in DMF (1× flow wash; 2×10 min batch treatments). Resin was washed again with DMF and DCM before the next split.

After synthesis, the library was separated into 3,000-member portions by mass. Each portion was conjugated to 5-azidopentanoic acid through a coupling with PyAOP as described above, Library peptides were then cleaved as described above and lyophilized to generate libraries of 3,000 peptide sequences.

Library Separation by Cation-Exchange Chromatography: The 3,000-member peptide library was dissolved in loading buffer (10 mM ammonium acetate, pH 5, 20% Acetonitrile). The peptides were then resolved on a Propac SCX-10 column (Thermo Fisher).

Solvent A was 10 mM ammonium acetate with 10% acetonitrile, pH 5, and Solvent B was 1M ammonium acetate with 10% acetonitrile, pH 5. A linear gradient from 1 to 75% B over 75 minutes was used, with a 10-minute hold at 1% B to allow compound to load onto the column. Fractions eluting off the column were collected every minute, with a total volume of 1 mL.

Library Desalting: All fractions were lyophilized overnight, and then re-dissolved in 1 mL of water. Fractions were then frozen and re-lyophilized to remove residual ammonium acetate buffer. This process was repeated for at least 3 lyophilization cycles to fully remove the volatile buffer.

Library pooling: After desalting, the peptide concentration on each fraction was measured spectroscopically at 280 nM, based on the absorbance of the single tryptophan residue in each peptide library member. Neighboring peptide fractions were combined based on peptide concentration to generate 10 pools with approximately equal peptide concentration. This resulted in pools containing about 300 peptide sequences, based on the original library of 3,000 peptide sequences.

### Example 5—Peptide Conjugation

These larger pools, numbered 01-10 based on their cation exchange elution time, were then conjugated to the PMO cargo via strain-promoted azide-alkyne cycloaddition (as described below).

Preparation of PMO-DBCO: PMO was modified with a dibenzocyclooctyne (DBCO) moiety and purified before attachment to the azido-peptides. PMO IVS2-654 (50 mg, 8 μmol) was dissolved in 150 μL dimethyl sulfoxide (DMSO). To the solution was added a solution containing 2 equivalents of dibenzocyclooctyne acid (5.3 mg, 16 μmol) activated with hexafluorophosphate benzotriazole tetramethyl uronium (HBTU) (37.5 μL of 0.4 M HBTU in DMF, 15 μmol) and DIEA (2.8 μL, 16 μmol) in 40 μL DMF (final reaction volume=0.23 mL). The reaction proceeded for 25 min before being quenched with 1 mL of water and 2 mL of ammonium hydroxide. The ammonium hydroxide hydrolyzed any ester formed during the reaction. After 1 hour, the solution was diluted to 40 mL in water/acetonitrile and purified using reverse-phase HPLC (Agilent Zorbax SB C3 column: 21.2×100 mm, 5 μm) and a linear gradient from 2 to 60% B (solvent A: water; solvent B: acetonitrile) over 58 min (1% B/min). Using mass data about each fraction from the instrument, only pure fractions were pooled and lyophilized. The purity of the fraction pool was confirmed by LC-MS.

Conjugation of PMO to Peptides: Peptides were conjugated to PMO-DBCO via N-term azidopentanoic acid. PMO-DBCO (1 eq, 5 mM, water) was conjugated to azido-peptides (1 eq, 5 mM, water) or azido-peptide library (1eq, 1 mM, water) at room temperature for 2 h, or 12 hours for peptide library. Reaction progress was monitored by LC-MS and additional stock of azido-peptide was added until all PMO-DBCO was consumed. The purity of the final construct was confirmed by LC-MS to be >95%.

### Example 6—Enhanced Green Fluorescent Protein Assay

Purified constructs were then tested using an activity-based readout in which nuclear delivery results in fluorescence. Briefly, HeLa cells stably transfected with an Enhanced Green Fluorescent Protein (eGFP) gene interrupted by a mutated intron of β-globin (IVS2-654) produce a non-fluorescent eGFP protein. As used herein, Hela-654 cells refers to the Hela cells transfected with an Enhanced Green Fluorescent Protein (eGFP) gene interrupted by a mutated intron of β-globin (IVS2-654) and Hela-654 eGFP Assay is used to refer to an Assay that uses the Hela-654 cells. Successful delivery of PMO IVS2-654 to the nucleus results in corrective splicing and eGFP synthesis.

The amount of PMO delivered to the nucleus is therefore correlated with eGFP fluorescence, quantified by flow cytometry. Activity was reported as mean fluorescence intensity (MFI) relative to PMO alone. This activity assay provides indirect information on how much active PMO is delivered to the nucleus. Relative efficiency of a PMO-CPP conjugate was characterized by comparing activity to internal concentration.

HeLa 654 cells obtained from the University of North Carolina Tissue Culture Core facility were maintained in methoxymethyl ether (MEM) supplemented with 10% (v/v) fetal bovine serum (FBS) and 1% (v/v) penicillin-streptomycin at 37° C. and 5% CO₂. 18 h prior to treatment, the cells were plated at a density of 5,000 cells per well in a 96-well plate in DMEM supplemented with 10% FBS and 1% penicillin-streptomycin.

For individual peptide testing, PPMOs were dissolved in phosphate buffered saline (PBS) without Ca²⁺ or Mg²⁺ at a concentration of 1 mM (determined by UV absorbance of the PMO) before being diluted in Dulbecco's modified eagle medium (DMEM). Cells were incubated at the designated concentrations in triplicate for 22 h at 37° C. and 5% CO₂. Next, the treatment media was removed, and the cells were washed once before being incubated with 0.25% Trypsin-EDTA for 15 min at 37° C. and 5% CO₂. Lifted cells were transferred to a V-bottom 96-well plate and washed once with PBS, before being resuspended in PBS containing 2% FBS and 2 μg/mL propidium iodide (PI). Flow cytometry analysis was carried out on a BD LSRII flow cytometer. Gates were applied to the data to ensure that cells that were positive for propidium iodide or had forward/side scatter readings that were sufficiently different from the main cell population were excluded. Each sample was capped at 5,000 gated events.

Analysis was conducted using Graphpad Prism 7 and FlowJo. For each sample, the mean fluorescence intensity (MFI) and the number of gated cells were measured. To report activity, triplicate MFI values were averaged and normalized to the PMO alone condition.

Library Fractions Demonstrated Pools with Significant PMO Delivery: eGFP fluorescence of HeLa 654 cells treated with 5 or 20 μM total PMO-peptide from the library fractions demonstrates 5 pools with significant PMO delivery (see FIG. 2). Delivery of PMO to the nucleus can be assessed by measuring eGFP fluorescence through flow cytometry (FIG. 2). PMO-peptide fractions 6 through 10 improved PMO delivery significantly over the PMO-654 alone at 20 μM (p<0.05) and these five most cationic peptide pools (based on cation exchange retention time) showed significant delivery of the PMO cargo.

The most penetrant pool of peptides, fraction 10, was sequenced. Almost 100 peptide sequences were recovered, and nine peptides were generated via solid-phase peptide synthesis and conjugated to the PMO cargo.

TABLE 4
Sequences of Nine Peptides Chosen from Pool 10

| | N-terminal | | |
|---|---|---|---|
| Compound | (Linking | SEQ ID | |
| Name | AA) | NO. | Peptide Sequences |
| CXP1 | Gly | 48 | Lys-Gln-Lys-Thr-Ser-Iph-Gly-Arg-Gly-Pip-Lys- |
| | | | Trp-Lys-Lys |
| CXP2 | Gly | 49 | Asn-Phe-Lys-Gln-Nap-His-Ala-Gly-Dab-Arg- |
| | | | Lys-Trp-Lys-Lys |
| CXP3 | Gly | 50 | Dab-Val-Ala-Arg-Asn-Asn-Dab-Thr-Lys-Gly- |
| | | | Lys-Trp-Lys-Lys |
| CXP4 | Gly | 51 | Cit-Met-Phe-Gly-Pip-Dab-Dab-Lys-Ala-Pro-Lys- |
| | | | Trp-Lys-Lys |
| CXP5 | Gly | 52 | Dab-Pro-Arg-Dab-Leu-His-Ser-Dab-Ala-Thr- |
| | | | Lys-Trp-Lys-Lys |
| CXP6 | Gly | 53 | Pro-Pip-Gly-Dab-Lys-Dab-Ser-Val-Iph-Nap-Lys- |
| | | | Trp-Lys-Lys |
| CXP7 | Gly | 54 | Val-Gln-His-Iph-Arg-Dab-Lys-Glu-Lys-Asn-Lys- |
| | | | Trp-Lys-Lys |
| CXP8 | Gly | 55 | Asn-Phe-Asn-Asn-Arg-Ser-Dab-Gly-His-Lys- |
| | | | Lys-Trp-Lys-Lys |
| CXP9 | Gly | 56 | Ala-Arg-Ser-Glu-Gln-His-His-Nap-Ser-Dab-Lys- |
| | | | Trp-Lys-Lys. |

*Peptides were conjugated to PMO-DBCO via N-term azidopentanoic acid.
**The peptides are amidated at the C-terminus.

PPMOs Showed Significant Delivery: The nine PPMOs (Table 3) were tested for PMO delivery into HeLa cells. All nine PPMOs improved PMO delivery significantly over the PMO-654 alone at 20 μM (FIG. 3). Penetratin, a known CPP, was included as a positive control. Four peptides in particular, CXP1, CXP2, CXP3, and CXP4, showed high levels of PMO delivery.

### Example 7—Lactate Dehydrogenase (LDH) Release Assay

Cytotoxicity assays were performed in HeLa 654 cells or TH-1 renal cells. TH-1 cells were maintained in DMEM supplemented with 10% (v/v) fetal bovine serum (FBS) and 1% (v/v) penicillin-streptomycin at 37° C. and 5% CO₂H-1. Cells were plated at 5,000 cells/well in a 96-well plate 18-hours prior to treatment and treated with PMO-peptide compounds as described above. HeLa cell LDH assays were performed concurrently on the cells treated for Flow cytometry, so all plating and treatment protocols match those described above.

17 hours after cell treatment, the Promega CytoTox lysis solution was added to 3 wells of each plate as a fully lysed control (100% LDH-release). 18 hours after treatment, cell supernatant was transferred to a new 96-well plate for analysis of LDH release. To each well of the 96-well plate containing supernatant was added CytoTox 96 Reagent (Promega). The plate was shielded from light and incubated at room temperature for 30 min. Equal volume of Stop Solution was added to each well, mixed, and the absorbance of each well was measured at 490 nm. The measurement of vehicle-treated cells was subtracted from each measurement, and % LDH release was calculated as % cytotoxicity=100×Experimental LDH Release (OD490)/Maximum LDH Release (OD490).

### Example 8—Dose-Response Studies

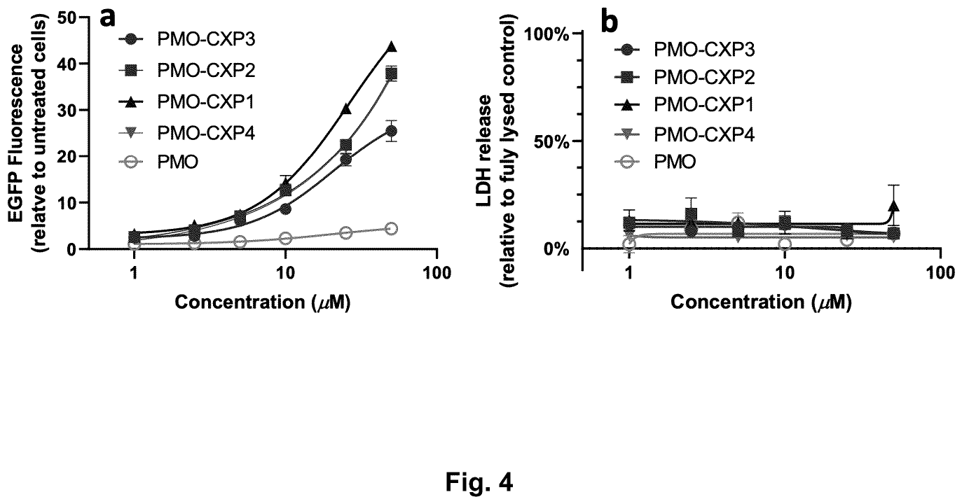

PMO-CXP1 conjugate, PMO-CXP2 conjugate, PMO-CXP3 conjugate, and PMO-CXP4 conjugate showed dose-dependent delivery of PMO. These PMO-peptide conjugates were tested at varying concentrations in the Hela-654 eGFPassay (FIG. 4A). Furthermore, the supernatant from these assays was tested for lactate dehydrogenase (LDH) release, which is indicative of membrane toxicity (Example 7). The constructs showed no significant toxicity within the tested concentration range (FIG. 4B). These four PMO-peptides demonstrate that the assay platform allows for discovery of novel and efficacious peptide sequences for PMO delivery. Table 5.

TABLE 5
CXP Hela-654 eGFP Assay Data

| | N-terminal | | | EC₅₀ |
|---|---|---|---|---|
| Compound | (Linking | SEQ ID | | (at |
| Name | AA) | No. | Peptide Sequence | 72 h) |
| PMO | — | - | | 22.7 |
| PMO-CXP1 | Gly | 48 | Lys-Gln-Lys-Thr-Ser-Iph- | 7.08 |
| | | | Gly-Arg-Gly-Pip-Lys-Trp- | |
| | | | Lys-Lys | |
| PMO-CXP2 | Gly | 49 | Asn-Phe-Lys-Gln-Nap- | 11.51 |
| | | | His-Ala-Gly-Dab-Arg-Lys- | |
| | | | Trp-Lys-Lys | |
| PMO-CXP3 | Gly | 50 | Dab-Val-Ala-Arg-Asn-Asn- | 13.76 |
| | | | Dab-Thr-Lys-Gly-Lys-Trp- | |
| | | | Lys-Lys | |
| PMO-CXP4 | Gly | 51 | Cit-Met-Phe-Gly-Pip-Dab- | 4.57 |
| | | | Dab-Lys-Ala-Pro-Lys-Trp- | |
| | | | Lys-Lys | |
| PMO-CXP5 | Gly | 52 | Dab-Pro-Arg-Dab-Leu- | 10.01 |
| | | | His-Ser-Dab-Ala-Thr-Lys- | |
| | | | Trp-Lys-Lys. | |

*Peptides were conjugated to PMO-DBCO via N-term azidopentanoic acid.
**The peptides are amidated at the C-terminus.

### Example 9—eGFP-654 Mouse Studies

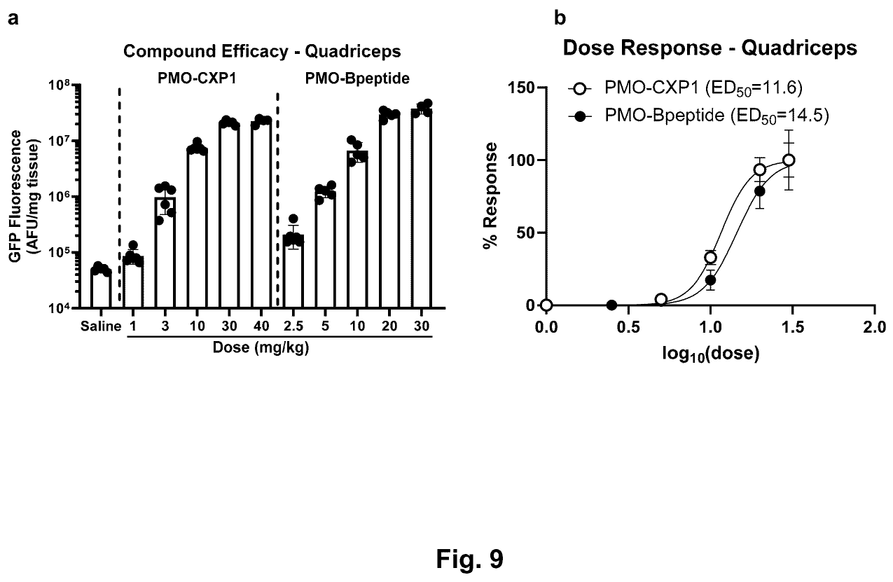

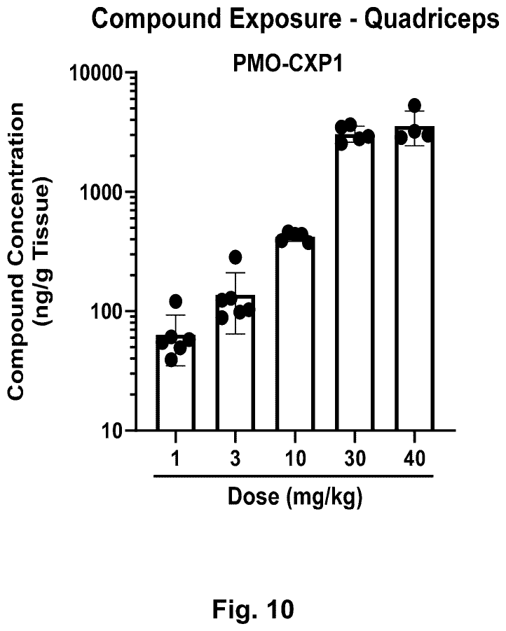

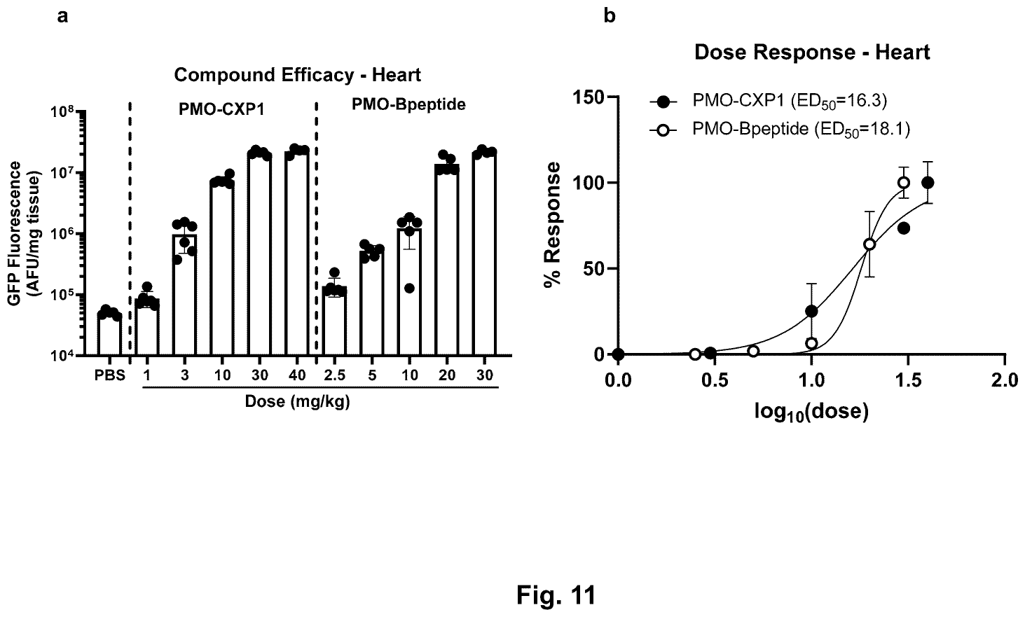

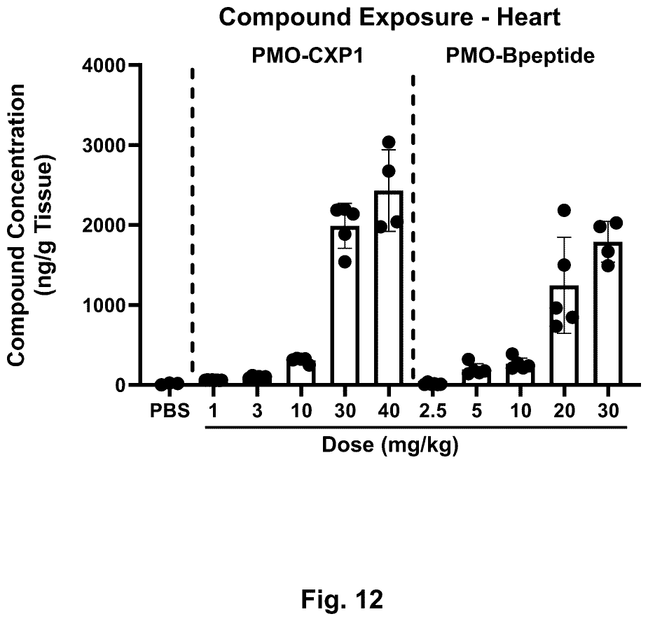

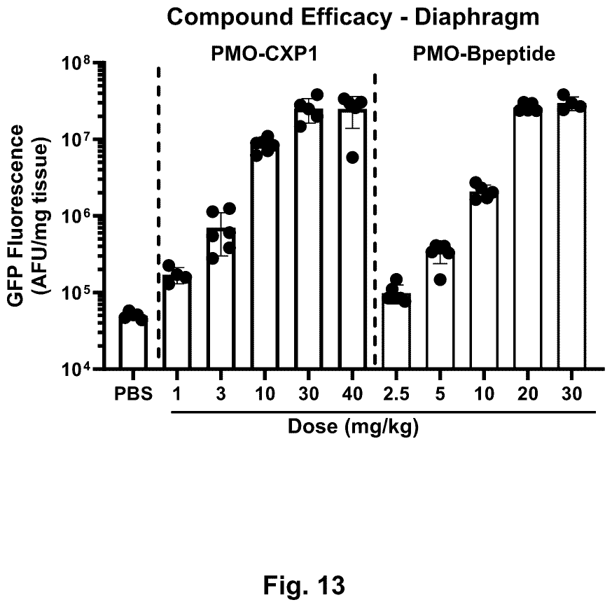

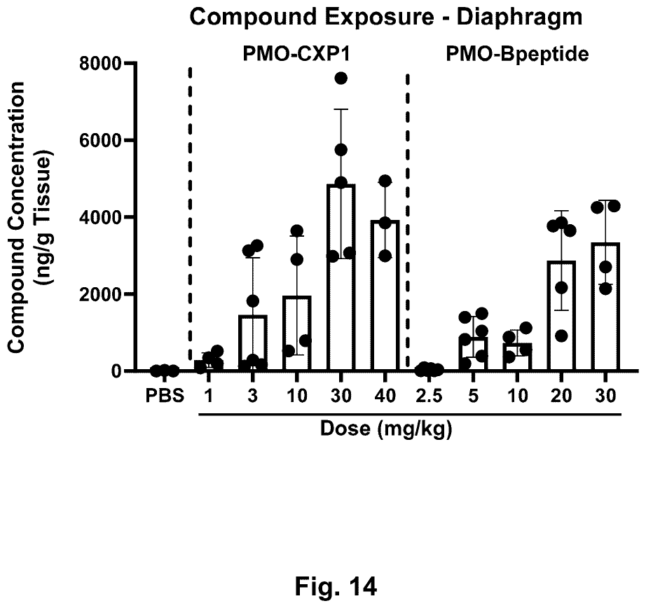

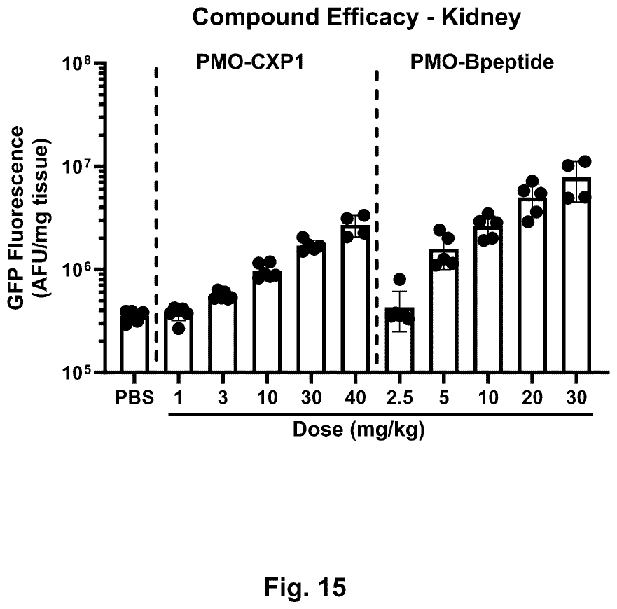

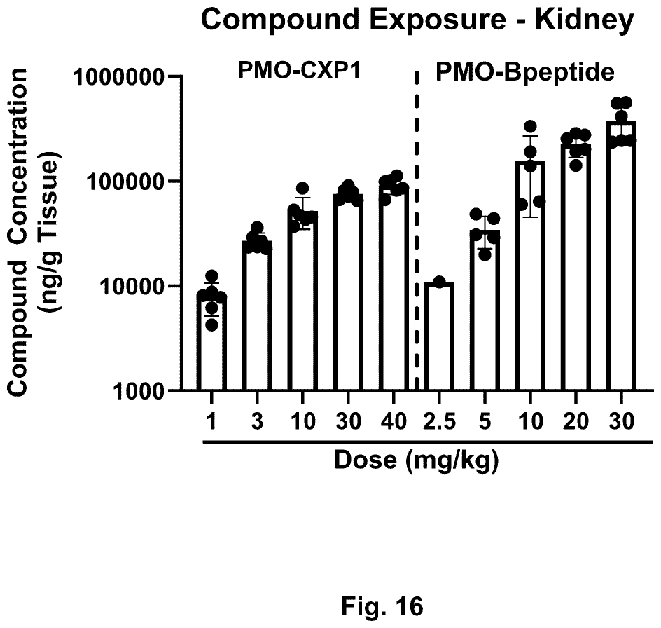

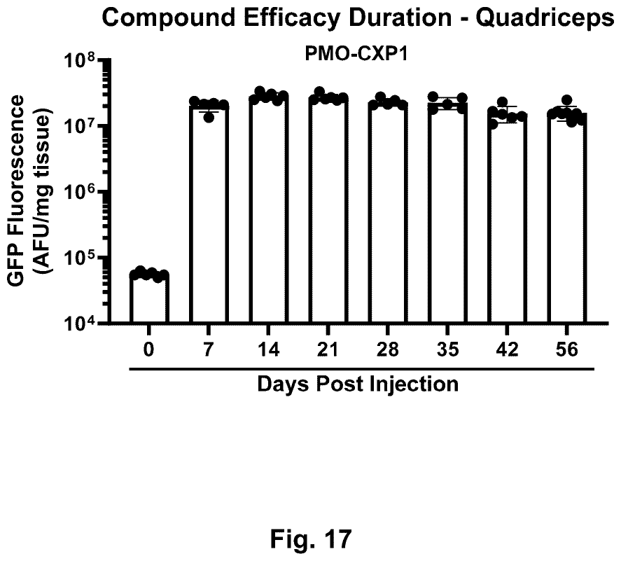

PMO-CXP1 conjugate was intravenously delivered to eGFP-654 mice at various doses and the tissues were collected for analysis 7 days after injection. The tissues from a series of organs were homogenized and GFP fluorescence was measured with a spectrophotometer and normalized to tissue quantity (FIG. 9-17). Median effective dose (ED50) was also derived from the dose-response curve. PMO-CXP1 compound exposure was calculated with an enzyme-linked oligonucleotide hybridization assay (ELOHA). Briefly, the ELOHA assay is a two-step hybridization assay where the analyte is bound to both an immobilized capture probe and biotinylated detection probe. Streptavidin-AP-conjugate is added to the mixture and AttoPhos substrate is added to the system and incubated for 10 minutes. Stop solution is added and the plate is read on the spectrophotometer. The compound concentrations were determined from a standard curve. This study demonstrated that PMO-CXP1 conjugate had similar efficacy to PMO-Bpeptide. Compound efficacy and exposure were calculated in the quadriceps (FIGS. 9-10), heart (FIGS. 11-12), diaphragm (FIGS. 13-14), and kidney (FIGS. 15-16). The compound was also found to have efficacy for up to 56 days in vivo (FIG. 17).

### Example 10—PMO-Chimeric Peptide Conjugates

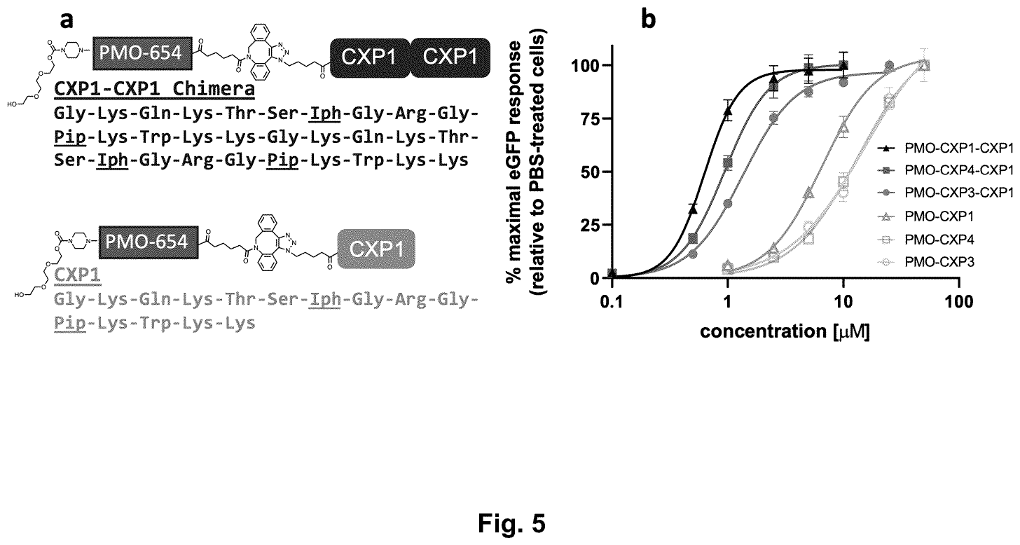

Synthesis of PMO-Chimeric Peptide Conjugates: The relationship between the peptide sequence and PMO delivery was probed to further increase the efficacy of the constructs. Chimeric peptide sequences were synthesized and then conjugated to the PMO cargo (FIG. 5A).

TABLE 6
Chimeric Peptides

| | N- | | |
|---|---|---|---|
| | terminal | | |
| Peptide | amino | SEQ ID | |
| Name | acid | NO. | Peptide Sequence |
| CXP1- | Gly | 67 | Lys-Gln-Lys-Thr-Ser-Iph-Gly-Arg-Gly-Pip-Lys-Trp-Lys- |
| CXP1 | | | Lys-Gly-Lys-Gln-Lys-Thr-Ser-Iph-Gly-Arg-Gly-Pip-Lys- |
| | | | Trp-Lys-Lys |
| CXP3- | Gly | 68 | Dab-Val-Ala-Arg-Asn-Asn-Dab-Thr-Lys-Gly-Lys-Trp-Lys- |
| CXP1 | | | Lys-Gly-Lys-Gln-Lys-Thr-Ser-Iph-Gly-Arg-Gly-Pip-Lys- |
| | | | Trp-Lys-Lys |
| CXP4- | Gly | 69 | Cit-Met-Phe-Gly-Pip-Dab-Dab-Lys-Ala-Pro-Lys-Trp-Lys- |
| CXP1 | | | Lys-Gly-Lys-Gln-Lys-Thr-Ser-Iph-Gly-Arg-Gly-Pip-Lys- |
| | | | Trp-Lys-Lys |
| SA-CXP1 | Gly | 70 | Iph-Gly-Arg-Gly-Pip-Lys-Gln-Lys-Thr-Ser-Iph-Gly-Arg-Gly- |
| | | | Pip-Lys-Trp-Lys-Lys |
| SB-CXP1 | Gly | 71 | Lys-Thr-Ser-Iph-Gly-Arg-Lys-Gln-Lys-Thr-Ser-Iph-Gly- |
| | | | Arg-Gly-Pip-Lys-Trp-Lys-Lys |
| SC-CXP1 | Gly | 72 | Lys-Gln-Lys-Thr-Ser-Lys-Gln-Lys-Thr-Ser-Iph-Gly-Arg- |
| | | | Gly-Pip-Lys-Trp-Lys-Lys |
| SD-CXP1 | Gly | 73 | Lys-Trp-Lys-Lys-Gln-Lys-Thr-Ser-Iph-Gly-Arg-Gly-Pip- |
| | | | Lys-Trp-Lys-Lys |
| CXD1A | Gly | 74 | Lys-Thr-Ser-Iph-Gly-Arg-Lys-Gln-Lys-Thr-Ser-Iph-Gly- |
| | | | Arg-Lys-Trp-Lys-Lys |
| CXD1B | Gly | 75 | Lys-Gln-Lys-Thr-Ser-Iph-Gly-Arg-Lys-Trp-Lys-Gln-Lys- |
| | | | Thr-Ser-Iph-Gly-Arg-Lys-Trp-Lys-Lys. |

*Peptides were conjugated to PMO-DBCO via N-term azidopentanoic acid.
**The peptides are amidated at the C-terminus.

Referring to Table 6, the peptides were conjugated to PMO-DBCO via N-terminus azidopentanoic acid and are amidated at the C-terminus.

The PMO-Chimeric Peptide Conjugates Demonstrated High Efficacy: The PMO-chimeric peptides were tested at varying concentrations in the Hela-654 eGFP assay. This study demonstrated that the chimeric peptides show high efficacy for PMO delivery (see FIG. 5B and Table 7).

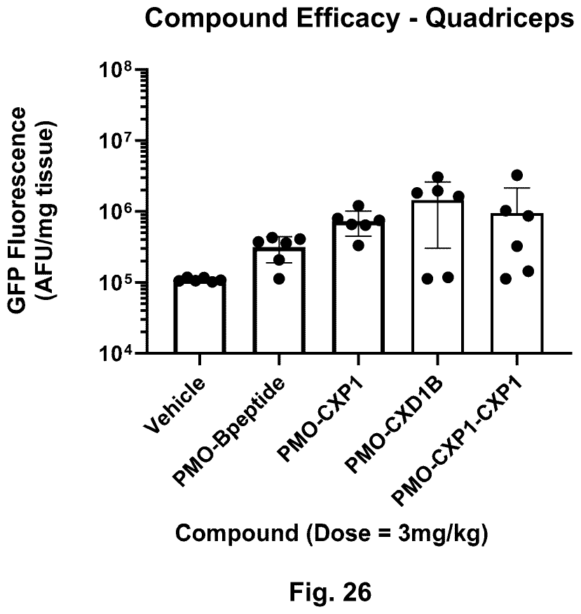

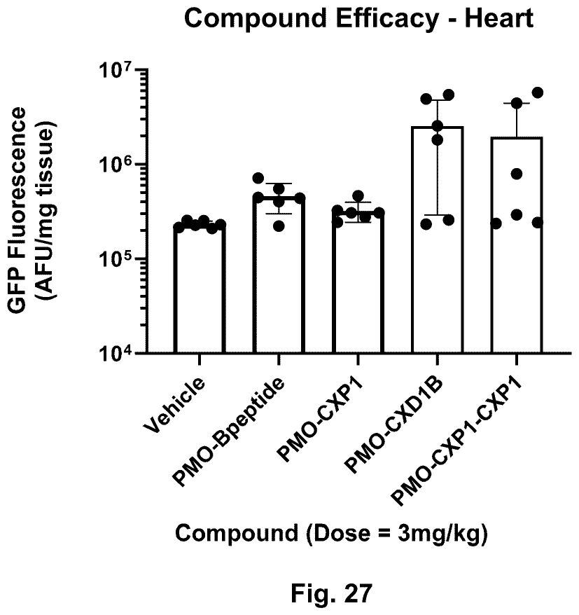

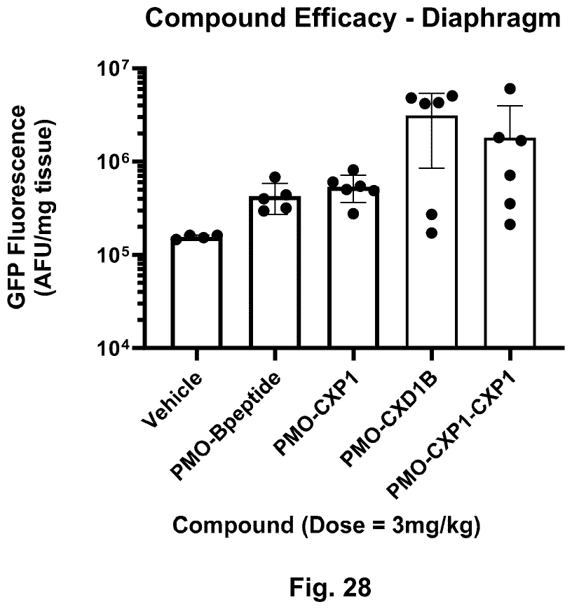

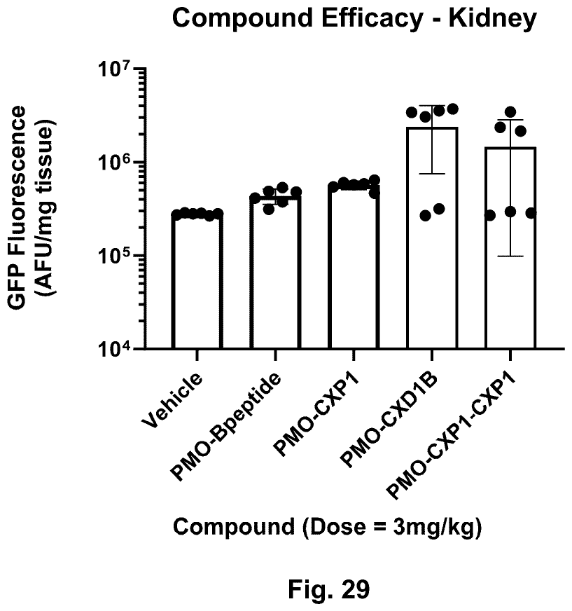

PPMOs at 3 mg/kg were intravenously delivered to eGFP-654 mice. Tissues were collected for analysis seven days after injection. The tissues were homogenized and GFP fluorescence measurements were read for a fluorescence spectrophotometer and normalized to tissue quantity to measure efficacy (FIGS. 26-29). Compound efficacy and exposure were calculated in the quadriceps (FIG. 26), heart (FIG. 27), diaphragm (FIG. 28), and kidney (FIG. 29). This study demonstrated that the chimeric peptides show high efficacy in all organs studied.

TABLE 7
Chimeric Peptide Hela-654 eGFP Assay Data

| | | N- | | | |
|---|---|---|---|---|---|
| | | terminal | SEQ | | EC50 |
| Compound | | amino | ID | | (at |
| Name | Description | acid | No. | Peptide Sequence | 72 h) |
| PMO-CXP1- | CXP1- | Gly | 67 | Lys-Gln-Lys-Thr-Ser-Iph-Gly-Arg- | 1.07 |
| CXP1 | CXP1 | | | Gly-Pip-Lys-Trp-Lys-Lys-Gly-Lys- | |
| | | | | Gln-Lys-Thr-Ser-Iph-Gly-Arg-Gly- | |
| | | | | Pip-Lys-Trp-Lys-Lys | |
| PMO-CXP3- | CXP3- | Gly | 68 | Dab-Val-Ala-Arg-Asn-Asn-Dab- | 0.026 |
| CXP1 | CXP1 | | | Thr-Lys-Gly-Lys-Trp-Lys-Lys-Gly- | |
| | | | | Lys-Gln-Lys-Thr-Ser-Iph-Gly-Arg- | |
| | | | | Gly-Pip-Lys-Trp-Lys-Lys | |
| PMO-CXP4- | CXP4- | Gly | 69 | Cit-Met-Phe-Gly-Pip-Dab-Dab- | 59.18 |
| CXP1 | CXP1 | | | Lys-Ala-Pro-Lys-Trp-Lys-Lys-Gly- | |
| | | | | Lys-Gln-Lys-Thr-Ser-Iph-Gly-Arg- | |
| | | | | Gly-Pip-Lys-Trp-Lys-Lys | |
| PMO-SA- | SA-CXP1 | Gly | 70 | Iph-Gly-Arg-Gly-Pip-Lys-Gln-Lys- | 1.57 |
| CXP1 | | | | Thr-Ser-Iph-Gly-Arg-Gly-Pip-Lys- | |
| | | | | Trp-Lys-Lys | |
| PMO-SB- | SB-CXP1 | Gly | 71 | Lys-Thr-Ser-Iph-Gly-Arg-Lys-Gln- | 2.08 |
| CXP1 | | | | Lys-Thr-Ser-Iph-Gly-Arg-Gly-Pip- | |
| | | | | Lys-Trp-Lys-Lys | |
| PMO-SC- | SC-CXP1 | Gly | 72 | Lys-Gln-Lys-Thr-Ser-Lys-Gln-Lys- | 4.01 |
| CXP1 | | | | Thr-Ser-Iph-Gly-Arg-Gly-Pip-Lys- | |
| | | | | Trp-Lys-Lys | |
| PMO-SD- | SD-CXP1 | Gly | 73 | Lys-Trp-Lys-Lys-Gln-Lys-Thr-Ser- | 2.28 |
| CXP1 | | | | Iph-Gly-Arg-Gly-Pip-Lys-Trp-Lys- | |
| | | | | Lys | |
| PMO- | CXD1A | Gly | 74 | Lys-Thr-Ser-Iph-Gly-Arg-Lys-Gln- | 3.35 |
| CXD1A | | | | Lys-Thr-Ser-Iph-Gly-Arg-Lys-Trp- | |
| | | | | Lys-Lys | |
| PMO- | CXD1B | Gly | 75 | Lys-Gln-Lys-Thr-Ser-Iph-Gly-Arg- | 3.54 |
| CXD1B | | | | Lys-Trp-Lys-Gln-Lys-Thr-Ser-Iph- | |
| | | | | Gly-Arg-Lys-Trp-Lys-Lys | |

*Peptides were conjugated to PMO-DBCO via N-term azidopentanoic acid.
**The peptides are amidated at the C-terminus.

### Example 11—M23D Studies

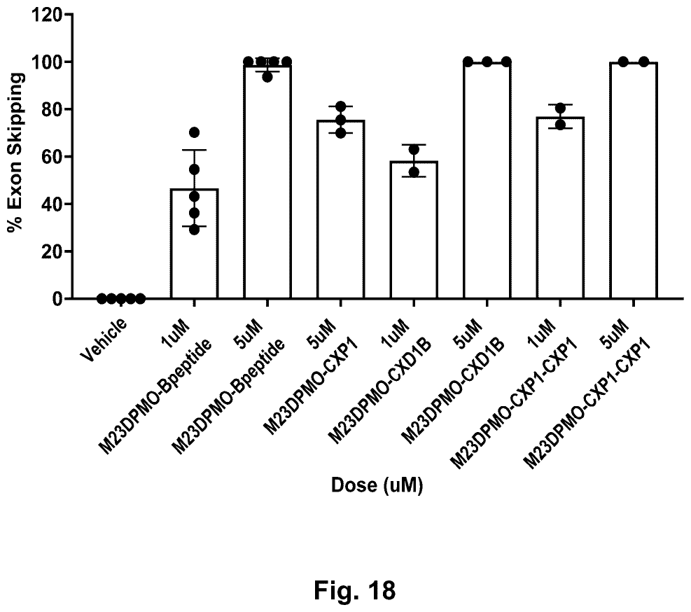

Peptides were conjugated to the M23D PMO GGCCAAACCTCGGCTTACCTGAAAT (SEQ ID NO.: 148) to make M23DPMO-CXP1, M23DPMO-CXPD1B, or M23DPMO-CXP1-CXP1. C2C12 cells were seeded in 96 well plates and were differentiated into myotubes for 3 days, before the cells were transfected with the indicated concentration of M23DPMO-CXP1, M23DPMO-CXPD1B, or M23DPMO-CXP1-CXP1 (FIG. 18). The cells were collected after 24 hours incubation. Endpoint PCR was performed and the percentage of exon skipping was calculated to be the percentage of skipped transcript to the total transcript. This study demonstrated that the chimeric peptides show increased efficacy over the M23DPMO-CXP1 conjugate and are similar in efficacy to M23DPMO-Bpeptide.

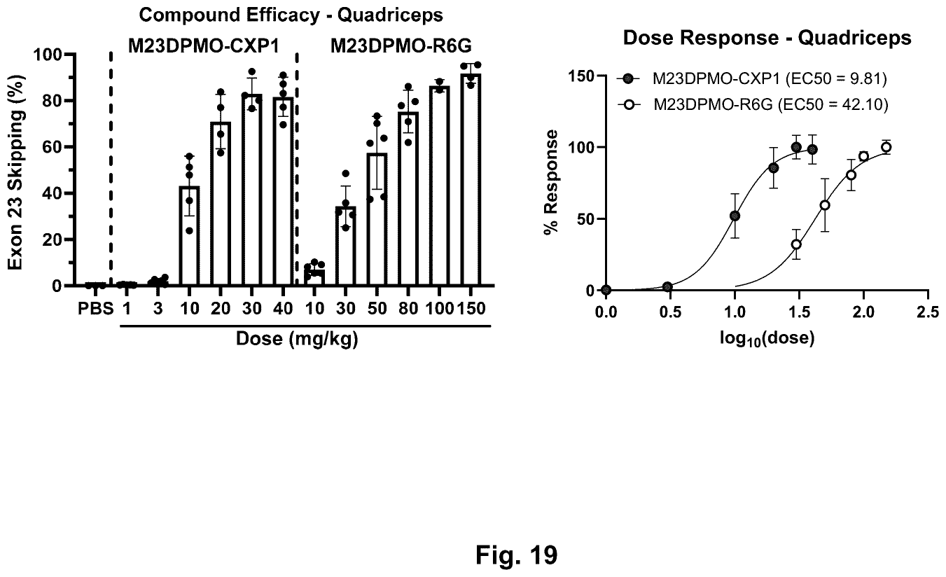

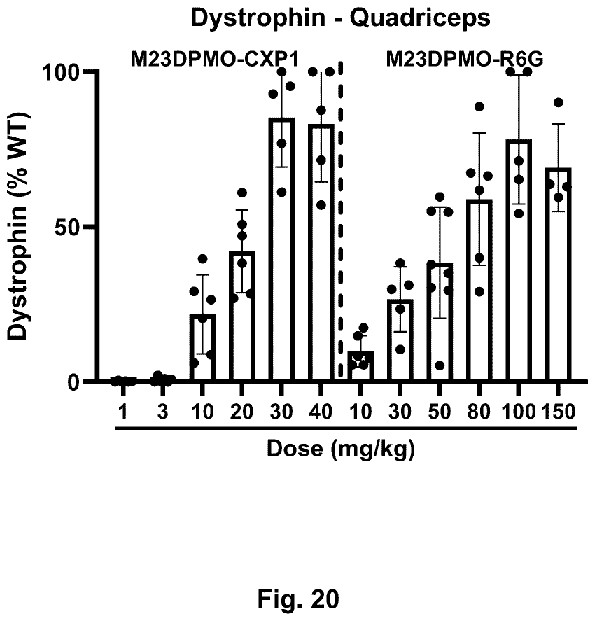

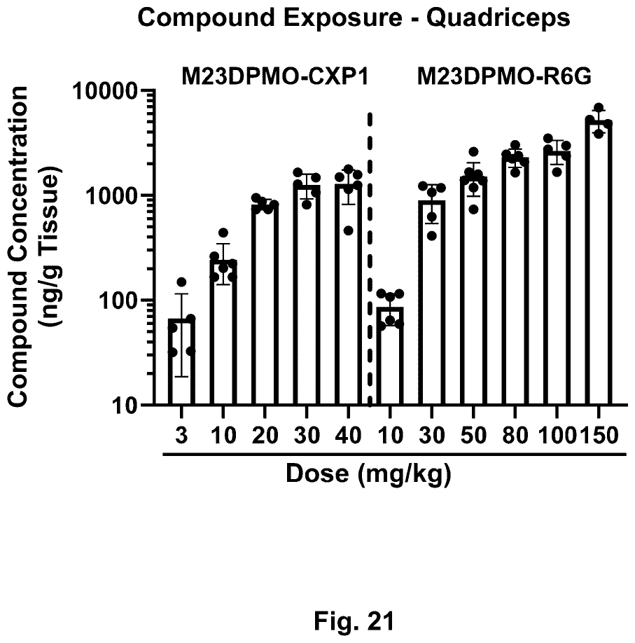

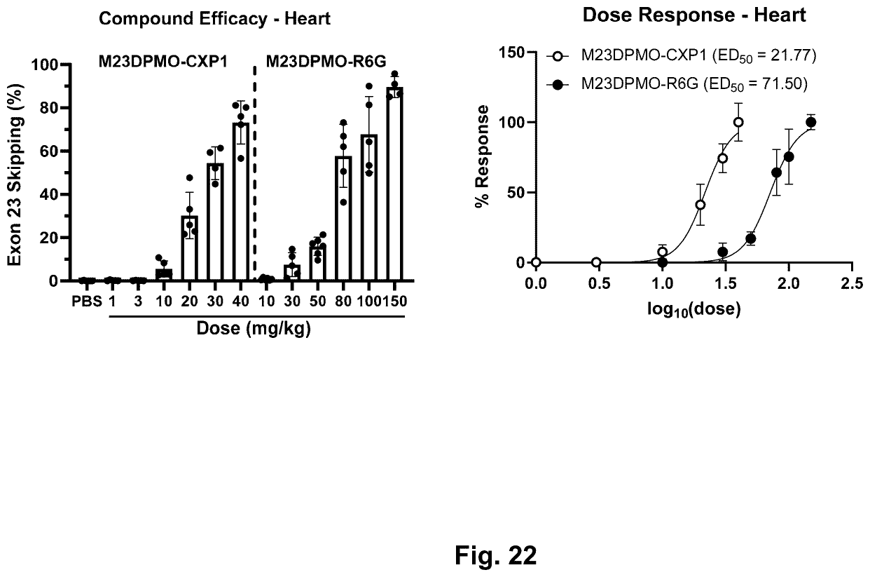

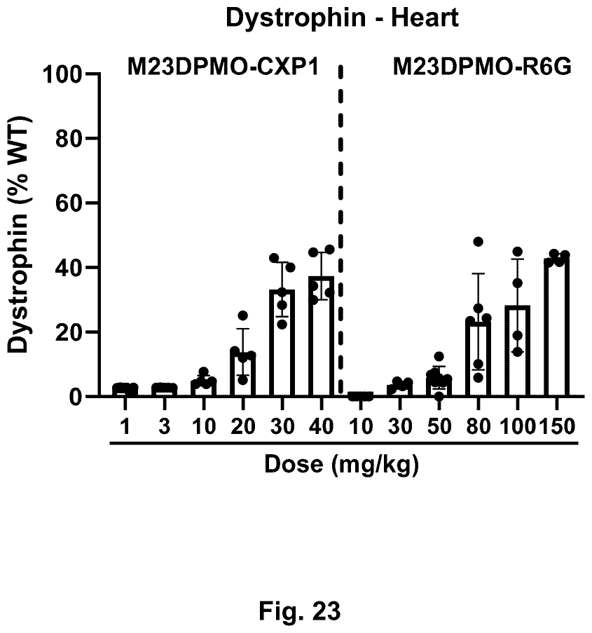

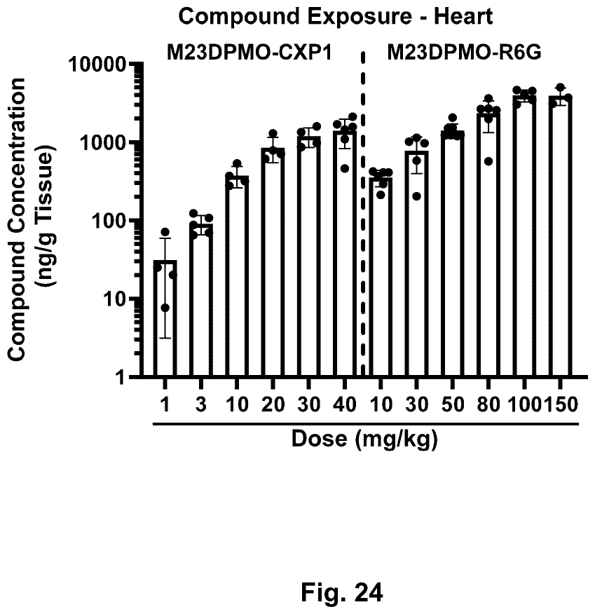

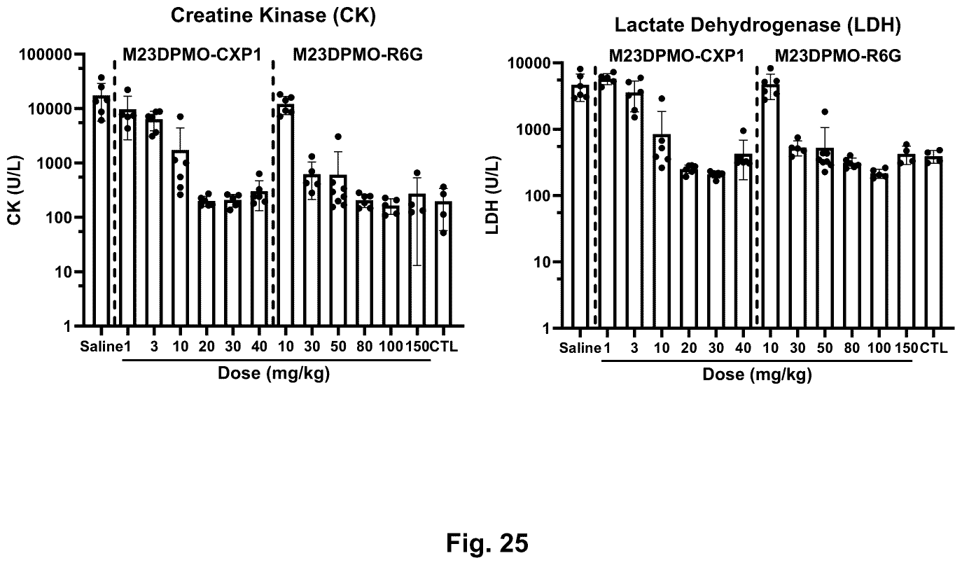

M23DPMO-CXP1 conjugate was intravenously delivered to MDX mice at various doses and the tissues were collected for analysis 7 days after injection. The tissues were homogenized and RNA was extracted from the samples. Droplet Digital PCR (ddPCR) was used to quantify skipped transcript versus unskipped transcript. The percentage of Exon 23 skipping was calculated to be the number of skipped transcript over the total transcripts. ED50s were also derived from the dose-response curve. Dystrophin protein expression was determined using an automated western blot system (Proteinsimple's Jess). The percentage of dystrophin observed in MDX tissue was calculated relative to a wildtype protein standard curve. M23DPMO-CXP1 compound exposure was calculated with an enzyme-linked oligonucleotide hybridization assay. This study demonstrated that M23DPMO-CXP1 conjugate was more efficacious than M23DPMO-R6G. Compound efficacy, dystrophin concentration, and exposure were calculated in the quadriceps (FIGS. 19-21) and heart (FIGS. 22-24). Blood serum was also collected from the MDX mice and serum biomarkers were analyzed, demonstrating a decrease in creatine kinase (CK) and lactate dehydrogenase (LDH) with increasing dose of M23D PMO-CXP1 (see FIG. 25 and Table 8).

TABLE 8
M23D CXP1 Peptide Sequences and Percentage of Exon 23 Skipping

| | | N- | | | |
|---|---|---|---|---|---|
| | | terminal | SEQ | | Percentage of |
| Compound | | amino | ID | | Exon Skipping |
| Name | Description | acid | No. | Peptide Sequence | at 5 μM |
| M23DPMO- | CXP1 | Gly | 48 | Lys-Gln-Lys-Thr-Ser-Iph- | 75.5% |
| CXP1 | | | | Gly-Arg-Gly-Pip-Lys-Trp- | |
| | | | | Lys-Lys | |
| M23DPMO- | CXD1B | Gly | 75 | Lys-Gln-Lys-Thr-Ser-Iph- | 100% |
| CXD1B | | | | Gly-Arg-Lys-Trp-Lys-Gln- | |
| | | | | Lys-Thr-Ser-Iph-Gly-Arg- | |
| | | | | Lys-Trp-Lys-Lys | |
| M23DPMO- | CXP1- | Gly | 67 | Lys-Gln-Lys-Thr-Ser-Iph- | 100% |
| CXP1-CXP1 | CXP1 | | | Gly-Arg-Gly-Pip-Lys-Trp- | |
| | | | | Lys-Lys-Gly-Lys-Gln-Lys- | |
| | | | | Thr-Ser-Iph-Gly-Arg-Gly- | |
| | | | | Pip-Lys-Trp-Lys-Lys | |

*Peptides were conjugated to PMO-DBCO via N-term azidopentanoic acid.
**The peptides are amidated at the C-terminus.

### Example 12—Segment-Chimeric Peptide Conjugates

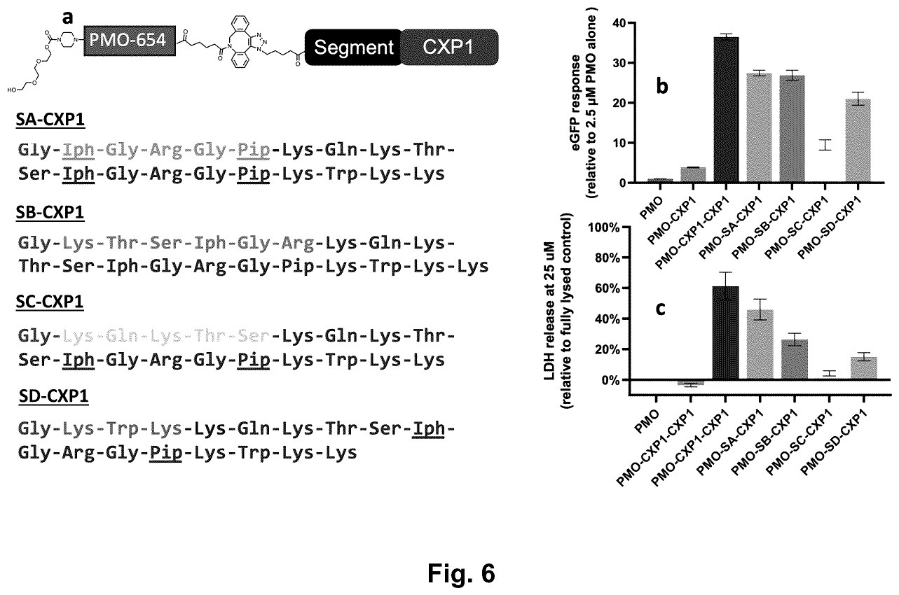

Synthesis of PMO-Segment-Chimeric Peptide Conjugates: Short, overlapping peptide segments (named SA, SB, SC, and SD) of the CXP1 sequence with a net positive charge of +2 were appended to PMO on the N-terminus and the CXP1 sequence on the C-terminus (FIG. 6A).

TABLE 9
Segments-Chimeric Peptide Sequences

| | N-terminal | | |
|---|---|---|---|
| Compound | (Linking | SEQ ID | |
| Name | AA) | NO. | Peptide Sequences |
| SA-CXP1 | Gly | 70 | Iph-Gly-Arg-Gly-Pip-Lys-Gln-Lys-Thr-Ser-Iph-Gly- |
| | | | Arg-Gly-Pip-Lys-Trp-Lys-Lys |
| SB-CXP1 | Gly | 71 | Lys-Thr-Ser-Iph-Gly-Arg-Lys-Gln-Lys-Thr-Ser-Iph- |
| | | | Gly-Arg-Gly-Pip-Lys-Trp-Lys-Lys |
| SC-CXP1 | Gly | 72 | Lys-Gln-Lys-Thr-Ser-Lys-Gln-Lys-Thr-Ser-Iph-Gly- |
| | | | Arg-Gly-Pip-Lys-Trp-Lys-Lys |
| SD-CXP1 | Gly | 73 | Lys-Trp-Lys-Lys-Gln-Lys-Thr-Ser-Iph-Gly-Arg-Gly- |
| | | | Pip-Lys-Trp-Lys-Lys |

*Peptides were conjugated to PMO-DBCO via N-term azidopentanoic acid.
**The peptides are amidated at the C-terminus.

The Segments of CXP1 Showed Key Regions for Efficacy and Toxicity: Each segment was tested for PMO response and toxicity to determine which portions of the sequence contribute to the penetration and toxicity of the CXP1-CXP1 chimera (Examples 4 and 5). SA-CXP1 and SB-CXP1 showed the highest PMO response compared to the other segment-CXP1 sequences (FIG. 6B). Peptide SA-CXP1 had the toxicity closest to the parent CXP1-CXP1 peptide (FIG. 6C). This suggests that the “Iph-Gly-Arg” motif across segments A and B is likely important for penetration, while the “Gly-Pip” motif unique to segment A may be contributing to peptide toxicity.

### Example 13—PMO-Cationic Peptide Conjugates

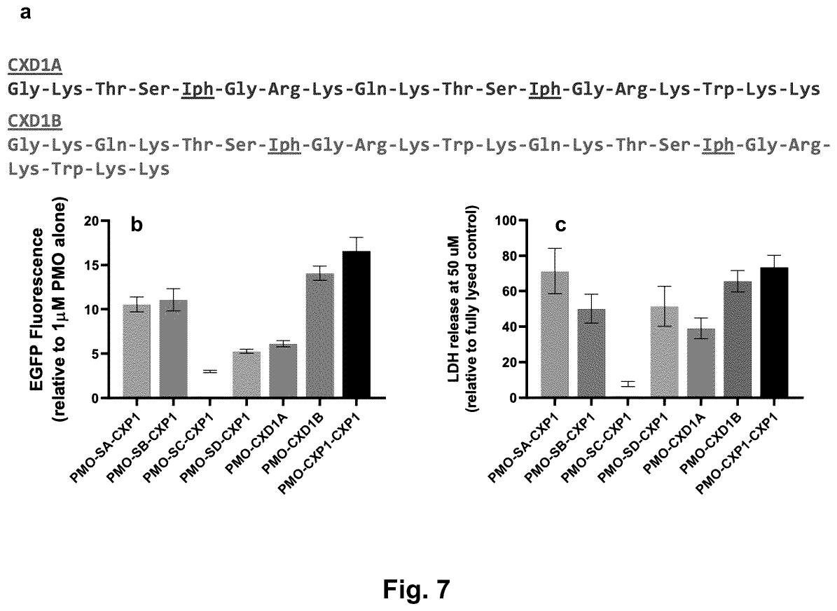

Synthesis of New, Cationic Peptide: Two new peptide sequences were designed based on the original CXP1. These peptide sequences are shorter than the CXP1-CXP1 chimera (SEQ ID No.: 10) and lack the potentially toxic “Gly-Pip” motif. These two sequences were synthesized and conjugated to the PMO cargo (FIG. 7A).

TABLE 10
Cationic Peptide Sequences

| | N- | | |
|---|---|---|---|
| | terminal | | |
| Compound | (Linking | SEQ ID | |
| Name | AA) | NO. | Peptide Sequences |
| CXD1A | Gly | 74 | Lys-Thr-Ser-Iph-Gly-Arg-Lys-Gln-Lys-Thr-Ser-Iph- |
| | | | Gly-Arg-Lys-Trp-Lys-Lys |
| CXD1B | Gly | 75 | Lys-Gln-Lys-Thr-Ser-Iph-Gly-Arg-Lys-Trp-Lys-Gln- |
| | | | Lys-Thr-Ser-Iph-Gly-Arg-Lys-Trp-Lys-Lys |

*Peptides were conjugated to PMO-DBCO via N-term azidopentanoic acid.
**The peptides are amidated at the C-terminus.

The new PMO-Cationic Peptide Conjugates Demonstrated High Efficacy and Low Toxicity: CXD1A and CXD1B were tested for PMO delivery and toxicity. Both demonstrated high PMO delivery, especially the CXD1B (FIG. 7B). This peptide showed high PMO delivery and lower toxicity than the CXP1-CXP1 sequence (FIG. 7C).

### Example 14—Therapeutic Windows of PMO-Peptides

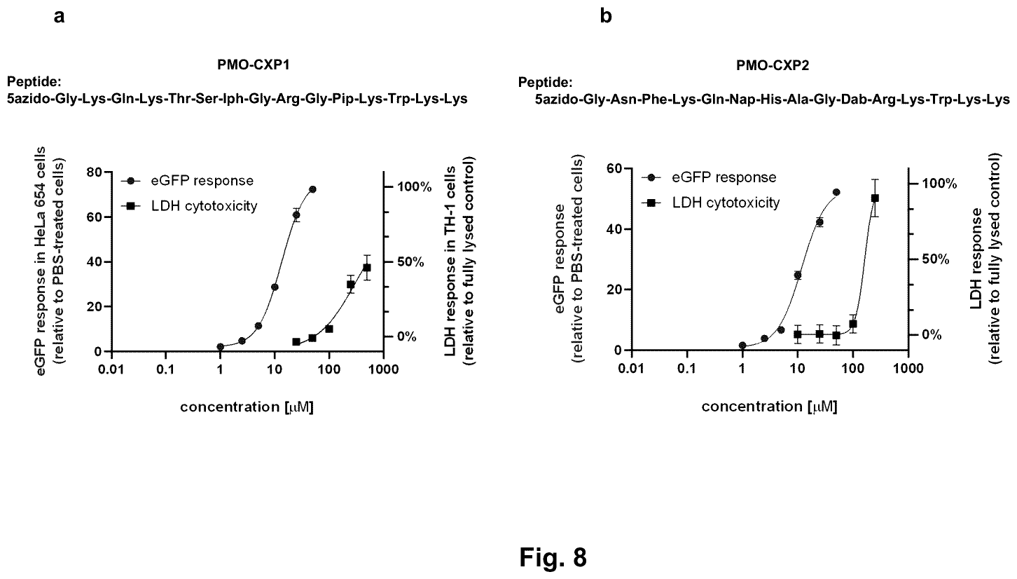

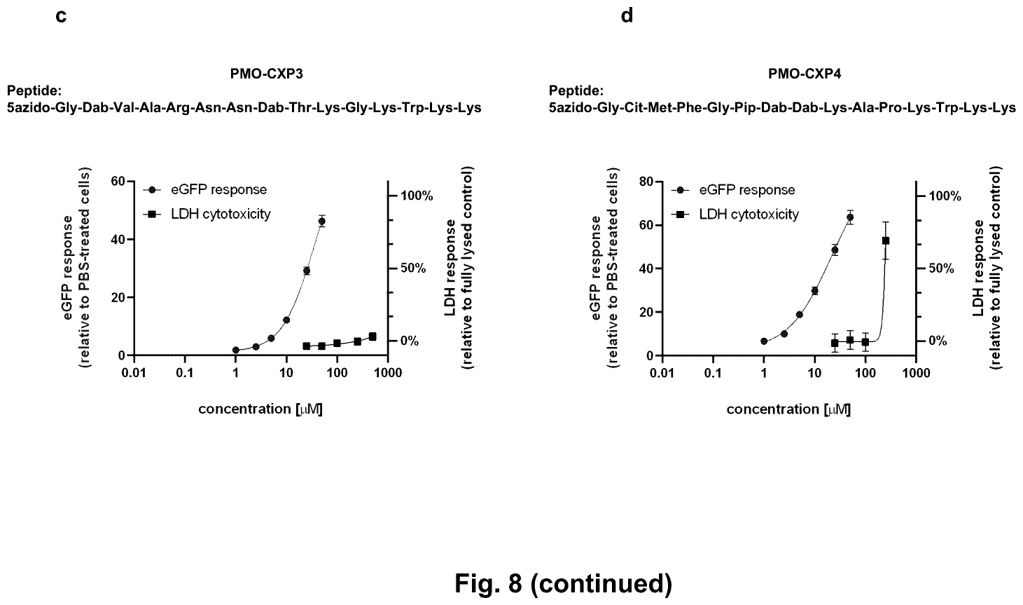

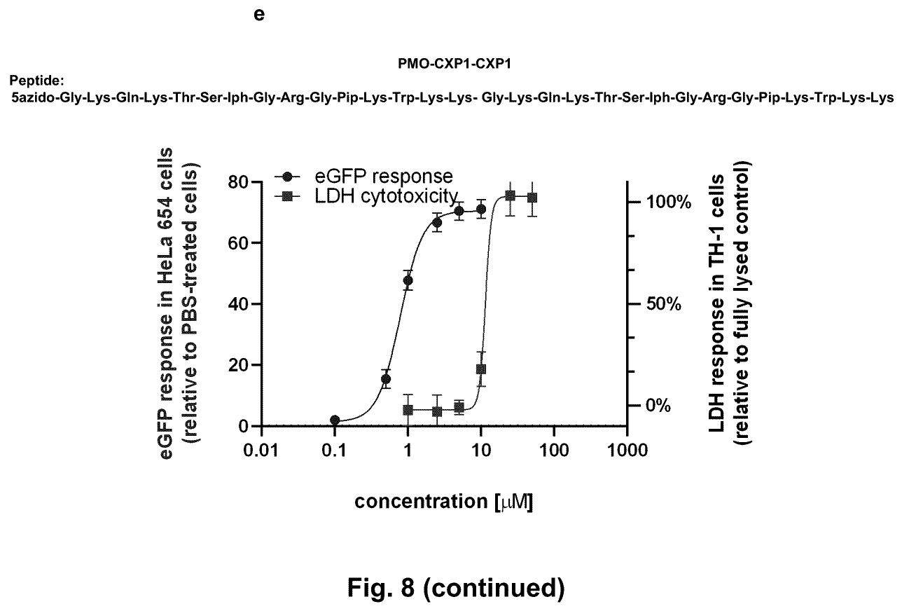

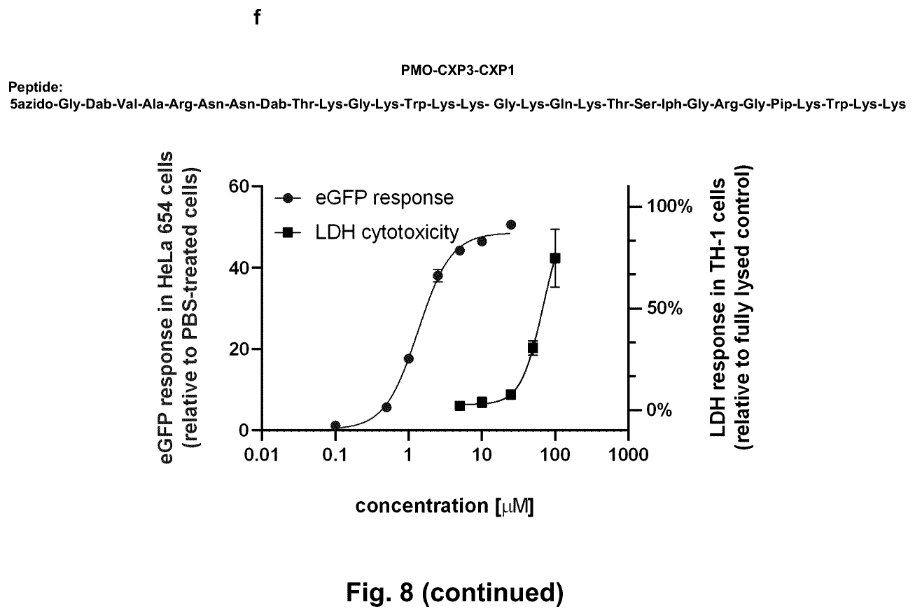

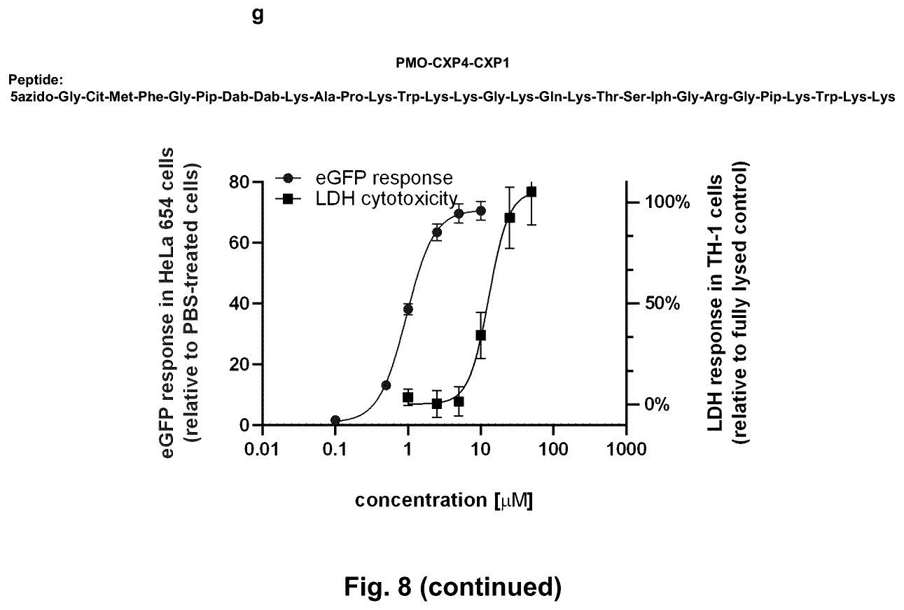

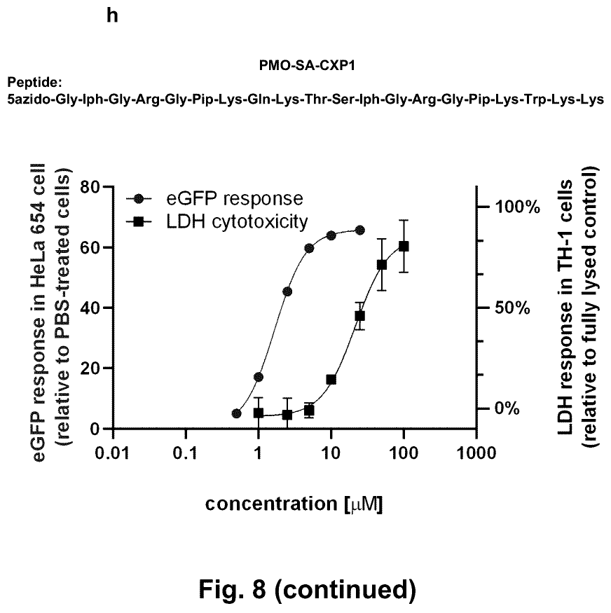

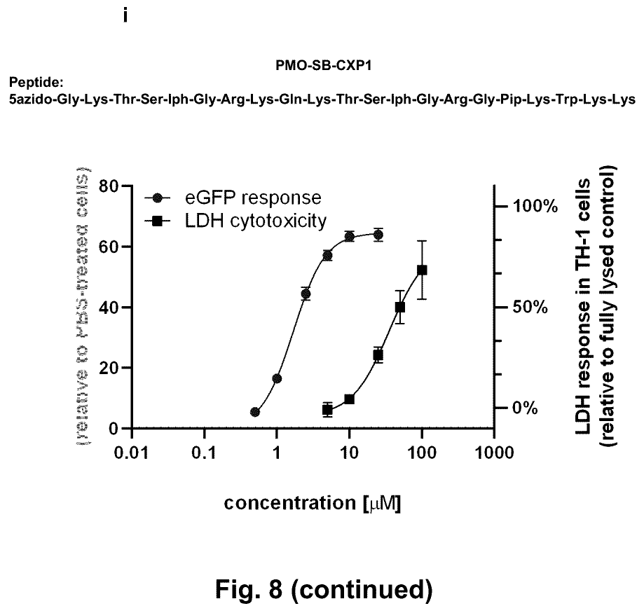

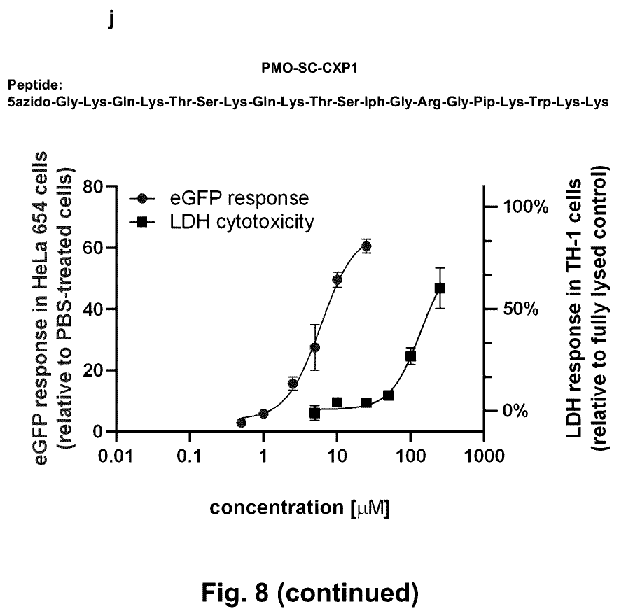

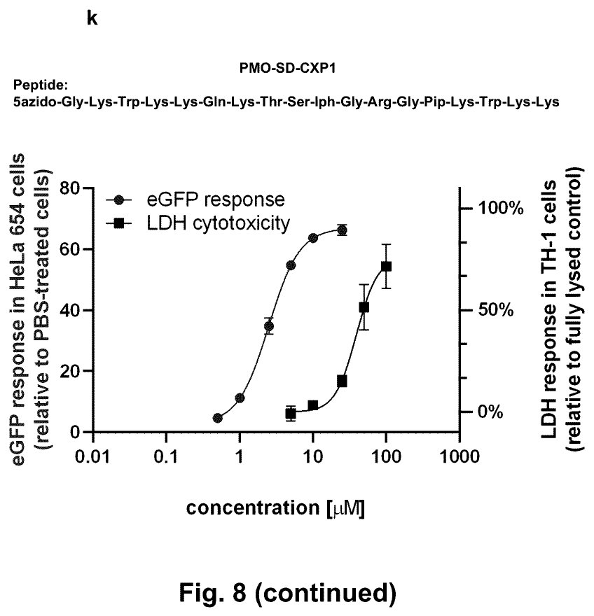

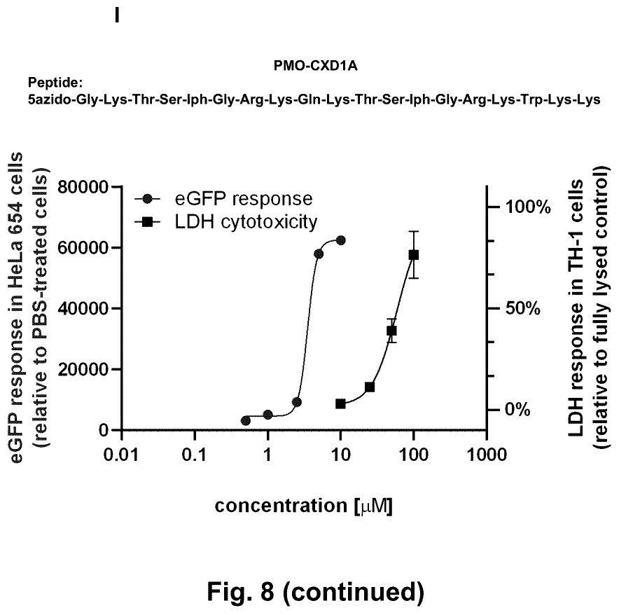

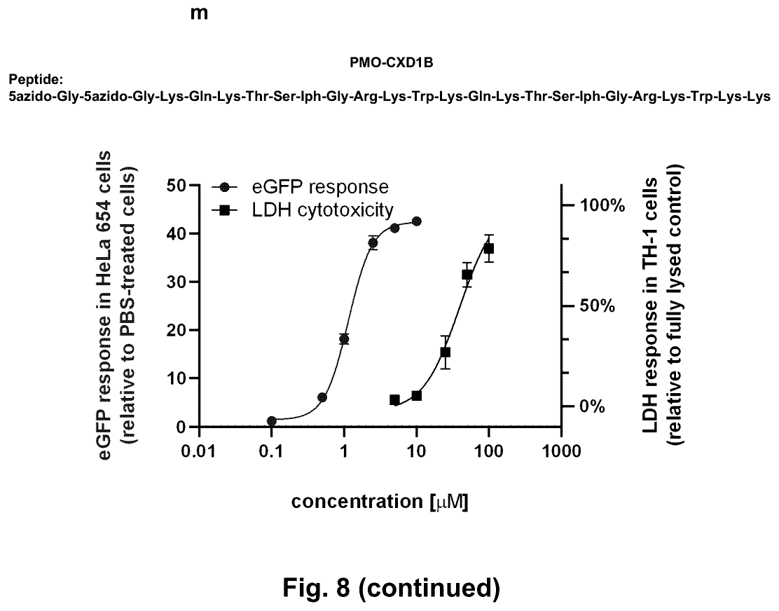

Therapeutic window studies were conducted on all PMO-peptide conjugates (SEQ ID NOs: 1-18) (FIGS. 8A-8M). PMO-efficacy was measured through the HeLa-654 eGFP assay, and toxicity was measured through LDH release at 18 hours in TH-1 renal cells. The PMO-CXD1B conjugate showed a wider therapeutic window in comparison to other PMO-peptide conjugates.

### Example 15: 5′ Versus 3′ Peptide Conjugation

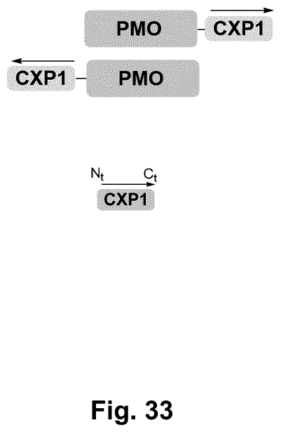

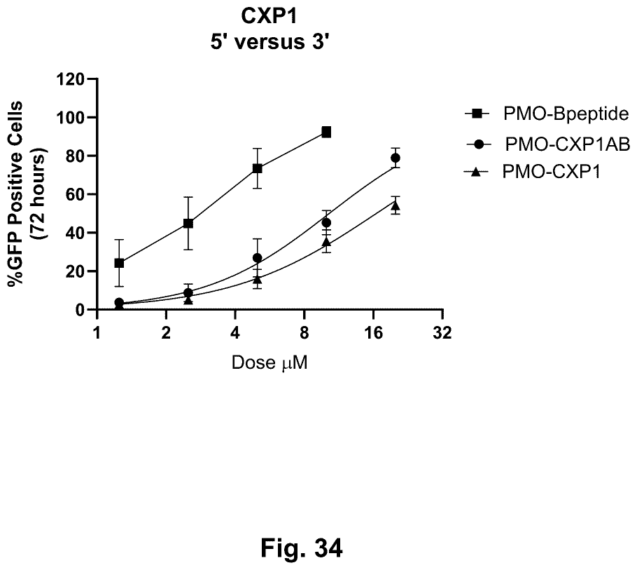

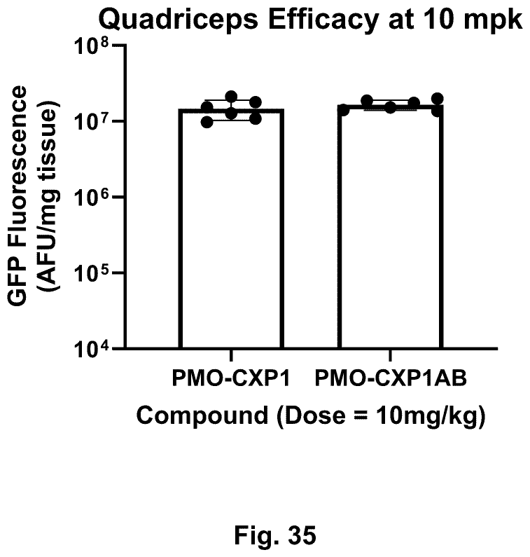

The peptide sequence CXP1 was conjugated to the 5′-end of the PMO GCTATTACCTTAACCCAG (SEQ ID NO.: 77) (FIG. 33). PMO-CXP1AB efficacy was measured in vitro at various doses with the Hela-654 eGFP assay (FIG. 34) and in vivo by dosing 10 mg/kg intravenously in a eGFP-654 mouse model and eGFP fluorescence was measured in the quadriceps (FIG. 35). PMO-CXP1AB showed similar efficacy to PMO-CXP1 both in vitro and in vivo.

### Example 16—Alanine Scan

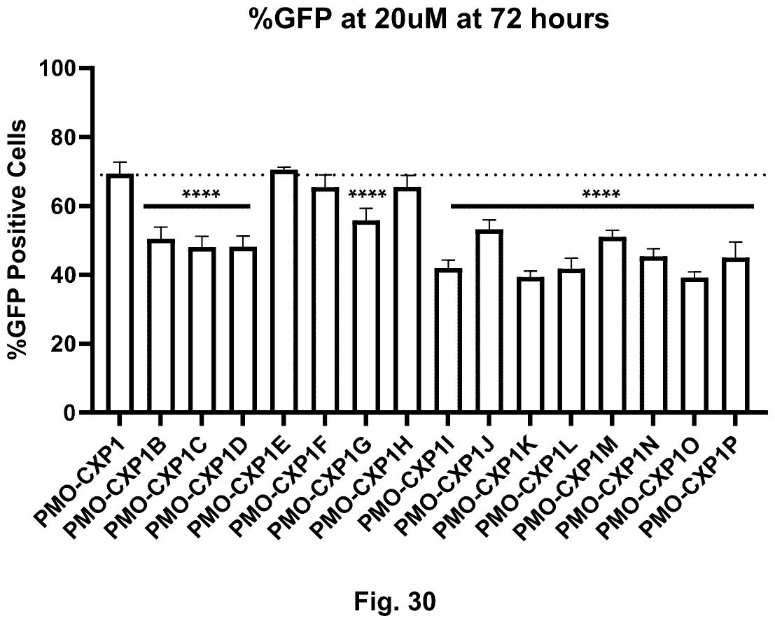

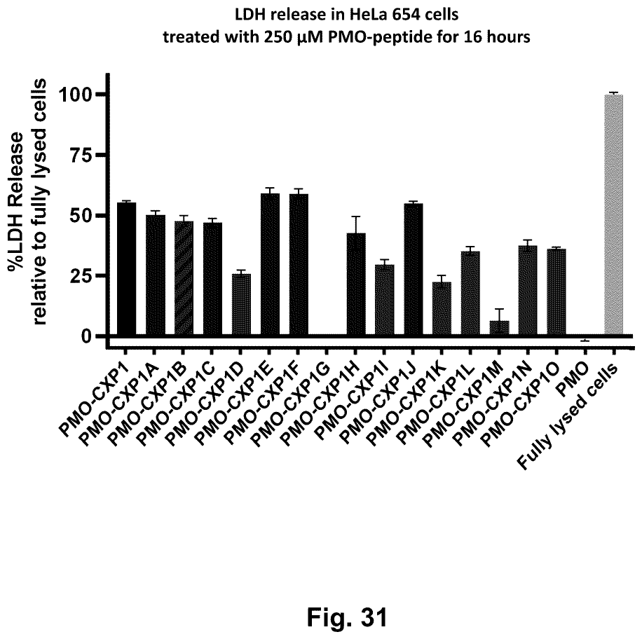

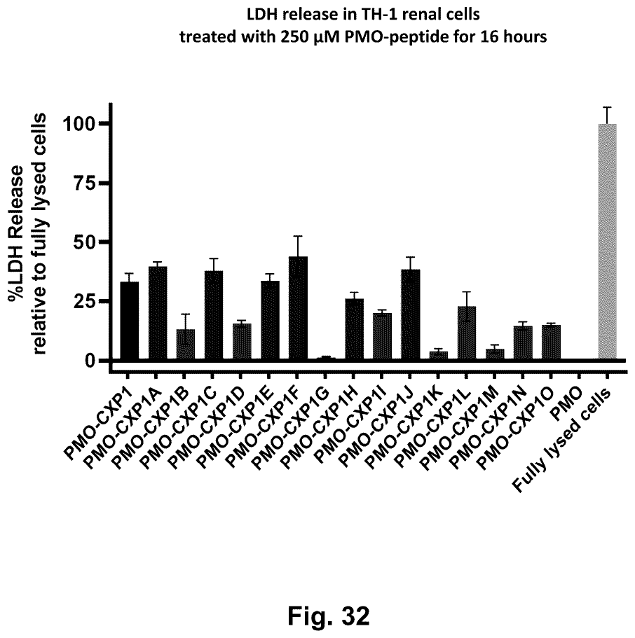

Peptide sequences were designed based on the original CXP1. Each amino acid in the original peptide sequence was replaced with an alanine to create a library of alanine-substituted analogs. The modification was conducted to understand the functional significance of each amino acid residue in the original sequence and helped determine which amino acid contributes to the activity and or cytotoxicity of the peptide. The compounds were tested at 20 μM of PPMO with the Hela-654 eGFP assay to determine efficacy. Cytotoxicity was determined using a 250 μM dose of PPMO in Hela-654 cells and TH-1 renal cells. Replacing certain amino acids with an alanine reduced the percentage of GFP positive cells (FIG. 30) and reduced the LDH release in both Hela-654 cells (FIG. 31) and TH-1 renal cells (FIG. 32), Table 11.

TABLE 11
Alanine Scan Peptide Sequences and Percentage of GFP Positive Cells

| | | | | | Percentage |
|---|---|---|---|---|---|
| | | N- | | | of GFP |
| | | terminal | SEQ | | Positive |
| Compound | | (Linking | ID | | Cells at 72 |
| Name | Description | AA) | No. | Peptide Sequence | hours (20 μM) |
| PMO- | CXP1AA | Gly | 81 | Lys-Gln-Lys-Thr-Ser-Iph-Gly- | — |
| CXP1AA | | | | Lys-Gly-Pip-Lys-Trp-Lys-Lys | |
| PMO- | CXP1B | Gly | 83 | Ala-Gln-Lys-Thr-Ser-Iph-Gly- | 50.5 |
| CXP1B | | | | Arg-Gly-Pip-Lys-Trp-Lys-Lys | |
| PMO- | CXP1C | Gly | 84 | Lys-Ala-Lys-Thr-Ser-Iph-Gly- | 50.0 |
| CXP1C | | | | Arg-Gly-Pip-Lys-Trp-Lys-Lys | |
| PMO- | CXP1D | Gly | 85 | Lys-Gln-Ala-Thr-Ser-Iph-Gly- | 48.2 |
| CXP1D | | | | Arg-Gly-Pip-Lys-Trp-Lys-Lys | |
| PMO- | CXP1E | Gly | 86 | Lys-Gln-Lys-Ala-Ser-Iph-Gly- | 70.5 |
| CXP1E | | | | Arg-Gly-Pip-Lys-Trp-Lys-Lys | |
| PMO- | CXP1F | Gly | 87 | Lys-Gln-Lys-Thr-Ala-Iph-Gly- | 65.5 |
| CXP1F | | | | Arg-Gly-Pip-Lys-Trp-Lys-Lys | |
| PMO- | CXP1G | Gly | 88 | Lys-Gln-Lys-Thr-Ser-Ala-Gly- | 55.8 |
| CXP1G | | | | Arg-Gly-Pip-Lys-Trp-Lys-Lys | |
| PMO- | CXP1H | Gly | 89 | Lys-Gln-Lys-Thr-Ser-Iph-Ala- | 65.5 |
| CXP1H | | | | Arg-Gly-Pip-Lys-Trp-Lys-Lys | |
| PMO- | CXP1I | Gly | 90 | Lys-Gln-Lys-Thr-Ser-Iph-Gly- | 42.0 |
| CXP1I | | | | Ala-Gly-Pip-Lys-Trp-Lys-Lys | |
| PMO- | CXP1J | Gly | 91 | Lys-Gln-Lys-Thr-Ser-Iph-Gly- | 53.2 |
| CXP1J | | | | Arg-Ala-Pip-Lys-Trp-Lys-Lys | |
| PMO- | CXP1K | Gly | 92 | Lys-Gln-Lys-Thr-Ser-Iph-Gly- | 39.3 |
| CXP1K | | | | Arg-Gly-Ala-Lys-Trp-Lys-Lys | |
| PMO- | CXP1L | Gly | 93 | Lys-Gln-Lys-Thr-Ser-Iph-Gly- | 41.8 |
| CXP1L | | | | Arg-Gly-Pip-Ala-Trp-Lys-Lys | |
| PMO- | CXP1M | Gly | 94 | Lys-Gln-Lys-Thr-Ser-Iph-Gly- | 51.0 |
| CXP1M | | | | Arg-Gly-Pip-Lys-Ala-Lys-Lys | |
| PMO- | CXP1N | Gly | 95 | Lys-Gln-Lys-Thr-Ser-Iph-Gly- | 45.4 |
| CXP1N | | | | Arg-Gly-Pip-Lys-Trp-Ala-Lys | |
| PMO- | CXP1O | Gly | 126 | Lys-Gln-Lys-Thr-Ser-Iph-Gly- | 39.2 |
| CXP1O | | | | Arg-Gly-Pip-Lys-Trp-Lys-Ala | |
| PMO- | CXP1P | Ala | 82 | Lys-Gln-Lys-Thr-Ser-Iph-Ala- | 41.6 |
| CXP1P | | | | Arg-Ala-Pip-Lys-Trp-Lys-Lys | |

*Peptides were conjugated to PMO-DBCO via N-term azidopentanoic acid.
**The peptides are amidated at the C-terminus.

### Example 17: Iph Scan

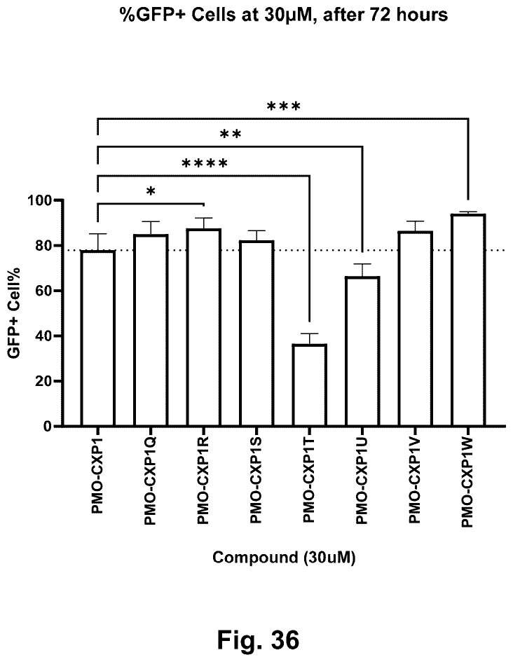

Peptide sequences were designed based on the original CXP1. Each amino acid in the original peptide sequence was replaced with an Iph to create Iph-substituted analogs. The modification was conducted to reduce the overall charge of original CXP1. The compounds were tested at 30 μM of compound with the Hela-654 eGFP assay to determine efficacy. Replacing certain amino acids with an IPH-substituted analog maintained the efficacy in the Hela-654 eGFP Assay (FIG. 36).

TABLE 12
CXP1 Iph Scan Peptide Sequences and Percentage of GFP Positive Cells

| | | N- | | | Percentage of |
|---|---|---|---|---|---|
| | | terminal | SEQ | | GFP Positive |
| Compound | | (Linking | ID | | Cells at 72 |
| Name | Description | AA) | No. | Sequences | hours (30 μM) |
| PMO-CXP1Q | CXP1Q | Gly | 97 | Iph-Gln-Lys-Thr-Ser-Iph-Gly- | 85 |
| | | | | Arg-Gly-Pip-Lys-Trp-Lys-Lys | |
| PMO-CXP1R | CXP1R | Gly | 98 | Lys-Gln-Iph-Thr-Ser-Iph-Gly- | 87.6 |
| | | | | Arg-Gly-Pip-Lys-Trp-Lys-Lys | |
| PMO-CXP1S | CXP1S | Gly | 99 | Lys-Gln-Lys-Thr-Ser-Iph-Gly- | 82.3 |
| | | | | Iph-Gly-Pip-Lys-Trp-Lys-Lys | |
| PMO-CXP1T | CXP1T | Gly | 100 | Lys-Gln-Lys-Thr-Ser-Iph-Gly- | 36.5 |
| | | | | Arg-Gly-Iph-Lys-Trp-Lys-Lys | |
| PMO-CXP1U | CXP1U | Gly | 101 | Lys-Gln-Lys-Thr-Ser-Iph-Gly- | 66.4 |
| | | | | Arg-Gly-Pip-Iph-Trp-Lys-Lys | |
| PMO-CXP1V | CXP1V | Gly | 102 | Lys-Gln-Lys-Thr-Ser-Iph-Gly- | 86.4 |
| | | | | Arg-Gly-Pip-Lys-Trp-Iph-Lys | |
| PMO-CXP1W | CXP1W | Gly | 133 | Lys-Gln-Lys-Thr-Ser-Iph-Gly- | 94.11 |
| | | | | Arg-Gly-Pip-Lys-Trp-Lys-Iph | |

*Peptides were conjugated to PMO-DBCO via N-term azidopentanoic acid.
**The peptides are amidated at the C-terminus.

### Example 18: Replacing Unnatural Amino Acids

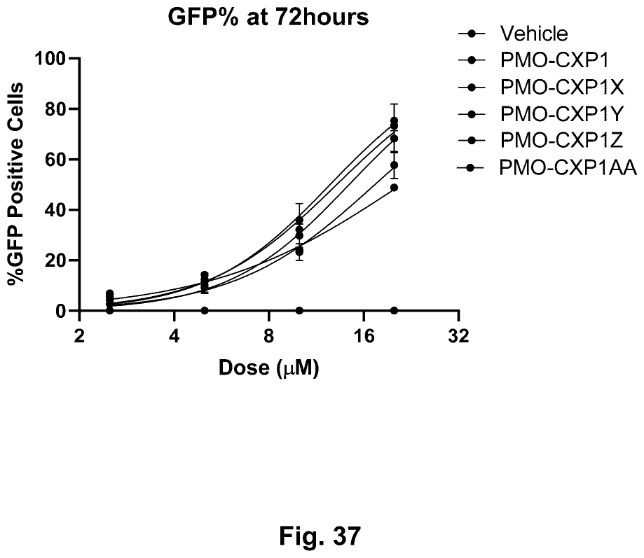

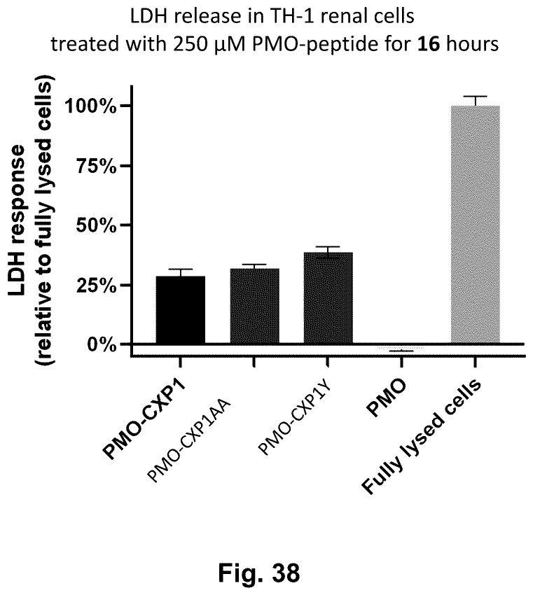

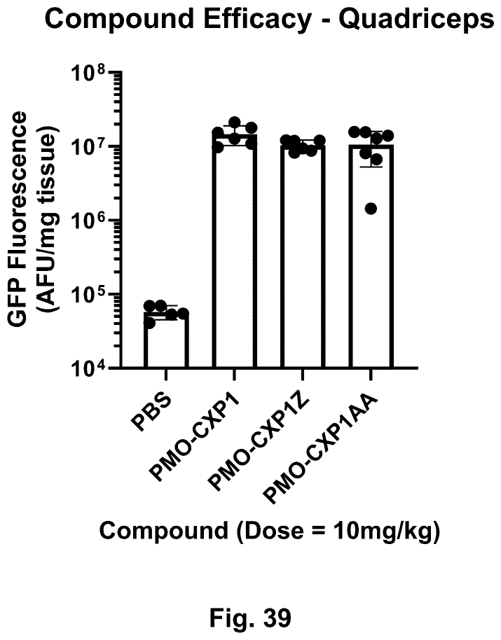

Peptide sequences were designed based on the original CXP1 with each unnatural amino acid in the original CXP1 peptide sequence being replaced individually or in combination to evaluate the contribution of the unnatural amino acids towards the efficacy and tolerability of CXP1. The compounds were tested at varying concentrations with the Hela-654 eGFP assay to determine efficacy and at 250 μM in TH-1 renal cells to determine cytotoxicity (FIGS. 37-38). Replacing the unnatural amino acids does not significantly alter efficacy or cytotoxicity. Compounds were then dosed at 10 mg/kg in eGFP-654 mice to determine their efficacy in vivo. No differences in efficacy were observed in the quadriceps of eGFP-654 mice after 7 days (see FIG. 39 and Table 13).

TABLE 13
CXP1 Analogs-Peptide Sequences and Hela-654 eGFP Assay Data

| | | N- | | | |
|---|---|---|---|---|---|
| | | terminal | | | |
| Compound | | (Linking | SEQ ID | | EC50 |
| Name | Description | AA) | No. | Sequence | (72 h) |
| PMO-CXP1X | CXP1X | Gly | 78 | Lys-Gln-Lys-Thr-Ser-Phe-Gly-Arg- | 17.35 |
| | | | | Gly-Pip-Lys-Trp-Lys-Lys | |
| PMO-CXP1Y | CXP1Y | Gly | 79 | Lys-Gln-Lys-Thr-Ser-Iph-Gly-Arg-Gly- | 14.37 |
| | | | | Lys-Lys-Trp-Lys-Lys | |
| PMO-CXP1Z | CXP1Z | Gly | 80 | Lys-Gln-Lys-Thr-Ser-Phe-Gly-Arg- | 21.06 |
| | | | | Gly-Lys-Lys-Trp-Lys-Lys | |
| PMO-CXP1AA | CXP1AA | Gly | 81 | Lys-Gln-Lys-Thr-Ser-Iph-Gly-Lys-Gly- | 13.12 |
| | | | | Pip-Lys-Trp-Lys-Lys | |

*Peptides were conjugated to PMO-DBCO via N-term azidopentanoic acid.
**The peptides are amidated at the C-terminus.

### Example 19: Reducing Hydrophobicity Through Shorter Side Chains with Lysine Analogs

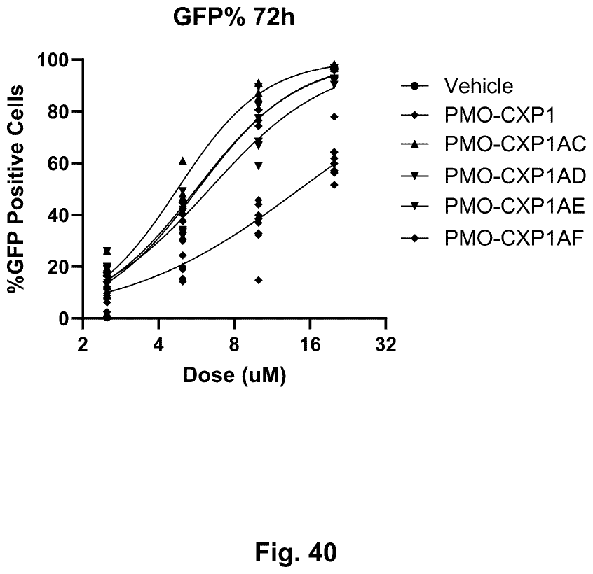

Peptide sequences were designed based on the original CXP1 peptide where each lysine and/or arginine in the original CXP1 peptide sequence was replaced with a Dab side chain to reduce the hydrophobicity through shorter side chains, and to improve the efficacy and protease stability of the peptide. The PPMOs were tested at varying concentrations with the Hela-654 eGFP assay to determine efficacy (FIG. 40). Replacing the lysine with Dab was observed to increase the efficacy in the Hela-654 eGFP assay as compared to PMO-CXP1, Table 14.

TABLE 14
CXP1 Dabs Peptide Sequences, Hela-654 eGFP Assay Data, and Percentage of
GFP Positive Cells

| | | N- | | | |
|---|---|---|---|---|---|
| | | terminal | | | EC50 |
| Compound | | (Linking | SEQ ID | | (at |
| Name | Description | AA) | No. | Sequence | 72 h) |
| PMO-CXP1AC | CXP1AC | Gly | 134 | Dab-Gln-Dab-Thr-Ser-Iph-Gly-Arg- | 4.75 |
| | | | | Gly-Pip-Dab-Trp-Dab-Dab | |
| PMO-CXP1AD | CXP1AD | Gly | 135 | Dab-Gln-Dab-Thr-Ser-Iph-Gly-Dab- | 5.64 |
| | | | | Gly-Pip-Dab-Trp-Dab-Dab | |
| PMO-CXP1AE | CXP1AE | Gly | 136 | Dab-Gln-Dab-Thr-Ser-Iph-Gly-Arg- | 6.38 |
| | | | | Gly-Dab-Dab-Trp-Dab-Dab | |
| PMO-CXP1AF | CXP1AF | Gly | 137 | Dab-Gln-Dab-Thr-Ser-Iph-Gly-Dab- | 5.73 |
| | | | | Gly-Dab-Dab-Trp-Dab-Dab | |

*Peptides were conjugated to PMO-DBCO via N-term azidopentanoic acid.
**The peptides are amidated at the C-terminus.

### IX. Additional Embodiments

Additional embodiments include embodiments P1 to P52 following.

Embodiment P1. A peptide-oligonucleotide conjugate of Formula (I):

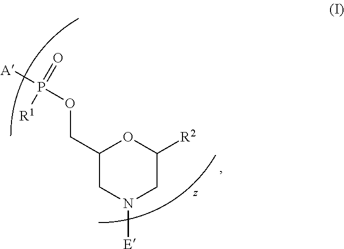

or a pharmaceutically acceptable salt thereof, wherein:
A′ is selected from —N(H)CH₂C(O)NH₂, —N(C₁₋₆-alkyl)CH₂C(O)NH₂,

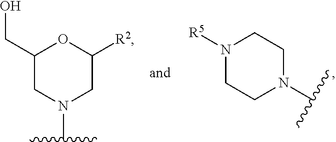

wherein
R⁵ is —C(O)(O-alkyl)ₓ-OH, wherein x is 3-10 and each alkyl group is, independently at each occurrence, C₂₋₆-alkyl, or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O-heteroaryl-R⁶,
   and

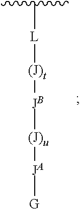

wherein R⁶ is selected from OH, SH, and NH₂, or R⁶ is selected from O, S, and NH, each of which is covalently linked to a solid support;
each R¹ is independently OH or —N(R³)(R⁴), wherein each R³ and R⁴ are, independently at each occurrence H or —C₁₋₆-alkyl;
each R² is independently, at each occurrence, selected from H, a nucleobase, and a nucleobase functionalized with a chemical protecting-group, wherein the nucleobase, independently at each occurrence, comprises a C₃₋₆-heterocyclic ring selected from pyridine, pyrimidine, purine, and deaza-purine;
z is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl,

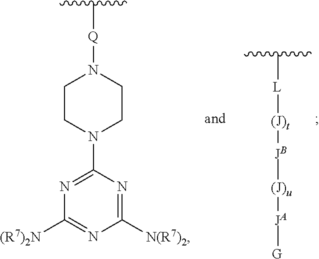

wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is covalently linked by an amide bond to J, and L is —C(O)(CH₂)₁₋₈C(O)—C₇₋₁₈-heteroaromatic-(CH₂)₁₋₈C(O)—, —C(O)(CH₂)₁₋₈C(O)—C₇₋₁₈-heteroaromatic-(CH₂)₁₋₈C(O)—(Y)—; —C(O)(CH₂)₁₋₈C(O)—C₇₋₁₈-heteroaromatic-(CH₂)₁₋₈C(O)—(Y—Y)—;
Y is independently at each occurrence a linking amino acid;
t is 3-20;
u is 0 or 3-20;
J is independently at each occurrence selected from arginine, histidine, lysine, aspartic acid, glutamic acid, serine, threonine, asparagine, glutamine, glycine, methionine, proline, valine, alanine, leucine, phenylalanine, tryptophan,

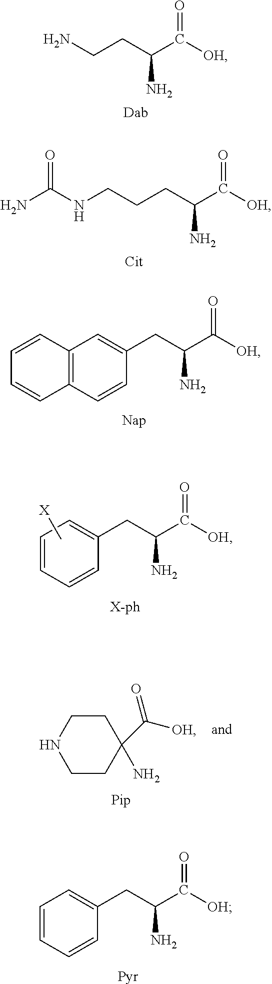

wherein at least one J is:

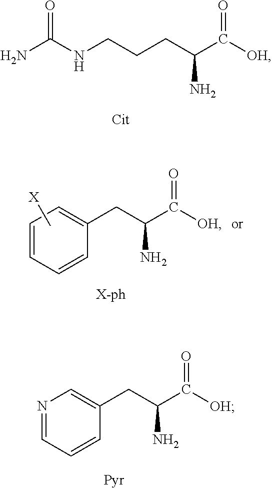

X is halogen;
J^{A} is the amino acid sequence selected from Lys-Trp-Lys-Lys, Lys-Lys-Trp-Lys, Lys-Trp-Trp-Lys-Lys, Trp-Trp-Lys-Lys, Lys-Trp-Lys, Trp-Lys-Lys, Lys-Lys-Trp, Lys-Lys-Lys-Lys, Lys-Lys, and Lys-Trp;
J^{B} is absent, or J^{B} is the amino acid sequence selected from Lys-Trp-Lys-Lys, Lys-Lys-Trp-Lys, Lys-Trp-Trp-Lys-Lys, Trp-Trp-Lys-Lys, Lys-Trp-Lys, Trp-Lys-Lys, Lys-Lys-Trp, Lys-Lys-Lys-Lys, Lys-Lys, and Lys-Trp;
G is covalently linked to J^{A}, and G is selected from H, —C(O)C₁₋₆-alkyl, —NH₂, —NH(C₁₋₆-alkyl), —N(C₁₋₆-alkyl)₂, benzoyl, and stearoyl;
wherein at least one of the following conditions is true:
   1) A′ is

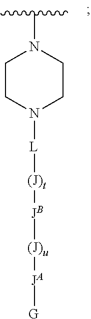

or 2) E′ is

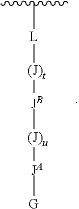

Embodiment P2. The peptide-oligonucleotide conjugate of embodiment 1, or a pharmaceutically acceptable salt thereof, wherein

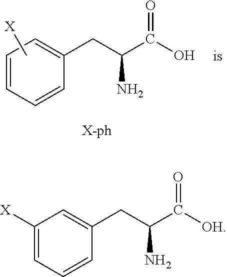

Embodiment P3. The peptide-oligonucleotide conjugate of embodiment 1 or 2, or a pharmaceutically acceptable salt thereof, wherein X is I

Embodiment P4. The peptide-oligonucleotide conjugate of any one of embodiments 1-3, or a pharmaceutically acceptable salt thereof, wherein:
u is 0; and
J^{B} is absent.

Embodiment P5. The peptide-oligonucleotide conjugate of any one of embodiments 1-4, or a pharmaceutically acceptable salt thereof, wherein
A′ is selected from —N(C₁₋₆-alkyl)CH₂C(O)NH₂,

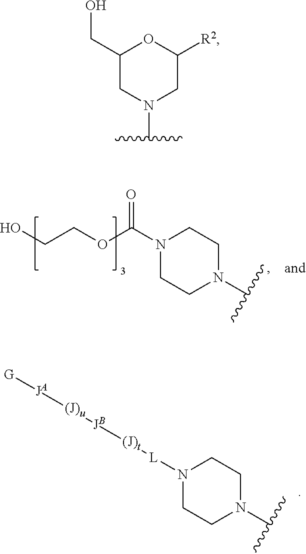

Embodiment P6. The peptide-oligonucleotide conjugate of any one of embodiments 1-5, or a pharmaceutically acceptable salt thereof, wherein E′ is selected from H, —C(O)CH₃, benzoyl, stearoyl, trityl, 4-methoxytrityl, and

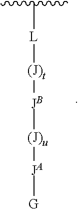

Embodiment P7. The peptide-oligonucleotide conjugate of any one of embodiments 1-4, or a pharmaceutically acceptable salt thereof, wherein the peptide-oligonucleotide conjugate of Formula (I) is a peptide-oligonucleotide conjugate selected from:

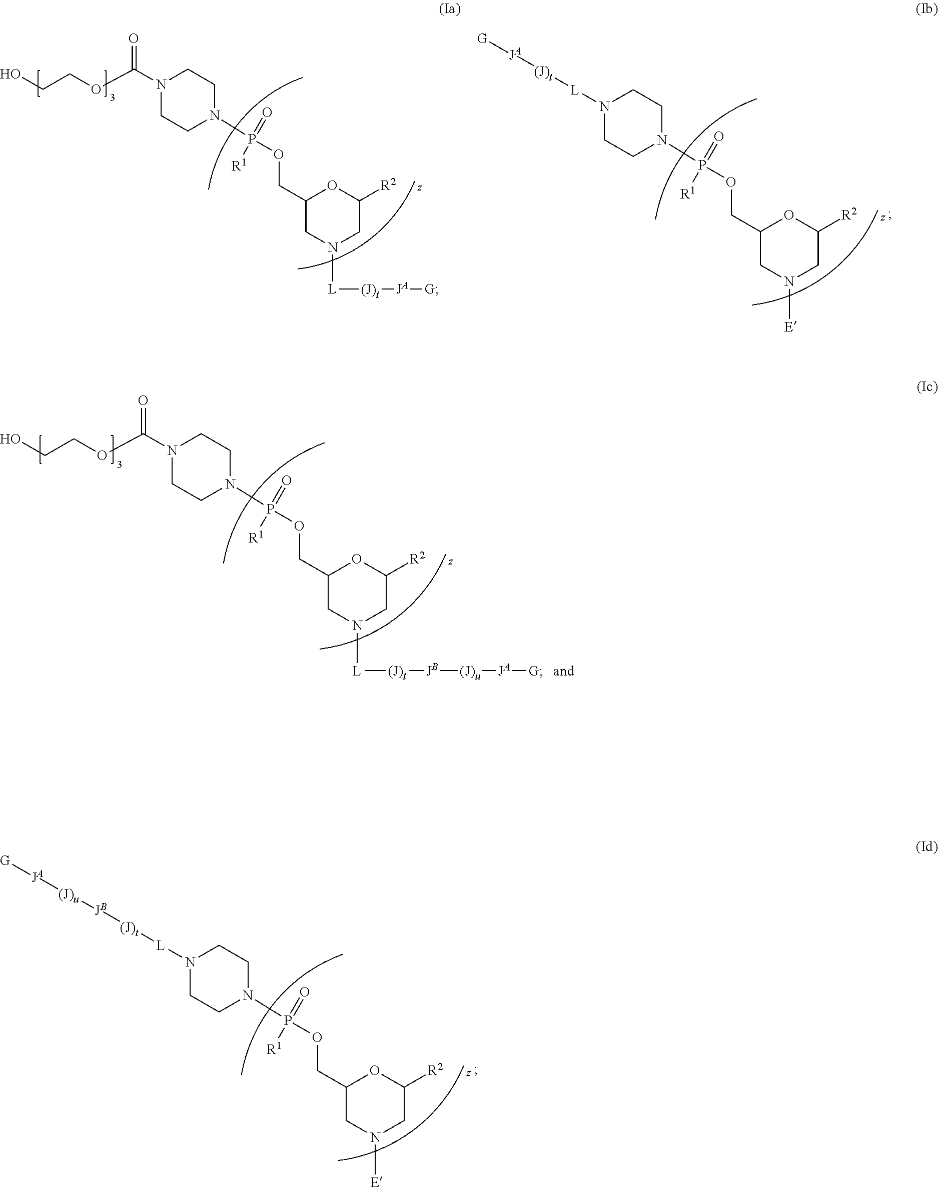

wherein E′ is selected from H, C₁₋₆-alkyl, —C(O)CH₃, benzoyl, and stearoyl; and
u is 3-20.

Embodiment P8. The peptide-oligonucleotide conjugate of embodiment 7, or a pharmaceutically acceptable salt thereof, wherein the peptide-oligonucleotide conjugate is of Formula (Ia).

Embodiment P9. The peptide-oligonucleotide conjugate of embodiment 7, or a pharmaceutically acceptable salt thereof, wherein the peptide-oligonucleotide conjugate is of Formula (Ib).

Embodiment P10. The peptide-oligonucleotide conjugate of embodiment 7, or a pharmaceutically acceptable salt thereof, wherein the peptide-oligonucleotide conjugate is of Formula (Ic).

Embodiment P11. The peptide-oligonucleotide conjugate of embodiment 7, or a pharmaceutically acceptable salt thereof, wherein the peptide-oligonucleotide conjugate is of Formula (Id).

Embodiment P12. The peptide-oligonucleotide conjugate of any one of embodiments 1-11, or a pharmaceutically acceptable salt thereof, wherein Y is selected from glycine, proline, beta-alanine, and 9-aminohexoic acid.

Embodiment P13. The peptide-oligonucleotide conjugate of any one of embodiments 1-12, or a pharmaceutically acceptable salt thereof, wherein Y is glycine.

Embodiment P14. The peptide-oligonucleotide conjugate of any one of embodiments 1-13, or a pharmaceutically acceptable salt thereof, wherein at least one J is:

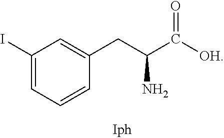 [SMILES: N[C@@H](Cc1cccc(I)c1)C(=O)O]

Embodiment P15. The peptide-oligonucleotide conjugate of any one of embodiments 1-14, or a pharmaceutically acceptable salt thereof, wherein (J)ₜ or (J)ᵤ comprises an (X-ph)-Gly-Arg motif.

Embodiment P16. The peptide-oligonucleotide conjugate of any one of embodiments 1-15, or a pharmaceutically acceptable salt thereof, wherein (J)ₜ or (J)ᵤ comprises an Iph-Gly-Arg motif.

Embodiment P17. The peptide-oligonucleotide conjugate of any one of embodiments 1-16, or a pharmaceutically acceptable salt thereof, wherein each R¹ is N(CH₃)₂.

Embodiment P18. The peptide-oligonucleotide conjugate of any one of embodiments 1-17, or a pharmaceutically acceptable salt thereof, wherein each R² is a nucleobase, independently at each occurrence, selected from adenine, guanine, cytosine, 5-methyl-cytosine, thymine, uracil, and hypoxanthine.

Embodiment P19. The peptide-oligonucleotide conjugate of any one of embodiments 1-18, wherein L is —C(O)(CH₂)₁₋₈C(O)-(DBCO derivative)-(CH₂)₁₋₈C(O)—.

Embodiment P20. The oligonucleotide conjugate of any one of embodiments 1-19, wherein L is:

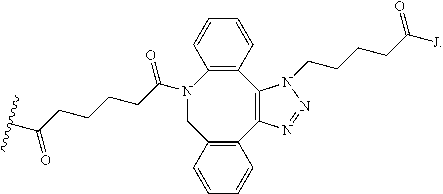

Embodiment P21. The oligonucleotide conjugate of any one of embodiments 1-18, wherein L is —C(O)(CH₂)₁₋₈C(O)-(DBCO derivative)-(CH₂)₁₋₈C(O)—(Y)—.

Embodiment P22. The oligonucleotide conjugate of any one of embodiments 1-18 and 22, wherein L is

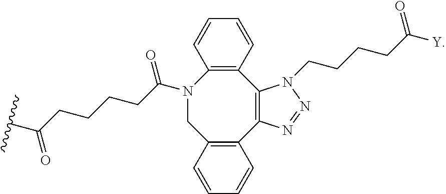

Embodiment P23. The peptide-oligonucleotide conjugate of any one of embodiments 1-22, or a pharmaceutically acceptable salt thereof, wherein G is selected from H, —C(O)CH₃, —NH₂, benzoyl, and stearoyl.

Embodiment P24. The peptide-oligonucleotide conjugate of any one of embodiments 1-23, or a pharmaceutically acceptable salt thereof, wherein G is —NH₂ or —C(O)CH₃.

Embodiment P25. The peptide-oligonucleotide conjugate of any one of embodiments 1-24, or a pharmaceutically acceptable salt thereof, wherein (J)ₜ and (J)ᵤ are each independently a peptide sequence selected from:

| SEQ | |
|---|---|
| ID | |
| NO. | Sequence |
| 1 | Lys-Gln-Lys-Thr-Ser-(X-ph)-Gly-Arg-Gly-Pip; |
| 2 | Asn-Phe-Lys-Gln-Nap-His-Ala-Gly-Dab-Arg; |
| 3 | Dab-Val-Ala-Arg-Asn-Asn-Dab-Thr-Lys-Gly; |
| 4 | Cit-Met-Phe-Gly-Pip-Dab-Dab-Lys-Ala-Pro; |
| 5 | Dab-Pro-Arg-Dab-Leu-His-Ser-Dab-Ala-Thr; |
| 6 | Pro-Pip-Gly-Dab-Lys-Dab-Ser-Val-(X-ph)-Nap; |
| 7 | Val-Gln-His-(X-ph)-Arg-Dab-Lys-Glu-Lys-Asn; |
| 8 | Asn-Phe-Asn-Asn-Arg-Ser-Dab-Gly-His-Lys; |
| 9 | Ala-Arg-Ser-Glu-Gln-His-His-Nap-Ser-Dab; |
| 10 | (X-ph)-Gly-Arg-Gly-Pip; |
| 11 | Lys-Thr-Ser-(X-ph)-Gly-Arg; |
| 12 | Lys-Gln-Lys-Thr-Ser; |
| 13 | Lys-Trp-Lys; |
| 14 | (X-ph)-Gly-Arg; |
| 15 | Lys-Gln-Lys-Thr-Ser-(X-ph)-Gly-Arg; |
| 16 | Ser-(X-ph)-Gly-Arg; |
| 17 | Lys-Thr-Ser; and |
| 18 | Gln-Lys-Thr-Ser-(X-ph)-Gly-Arg. |

Embodiment P26. The peptide-oligonucleotide conjugate of any one of embodiments 1-24, or a pharmaceutically acceptable salt thereof, wherein (J)ₜ is a peptide sequence selected from:

| SEQ ID | |
|---|---|
| NO. | Sequence |
| 20 | Lys-Gln-Lys-Thr-Ser-Iph-Gly-Arg-Gly-Pip; |
| 4 | Cit-Met-Phe-Gly-Pip-Dab-Dab-Lys-Ala-Pro; |
| 21 | Pro-Pip-Gly-Dab-Lys-Dab-Ser-Val-Iph-Nap; |
| 22 | Val-Gln-His-Iph-Arg-Dab-Lys-Glu-Lys-Asn; |
| 23 | Iph-Gly-Arg-Gly-Pip; |
| 24 | Lys-Thr-Ser-Iph-Gly-Arg; |
| 25 | Iph-Gly-Arg; |
| 26 | Lys-Gln-Lys-Thr-Ser-Iph-Gly-Arg; |
| 27 | Ser-Iph-Gly-Arg; |
| 28 | Gln-Lys-Thr-Ser-Iph-Gly-Arg; and |
| 29 | Lys-Gln-Lys-Thr-Ser-Iph-Gly-Arg. |

wherein:
u is 0; and
J^{B} is absent.

Embodiment P27. The peptide-oligonucleotide conjugate of any one of embodiments 1-26, or a pharmaceutically acceptable salt thereof, wherein J^{A} is the amino acid sequence KWKK.

Embodiment P28. The peptide-oligonucleotide conjugate of any one of embodiments 1-25 and 27, or a pharmaceutically acceptable salt thereof, wherein J^{B} is absent, or J^{B} is the amino acid sequence KWKK or KWK.

Embodiment P29. The peptide-oligonucleotide conjugate of any one of embodiments 1-25 and 27-28, or a pharmaceutically acceptable salt thereof, wherein u is 3-20 and (J)ₜ-J^{B}-(J)ᵤ is a peptide sequence selected from:

| SEQ ID | |
|---|---|
| NO. | Sequence |
| 30 | Lys-Gln-Lys-Thr-Ser-(X-ph)-Gly-Arg-Gly-Pip-Lys-Trp-Lys-Lys-Gly-Lys-Gln-Lys- |
| | Thr-Ser-(X-ph)-Gly-Arg-Gly-Pip; |
| 31 | Dab-Val-Ala-Arg-Asn-Asn-Dab-Thr-Lys-Gly-Lys-Trp-Lys-Lys-Gly-Lys-Gln-Lys- |
| | Thr-Ser-(X-ph)-Gly-Arg-Gly-Pip; |
| 32 | Cit-Met-Phe-Gly-Pip-Dab-Dab-Lys-Ala-Pro-Lys-Trp-Lys-Lys-Gly-Lys-Gln-Lys-Thr- |
| | Ser-(X-ph)-Gly-Arg-Gly-Pip; |
| 33 | (X-ph)-Gly-Arg-Gly-Pip-Lys-Gln-Lys-Thr-Ser-(X-ph)-Gly-Arg-Gly-Pip; |
| 34 | Lys-Thr-Ser-(X-ph)-Gly-Arg-Lys-Gln-Lys-Thr-Ser-(X-ph)-Gly-Arg-Gly-Pip; |
| 35 | Lys-Gln-Lys-Thr-Ser-Lys-Gln-Lys-Thr-Ser-(X-ph)-Gly-Arg-Gly-Pip; |
| 36 | Lys-Trp-Lys-Lys-Gln-Lys-Thr-Ser-(X-ph)-Gly-Arg-Gly-Pip; |
| 37 | Lys-Thr-Ser-(X-ph)-Gly-Arg-Lys-Gln-Lys-Thr-Ser-(X-ph)-Gly-Arg; and |
| 38 | Lys-Gln-Lys-Thr-Ser-(X-ph)-Gly-Arg-Lys-Trp-Lys-Gln-Lys-Thr-Ser-(X-ph)-Gly-Arg. |

Embodiment P30. A peptide-oligonucleotide conjugate of Formula (II):

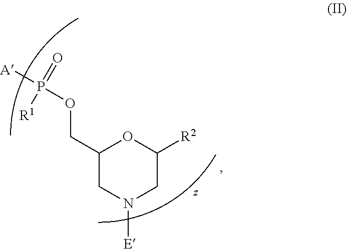

or a pharmaceutically acceptable salt thereof,
wherein:
A′ is selected from —N(H)CH₂C(O)NH₂, —N(C₁₋₆-alkyl)CH₂C(O)NH₂,

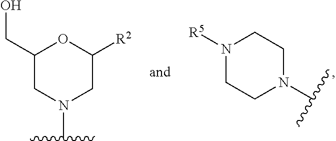

wherein
R⁵ is —C(O)(O-alkyl)ᵣ-OH, wherein x is 3-10 and each alkyl group is, independently at each occurrence, C₂₋₆-alkyl, or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O-heteroaryl-R⁶, and

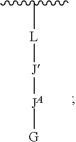

wherein R⁶ is selected from OH, SH, and NH₂, or R⁶ is selected from O, S, and NH, each of which is covalently linked to a solid support;
each R¹ is independently OH or —N(R³)(R⁴), wherein each R³ and R⁴ are, independently at each occurrence H or —C₁₋₆-alkyl;
each R² is independently, at each occurrence, selected from H, a nucleobase, and a nucleobase functionalized with a chemical protecting-group, wherein the nucleobase, independently at each occurrence, comprises a C₃₋₆-heterocyclic ring selected from pyridine, pyrimidine, purine, and deaza-purine;
z is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl,

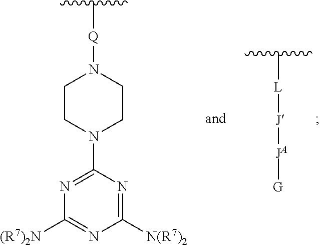

wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is covalently linked by an amide bond to J′, and L is —C(O)(CH₂)₁₋₈C(O)-heteroaromatic-(CH₂)₁₋₈C(O)—, —C(O)(CH₂)₁₋₈C(O)-heteroaromatic-(CH₂)₁₋₈C(O)—(Y)—; —C(O)(CH₂)₁₋₈C(O)-heteroaromatic-(CH₂)₁₋₈C(O)—(Y—Y)—;
Y is independently at each occurrence a linking amino acid;
X is halogen;
J^{A} is absent or the amino acid sequence selected from KWKK, KKWK, KWWKK, WWKK, KWK, WKK, KKW, KKKK, KK, and KW;
G is covalently linked to J^{A}, and G is selected from H, —C(O)C₁₋₆-alkyl, —NH₂, —NH(C₁₋₆-alkyl), —N(C₁₋₆-alkyl)₂, benzoyl, and stearoyl; and
J′ is a peptide sequence selected from:

| SEQ ID | |
|---|---|
| NO. | Sequence |
| 149 | Lys-Gln-Lys-Thr-Ser-(X-ph)-Gly-Arg-Gly-Pip; |
| 2 | Asn-Phe-Lys-Gln-Nap-His-Ala-Gly-Dab-Arg; |
| 3 | Dab-Val-Ala-Arg-Asn-Asn-Dab-Thr-Lys-Gly; |
| 4 | Cit-Met-Phe-Gly-Pip-Dab-Dab-Lys-Ala-Pro; |
| 5 | Dab-Pro-Arg-Dab-Leu-His-Ser-Dab-Ala-Thr; |
| 150 | Pro-Pip-Gly-Dab-Lys-Dab-Ser-Val-(X-ph)-Nap; |
| 151 | Val-Gln-His-(X-ph)-Arg-Dab-Lys-Glu-Lys-Asn; |
| 8 | Asn-Phe-Asn-Asn-Arg-Ser-Dab-Gly-His-Lys; |
| 9 | Ala-Arg-Ser-Glu-Gln-His-His-Nap-Ser-Dab; |
| 152 | (X-ph)-Gly-Arg-Gly-Pip; |
| 153 | Lys-Thr-Ser-(X-ph)-Gly-Arg; |
| 154 | (X-ph)-Gly-Arg; |
| 155 | Lys-Gln-Lys-Thr-Ser-(X-ph)-Gly-Arg; |
| 156 | Ser-(X-ph)-Gly-Arg; |
| 157 | Gln-Lys-Thr-Ser-(X-ph)-Gly-Arg; |
| 158 | Lys-Gln-Lys-Thr-Ser-(X-ph)-Gly-Arg; |
| 30 | Lys-Gln-Lys-Thr-Ser-(X-ph)-Gly-Arg-Gly-Pip-Lys-Trp-Lys-Lys-Gly-Lys-Gln-Lys- |
| | Thr-Ser-(X-ph)-Gly-Arg-Gly-Pip; |
| 31 | Dab-Val-Ala-Arg-Asn-Asn-Dab-Thr-Lys-Gly-Lys-Trp-Lys-Lys-Gly-Lys-Gln-Lys- |
| | Thr-Ser-(X-ph)-Gly-Arg-Gly-Pip; |
| 32 | Cit-Met-Phe-Gly-Pip-Dab-Dab-Lys-Ala-Pro-Lys-Trp-Lys-Lys-Gly-Lys-Gln-Lys- |
| | Thr-Ser-(X-ph)-Gly-Arg-Gly-Pip; |
| 33 | (X-ph)-Gly-Arg-Gly-Pip-Lys-Gln-Lys-Thr-Ser-(X-ph)-Gly-Arg-Gly-Pip; |
| 34 | Lys-Thr-Ser-(X-ph)-Gly-Arg-Lys-Gln-Lys-Thr-Ser-(X-ph)-Gly-Arg-Gly-Pip; |
| 35 | Lys-Gln-Lys-Thr-Ser-Lys-Gln-Lys-Thr-Ser-(X-ph)-Gly-Arg-Gly-Pip; |
| 36 | Lys-Trp-Lys-Lys-Gln-Lys-Thr-Ser-(X-ph)-Gly-Arg-Gly-Pip; |
| 37 | Lys-Thr-Ser-(X-ph)-Gly-Arg-Lys-Gln-Lys-Thr-Ser-(X-ph)-Gly-Arg; and |
| 38 | Lys-Gln-Lys-Thr-Ser-(X-ph)-Gly-Arg-Lys-Trp-Lys-Gln-Lys-Thr-Ser-(X-ph)-Gly- |
| | Arg; |

wherein Dab, Cit, Nap, X-ph, Pip, and Pyr have the following definitions:

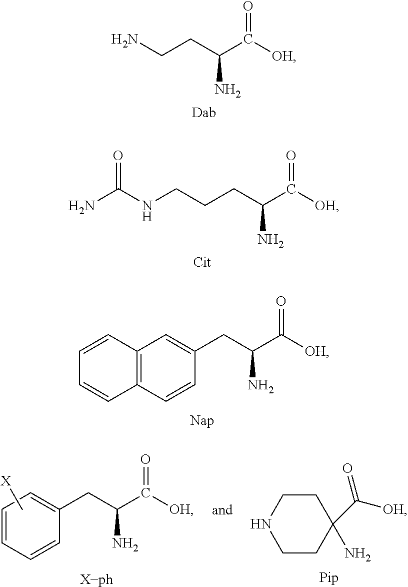

wherein X is halogen; and
wherein at least one of the following conditions is true:
   1) A′ is

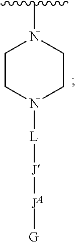

or 2) E′ is

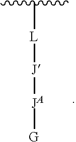

Embodiment P31. The peptide-oligonucleotide conjugate of embodiment 30, or a pharmaceutically acceptable salt thereof, wherein

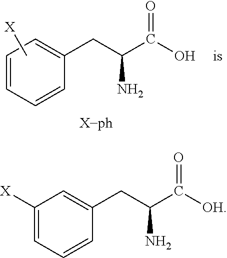

Embodiment P32. The peptide-oligonucleotide conjugate of embodiment 30 or 31, or a pharmaceutically acceptable salt thereof, wherein X is I.

Embodiment P33. The peptide-oligonucleotide conjugate of any one of embodiments 30-32, or a pharmaceutically acceptable salt thereof, wherein J^{A} is the amino acid sequence KWKK.

Embodiment P34. The peptide-oligonucleotide conjugate of any one of embodiments 30-33, or a pharmaceutically acceptable salt thereof, wherein
A′ is selected from —N(C₁₋₆-alkyl)CH₂C(O)NH₂,

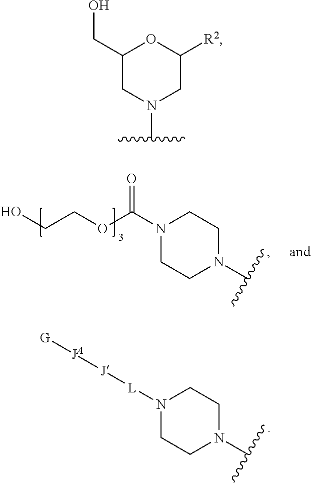

Embodiment P35. The peptide-oligonucleotide conjugate of any one of embodiments 30-34, or a pharmaceutically acceptable salt thereof, wherein E′ is selected from H, —C(O)CH₃, benzoyl, stearoyl, trityl, 4-methoxytrityl, and

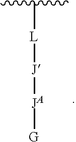

Embodiment P36. The peptide-oligonucleotide conjugate of any one of embodiments 30-33, or a pharmaceutically acceptable salt thereof, wherein the peptide-oligonucleotide conjugate of Formula (II) is a peptide-oligonucleotide conjugate selected from:

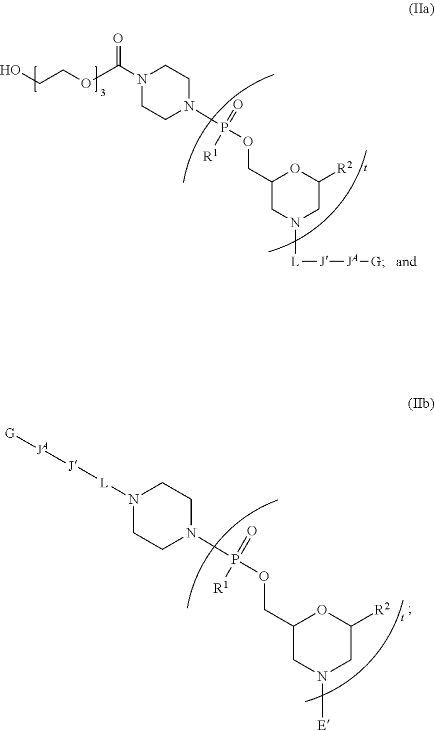

wherein E′ is selected from H, C₁₋₆-alkyl, —C(O)CH₃, benzoyl, and stearoyl.

Embodiment P37. The peptide-oligonucleotide conjugate of embodiment 36, or a pharmaceutically acceptable salt thereof, wherein the peptide-oligonucleotide conjugate is of Formula (IIa).

Embodiment P38. The peptide-oligonucleotide conjugate of embodiment 36, or a pharmaceutically acceptable salt thereof, wherein the peptide-oligonucleotide conjugate is of Formula (IIb).

Embodiment P39. The peptide-oligonucleotide conjugate of any one of embodiments 30-38, or a pharmaceutically acceptable salt thereof, wherein Y is selected from glycine, proline, beta-alanine, and 9-aminohexoic acid.

Embodiment P40. The peptide-oligonucleotide conjugate of any one of embodiments 30-39, or a pharmaceutically acceptable salt thereof, wherein Y is glycine.

Embodiment P41. The peptide-oligonucleotide conjugate of any one of embodiments 30-40, or a pharmaceutically acceptable salt thereof, wherein each R¹ is N(CH₃)₂.

Embodiment P42. The peptide-oligonucleotide conjugate of any one of embodiments 30-41, or a pharmaceutically acceptable salt thereof, wherein each R² is a nucleobase, independently at each occurrence, selected from adenine, guanine, cytosine, 5-methyl-cytosine, thymine, uracil, and hypoxanthine.

Embodiment P43. The peptide-oligonucleotide conjugate of any one of embodiments 30-42, wherein L is —C(O)(CH₂)₁₋₈C(O)-(DBCO derivative)-(CH₂)₁₋₈C(O)—.

Embodiment P44. The oligonucleotide conjugate of any one of embodiments 30-43, wherein L is

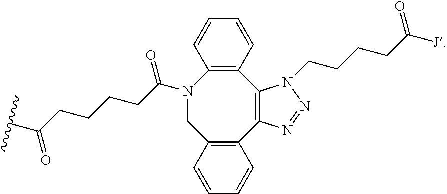

Embodiment P45. The oligonucleotide conjugate of any one of embodiments 30-42, wherein L is —C(O)(CH₂)₁₋₈C(O)-(DBCO derivative)-(CH₂)₁₋₃C(O)—(Y)—.

Embodiment P46. The oligonucleotide conjugate of any one of embodiments 30-42 and 45, wherein L is

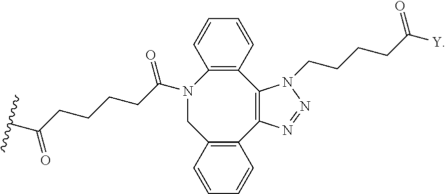

Embodiment P47. The peptide-oligonucleotide conjugate of any one of embodiments 30-46, or a pharmaceutically acceptable salt thereof, wherein G is selected from H, —C(O)CH₃, —NH₂, benzoyl, and stearoyl.

Embodiment P48. The peptide-oligonucleotide conjugate of any one of embodiments 30-47, or a pharmaceutically acceptable salt thereof, wherein G is —NH₂ or —C(O)CH₃.

Embodiment P49. A pharmaceutical composition comprising a compound of any one of embodiments 1-48, or a pharmaceutically acceptable salt thereof, and at least one pharmaceutically acceptable carrier.

Embodiment P50. A method of treating a disease in a subject in need thereof, comprising administering to the subject a compound of any one of embodiments 1-48, or a composition of embodiment 49 to the subject.

Embodiment P51. The method of embodiment 50, wherein the disease is a neuromuscular disease.

Embodiment P52. The method of embodiment 51, wherein the neuromuscular disease is Duchenne muscular dystrophy (DMD).

1. An antisense oligomer conjugate of Formula (III): 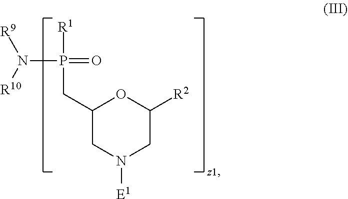
or a pharmaceutically acceptable salt thereof,
wherein:
z1 is an integer from 8 to 40;
each R¹ is independently —OH or —NR³R⁴;
each R³ and each R⁴ are independently hydrogen or C₁₋₆alkyl;
E¹ is hydrogen, —C₁₋₆alkyl, —C(O)C₁₋₆alkyl, stearoyl, trityl, monomethoxytrityl, dimethoxytrityl, trimethoxytrityl, -L²-(J^{2C})ₜ₂-(J^{2B})_{z5}-(J^{2D})ᵤ₂-(J^{2A})_{z6}-G², or 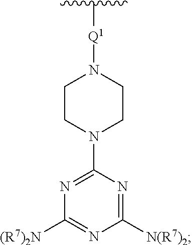
Q¹ is —C(O)(CH₂)₆C(O)— or —C(O)(CH₂)₂S₂(CH₂)₂C(O)—;
each R⁷ is independently —[(CH₂)₂OC(O)]N[(CH₂)₆NHC(═NH)NH₂]₂;
R⁹ is hydrogen or —C₁₋₆alkyl;
R¹⁰ is —CH₂C(O)NH₂, or R⁹ and R¹⁰ together with the nitrogen to which they are attached form a ring selected from: 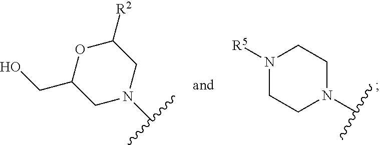
each R² is independently hydrogen, or an unsubstituted or R⁸-substituted nucleobase comprising a pyridine, pyrimidine, purine, or deaza-purine;
R⁸ is a protecting group;
R⁵ is hydrogen, —C(O)R^{5A}, —C(O)OR^{5B}, R⁶-substituted or unsubstituted C₁₋₆alkyl, R⁶-substituted or unsubstituted —C₁₋₆heteroalkyl, R⁶-substituted or unsubstituted aryl, R⁶-substituted or unsubstituted heteroaryl, trityl, monomethoxytrityl, —C(O)[O(CH₂)₂₋₆]_{z2}—OH, or -L¹-(J^{1C})ₜ₁-(J^{1B})_{z3}-(J^{1D})ᵤ₁-(J^{1A})_{z4}-G¹;
R^{5A} is hydrogen or substituted or unsubstituted C₁₋₆alkyl;
R^{5B} is hydrogen, substituted or unsubstituted C₁₋₆alkyl, substituted or unsubstituted aryl, or substituted or unsubstituted heteroaryl;
R⁶ is selected from —OR^{6A}, —SR^{6A}, —NHR^{6A};
R^{6A} is hydrogen or a solid support;
L¹ is —C(O)(CH₂)₁₋₈C(O)-heteroaryl-(CH₂)₁₋₈C(O)—[Y¹]_{z7};
L² is —C(O)(CH₂)₁₋₈C(O)-heteroaryl-(CH₂)₁₋₈C(O)—[Y²]_{z8};
Y¹ and Y² are each independently a linking amino acid;
z2 is an integer from 3 to 10;
z3, z4, z5, and z6 are each independently 0 or 1;
z7 and z8 are each independently 0, 1, or 2;
t1 and t2 are each independently an integer from 3 to 20;
u1 and u2 are each independently an integer selected from 0 and 3 to 20;
each J^{1A}, J^{1B}, J^{2A}, and J^{2B} is independently selected from Lys-Lys (SEQ ID NO: 138), Lys-Trp (SEQ ID NO: 139), Lys-Trp-Lys (SEQ ID NO: 140), Trp-Lys-Lys (SEQ ID NO: 141), Lys-Lys-Trp (SEQ ID NO: 142), Lys-Trp-Lys-Lys (SEQ ID NO: 143), Lys-Lys-Trp-Lys (SEQ ID NO: 144), Lys-Trp-Trp-Lys-Lys (SEQ ID NO: 145), Trp-Trp-Lys-Lys (SEQ ID NO: 146), and Lys-Lys-Lys-Lys (SEQ ID NO: 147);
each J^{1C}, J^{1D}, J^{2C} and J^{2D} is independently selected from Arg, His, Lys, Asp, Glu, Ser, Thr, Asn, Gln, Gly, Met, Pro, Val, Ala, Leu, Phe, Trp, Cys, Ile, Tyr, 2,4-diaminobutyric acid (Dab), 2-amino-5-ureidopentanoic acid (citrulline (Cit)), 2-amino-3-(naphthalen-2-yl)propanoic acid (Nap), 4-aminopiperidine-4-carboxylic acid (Pip), 2-amino-3-(pyridin-3-yl)propanoic acid (Pyr), and 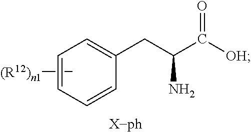
n1 is an integer from 1 to 5;
R¹² is —F, —Cl, —Br, or —I;
G¹ is hydrogen, —C(O)R^{13A}, NR^{13A}R^{13B} benzoyl, and stearoyl;
G² is hydrogen, —C(O)R^{14A}, NR^{14A}R^{14B} benzoyl, and stearoyl; and
R^{13A}, R^{13B}, R^{14A}, and R^{14B} are each independently hydrogen or alkyl;
provided that:
i) when u2 and z6 are each independently zero, or when u1 and z4 are each independently zero, then at least one of J^{1C} and J^{2C} is 2-amino-5-ureidopentanoic acid (citrulline (Cit)), 2-amino-3-(pyridin-3-yl)propanoic acid (Pyr), or 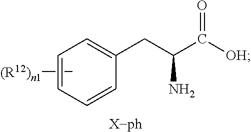
ii) E¹ is -L²-(J^{2C})ₜ₂-(J^{2B})_{z5}-(J^{2D})ᵤ₂-(J^{2A})_{z6}-G²; or R⁹ and R¹⁰ together with the nitrogen to which they are attached form: 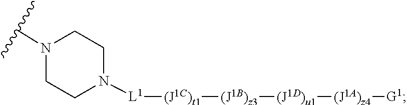
and
iii) at least one R² is not hydrogen.

2. The antisense oligomer conjugate of claim 1, or a pharmaceutically acceptable salt thereof, wherein the antisense oligomer conjugate is of Formula (IIIa):

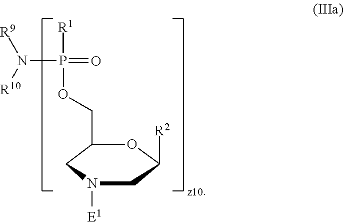

3. The antisense oligomer conjugate of claim 1, or a pharmaceutically acceptable salt thereof, wherein u2 and z6 are both zero, and u1 and z4 are both zero.

4. An antisense oligomer conjugate of Formula (IV): 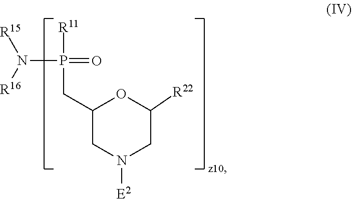
or a pharmaceutically acceptable salt thereof,
wherein:
z10 is an integer from 8 to 40;
each R¹¹ is independently —OH or —NR¹⁷R¹⁸,
each R¹⁷ and each R¹⁸ are independently hydrogen or C₁₋₆alkyl;
R¹⁵ is hydrogen or —C₁₋₆alkyl;
R¹⁶ is —CH₂C(O)NH₂, or R¹⁵ and R¹⁶ together with the nitrogen to which they are attached join to form a ring selected from: 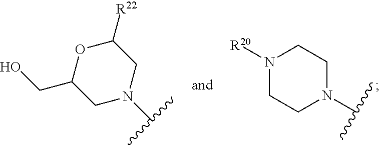
R²⁰ is hydrogen, —C(O)R^{20A}, —C(O)OR^{20B}, R²¹-substituted or unsubstituted C₁₋₆alkyl, R²¹-substituted or unsubstituted —C₁₋₆heteroalkyl, R²¹-substituted or unsubstituted aryl, R²¹-substituted or unsubstituted heteroaryl, trityl, monomethoxytrityl, —C(O)[O(CH₂)₂₋₆]_{z9}—OH, or -L⁴-(J^{4B})ₜ₄-(J^{4A})_{z12}-G⁴;
z9 is an integer from 3 to 10;
R^{20A} is hydrogen or substituted or unsubstituted C₁₋₆alkyl;
R^{20B} is hydrogen, substituted or unsubstituted C₁₋₆alkyl, substituted or unsubstituted aryl, or substituted or unsubstituted heteroaryl;
R²¹ is selected from —OR^{21A}, —SR^{21A}, —NHR^{21A};
R^{21A} is hydrogen or a solid support;
each R²² is independently hydrogen or R²³-substituted or unsubstituted nucleobase comprising a pyridine, pyrimidine, purine, or deaza-purine,
R²³ is a protecting group;
E² is hydrogen, —C₁₋₆alkyl, —C(O)C₁₋₆alkyl, stearoyl, trityl, monomethoxytrityl, dimethoxytrityl, trimethoxytrityl, -L³-(J^{3B})ₜ₃-(J^{3A})_{z11}-G³, or 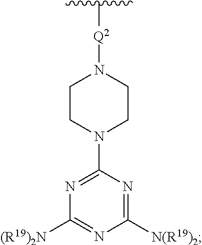
each R¹⁹ is independently —[(CH₂)₂OC(O)]N[(CH₂)₆NHC(═NH)NH₂]₂;
Q² is —C(O)(CH₂)₆C(O)— or —C(O)(CH₂)₂S₂(CH₂)₂C(O)—;
L³ is —C(O)(CH₂)₁₋₈C(O)— heteroaryl-(CH₂)₁₋₈C(O)—[Y³]_{z13};
L⁴ is —C(O)(CH₂)₁₋₈C(O)-heteroaryl-(CH₂)₁₋₈C(O)—[Y⁴]_{z14};
Y³ and Y⁴ are each independently a linking amino acid;
z11 and z12 are each independently 0 or 1;
z13 and z14 are each independently 0, 1, or 2;
t3 and t4 are each independently an integer from 3 to 20;
J^{3A} and J^{4A} are each independently selected Lys-Lys (SEQ ID NO: 138), Lys-Trp (SEQ ID NO: 139), Lys-Trp-Lys (SEQ ID NO: 140), Trp-Lys-Lys (SEQ ID NO: 141), Lys-Lys-Trp (SEQ ID NO: 142), Lys-Trp-Lys-Lys (SEQ ID NO: 143), Lys-Lys-Trp-Lys (SEQ ID NO: 144), Lys-Trp-Trp-Lys-Lys (SEQ ID NO: 145), Trp-Trp-Lys-Lys (SEQ ID NO: 146), and Lys-Lys-Lys-Lys (SEQ ID NO: 147);
each J^{3B} and each J^{4B} is independently an L-amino acid selected from Arg, His, Lys, Asp, Glu, Ser, Thr, Asn, Gln, Gly, Met, Pro, Val, Ala, Leu, Phe, Trp, Cys, Ile, Tyr, 2,4-diaminobutyric acid (Dab), 2-amino-5-ureidopentanoic acid (citrulline (Cit)), 2-amino-3-(naphthalen-2-yl)propanoic acid (Nap), 4-aminopiperidine-4-carboxylic acid (Pip), 2-amino-3-(pyridin-3-yl)propanoic acid (Pyr), and 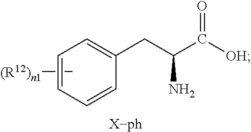
n2 is an integer from 1 to 5;
R²⁴ is —F, —Cl, —Br, or —I;
G³ is hydrogen, —C(O)R^{25A}, —NR^{25A}R^{25B} benzoyl, and stearoyl;
G⁴ is hydrogen, —C(O)R^{26A}, —NR^{26A}R^{26B} benzoyl, and stearoyl;
R^{25A}, R^{25B}, R^{26A}, and R^{26B} are each independently hydrogen or alkyl; and
provided that:
i) E² is -L³-(J^{3B})₃-(J^{3A})_{z11}-G³; or R¹⁶ and R¹⁶ together with the nitrogen to which they are attached join to form 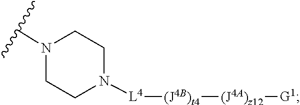
ii) at least one R²² is not hydrogen; and
iii) each J^{3B} and each J^{4B} are not: a D-amino acid, 9-aminohexanoic acid, γ-aminobutyric acid (Abu), homoleucine (Hle), norleucine (Nle), Dpa (diphenylalanine), aminomethylphenylalanine (Amf), or 2-amino-4-guanidinobutanoic acid (Gba).

5. The antisense oligomer conjugate of claim 4, or a pharmaceutically acceptable salt thereof, wherein the conjugate is of Formula (IVa):

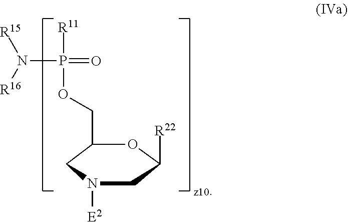

6. The peptide-oligonucleotide conjugate of claim 4, or a pharmaceutically acceptable salt thereof, wherein -(J^{3B})ₜ₃-(J^{3A})_{z11}- or -(J^{4B})ₜ₄-(J^{4A})_{z12}- each independently form a peptide sequence selected from:

| SEQ ID | |
|---|---|
| NO. | Sequence |
| 48 | Lys-Gln-Lys-Thr-Ser-Iph-Gly-Arg-Gly-Pip-Lys-Trp-Lys-Lys; |
| 49 | Asn-Phe-Lys-Gln-Nap-His-Ala-Gly-Dab-Arg-Lys-Trp-Lys-Lys; |
| 50 | Dab-Val-Ala-Arg-Asn-Asn-Dab-Thr-Lys-Gly-Lys-Trp-Lys-Lys; |
| 51 | Cit-Met-Phe-Gly-Pip-Dab-Dab-Lys-Ala-Pro-Lys-Trp-Lys-Lys; |
| 52 | Dab-Pro-Arg-Dab-Leu-His-Ser-Dab-Ala-Thr-Lys-Trp-Lys-Lys; |
| 53 | Pro-Pip-Gly-Dab-Lys-Dab-Ser-Val-Iph-Nap-Lys-Trp-Lys-Lys; |
| 54 | Val-Gln-His-Iph-Arg-Dab-Lys-Glu-Lys-Asn-Lys-Trp-Lys-Lys; |
| 55 | Asn-Phe-Asn-Asn-Arg-Ser-Dab-Gly-His-Lys-Lys-Trp-Lys-Lys; |
| 56 | Ala-Arg-Ser-Glu-Gln-His-His-Nap-Ser-Dab-Lys-Trp-Lys-Lys; |
| 57 | Iph-Gly-Arg-Gly-Pip-Lys-Trp-Lys-Lys; |
| 58 | Lys-Thr-Ser-Iph-Gly-Arg-Lys-Trp-Lys-Lys; |
| 61 | Iph-Gly-Arg-Lys-Trp-Lys-Lys; |
| 62 | Lys-Gln-Lys-Thr-Ser-Iph-Gly-Arg-Lys-Trp-Lys-Lys; |
| 63 | Ser-Iph-Gly-Arg-Lys-Trp-Lys-Lys; |
| 65 | Gln-Lys-Thr-Ser-Iph-Gly-Arg-Lys-Trp-Lys-Lys; |
| 66 | Lys-Gln-Lys-Thr-Ser-Iph-Gly-Arg-Lys-Trp-Lys-Lys; |
| 67 | Lys-Gln-Lys-Thr-Ser-Iph-Gly-Arg-Gly-Pip-Lys-Trp-Lys-Lys-Gly-Lys-Gln-Lys- |
| | Thr-Ser-Iph-Gly-Arg-Gly-Pip-Lys-Trp-Lys-Lys; |
| 68 | Dab-Val-Ala-Arg-Asn-Asn-Dab-Thr-Lys-Gly-Lys-Trp-Lys-Lys-Gly-Lys-Gln-Lys- |
| | Thr-Ser-Iph-Gly-Arg-Gly-Pip-Lys-Trp-Lys-Lys; |
| 69 | Cit-Met-Phe-Gly-Pip-Dab-Dab-Lys-Ala-Pro-Lys-Trp-Lys-Lys-Gly-Lys-Gln-Lys- |
| | Thr-Ser-Iph-Gly-Arg-Gly-Pip-Lys-Trp-Lys-Lys; |
| 70 | Iph-Gly-Arg-Gly-Pip-Lys-Gln-Lys-Thr-Ser-Iph-Gly-Arg-Gly-Pip-Lys-Trp-Lys- |
| | Lys; |
| 71 | Lys-Thr-Ser-Iph-Gly-Arg-Lys-Gln-Lys-Thr-Ser-Iph-Gly-Arg-Gly-Pip-Lys-Trp- |
| | Lys-Lys; |
| 72 | Lys-Gln-Lys-Thr-Ser-Lys-Gln-Lys-Thr-Ser-Iph-Gly-Arg-Gly-Pip-Lys-Trp-Lys- |
| | Lys; |
| 73 | Lys-Trp-Lys-Lys-Gln-Lys-Thr-Ser-Iph-Gly-Arg-Gly-Pip-Lys-Trp-Lys-Lys; |
| 74 | Lys-Thr-Ser-Iph-Gly-Arg-Lys-Gln-Lys-Thr-Ser-Iph-Gly-Arg-Lys-Trp-Lys-Lys; |
| 75 | Lys-Gln-Lys-Thr-Ser-Iph-Gly-Arg-Lys-Trp-Lys-Gln-Lys-Thr-Ser-Iph-Gly-Arg- |
| | Lys-Trp-Lys-Lys; |
| 78 | Lys-Gln-Lys-Thr-Ser-Phe-Gly-Arg-Gly-Pip-Lys-Trp-Lys-Lys; |
| 79 | Lys-Gln-Lys-Thr-Ser-Iph-Gly-Arg-Gly-Lys-Lys-Trp-Lys-Lys; |
| 80 | Lys-Gln-Lys-Thr-Ser-Phe-Gly-Arg-Gly-Lys-Lys-Trp-Lys-Lys; |
| 81 | Lys-Gln-Lys-Thr-Ser-Iph-Gly-Lys-Gly-Pip-Lys-Trp-Lys-Lys; |
| 82 | Lys-Gln-Lys-Thr-Ser-Iph-Ala-Arg-Ala-Pip-Lys-Trp-Lys-Lys; |
| 83 | Ala-Gln-Lys-Thr-Ser-Iph-Gly-Arg-Gly-Pip-Lys-Trp-Lys-Lys; |
| 84 | Lys-Ala-Lys-Thr-Ser-Iph-Gly-Arg-Gly-Pip-Lys-Trp-Lys-Lys; |
| 85 | Lys-Gln-Ala-Thr-Ser-Iph-Gly-Arg-Gly-Pip-Lys-Trp-Lys-Lys; |
| 86 | Lys-Gln-Lys-Ala-Ser-Iph-Gly-Arg-Gly-Pip-Lys-Trp-Lys-Lys; |
| 87 | Lys-Gln-Lys-Thr-Ala-Iph-Gly-Arg-Gly-Pip-Lys-Trp-Lys-Lys; |
| 88 | Lys-Gln-Lys-Thr-Ser-Ala-Gly-Arg-Gly-Pip-Lys-Trp-Lys-Lys; |
| 89 | Lys-Gln-Lys-Thr-Ser-Iph-Ala-Arg-Gly-Pip-Lys-Trp-Lys-Lys; |
| 90 | Lys-Gln-Lys-Thr-Ser-Iph-Gly-Ala-Gly-Pip-Lys-Trp-Lys-Lys; |
| 91 | Lys-Gln-Lys-Thr-Ser-Iph-Gly-Arg-Ala-Pip-Lys-Trp-Lys-Lys; |
| 92 | Lys-Gln-Lys-Thr-Ser-Iph-Gly-Arg-Gly-Ala-Lys-Trp-Lys-Lys; |
| 93 | Lys-Gln-Lys-Thr-Ser-Iph-Gly-Arg-Gly-Pip-Ala-Trp-Lys-Lys; |
| 94 | Lys-Gln-Lys-Thr-Ser-Iph-Gly-Arg-Gly-Pip-Lys-Ala-Lys-Lys; |
| 95 | Lys-Gln-Lys-Thr-Ser-Iph-Gly-Arg-Gly-Pip-Lys-Trp-Ala-Lys; |
| 96 | Lys-Gln-Lys-Thr-Ser-Iph-Gly-Arg-Gly-Pip-Lys-Trp-Lys-Ala-Lys-Trp-Lys-Lys; |
| 97 | Iph-Gln-Lys-Thr-Ser-Iph-Gly-Arg-Gly-Pip-Lys-Trp-Lys-Lys; |
| 98 | Lys-Gln-Iph-Thr-Ser-Iph-Gly-Arg-Gly-Pip-Lys-Trp-Lys-Lys; |
| 99 | Lys-Gln-Lys-Thr-Ser-Iph-Gly-Iph-Gly-Pip-Lys-Trp-Lys-Lys; |
| 100 | Lys-Gln-Lys-Thr-Ser-Iph-Gly-Arg-Gly-Iph-Lys-Trp-Lys-Lys; |
| 101 | Lys-Gln-Lys-Thr-Ser-Iph-Gly-Arg-Gly-Pip-Iph-Trp-Lys-Lys; |
| 102 | Lys-Gln-Lys-Thr-Ser-Iph-Gly-Arg-Gly-Pip-Lys-Trp-Iph-Lys-Lys-Trp-Lys-Lys; |
| 103 | Lys-Gln-Lys-Thr-Ser-Iph-Gly-Arg-Gly-Pip-Lys-Trp-Lys-Iph-Lys-Trp-Lys-Lys; |
| 104 | Dab-Gln-Dab-Thr-Ser-Iph-Gly-Arg-Gly-Pip-Dab-Trp-Dab-Dab-Lys-Trp-Lys-Lys; |
| 105 | Dab-Gln-Dab-Thr-Ser-Iph-Gly-Dab-Gly-Pip-Dab-Trp-Dab-Dab-Lys-Trp-Lys-Lys; |
| 106 | Dab-Gln-Dab-Thr-Ser-Iph-Gly-Arg-Gly-Dab-Dab-Trp-Dab-Dab-Lys-Trp-Lys-Lys; |
| | and |
| 107 | Dab-Gln-Dab-Thr-Ser-Iph-Gly-Dab-Gly-Dab-Dab-Trp-Dab-Dab-Lys-Trp-Lys-Lys. |

7. The antisense oligomer conjugate of claim 4, or a pharmaceutically acceptable salt thereof, wherein -(J^{3B})ₜ₃- or -(J^{4B})ₜ₄- each independently form a peptide sequence selected from:

| SEQ ID NO. | Sequence |
|---|---|
| 1 | Lys-Gln-Lys-Thr-Ser-Iph-Gly-Arg-Gly-Pip; |
| 2 | Asn-Phe-Lys-Gln-Nap-His-Ala-Gly-Dab-Arg; |
| 3 | Dab-Val-Ala-Arg-Asn-Asn-Dab-Thr-Lys-Gly; |
| 4 | Cit-Met-Phe-Gly-Pip-Dab-Dab-Lys-Ala-Pro; |
| 5 | Dab-Pro-Arg-Dab-Leu-His-Ser-Dab-Ala-Thr; |
| 6 | Pro-Pip-Gly-Dab-Lys-Dab-Ser-Val-Iph-Nap; |
| 7 | Val-Gln-His-Iph-Arg-Dab-Lys-Glu-Lys-Asn; |
| 8 | Asn-Phe-Asn-Asn-Arg-Ser-Dab-Gly-His-Lys; |
| 9 | Ala-Arg-Ser-Glu-Gln-His-His-Nap-Ser-Dab; |
| 10 | Iph-Gly-Arg-Gly-Pip; |
| 11 | Lys-Thr-Ser-Iph-Gly-Arg; |
| 12 | Lys-Gln-Lys-Thr-Ser; |
| 13 | Lys-Trp-Lys; |
| 14 | Iph-Gly-Arg; |
| 15 | Lys-Gln-Lys-Thr-Ser-Iph-Gly-Arg; |
| 16 | Ser-Iph-Gly-Arg; |
| 18 | Gln-Lys-Thr-Ser-Iph-Gly-Arg; |
| 19 | Lys-Gln-Lys-Thr-Ser-Iph-Gly-Arg; |
| 39 | Lys-Gln-Lys-Thr-Ser-Iph-Gly-Arg-Gly-Pip-Lys-Trp-Lys-Lys-Gly-Lys-Gln- |
| | Lys-Thr-Ser-Iph-Gly-Arg-Gly-Pip; |
| 40 | Dab-Val-Ala-Arg-Asn-Asn-Dab-Thr-Lys-Gly-Lys-Trp-Lys-Lys-Gly-Lys-Gln- |
| | Lys-Thr-Ser-Iph-Gly-Arg-Gly-Pip; |
| 41 | Cit-Met-Phe-Gly-Pip-Dab-Dab-Lys-Ala-Pro-Lys-Trp-Lys-Lys-Gly-Lys-Gln- |
| | Lys-Thr-Ser-Iph-Gly-Arg-Gly-Pip; |
| 42 | Iph-Gly-Arg-Gly-Pip-Lys-Gln-Lys-Thr-Ser-Iph-Gly-Arg-Gly-Pip; |
| 43 | Lys-Thr-Ser-Iph-Gly-Arg-Lys-Gln-Lys-Thr-Ser-Iph-Gly-Arg-Gly-Pip; |
| 44 | Lys-Gln-Lys-Thr-Ser-Lys-Gln-Lys-Thr-Ser-Iph-Gly-Arg-Gly-Pip; |
| 45 | Lys-Trp-Lys-Lys-Gln-Lys-Thr-Ser-Iph-Gly-Arg-Gly-Pip; |
| 46 | Lys-Thr-Ser-Iph-Gly-Arg-Lys-Gln-Lys-Thr-Ser-Iph-Gly-Arg; |
| 47 | Lys-Gln-Lys-Thr-Ser-Iph-Gly-Arg-Lys-Trp-Lys-Gln-Lys-Thr-Ser-Iph-Gly- |
| | Arg; |
| 108 | Lys-Gln-Lys-Thr-Ser-Phe-Gly-Arg-Gly-Pip; |
| 109 | Lys-Gln-Lys-Thr-Ser-Iph-Gly-Arg-Gly-Lys; |
| 110 | Lys-Gln-Lys-Thr-Ser-Phe-Gly-Arg-Gly-Lys; |
| 111 | Lys-Gln-Lys-Thr-Ser-Iph-Gly-Lys-Gly-Pip; |
| 112 | Lys-Gln-Lys-Thr-Ser-Iph-Ala-Arg-Ala-Pip; |
| 113 | Ala-Gln-Lys-Thr-Ser-Iph-Gly-Arg-Gly-Pip; |
| 114 | Lys-Ala-Lys-Thr-Ser-Iph-Gly-Arg-Gly-Pip; |
| 115 | Lys-Gln-Ala-Thr-Ser-Iph-Gly-Arg-Gly-Pip; |
| 116 | Lys-Gln-Lys-Ala-Ser-Iph-Gly-Arg-Gly-Pip; |
| 117 | Lys-Gln-Lys-Thr-Ala-Iph-Gly-Arg-Gly-Pip; |
| 118 | Lys-Gln-Lys-Thr-Ser-Ala-Gly-Arg-Gly-Pip; |
| 119 | Lys-Gln-Lys-Thr-Ser-Iph-Ala-Arg-Gly-Pip; |
| 120 | Lys-Gln-Lys-Thr-Ser-Iph-Gly-Ala-Gly-Pip; |
| 121 | Lys-Gln-Lys-Thr-Ser-Iph-Gly-Arg-Ala-Pip; |
| 122 | Lys-Gln-Lys-Thr-Ser-Iph-Gly-Arg-Gly-Ala; |
| 123 | Lys-Gln-Lys-Thr-Ser-Iph-Gly-Arg-Gly-Pip-Ala; |
| 124 | Lys-Gln-Lys-Thr-Ser-Iph-Gly-Arg-Gly-Pip-Lys-Ala; |
| 125 | Lys-Gln-Lys-Thr-Ser-Iph-Gly-Arg-Gly-Pip-Lys-Trp-Ala; |
| 126 | Lys-Gln-Lys-Thr-Ser-Iph-Gly-Arg-Gly-Pip-Lys-Trp-Lys-Ala; |
| 127 | Iph-Gln-Lys-Thr-Ser-Iph-Gly-Arg-Gly-Pip; |
| 128 | Lys-Gln-Iph-Thr-Ser-Iph-Gly-Arg-Gly-Pip; |
| 129 | Lys-Gln-Lys-Thr-Ser-Iph-Gly-Iph-Gly-Pip; |
| 130 | Lys-Gln-Lys-Thr-Ser-Iph-Gly-Arg-Gly-Iph; |
| 131 | Lys-Gln-Lys-Thr-Ser-Iph-Gly-Arg-Gly-Pip-Iph-Trp-Lys-Lys; |
| 132 | Lys-Gln-Lys-Thr-Ser-Iph-Gly-Arg-Gly-Pip-Lys-Trp-Iph-Lys; |
| 133 | Lys-Gln-Lys-Thr-Ser-Iph-Gly-Arg-Gly-Pip-Lys-Trp-Lys-Iph; |
| 134 | Dab-Gln-Dab-Thr-Ser-Iph-Gly-Arg-Gly-Pip-Dab-Trp-Dab-Dab; |
| 135 | Dab-Gln-Dab-Thr-Ser-Iph-Gly-Dab-Gly-Pip-Dab-Trp-Dab-Dab; |
| 136 | Dab-Gln-Dab-Thr-Ser-Iph-Gly-Arg-Gly-Dab-Dab-Trp-Dab-Dab; and |
| 137 | Dab-Gln-Dab-Thr-Ser-Iph-Gly-Dab-Gly-Dab-Dab-Trp-Dab-Dab. |

8. The antisense oligomer conjugate of claim 4, or a pharmaceutically acceptable salt thereof, wherein X-ph has the formula:

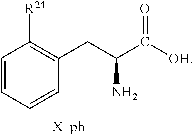

9. The antisense oligomer conjugate of claim 8, or a pharmaceutically acceptable salt thereof, wherein R²⁴ is —I.

10. The antisense oligomer conjugate of claim 4, or a pharmaceutically acceptable salt thereof, wherein J^{3B} or J^{4B} are each independently the amino acid sequence Lys-Trp-Lys-Lys (SEQ ID NO: 143).

11-12. (canceled)

13. The antisense oligomer conjugate of claim 4, or a pharmaceutically acceptable salt thereof, wherein the antisense oligomer conjugate is selected from Formula (Va) and (Vb): 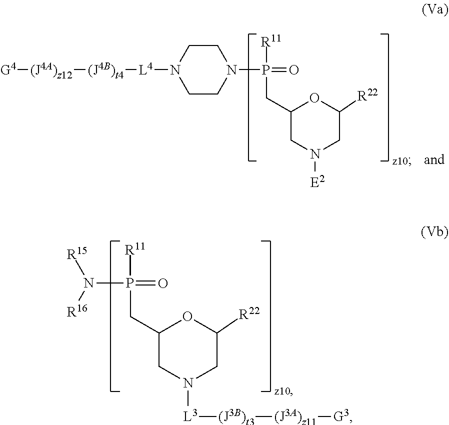
wherein:
E² is selected from hydrogen, —C₁₋₆alkyl, —C(O)CH₃, benzoyl, and stearoyl; and
R¹⁵ together and R¹⁶ together with the nitrogen to which they are attached join to form a ring selected from:

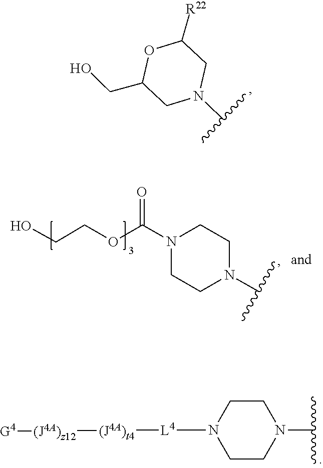

14. The antisense oligomer conjugate of claim 4, or a pharmaceutically acceptable salt thereof, wherein the antisense oligomer conjugate is of Formula (Vc) or (Vd):

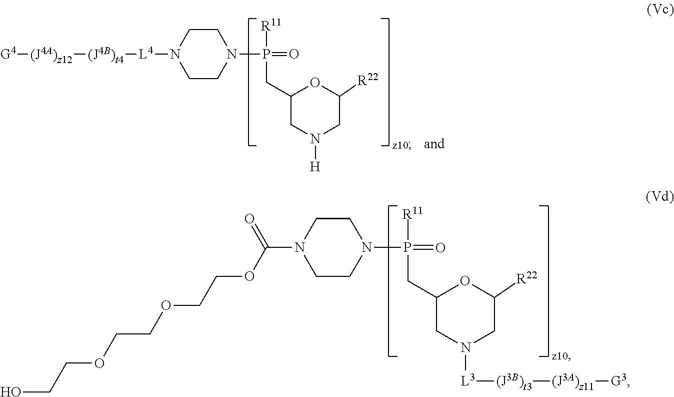

15-18. (canceled)

19. The antisense oligomer conjugate of claim 4, or a pharmaceutically acceptable salt thereof, wherein z13 and z14 are each independently selected from 1 and 2 and Y³ and Y⁴ are each independently selected from Gly and Ala.

20. The antisense oligomer conjugate of claim 19, or a pharmaceutically acceptable salt thereof, wherein Y³ is Gly.

21. The antisense oligomer conjugate of claim 19, or a pharmaceutically acceptable salt thereof, wherein Y⁴ is Gly.

22-23. (canceled)

24. The antisense oligomer conjugate of claim 4, or a pharmaceutically acceptable salt thereof, wherein L³ is

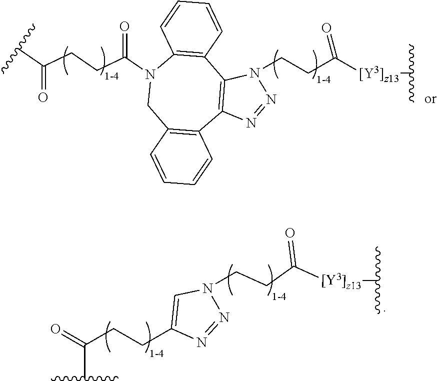

25. The antisense oligomer conjugate of claim 4, or a pharmaceutically acceptable salt thereof, wherein L⁴ is

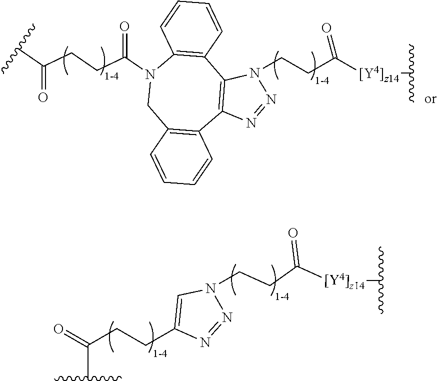

26. The antisense oligomer conjugate of claim 4, or a pharmaceutically acceptable salt thereof, wherein L³ is

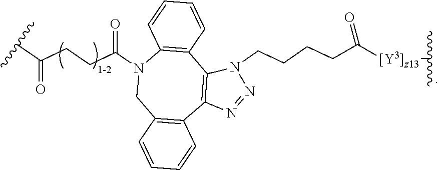

27. The antisense oligomer conjugate of claim 4, or a pharmaceutically acceptable salt thereof, wherein L³ is

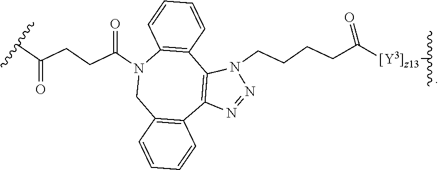

28. The antisense oligomer conjugate of claim 4, or a pharmaceutically acceptable salt thereof, wherein L³ is

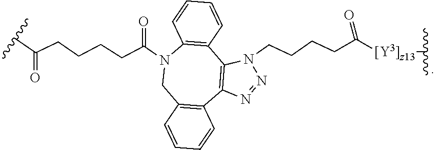

29-30. (canceled)

31. The antisense oligomer conjugate of claim 4, or a pharmaceutically acceptable salt thereof, wherein J^{3B} and each J^{4B} are each independently an L-amino acid selected from Arg, His, Lys, Asp, Glu, Ser, Thr, Asn, Gln, Gly, Met, Pro, Val, Ala, Leu, Phe, Trp, 2,4-diaminobutyric acid (Dab), 2-amino-5-ureidopentanoic acid (citrulline (Cit)), 2-amino-3-(naphthalen-2-yl)propanoic acid (Nap), 4-aminopiperidine-4-carboxylic acid (Pip), 2-amino-3-(pyridin-3-yl)propanoic acid (Pyr), and

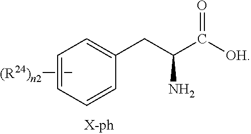

32. The antisense oligomer conjugate of claim 4, or a pharmaceutically acceptable salt thereof, wherein the peptide of J^{3B} or J^{4B} each independently comprise the sequence -(X-ph)-Gly-Arg.

33. The antisense oligomer conjugate of n4 claim 4, or a pharmaceutically acceptable salt thereof, wherein the peptide of J^{3B} or J^{4B} each independently comprise at least one unnatural amino acid selected from 2,4-diaminobutyric acid (Dab), 2-amino-5-ureidopentanoic acid (citrulline (Cit)), 2-amino-3-(naphthalen-2-yl)propanoic acid (Nap), 4-aminopiperidine-4-carboxylic acid (Pip), 2-amino-3-(pyridin-3-yl)propanoic acid (Pyr), and

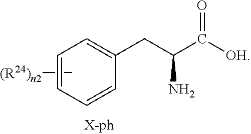

34. The antisense oligomer conjugate of claim 4, or a pharmaceutically acceptable salt thereof, wherein the peptide of J^{3B} or J^{4B} each independently comprise at least one unnatural amino acid selected from 2-amino-5-ureidopentanoic acid (citrulline (Cit)), 2-amino-3-(pyridin-3-yl)propanoic acid (Pyr), and

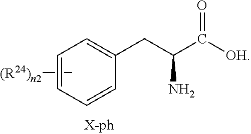

35. The antisense oligomer conjugate of claim 4, or a pharmaceutically acceptable salt thereof, wherein the peptide of J^{3B} or J^{4B} each independently comprise at least one

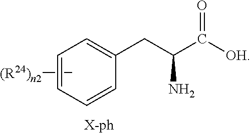

36. The antisense oligomer conjugate of claim 35, or a pharmaceutically acceptable salt thereof, wherein the peptide of J^{3B} or J^{4B} each independently further comprise at least one 2,4-diaminobutyric acid (Dab) or 4-aminopiperidine-4-carboxylic acid (Pip).

37. The antisense oligomer conjugate of claim 4, or a pharmaceutically acceptable salt thereof, wherein the antisense oligomer conjugate is an antisense oligomer conjugate of Formula (VI): 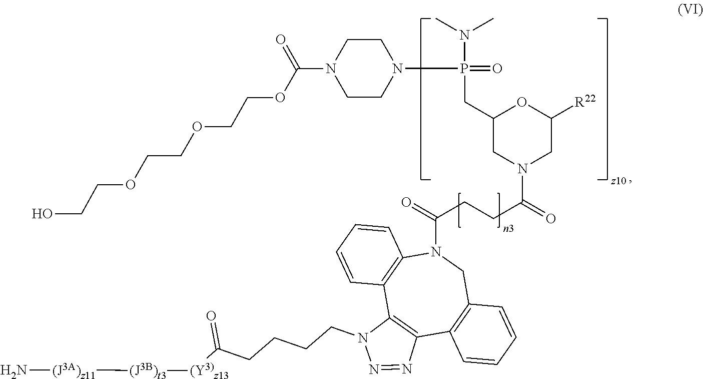
wherein n3 is 1 or 2.

38. The antisense oligomer conjugate of claim 37, or a pharmaceutically acceptable salt thereof, wherein the antisense oligomer conjugate is of Formula (VIa):

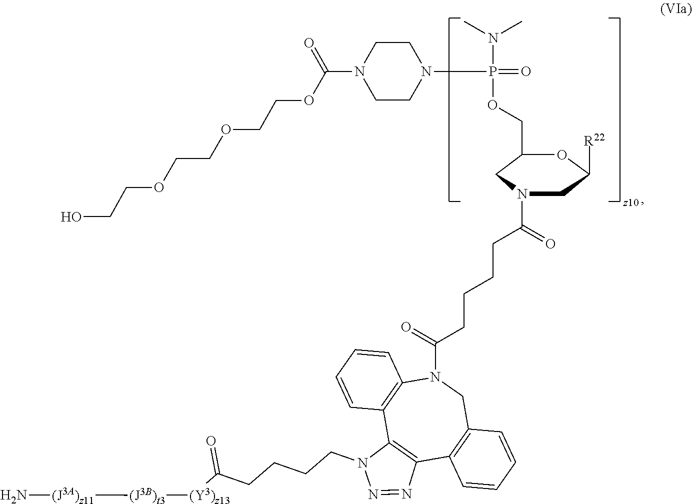

39. The antisense oligomer conjugate of claim 37, or a pharmaceutically acceptable salt thereof, wherein n3 is 1.

40. The antisense oligomer conjugate of claim 37, or a pharmaceutically acceptable salt thereof, wherein n3 is 2.

41. The antisense oligomer conjugate of claim 4, or a pharmaceutically acceptable salt thereof, wherein the antisense oligomer conjugate is of Formula (VII): 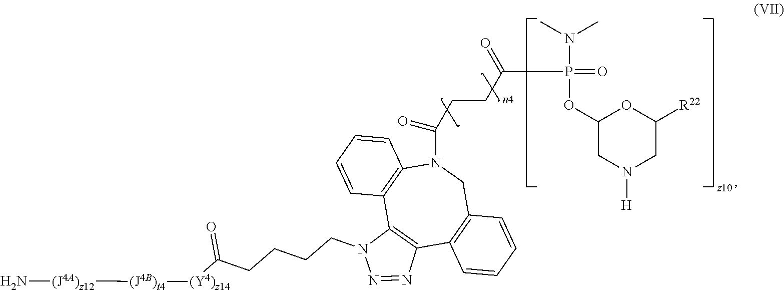
wherein n4 is 1 or 2.

42. The antisense oligomer conjugate of claim 41, or a pharmaceutically acceptable salt thereof, wherein the antisense oligomer conjugate is an antisense oligomer conjugate of Formula (VIIa):

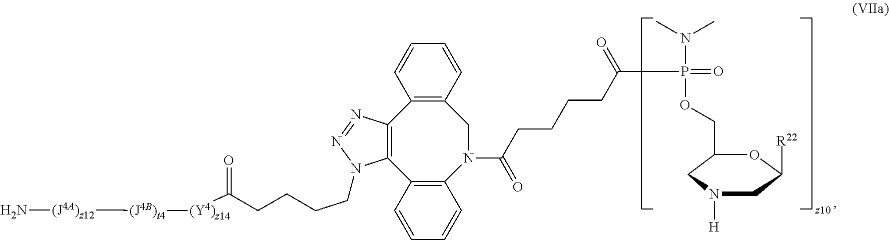

43. The antisense oligomer conjugate of claim 41, or a pharmaceutically acceptable salt thereof, wherein n4 is 1.

44. The antisense oligomer conjugate of claim 41, or a pharmaceutically acceptable salt thereof, wherein n4 is 2.

45. The antisense oligomer conjugate of claim 4, or a pharmaceutically acceptable salt thereof, wherein z13 is 1.

46. The antisense oligomer conjugate of claim 4, or a pharmaceutically acceptable salt thereof, wherein z14 is 1.

47. The antisense oligomer conjugate of claim 4, or a pharmaceutically acceptable salt thereof, wherein Y³ is selected from Gly and Ala.

48. The antisense oligomer conjugate of claim 4, or a pharmaceutically acceptable salt thereof, wherein Y⁴ is selected from Gly and Ala.

49. The antisense oligomer conjugate of claim 4, or a pharmaceutically acceptable salt thereof, wherein Y³ is Gly.

50. The antisense oligomer conjugate of claim 4, or a pharmaceutically acceptable salt thereof, wherein Y⁴ is Gly.

51. The peptide-oligonucleotide conjugate of claim 4, or a pharmaceutically acceptable salt thereof, wherein -(J^{3B})ₜ₃-(J^{3A})_{z11}- or -(J^{4B})ₜ₄-(J^{4A})_{z12}- each independently form a peptide sequence selected from:

| SEQ ID | |
|---|---|
| NO. | Sequence |
| 48 | Lys-Gln-Lys-Thr-Ser-Iph-Gly-Arg-Gly-Pip-Lys-Trp-Lys-Lys; |
| 49 | Asn-Phe-Lys-Gln-Nap-His-Ala-Gly-Dab-Arg-Lys-Trp-Lys-Lys; |
| 50 | Dab-Val-Ala-Arg-Asn-Asn-Dab-Thr-Lys-Gly-Lys-Trp-Lys-Lys; |
| 51 | Cit-Met-Phe-Gly-Pip-Dab-Dab-Lys-Ala-Pro-Lys-Trp-Lys-Lys; |
| 52 | Dab-Pro-Arg-Dab-Leu-His-Ser-Dab-Ala-Thr-Lys-Trp-Lys-Lys; |
| 53 | Pro-Pip-Gly-Dab-Lys-Dab-Ser-Val-Iph-Nap-Lys-Trp-Lys-Lys; |
| 54 | Val-Gln-His-Iph-Arg-Dab-Lys-Glu-Lys-Asn-Lys-Trp-Lys-Lys; |
| 55 | Asn-Phe-Asn-Asn-Arg-Ser-Dab-Gly-His-Lys-Lys-Trp-Lys-Lys; |
| 56 | Ala-Arg-Ser-Glu-Gln-His-His-Nap-Ser-Dab-Lys-Trp-Lys-Lys; |
| 57 | Iph-Gly-Arg-Gly-Pip-Lys-Trp-Lys-Lys; |
| 58 | Lys-Thr-Ser-Iph-Gly-Arg-Lys-Trp-Lys-Lys; |
| 61 | Iph-Gly-Arg-Lys-Trp-Lys-Lys; |
| 62 | Lys-Gln-Lys-Thr-Ser-Iph-Gly-Arg-Lys-Trp-Lys-Lys; |
| 63 | Ser-Iph-Gly-Arg-Lys-Trp-Lys-Lys; |
| 65 | Gln-Lys-Thr-Ser-Iph-Gly-Arg-Lys-Trp-Lys-Lys; |
| 66 | Lys-Gln-Lys-Thr-Ser-Iph-Gly-Arg-Lys-Trp-Lys-Lys; |
| 67 | Lys-Gln-Lys-Thr-Ser-Iph-Gly-Arg-Gly-Pip-Lys-Trp-Lys-Lys-Gly-Lys-Gln-Lys- |
| | Thr-Ser-Iph-Gly-Arg-Gly-Pip-Lys-Trp-Lys-Lys; |
| 68 | Dab-Val-Ala-Arg-Asn-Asn-Dab-Thr-Lys-Gly-Lys-Trp-Lys-Lys-Gly-Lys-Gln-Lys- |
| | Thr-Ser-Iph-Gly-Arg-Gly-Pip-Lys-Trp-Lys-Lys; |
| 69 | Cit-Met-Phe-Gly-Pip-Dab-Dab-Lys-Ala-Pro-Lys-Trp-Lys-Lys-Gly-Lys-Gln-Lys- |
| | Thr-Ser-Iph-Gly-Arg-Gly-Pip-Lys-Trp-Lys-Lys; |
| 70 | Iph-Gly-Arg-Gly-Pip-Lys-Gln-Lys-Thr-Ser-Iph-Gly-Arg-Gly-Pip-Lys-Trp-Lys- |
| | Lys; |
| 71 | Lys-Thr-Ser-Iph-Gly-Arg-Lys-Gln-Lys-Thr-Ser-Iph-Gly-Arg-Gly-Pip-Lys-Trp- |
| | Lys-Lys; |
| 72 | Lys-Gln-Lys-Thr-Ser-Lys-Gln-Lys-Thr-Ser-Iph-Gly-Arg-Gly-Pip-Lys-Trp-Lys- |
| | Lys; |
| 73 | Lys-Trp-Lys-Lys-Gln-Lys-Thr-Ser-Iph-Gly-Arg-Gly-Pip-Lys-Trp-Lys-Lys; |
| 74 | Lys-Thr-Ser-Iph-Gly-Arg-Lys-Gln-Lys-Thr-Ser-Iph-Gly-Arg-Lys-Trp-Lys-Lys; |
| 75 | Lys-Gln-Lys-Thr-Ser-Iph-Gly-Arg-Lys-Trp-Lys-Gln-Lys-Thr-Ser-Iph-Gly-Arg- |
| | Lys-Trp-Lys-Lys; |
| 78 | Lys-Gln-Lys-Thr-Ser-Phe-Gly-Arg-Gly-Pip-Lys-Trp-Lys-Lys; |
| 79 | Lys-Gln-Lys-Thr-Ser-Iph-Gly-Arg-Gly-Lys-Lys-Trp-Lys-Lys; |
| 80 | Lys-Gln-Lys-Thr-Ser-Phe-Gly-Arg-Gly-Lys-Lys-Trp-Lys-Lys; |
| 81 | Lys-Gln-Lys-Thr-Ser-Iph-Gly-Lys-Gly-Pip-Lys-Trp-Lys-Lys; |
| 82 | Lys-Gln-Lys-Thr-Ser-Iph-Ala-Arg-Ala-Pip-Lys-Trp-Lys-Lys; |
| 83 | Ala-Gln-Lys-Thr-Ser-Iph-Gly-Arg-Gly-Pip-Lys-Trp-Lys-Lys; |
| 84 | Lys-Ala-Lys-Thr-Ser-Iph-Gly-Arg-Gly-Pip-Lys-Trp-Lys-Lys; |
| 85 | Lys-Gln-Ala-Thr-Ser-Iph-Gly-Arg-Gly-Pip-Lys-Trp-Lys-Lys; |
| 86 | Lys-Gln-Lys-Ala-Ser-Iph-Gly-Arg-Gly-Pip-Lys-Trp-Lys-Lys; |
| 87 | Lys-Gln-Lys-Thr-Ala-Iph-Gly-Arg-Gly-Pip-Lys-Trp-Lys-Lys; |
| 88 | Lys-Gln-Lys-Thr-Ser-Ala-Gly-Arg-Gly-Pip-Lys-Trp-Lys-Lys; |
| 89 | Lys-Gln-Lys-Thr-Ser-Iph-Ala-Arg-Gly-Pip-Lys-Trp-Lys-Lys; |
| 90 | Lys-Gln-Lys-Thr-Ser-Iph-Gly-Ala-Gly-Pip-Lys-Trp-Lys-Lys; |
| 91 | Lys-Gln-Lys-Thr-Ser-Iph-Gly-Arg-Ala-Pip-Lys-Trp-Lys-Lys; |
| 92 | Lys-Gln-Lys-Thr-Ser-Iph-Gly-Arg-Gly-Ala-Lys-Trp-Lys-Lys; |
| 93 | Lys-Gln-Lys-Thr-Ser-Iph-Gly-Arg-Gly-Pip-Ala-Trp-Lys-Lys; |
| 94 | Lys-Gln-Lys-Thr-Ser-Iph-Gly-Arg-Gly-Pip-Lys-Ala-Lys-Lys; |
| 95 | Lys-Gln-Lys-Thr-Ser-Iph-Gly-Arg-Gly-Pip-Lys-Trp-Ala-Lys; |
| 96 | Lys-Gln-Lys-Thr-Ser-Iph-Gly-Arg-Gly-Pip-Lys-Trp-Lys-Ala-Lys-Trp-Lys-Lys; |
| 97 | Iph-Gln-Lys-Thr-Ser-Iph-Gly-Arg-Gly-Pip-Lys-Trp-Lys-Lys; |
| 98 | Lys-Gln-Iph-Thr-Ser-Iph-Gly-Arg-Gly-Pip-Lys-Trp-Lys-Lys; |
| 99 | Lys-Gln-Lys-Thr-Ser-Iph-Gly-Iph-Gly-Pip-Lys-Trp-Lys-Lys; |
| 100 | Lys-Gln-Lys-Thr-Ser-Iph-Gly-Arg-Gly-Iph-Lys-Trp-Lys-Lys; |
| 101 | Lys-Gln-Lys-Thr-Ser-Iph-Gly-Arg-Gly-Pip-Iph-Trp-Lys-Lys; |
| 102 | Lys-Gln-Lys-Thr-Ser-Iph-Gly-Arg-Gly-Pip-Lys-Trp-Iph-Lys-Lys-Trp-Lys-Lys; |
| 103 | Lys-Gln-Lys-Thr-Ser-Iph-Gly-Arg-Gly-Pip-Lys-Trp-Lys-Iph-Lys; |
| 104 | Dab-Gln-Dab-Thr-Ser-Iph-Gly-Arg-Gly-Pip-Dab-Trp-Dab-Dab-Lys-Trp-Lys-Lys; |
| 105 | Dab-Gln-Dab-Thr-Ser-Iph-Gly-Dab-Gly-Pip-Dab-Trp-Dab-Dab-Lys-Trp-Lys-Lys; |
| 106 | Dab-Gln-Dab-Thr-Ser-Iph-Gly-Arg-Gly-Dab-Dab-Trp-Dab-Dab-Lys-Trp-Lys-Lys; |
| | and |
| 107 | Dab-Gln-Dab-Thr-Ser-Iph-Gly-Dab-Gly-Dab-Dab-Trp-Dab-Dab-Lys-Trp-Lys- |
| | Lys. |

52. The antisense oligomer conjugate of claim 4, or a pharmaceutically acceptable salt thereof, wherein -(J^{3B})ₜ₃- or -(J^{4B})ₜ₄- each independently form a peptide sequence selected from:

| SEQ ID | |
|---|---|
| NO. | Sequence |
| 1 | Lys-Gln-Lys-Thr-Ser-Iph-Gly-Arg-Gly-Pip; |
| 2 | Asn-Phe-Lys-Gln-Nap-His-Ala-Gly-Dab-Arg; |
| 3 | Dab-Val-Ala-Arg-Asn-Asn-Dab-Thr-Lys-Gly; |
| 4 | Cit-Met-Phe-Gly-Pip-Dab-Dab-Lys-Ala-Pro; |
| 5 | Dab-Pro-Arg-Dab-Leu-His-Ser-Dab-Ala-Thr; |
| 6 | Pro-Pip-Gly-Dab-Lys-Dab-Ser-Val-Iph-Nap; |
| 7 | Val-Gln-His-Iph-Arg-Dab-Lys-Glu-Lys-Asn; |
| 8 | Asn-Phe-Asn-Asn-Arg-Ser-Dab-Gly-His-Lys; |
| 9 | Ala-Arg-Ser-Glu-Gln-His-His-Nap-Ser-Dab; |
| 10 | Iph-Gly-Arg-Gly-Pip; |
| 11 | Lys-Thr-Ser-Iph-Gly-Arg; |
| 12 | Lys-Gln-Lys-Thr-Ser; |
| 13 | Lys-Trp-Lys; |
| 14 | Iph-Gly-Arg; |
| 15 | Lys-Gln-Lys-Thr-Ser-Iph-Gly-Arg; |
| 16 | Ser-Iph-Gly-Arg; |
| 18 | Gln-Lys-Thr-Ser-Iph-Gly-Arg; |
| 19 | Lys-Gln-Lys-Thr-Ser-Iph-Gly-Arg; |
| 39 | Lys-Gln-Lys-Thr-Ser-Iph-Gly-Arg-Gly-Pip-Lys-Trp-Lys-Lys-Gly-Lys-Gln-Lys- |
| | Thr-Ser-Iph-Gly-Arg-Gly-Pip; |
| 40 | Dab-Val-Ala-Arg-Asn-Asn-Dab-Thr-Lys-Gly-Lys-Trp-Lys-Lys-Gly-Lys-Gln-Lys- |
| | Thr-Ser-Iph-Gly-Arg-Gly-Pip; |
| 41 | Cit-Met-Phe-Gly-Pip-Dab-Dab-Lys-Ala-Pro-Lys-Trp-Lys-Lys-Gly-Lys-Gln-Lys- |
| | Thr-Ser-Iph-Gly-Arg-Gly-Pip; |
| 42 | Iph-Gly-Arg-Gly-Pip-Lys-Gln-Lys-Thr-Ser-Iph-Gly-Arg-Gly-Pip; |
| 43 | Lys-Thr-Ser-Iph-Gly-Arg-Lys-Gln-Lys-Thr-Ser-Iph-Gly-Arg-Gly-Pip; |
| 44 | Lys-Gln-Lys-Thr-Ser-Lys-Gln-Lys-Thr-Ser-Iph-Gly-Arg-Gly-Pip; |
| 45 | Lys-Trp-Lys-Lys-Gln-Lys-Thr-Ser-Iph-Gly-Arg-Gly-Pip; |
| 46 | Lys-Thr-Ser-Iph-Gly-Arg-Lys-Gln-Lys-Thr-Ser-Iph-Gly-Arg; |
| 47 | Lys-Gln-Lys-Thr-Ser-Iph-Gly-Arg-Lys-Trp-Lys-Gln-Lys-Thr-Ser-Iph-Gly-Arg; |
| 108 | Lys-Gln-Lys-Thr-Ser-Phe-Gly-Arg-Gly-Pip; |
| 109 | Lys-Gln-Lys-Thr-Ser-Iph-Gly-Arg-Gly-Lys; |
| 110 | Lys-Gln-Lys-Thr-Ser-Phe-Gly-Arg-Gly-Lys; |
| 111 | Lys-Gln-Lys-Thr-Ser-Iph-Gly-Lys-Gly-Pip; |
| 112 | Lys-Gln-Lys-Thr-Ser-Iph-Ala-Arg-Ala-Pip; |
| 113 | Ala-Gln-Lys-Thr-Ser-Iph-Gly-Arg-Gly-Pip; |
| 114 | Lys-Ala-Lys-Thr-Ser-Iph-Gly-Arg-Gly-Pip; |
| 115 | Lys-Gln-Ala-Thr-Ser-Iph-Gly-Arg-Gly-Pip; |
| 116 | Lys-Gln-Lys-Ala-Ser-Iph-Gly-Arg-Gly-Pip; |
| 117 | Lys-Gln-Lys-Thr-Ala-Iph-Gly-Arg-Gly-Pip; |
| 118 | Lys-Gln-Lys-Thr-Ser-Ala-Gly-Arg-Gly-Pip; |
| 119 | Lys-Gln-Lys-Thr-Ser-Iph-Ala-Arg-Gly-Pip; |
| 120 | Lys-Gln-Lys-Thr-Ser-Iph-Gly-Ala-Gly-Pip; |
| 121 | Lys-Gln-Lys-Thr-Ser-Iph-Gly-Arg-Ala-Pip; |
| 122 | Lys-Gln-Lys-Thr-Ser-Iph-Gly-Arg-Gly-Ala; |
| 123 | Lys-Gln-Lys-Thr-Ser-Iph-Gly-Arg-Gly-Pip-Ala; |
| 124 | Lys-Gln-Lys-Thr-Ser-Iph-Gly-Arg-Gly-Pip-Lys-Ala; |
| 125 | Lys-Gln-Lys-Thr-Ser-Iph-Gly-Arg-Gly-Pip-Lys-Trp-Ala; |
| 126 | Lys-Gln-Lys-Thr-Ser-Iph-Gly-Arg-Gly-Pip-Lys-Trp-Lys-Ala; |
| 127 | Iph-Gln-Lys-Thr-Ser-Iph-Gly-Arg-Gly-Pip; |
| 128 | Lys-Gln-Iph-Thr-Ser-Iph-Gly-Arg-Gly-Pip; |
| 129 | Lys-Gln-Lys-Thr-Ser-Iph-Gly-Iph-Gly-Pip; |
| 130 | Lys-Gln-Lys-Thr-Ser-Iph-Gly-Arg-Gly-Iph; |
| 131 | Lys-Gln-Lys-Thr-Ser-Iph-Gly-Arg-Gly-Pip-Iph-Trp-Lys-Lys; |
| 132 | Lys-Gln-Lys-Thr-Ser-Iph-Gly-Arg-Gly-Pip-Lys-Trp-Iph-Lys; |
| 133 | Lys-Gln-Lys-Thr-Ser-Iph-Gly-Arg-Gly-Pip-Lys-Trp-Lys-Iph; |
| 134 | Dab-Gln-Dab-Thr-Ser-Iph-Gly-Arg-Gly-Pip-Dab-Trp-Dab-Dab; |
| 135 | Dab-Gln-Dab-Thr-Ser-Iph-Gly-Dab-Gly-Pip-Dab-Trp-Dab-Dab; |
| 136 | Dab-Gln-Dab-Thr-Ser-Iph-Gly-Arg-Gly-Dab-Dab-Trp-Dab-Dab; and |
| 137 | Dab-Gln-Dab-Thr-Ser-Iph-Gly-Dab-Gly-Dab-Dab-Trp-Dab-Dab. |

53. (canceled)

54. The antisense oligomer conjugate of claim 4, or a pharmaceutically acceptable salt thereof, of Formula (VIIIa) or (VIIIb):

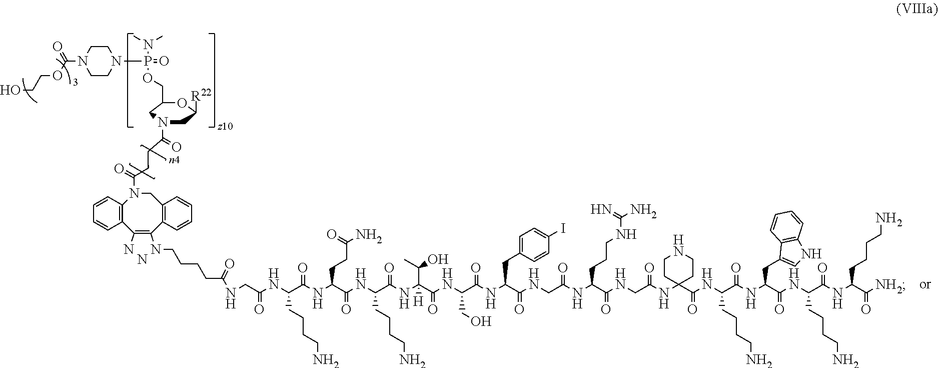

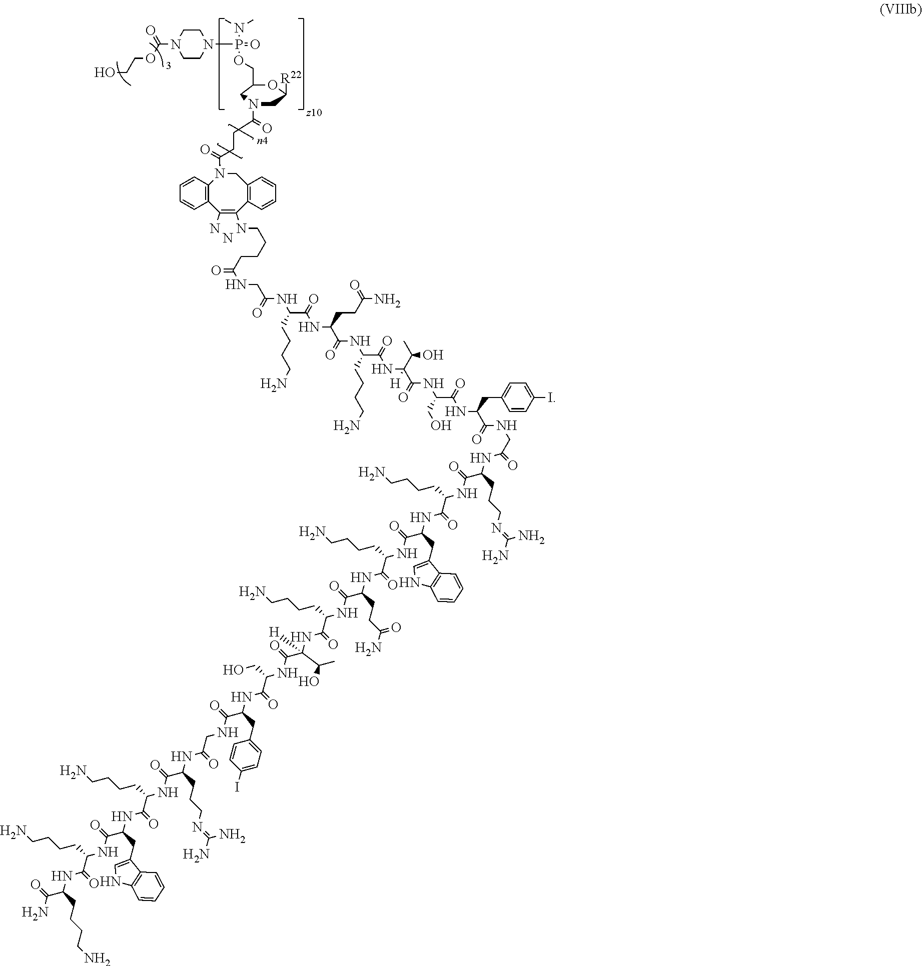

55-60. (canceled)
